SEQUENCE LISTING (211 sequences)

SEQ ID NO: 1 - protein, 191 aa, Staphylococcus epidermidis
MKKEILEWIVAIAVAIALIAIITKFVGKSYSIKGDSMDPTLKDGERVVVNIIGYKLGGVEKGNVIVFHANKKDDYVKRVIGTPGDSVEYKNDTLYVNGKKQSEPYLNYNEKRKQTEYITGSFKTKNLPNANPQSNVIPKGKYLVLGDNREVSKDSRSFGLIDKDQIVGKVSLRYWPFSEFKSNFNPNNTKN

SEQ ID NO: 2 - DNA, 576 bp, Staphylococcus epidermidis
ttgaaaaaagaaattttagagtggattgttgccatagccgttgccattgcacttattgccataatcactaaatttgtcggaaaatcatattctattaaaggtgattcaatggatcctacattaaaagatggggagcgtgtagtggtaaatattattggctataaattaggtggcgttgaaaaaggaaatgtcattgtatttcatgctaataaaaaagatgattatgttaaaagagttattggaactccaggagatagtgttgaatataaaaatgatacactctatgttaatggtaaaaagcaatcagaaccatacttgaactataatgaaaaacgtaagcaaactgagtatatcacaggtagtttcaaaacaaaaaatttaccaaatgctaatcctcaatctaatgttattcctaaaggtaaatatttagttttgggggataaccgtgaggtaagtaaagatagtcgttcattcggtttaattgacaaagaccaaattgttggaaaggtatcgctcagatattggcctttcagtgaatttaaatctaactttaatccaaataacactaaaaattaa

SEQ ID NO: 3 - DNA, 33 bp, Staphylococcus epidermidis
gtcggaaaatcatattctattaaaggtgattca

SEQ ID NO: 4 - protein, 11 aa, Staphylococcus epidermidis
VGKSYSIKGDS

SEQ ID NO: 5 - DNA, 33 bp, Staphylococcus epidermidis
gtcggaaaaccatattctattaaaggtgattca

SEQ ID NO: 6 - protein, 11 aa, Staphylococcus epidermidis
VGKPYSIKGDS

SEQ ID NO: 7 - DNA, 33 bp, Staphylococcus epidermidis
gtcggaaaaccgtattctattaaaggtgattca

SEQ ID NO: 8 - DNA, 33 bp, Staphylococcus epidermidis
gtcggaaaaccctattctattaaaggtgattca

SEQ ID NO: 9 - DNA, 33 bp, Staphylococcus epidermidis
gtcggaaaaccttattctattaaaggtgattca

SEQ ID NO: 10 - DNA, 33 bp, Staphylococcus epidermidis
gtcggaaaatcatatcctattaaaggtgattca

SEQ ID NO: 11 - protein, 11 aa, Staphylococcus epidermidis
VGKSYPIKGDS

SEQ ID NO: 12 - DNA, 33 bp, Staphylococcus epidermidis
gtcggaaaatcatatcccattaaaggtgattca

SEQ ID NO: 13 - DNA, 33 bp, Staphylococcus epidermidis
gtcggaaaatcatatccaattaaaggtgattca

SEQ ID NO: 14 - DNA, 33 bp, Staphylococcus epidermidis
gtcggaaaatcatatccgattaaaggtgattca

SEQ ID NO: 15 - protein, 192 aa, Staphylococcus epidermidis
MKKEIIEWIVAIIVAIVIVTLVQKFLFASYTVKGASMHPTFENREKVIVSRIAKTLDHIDTGDVVIFHANAKQDYIKRLIGKPGDSVEYKKDQLYLNGKKVDEPYLSENKKHKVGEYLTENFKSRDLKGTNGNMKIPSGKYLVLGDNRQNSIDSRMDEVGLLDKNQVVGKVVLRYWPFNRWGGSFNPGTFPN

SEQ ID NO: 16 - DNA, 579 bp, Staphylococcus epidermidis
atgaagaaagaaataatagaatggattgtagccataatcgttgcaattgttatcgtcacacttgtgcaaaagtttttatttgcttcttatacagtcaaaggagcatctatgcatccaacatttgaaaatcgagaaaaagtgatagtaagtcgtatagcaaaaacgcttgatcatattgatacaggagatgtagtgatttttcatgctaacgcgaagcaagattatattaagcgacttattggtaaaccaggtgattcagtagaatataaaaaagatcaactatatttaaacggtaaaaaagtagatgagccttatttaagtgaaaataaaaaacataaagttggagaatatctaacggaaaactttaagtctagagatcttaagggtacgaatggcaatatgaaaattcctagtggtaaatacttggttttaggtgataatcgtcaaaacagtattgacagtcgcatggatgaagtaggtcttttagataaaaatcaagttgttggaaaagtagttttgagatactggccatttaatcggtggggcggtagttttaatcctggaacatttcctaactaa

SEQ ID NO: 17 - protein, 11 aa, Staphylococcus epidermidis
VGKSYSIKGDS

SEQ ID NO: 18 - protein, 11 aa, Staphylococcus epidermidis
VGKPYSIKGDS

SEQ ID NO: 19 - protein, 11 aa, Staphylococcus epidermidis
VGKSYPIKGDS

SEQ ID NO: 20 - protein, 11 aa, S. aureus
VAKPYTVKGDS

SEQ ID NO: 21 - protein, 11 aa, S. aureus
VAKSYTVKGDS

SEQ ID NO: 22 - protein, 11 aa, E. coli
IYEPFQIPSGS

SEQ ID NO: 23 - protein, 11 aa, E. coli
IYESFQIPSGS

SEQ ID NO: 24 - protein, 11 aa, P. aeruginosa
LFEPFQIPSGS

SEQ ID NO: 25 - protein, 11 aa, P. aeruginosa
LFESFQIPSGS

SEQ ID NO: 26 - DNA, 22 bp, Artificial Sequence
  A synthetic primer
tcccgttcgctggctgcctgtg

SEQ ID NO: 27 - DNA, 44 bp, Artificial Sequence
  A synthetic primer
cggcggctttgttgaataaatcgttaatggatgccgccaatgcg

SEQ ID NO: 28 - DNA, 44 bp, Artificial Sequence
  A synthetic primer
gagacacaacgtggctttcccattaatagccatcttcgttcacg

SEQ ID NO: 29 - DNA, 32 bp, Artificial Sequence
  A synthetic primer
ttggtttctagaccagcgtattgccacggacc

SEQ ID NO: 30 - DNA, 29 bp, Artificial Sequence
  A synthetic primer
ttggtttctagactttatcgacaccccgg

SEQ ID NO: 31 - DNA, 20 bp, Artificial Sequence
  A synthetic primer
ggttgtaacactggcagagc

SEQ ID NO: 32 - DNA, 39 bp, Artificial Sequence
  A synthetic primer
cgttcgtttatttatgaagcgttccagatcccgtcaggt

SEQ ID NO: 33 - DNA, 39 bp, Artificial Sequence
  A synthetic primer
acctgacgggatctggaacgcttcataaataaacgaacg

SEQ ID NO: 34 - DNA, 39 bp, Artificial Sequence
  A synthetic primer
cgttcgtttatttatgaatgcttccagatcccgtcaggt

SEQ ID NO: 35 - DNA, 39 bp, Artificial Sequence
  A synthetic primer
acctgacgggatctggaagcattcataaataaacgaacg

SEQ ID NO: 36 - DNA, 39 bp, Artificial Sequence
  A synthetic primer
cgttcgtttatttatgaagatttccagatcccgtcaggt

SEQ ID NO: 37 - DNA, 39 bp, Artificial Sequence
  A synthetic primer
acctgacgggatctggaaatcttcataaataaacgaacg

SEQ ID NO: 38 - DNA, 39 bp, Artificial Sequence
  A synthetic primer
cgttcgtttatttatgaagagttccagatcccgtcaggt

SEQ ID NO: 39 - DNA, 39 bp, Artificial Sequence
  A synthetic primer
acctgacgggatctggaactcttcataaataaacgaacg

SEQ ID NO: 40 - DNA, 39 bp, Artificial Sequence
  A synthetic primer
cgttcgtttatttatgaattcttccagatcccgtcaggt

SEQ ID NO: 41 - DNA, 39 bp, Artificial Sequence
  A synthetic primer
acctgacgggatctggaagaattcataaataaacgaacg

SEQ ID NO: 42 - DNA, 39 bp, Artificial Sequence
  A synthetic primer
cgttcgtttatttatgaaggcttccagatcccgtcaggt

SEQ ID NO: 43 - DNA, 39 bp, Artificial Sequence
  A synthetic primer
acctgacgggatctggaagccttcataaataaacgaacg

SEQ ID NO: 44 - DNA, 39 bp, Artificial Sequence
  A synthetic primer
cgttcgtttatttatgaacatttccagatcccgtcaggt

SEQ ID NO: 45 - DNA, 39 bp, Artificial Sequence
  A synthetic primer
acctgacgggatctggaaatgttcataaataaacgaacg

SEQ ID NO: 46 - DNA, 39 bp, Artificial Sequence
  A synthetic primer
cgttcgtttatttatgaaatcttccagatcccgtcaggt

SEQ ID NO: 47 - DNA, 39 bp, Artificial Sequence
  A synthetic primer
acctgacgggatctggaagatttcataaataaacgaacg

SEQ ID NO: 48 - DNA, 39 bp, Artificial Sequence
  A synthetic primer
cgttcgtttatttatgaaaaattccagatcccgtcaggt

SEQ ID NO: 49 - DNA, 39 bp, Artificial Sequence
  A synthetic primer
acctgacgggatctggaatttttcataaataaacgaacg

SEQ ID NO: 50 - DNA, 39 bp, Artificial Sequence
  A synthetic primer
cgttcgtttatttatgaactgttccagatcccgtcaggt

SEQ ID NO: 51 - DNA, 39 bp, Artificial Sequence
  A synthetic primer
acctgacgggatctggaacagttcataaataaacgaacg

SEQ ID NO: 52 - DNA, 39 bp, Artificial Sequence
  A synthetic primer
cgttcgtttatttatgaaatgttccagatcccgtcaggt

SEQ ID NO: 53 - DNA, 39 bp, Artificial Sequence
  A synthetic primer
acctgacgggatctggaacatttcataaataaacgaacg

SEQ ID NO: 54 - DNA, 39 bp, Artificial Sequence
  A synthetic primer
cgttcgtttatttatgaaaacttccagatcccgtcaggt

SEQ ID NO: 55 - DNA, 39 bp, Artificial Sequence
  A synthetic primer
acctgacgggatctggaagttttcataaataaacgaacg

SEQ ID NO: 56 - DNA, 39 bp, Artificial Sequence
  A synthetic primer
cgttcgtttatttatgaacagttccagatcccgtcaggt

SEQ ID NO: 57 - DNA, 39 bp, Artificial Sequence
  A synthetic primer
acctgacgggatctggaactgttcataaataaacgaacg

SEQ ID NO: 58 - DNA, 39 bp, Artificial Sequence
  A synthetic primer
cgttcgtttatttatgaacggttccagatcccgtcaggt

SEQ ID NO: 59 - DNA, 39 bp, Artificial Sequence
  A synthetic primer
acctgacgggatctggaaccgttcataaataaacgaacg

SEQ ID NO: 60 - DNA, 45 bp, Artificial Sequence
  A synthetic primer
gtgcgttcgtttatttatgaatcgttccagatcccgtcaggttcg

SEQ ID NO: 61 - DNA, 45 bp, Artificial Sequence
  A synthetic primer
cgaacctgacgggatctggaacgattcataaataaacgaacgcac

SEQ ID NO: 62 - DNA, 39 bp, Artificial Sequence
  A synthetic primer
cgttcgtttatttatgaaaccttccagatcccgtcaggt

SEQ ID NO: 63 - DNA, 39 bp, Artificial Sequence
  A synthetic primer
acctgacgggatctggaaggtttcataaataaacgaacg

SEQ ID NO: 64 - DNA, 39 bp, Artificial Sequence
  A synthetic primer
cgttcgtttatttatgaagtgttccagatcccgtcaggt

SEQ ID NO: 65 - DNA, 39 bp, Artificial Sequence
  A synthetic primer
acctgacgggatctggaacacttcataaataaacgaacg

SEQ ID NO: 66 - DNA, 39 bp, Artificial Sequence
  A synthetic primer
cgttcgtttatttatgaatggttccagatcccgtcaggt

SEQ ID NO: 67 - DNA, 39 bp, Artificial Sequence
  A synthetic primer
acctgacgggatctggaaccattcataaataaacgaacg

SEQ ID NO: 68 - DNA, 39 bp, Artificial Sequence
  A synthetic primer
cgttcgtttatttatgaatatttccagatcccgtcaggt

SEQ ID NO: 69 - DNA, 39 bp, Artificial Sequence
  A synthetic primer
acctgacgggatctggaaatattcataaataaacgaacg

SEQ ID NO: 70 - DNA, 34 bp, Artificial Sequence
  A synthetic primer
ttggttggatcctggtgctcgacttcttcgatcg

SEQ ID NO: 71 - DNA, 34 bp, Artificial Sequence
  A synthetic primer
ttggttactagtgtcggacctcatgtcagtgtag

SEQ ID NO: 72 - DNA, 39 bp, Artificial Sequence
  A synthetic primer
cgttccttcctggtcgagagcttccagattccctcgggg

SEQ ID NO: 73 - DNA, 39 bp, Artificial Sequence
  A synthetic primer
ccccgagggaatctggaagctctcgaccaggaaggaacg

SEQ ID NO: 74 - DNA, 21 bp, Artificial Sequence
  A synthetic primer
gtggcgatccaggcagccatc

SEQ ID NO: 75 - DNA, 36 bp, Artificial Sequence
  A synthetic primer
ttggttgaattcgatctgtaaacgattggtgaacac

SEQ ID NO: 76 - DNA, 38 bp, Artificial Sequence
  A synthetic primer
ttggttgaattcgttcgctataactaccaacttcttgg

SEQ ID NO: 77 - DNA, 44 bp, Artificial Sequence
  A synthetic primer
gtaggtaaatttattgttacgtcatatacaattaaaggtgaatc

SEQ ID NO: 78 - DNA, 44 bp, Artificial Sequence
  A synthetic primer
gattcacctttaattgtatatgacgtaacaataaatttacctac

SEQ ID NO: 79 - DNA, 26 bp, Artificial Sequence
  A synthetic primer
caaggaaagcgtgtcgttgttgtacc

SEQ ID NO: 80 - DNA, 30 bp, Artificial Sequence
  A synthetic primer
ccaatcattcttgctgcagtaggtctaacg

SEQ ID NO: 81 - DNA, 27 bp, Artificial Sequence
  A synthetic primer
tgatggtgatacgattccaccgggagc

SEQ ID NO: 82 - DNA, 28 bp, Artificial Sequence
  A synthetic primer
gcatggctgttgactttcctgtacctgc

SEQ ID NO: 83 - DNA, 33 bp, Artificial Sequence
  A synthetic primer
ttggttccatggtgcgttcgtttatttatgaac

SEQ ID NO: 84 - DNA, 38 bp, Artificial Sequence
  A synthetic primer
ttggttggatcctggcatttaatggatgccgccaatgc

SEQ ID NO: 85 - DNA, 36 bp, Artificial Sequence
  A synthetic primer
ttggttggtaccttgaaaaaagaaatattggaatgg

SEQ ID NO: 86 - DNA, 41 bp, Artificial Sequence
  A synthetic primer
ttggttctcgagttaatttttagtattttcaggattgaaat

SEQ ID NO: 87 - protein, 8 aa, E. coli
PFQIPSGS

SEQ ID NO: 88 - protein, 8 aa, Acinetobacter baumanii
PFNIPSDS

SEQ ID NO: 89 - protein, 8 aa, Neiserria meningitidis
PFQIPSSS

SEQ ID NO: 90 - protein, 8 aa, Bordetella pertussis
PFHIPSGS

SEQ ID NO: 91 - protein, 8 aa, Mycobacterium tuberculosis
PYLIPSES

SEQ ID NO: 92 - protein, 8 aa, S. aurues
PYTIKGES

SEQ ID NO: 93 - protein, 8 aa, Bacillus anthracis
PSLVQGES

SEQ ID NO: 94 - protein, 8 aa, Bacillus anthracis
LCKVEGKS

SEQ ID NO: 95 - protein, 8 aa, Streptococcus mutans
PVQVDGHS

SEQ ID NO: 96 - protein, 8 aa, Clostridium difficile
PSIVSGES

SEQ ID NO: 97 - protein, 8 aa, Clostridium difficile
PTIVKGES

SEQ ID NO: 98 - protein, 8 aa, Clostridium difficile
PTLVNGES

SEQ ID NO: 99 - protein, 8 aa, Enterococcus faecalis
PAAVNGSS

SEQ ID NO: 100 - protein, 8 aa, Enterococcus faecalis
SYPIAGQS

SEQ ID NO: 101 - protein, 8 aa, Enterococcus faecalis
PAAVNGSS

SEQ ID NO: 102 - protein, 8 aa, Enterococcus faecalis
PVRVDGHS

SEQ ID NO: 103 - protein, 8 aa, Listeria monocytogenes
PVKVEGTS

SEQ ID NO: 104 - protein, 8 aa, Listeria monocytogenes
PVTVNGKS

SEQ ID NO: 105 - protein, 8 aa, Listeria monocytogenes
PILVDGIS

SEQ ID NO: 106 - protein, 266 aa, Rhodococcus equi
VADAPQDPDVTPDETEQEQTGGRSRRRRGKDKKPRSFWREIPILIVVALLLSFLLQTFIARVYLIPSESMEPTLHGCPGCTGDRIVVEKISYRFGDPKPGDVVVFRGPESWSEGYSSTRSDNVVVRGLQEVGSLVGVVPPDENDLVKRVIATGGQTVECCDDQGRVLVDGKPLDEPYITMDFPFIPGVQTCDTAVKSGRCFGPVTVPDGHLWVMGDNRSNSADSRYHVSDEMQGTIPVDNVIGKATFIVLPPGRWGSISSPDIRQQ

SEQ ID NO: 107 - DNA, 801 bp, Rhodococcus equi
gtggcagatgcaccgcaggacccggacgtgacgccggacgagaccgagcaggagcagaccgggggacggtcccgcaggcgcagggggaaggacaagaaacctcgatcgttctggcgtgagatcccgatcctcatcgtcgtcgccttgctgttgagtttcctgctgcagacgttcatcgcccgggtctacctcattccgtccgagtcgatggagccgacgctgcacggttgccccgggtgcaccggtgaccgcatcgtcgtcgagaagatcagttaccgattcggcgacccgaagcccggcgacgtcgtggtcttccgcggcccggagtcgtggtccgagggatattcgtcgacacgctcggacaacgtggtggtccgcggtctgcaggaggtcggctcgctcgtcggtgtcgtgccgccggacgagaacgatctcgtcaaacgcgtcatcgcgacgggtgggcagaccgtcgagtgctgcgacgaccagggccgtgtcctggtcgacggaaagccgctcgacgagccgtacatcacgatggacttccctttcatccccggcgtgcagacgtgtgacaccgccgtgaagtccggacgctgcttcggacccgtcacggttcccgatgggcacctgtgggtgatgggcgacaaccgcagcaattccgcggattcgcggtaccacgtctccgacgagatgcagggcacgattccggtggacaatgtgatcggtaaggcgaccttcatcgtcctgcccccgggccggtggggatcgatctcgtctcccgacatccggcagcagtga

SEQ ID NO: 108 - protein, 269 aa, Rhodococcus opacus
VTDSSKERALSSESETTGDSAATSAVNGGAAETEKKPRSFLRELPILILVALVLSFLLQTFVARVYLIPSESMEPTLHGCAGCTGDRIVVEKIGYRFGDPQPGDVIVFRGPDSWSQDFVSTRSSNVVIRGAQEVGSLVGLVPPDENDLVKRVIATGGQTVECCDDQGRILVDGQPIDEPYVVMDFPFVPGSQACDTALKSARCFGPVTVPEGHLWVMGDNRSNSADSRYHVGDDMQGTIPLDNVIGKAVFIALPPSRMGTISSPDIQGK

SEQ ID NO: 109 - DNA, 810 bp, Rhodococcus opacus
gtgacagattcttcgaaggagcgggcattgtcgtcggaatccgagaccaccggcgattcggccgccacctccgcagtgaacggcggtgcggcggagaccgagaagaaaccccgctccttcctccgcgagttgccgatcctgatcctggtcgcgctcgtcctgagtttcctgctgcagacgttcgtcgcccgcgtgtatctcattccgtcggagtcgatggaaccgacgctgcacgggtgcgcgggctgcaccggcgaccgcatcgtggtcgagaagatcggctaccgtttcggggacccgcaacccggtgacgtcatcgtgttccgcgggcccgactcgtggtcacaggatttcgtctccacccgttcctccaacgtggtgatccgcggtgcgcaggaagtcggttccctcgtcggactcgtcccgccggacgagaacgacctcgtcaagcgtgtgatcgccaccggcggtcagaccgtcgaatgctgcgacgaccagggccgcatcctggtggacggacaaccgatcgacgagccctacgtcgtcatggacttccccttcgtccccggctcccaggcctgcgacacggcgctgaagtcggcgcgctgcttcggtcccgtcaccgtccccgaggggcacctgtgggtgatgggcgacaaccgcagcaactccgcggactcccgctaccacgtcggcgacgacatgcaaggcaccatcccgctcgacaacgtgatcggcaaggcggtcttcatcgcgttgccgccgtcgcgaatgggcacgatcagttcacccgatatccagggcaagtga

SEQ ID NO: 110 - protein, 285 aa, Corynebacterium diphtheriae
MKRSVFSFCMMQQASLGVFHSMAETAARVLKVSSANNETVSPTEGVETHDKEKKQLPWFVEIPVVVVVTLLVITLLQTFVGRVYMIPSQSMEPTLHGCAGCTGDRIYVDKLAYRFGEPEAGDVVVFAGTESWNTGFTTSRSENPLVRGIQNAGAFVGLVAPDENDLVKRIVATGGQTVQCLEGDEGVKVDGKVIDSSYTLMPPAYPVDQTTGSEACGGFYFGPIKVPEGNYFMMGDNRTNSADSRYHIGDQYQGTIPKENLKGKVQFKIFPFNRIGAVEDYDIQQ

SEQ ID NO: 111 - DNA, 858 bp, Corynebacterium diphtheriae
atgaagcgctcagttttctctttttgtatgatgcagcaagcgtcgctgggcgtttttcattcgatggctgaaacagctgctagagttctcaaagtgagttcagctaataacgagactgtgtcccccacggaaggcgtcgaaacgcacgacaaggaaaagaagcaactgccatggtttgtggaaatccctgtcgtcgtagtggtgacccttcttgtgatcaccttgcttcaaacgttcgttggacgggtctatatgatcccaagtcagtcaatggagccgacacttcatggatgtgcagggtgtaccggagaccgaatttatgtagataagctggcttatcgttttggtgaaccagaagccggcgacgttgtagtttttgcaggtacagaatcatggaacaccggatttaccacttcacggtcagaaaatcctctggttcgtggaatacaaaatgcgggtgctttcgtcggattagtagcaccagacgaaaacgaccttgtaaaacgcatcgtagcaacagggggtcaaacggtgcagtgccttgaaggcgatgaaggtgtcaaagtagacggtaaagtcatcgactcgtcatatactctgatgccaccagcgtatccggtcgaccagaccacaggatcagaggcgtgcggcggcttttacttcggacctatcaaggtacctgaaggaaattacttcatgatgggcgataaccggacaaactccgcggattctcgttaccacattggtgatcagtatcaaggcaccatccctaaagaaaacctcaaggggaaagttcagttcaagattttcccatttaaccgtattggtgcagtcgaggattacgatatccaacagtga

SEQ ID NO: 112 - protein, 208 aa, Lactococcus lactis
MMKFLKEWGLFIFIIAAVLLSRVFIWSLVVVDGHSMDPTLADKERLVIVRTTKINRFDIVVAKENAADGSTKDIVKRVVGMPGDTIKFDHDQLTINNKVYPENYLKDYQKQLADGQLEKTYGNYPLTKALTDQNRSLFVSLAQSTKAFTTDSTGNPTFTVKVPDGQYFLMGDNRVVSQDSRAVGSFKRSAIIGEAKLRVWPLNKISFF

SEQ ID NO: 113 - DNA, 627 bp, Lactococcus lactis
atgatgaaatttttaaaagaatggggattatttatctttataattgccgctgtccttctctcgcgcgtctttatttggtcactagttgtcgttgatggccattcaatggaccctactttagccgataaagaaagacttgtaattgttagaacgacaaaaattaatcgttttgatattgtagttgctaaagaaaacgcggctgatggttcaaccaaagatattgtcaaacgtgtcgttgggatgcctggggacactataaaattcgaccatgaccaacttactatcaataataaggtttatccagaaaactatctcaaagactatcaaaaacaattggctgatggtcaattggaaaaaacttacgggaactatcctttgacaaaagcattaactgatcaaaatcgtagtttatttgtaagcttagctcagagcaccaaagcttttacaacggatagtactggtaatccaacctttacagtcaaagtccctgacggacaatacttcttgatgggagataatcgtgttgtgtctcaagatagccgagcagttggaagtttcaaacgttcagcgattattggtgaagccaaattacgagtttggccactcaataaaatttctttcttttaa

SEQ ID NO: 114 - protein, 262 aa, Corynebacterium glutamicum
VTDFSSASNADDSTQDGRPGRRAGKSKKESKPTPWYIEIPVVVVLTLALIFVLQTFVGRMYMIPSGSMEPTLHGCEGCTGDRILVEKVSYYFTDPEPGDVVVFKGTDSWNVGFTTQRSDNSVIRGLQNLGSYVGLVAPDENDLVKRIIATGGQTVSCQAGDPGIMVDGKEVDDSYTLQPAQFPIDETSGSTECGGNYFGPITVPGGNYFMMGDNRTNSMDSRYHLGDQYQGTIPEENIKGKVQAIILPFSRIGGVDDPAIKG

SEQ ID NO: 115 - DNA, 789 bp, Corynebacterium glutamicum
gtgactgatttttctagtgcttcaaatgctgacgattccacgcaggacggtcgtcctggtcgacgtgctggaaagtctaagaaggaatcgaagccaactccgtggtacatcgaaattccagtggttgtggttttgaccctcgcgctgattttcgtgctccagacgtttgtcggacgcatgtacatgattccgagtggttcgatggaacctactttgcacggatgtgagggctgcacgggtgaccgcatcctggtggagaaggtttcttactacttcacggatccagagccgggcgatgttgtggtgttcaagggtactgattcctggaacgttggattcactacgcagcgttccgataattcggtgatccgcggcctgcagaacctgggttcttacgtgggtcttgtcgcacctgatgaaaatgacctggtcaagcgcattatcgccaccggcggtcagactgtttcgtgccaagccggtgatcctggaatcatggttgacggcaaggaagtcgatgacagctacacgctgcaacctgcgcaattccccatcgatgagacctccggttccaccgaatgcggcggcaactatttcggccccatcaccgtgcctggcggcaactacttcatgatgggtgacaaccgcaccaactccatggattcccgctaccacctgggcgatcagtaccaaggaaccatccctgaggaaaacatcaagggcaaagttcaagcaattatcctgccatttagccgaatcggtggcgtcgacgaccctgccatcaaaggctag

SEQ ID NO: 116 - protein, 287 aa, Francisella tularensis
MEILNYILNLSFTFWLLFLTIASGLIYIIDFVFFQKSRLAAYTDELKGLSKKQKRQFYKDRGLKAPFIADQARSLFSVFFVVFLLRTFLIGNFLIPTASMTPTLPVGDFIFVNKTAYGIRAPFTNETLIKVGEPKRGDIVVFHFPVNPNVDFVKRVIGLPGDVISYKDKMLTINGKKLEYTNCNRDAMNYYNQSLAAGSGDTVCTENLDGVKHEVDWIESIKGTDFENLKVPAGQYFVMGDNRDNSEDSRYWGFVPDKDLVGKAKVVWMSWDKIDKKVRWDEIGKVF

SEQ ID NO: 117 - DNA, 864 bp, Francisella tularensis
atggaaatcttaaactatattttaaacttgagctttactttttggcttttattcttaaccattgccagtggtttaatttatattattgattttgtgttcttccaaaaatcaagattagcagcatatacagatgaattaaaaggtctttctaagaagcaaaaacgtcagttctataaagatagaggattaaaagcaccttttattgctgatcaggcgagatctttatttagtgtattttttgtagtttttctacttagaaccttcttgattggtaattttttaattccaactgcatcaatgacaccaacacttccagttggtgattttatttttgtcaataaaactgcttatggtatcagagcaccatttaccaatgagactttaataaaagttggtgaacccaaaagaggtgatattgtagtatttcattttccagttaatcctaatgttgattttgtaaaacgagtgatcggtttgcctggcgatgtaatttcgtataaagacaaaatgttgacaataaatggtaaaaaacttgaatatactaattgtaatcgtgatgcaatgaactattataatcagtctttagctgctggtagtggcgatacagtatgtacggaaaaccttgatggagttaaacatgaggttgattggatagagtctataaagggaactgattttgaaaaccttaaagtcccagcaggtcaatactttgtcatgggagataatcgtgataatagtgaagatagtcgttattggggttttgtacctgacaaagatctagttggtaaagcaaaagttgtttggatgagctgggataagatagataaaaaggttcgctgggatgaaattggtaaggtcttttaa

SEQ ID NO: 118 - protein, 282 aa, Campylobacter jejuni
MEILKKLYKFSQSWTGTVVIVLLVIFFFIQAFVIPSGSMKNTLLVGDFLFVKKFSYGIPTPHIPWLEIPVLPDFNKDGHLIKAQGSQRGDIVVFRNPRNEKEHFVKRCVGTGGDRIVYANKTLYVRMHEGDEFMKEHYPNDLVTLGGQIYVKEPYKQKGIHYDPKKDIESDILRFLSIGDFAMSPTYIKELGNHIGFSGGNAYVFDVPENEYFMMGDNRDYSYDSRFWGSVPYRLIVGKPWFVYFSWDKDKNVRWERIGRFVDTLENDEQYIHDHDDEDKLS

SEQ ID NO: 119 - DNA, 849 bp, Campylobacter jejuni
atggaaattttaaagaaattatataaattttcacagtcttggactggaactgtagttattgttcttttggtgatttttttctttatacaagcttttgttattccttctggttctatgaaaaacaccttattggtaggggattttttatttgttaaaaaatttagctatggtatcccaactcctcatattccttggttggaaattcctgttttgccagatttcaataaagatgggcatttgataaaagcacaagggtcacaaagaggagatatagttgtttttagaaatcctagaaatgaaaaagaacactttgtaaagcgttgtgtaggcacaggaggagataggatagtttatgcaaataaaacactttatgtaagaatgcatgagggtgatgaatttatgaaagaacattatccgaatgatcttgttactcttggagggcaaatttatgtaaaagaaccttataaacaaaaaggtattcattatgatccaaaaaaagatatagaaagcgatattttacgctttcttagcataggtgattttgctatgtctccaacttatattaaagaacttggaaatcatataggttttagcggcggaaatgcttatgtttttgatgtgcctgaaaatgagtatttcatgatgggtgataatcgcgattattcttatgatagtcgtttttggggttctgttccttataggttgatagtaggtaaaccttggtttgtatatttctcttgggataaagataaaaatgttcgctgggaaaggatagggcgttttgttgataccttggaaaatgatgaacaatatatccatgatcatgatgatgaggataaattaagctaa

SEQ ID NO: 120 - protein, 290 aa, Helicobacter pylori
MKFLRSVYAFCSSWVGTIVIVLLVIFFVAQAFIIPSRSMVGTLYEGDMLFVKKFSYGIPIPKIPWIELPIMPDFKNNGHLIEGDRPKRGEVVVFIPPHEKKSYYVKRNFAIGGDEVLFTNEGFYLHPFESGNDKDYIAKHYPNAMTKEFMGKIFVLNPYKSKHPGIHYQKDNETFHLMEQLATQGAEANISMQLIQMEGEKVFYKKINSDEFFMIGDNRDNSSDSRFWGSVAYKNIVGSPWFVYFSLSLKNSLEMDAENNPKKRYLVRWERMFKSVEGLEKIIKKEKATH

SEQ ID NO: 121 - DNA, 873 bp, Helicobacter pylori
atgaaatttttacgctctgtttatgcattttgctccagttgggtagggacgattgttattgtgctgttggttatcttttttgttgcgcaagctttcatcattccctctcgctctatggtaggcacgctctatgagggcgacatgctctttgtcaaaaaattttcttacggcatacccattcctaaaatcccatggattgagcttcctattatgcctgattttaaaaataacgggcatttgatagagggggatcgccctaagcgcggcgaagtggtcgtatttatccccccccatgaaaaaaaatcttactatgtcaaaaggaattttgccattgggggcgatgaggtgctattcactaatgaggggttttatttgcacccttttgagagcggcaacgataaagattatattgctaaacattaccctaacgccatgactaaagaatttatgggtaaaatttttgttttaaacccttataaaagtaagcatccgggtatccattaccaaaaagacaatgaaaccttccacttaatggagcagttagccactcaaggtgcggaagctaatatcagcatgcaactcattcaaatggagggcgaaaaggtgttttacaagaaaatcaatagcgatgaatttttcatgatcggcgataacagagacaattctagcgactcgcgcttttgggggagtgtggcttataaaaacatcgtgggttcgccatggtttgtttatttcagtttgagtttaaaaaatagcctggaaatggatgcagaaaacaaccccaaaaaacgctatttggtgcgttgggaacgcatgtttaaaagcgttgaaggcttagaaaaaatcattaaaaaagaaaaagcaacgcattaa

SEQ ID NO: 122 - protein, 274 aa, Propionibacterium acnes
VADDYRARRAANGDTRDSDDATARGEQASGWQRFRSGAIEVVLIVVGALIISAVLRGFVAQMFVIPSKSMQNTLQVGDRVIAVKAADFHRGDVVVFKDTEHWLPAVQDRRSVPGQILEFVGLLPNKSSNYLIKRVIGMPGDTVACCNVNGQVTVNGKALDERSYLYSENGEMVKPSAMEFRVTVPRGRMFVLGDHRNASGDSRYHLQDLDPGEYTGAPAFVPLDDVVGPAKAILMPLNRIEGLGTPNTFRGIPDRSSSAPAKARICVGNTCCPK

SEQ ID NO: 123 - DNA, 825 bp, Propionibacterium acnes
gtggcggatgactaccgggcgaggcgggctgcaaacggcgacaccagggactctgacgatgcaacagcacgtggggaacaggcgtctgggtggcagcgctttcggtcgggggccatcgaagttgttctcatcgtcgttggtgccctcatcatctcagctgtgctgcgtggtttcgtcgctcagatgtttgtcatcccgtcgaagtccatgcaaaacaccttgcaggtgggtgaccgcgtgatcgcggtgaaagccgccgattttcatcggggcgacgtcgtcgtgttcaaagacaccgaacattggttacctgctgttcaggatcgccgctctgttccaggacagatcctcgaattcgtcgggttgttgcctaacaagagctcgaactacctcattaagcgagtgatcggcatgcctggggacaccgttgcctgctgcaacgtcaacggccaggtgaccgtcaacggtaaggcgcttgacgagcggtcatacctgtactccgaaaatggtgaaatggttaaaccctcggcgatggaattccgggtcactgttcctcgggggcggatgttcgtcttgggggaccatcgcaatgcctcgggtgactcgcgctatcacctccaagaccttgatccgggtgagtatacgggcgctcctgcgtttgtgccgctcgatgacgtcgttgggccggcaaaggccattcttatgcctctcaatcgcattgagggactggggactcctaacactttccggggaatcccggataggtcgtcgtcagctccagccaaggcgcgcatctgcgtcggtaacacgtgctgccctaagtga

SEQ ID NO: 124 - protein, 628 aa, Chlamydia trachomatis
MTSSYMSRLYSLNKSRRILHSSFRLLKSTKMLSHPETQKELQEVLKQLEEAILDQNREDASLFAKQAQAIQKRFPKSKLRATFDLIYALTFAAILAFLIRQFWFELYEVPTGSMRPTILEQDRILVSKTTFGLRLPFSNRSIGYTPEAITRGELVVFTVGDLPIPNADTKYFGIIPGKKRYIKRCMGKPGDTVYFYGGKIYGIDCDGEPIFPQNTENLYHVPYISFDGTPEILTHSEEQTDVIFNQFHTPCGKISLPQQASYGQFFYKNAWHNDTPYALKDPHNEPVSYADLFGIKNFAMVRILTKKQAALTHVLPSPLSDTYLEIAHTPNVSYPHPHLRPFETQLIPTIEPMKTLLPLRKEHIHLIRNNLTTSRFTVVDGYAYKYQPAPMNTSGMVRMFALPMPNIPDGCYEFSKGDVFKINMGGFRTKLKQPHPLTQLSNSQVIDLFNCGISFHTIYIPKNPQYAPFPNRYAFFNQGNLFVMDSPVFIDSDPALQKFIVSEEEKELQSSEDKPYIAFIDRGPPPESTEEFVSFITNFGLKIPEGHVLVLGDNCPMSADSRDFGFVPVENLLGSPVGIFWPINRLGLLSSNITPLSLPGYLVNGLALGAFLYCIGLWYYRKNHRLFP

SEQ ID NO: 125 - DNA, 1887 bp, Chlamydia trachomatis
atgacgagcagttacatgagtcgcttatattccctgaataagagtcgtcgcattcttcattcttcctttagattgctgaaaagcacaaaaatgctctctcatccggaaactcaaaaagaactacaagaagtcttgaaacagcttgaagaggctattttggatcagaatagggaagatgcttccctttttgctaagcaagctcaagccatacaaaaaagattccctaaatccaaactccgagctacttttgatcttatctatgctttgacgtttgctgccattcttgcttttttaatccgccagttctggtttgagctatatgaagttcctacaggatctatgcggcctactattcttgaacaagatcgtattcttgtttccaaaacaacatttggactccggctaccttttagtaacagaagtattggctatacacctgaggctatcactcgaggagaactggtagtcttcactgttggagatcttcctatccctaatgccgacactaagtattttggaatcatccctgggaaaaaacgctatataaaacggtgcatgggtaaacctggagataccgtatatttttatggagggaaaatttatgggatcgattgcgacggagagcccatcttcccccaaaatacagagaatctctaccacgtcccctatatttcttttgacggaactccagaaattcttacccattcagaagagcaaacagatgtgatctttaaccaatttcacacaccttgtggaaagatttctctccctcaacaggcttcttatggacaatttttctataagaatgcttggcataatgatactccctatgctttaaaagatcctcataatgagcctgttagctatgccgatctattcggaataaaaaattttgcaatggttcgcatccttaccaaaaaacaagctgctcttactcatgtccttccctctcctctttcggacacctacctagaaattgcccacactcctaatgtttcctatcctcaccctcacttacgtccatttgaaacacagcttattcctactatcgaacctatgaaaaccttgcttcctttaaggaaggaacatattcatttgattcgtaataacctcacaacatcccgttttacagttgtagatggatatgcttacaagtaccaacctgctcccatgaatacctcaggcatggtcaggatgtttgccctacctatgccaaatattcctgacggatgttatgaattttctaaaggagacgtgtttaaaatcaatatgggtggctttcgaacaaaactcaaacagccgcatcctttaacgcaattaagcaattctcaggtcattgacttatttaattgcggcattagtttccacacgatctatattcctaaaaaccctcaatatgctccgttccctaatcgctatgcatttttcaatcaagggaacctgttcgttatggattctccagtttttattgatagcgatcctgccttacagaaattcattgtgtctgaagaggaaaaagaacttcaatcatctgaagacaaaccttacatcgcatttattgacagaggtcctcctccagaatctacagaggaatttgtttcctttattactaatttcggtcttaaaattccggaaggccacgtgcttgtcttaggagataattgtcctatgagcgctgatagccgtgattttggttttgttcccgttgaaaatcttttgggatctcctgttgggatcttctggcctattaatcgtctaggattgttatcttccaatataacgcccttgagtttacctggctacctcgtaaatggattggctctaggagcttttctttactgcataggattatggtactatcgaaaaaaccataggctattcccttaa

SEQ ID NO: 126 - protein, 636 aa, Chlamydophila pneumoniae
MKQHYSLNKSRHILRSTYKLLKSKKLAHSPADKKQLQELLEQLEEAIFEHDQETASDLAQQALAFSNRYPNSFGRKTYELIKALLFAGVVAFLVRQFWFELYEVPTGSMRPTILEQDRILVSKTTFGLHCPFAKKPLAFNPESVTRGGLVVFTVGDLPIPDADTKYFGLIPGKKRYIKRCMGRPGDFLYFYGGKIYGLDDAGKRIEFPSVHGLENLYHVPYISFDGTTSSHTEGQKTIIDFKQFNQSYGRLIFPQTSMYGQFFDHKEWHQDEPNKLKDPHLSPVSYADLFGMGNYAMVRILTEHQARTSHLLPNPGSPTKVYLEICHTANLSYPKPLLRHYEHQLSPAIQPMKTLLPLRKEHLHLIRNNLTTSRFIVAQGCAYKYHQFKINTSGIAKAYAILLPKVPDGCYEYSKGEAYQIGFGEIRYKLKSSHPLTQLNDKQVIELFNCGINFSSIYNPVNPLQAPLPNRYAFFNQGNLYIMDSPVFIKNDPTLQKFVTSETEKQEGSSETQPYIAFVDKGLPPEDFKEFVEFIHNFGIQVPKGHVLVLGDNYPMSADSREFGFVPMENLLGSPLCTFWPIGRMGRLTGVSAPTTLSGYLVSGIALATGLSLIGYVYYQKRRRLFPKKEEKNHKK

SEQ ID NO: 127 - DNA, 1911 bp, Chlamydophila pneumoniae
atgaaacaacactattctctaaataaaagtcgtcatatcctccgcagtacttataagcttttaaaaagtaaaaaactcgcccattcccctgcagataaaaagcaactgcaagaactactagaacaactagaagaggctatctttgaacatgatcaagaaactgcaagcgacttagctcagcaagcattagcattttccaaccgttatcctaattccttcggacgcaaaacctatgagcttatcaaggcccttctttttgctggtgttgtagccttcttagttcggcaattttggtttgaactttatgaagtgcctacaggatccatgaggcctacaattttagaacaggatcggattcttgtatccaaaacaacatttggtctccattgcccttttgctaagaaaccacttgccttcaatcctgaatccgtaactcgcgggggtcttgttgttttcactgtaggcgacctccctatcccagatgctgatacaaagtacttcggattgattccaggaaaaaagcgttacattaaacgttgcatgggaagacctggggacttcttatatttctatggaggaaaaatttatggtcttgatgatgcaggtaaacgcatagagtttccttctgtccatggtttagaaaacttatatcacgtcccctatatatcctttgatggcactaccagcagccatacagaagggcagaaaacaattatagattttaagcagttcaatcaaagttatggtcggctgattttccctcaaacctccatgtatggacaattctttgaccataaagaatggcatcaagacgagcctaataaattaaaagatcctcatctttcgccagtcagctatgccgatctttttggtatgggtaactatgctatggtgcgcatcttaacagaacatcaggcacgaacatcccatctacttccgaatccaggaagtccaactaaagtctacttagaaatttgccatacagcgaacctttcctacccaaagcctctgttgcgtcactatgagcatcagctctcgcctgcgattcaacctatgaagactttacttcctttgcgtaaggaacatttgcacttaattcggaacaatcttactacctctcgttttattgttgctcaaggatgtgcgtataaataccatcaattcaagattaacacttcaggaattgccaaagcctatgcaattctcctgcccaaggtccctgatggttgttatgaatattctaaaggcgaagcgtatcaaattggctttggagagattcgttataagctaaaatcttctcacccccttactcagctcaatgataagcaagtgattgaactttttaactgcgggatcaactttagttctatttataatcctgtgaatccgctgcaagcacctttacctaaccgttatgcattctttaaccaagggaatctttatatcatggattctcctgtatttataaagaatgatccaactctgcaaaaatttgtgacttctgaaacggaaaagcaagaggggtcttcagagacacaaccctatatagcttttgttgacaagggactccctccagaagattttaaagaattcgtggagtttatacataattttggtattcaagttcctaaaggtcatgttctcgtcttgggagataactaccctatgagtgcggatagtcgagaatttggctttgttcctatggaaaatctcttaggatctcctctatgtacattctggcctattggacgcatgggacggttaactggagtttctgctccaacaacactctcaggttatcttgttagtgggatagcattagcgacgggtctctctctcattggatatgtctactatcaaaaacgacgcagactctttcctaagaaagaggagaaaaaccacaagaaataa

SEQ ID NO: 128 - protein, 189 aa, Staphylococcus carnosus
VKKEIKEWIIAIAIALVLVLVITNFIAKSYTVRGDSMYPTLKDGEKVIVNMIGFKTGGLEKGNVIVFHATKNSDYVKRVIGMPGDSIEYKHDQLYVNGKKVKEPYLDYNEKHKSYDEITGSFKVKNLPNANGSNTIPKNKLLVLGDNREVSKDSRSFGLIDEDQVVGKVSLRYWPFTSFKVNFNPDTKY

SEQ ID NO: 129 - DNA, 570 bp, Staphylococcus carnosus
gtgaagaaagaaattaaagagtggataatagccatagcaatagctttggtattagttctagtcataacaaatttcattgcgaaatcatatacggttcgtggtgattcaatgtatccaacgctaaaagacggagaaaaagttatcgttaatatgattggatttaaaactggcggtttagaaaaaggtaatgtgattgtattccacgctactaaaaacagcgactacgttaaacgtgttatcggtatgcctggtgacagtattgaatataaacatgatcaattgtatgttaatggtaaaaaagtgaaagaaccttatttagattataatgaaaaacataaaagctatgatgaaattacaggtagctttaaagtgaaaaatttacctaatgcaaatggttcaaacacaattcctaaaaacaaacttcttgtattaggagataaccgtgaagtcagtaaagacagccgttcattcggtttaattgatgaagatcaagttgttggtaaagtaagcttgcgttattggccgtttacatctttcaaagtaaactttaatccggatacaaaatattaa

SEQ ID NO: 130 - protein, 190 aa, Staphylococcus haemolyticus
LKKEIVEWIVAIAVGLLLVWVMVNFVAKSYTIKGDSMDPTLKDGEHVMVNILGYKVGDIKKGNVIVFHANQQDDYVKRVIGVPGDNVIYKNDKLYVNGKKINEPYLDYNEKRKQGEYITGSFETKDLLNANPKSNIIPKGKYLVLGDNREVSKDSRAFGLIDRDQIVGKVSFRFWPFSEFKFNFNPDNEK

SEQ ID NO: 131 - DNA, 573 bp, Staphylococcus haemolyticus
ttgaagaaagaaatagttgaatggattgttgccatagcggtaggtttattacttgtatgggtaatggttaacttcgtagctaaatcatatactataaaaggcgattcaatggatccaacactaaaagatggcgaacacgtcatggttaacattctaggatataaagttggagacataaaaaaaggtaatgtaatcgtatttcatgcgaatcaacaagacgattatgttaaacgtgtcattggtgtacctggcgataacgttatttataaaaatgataaactatatgttaatggtaaaaagataaatgaaccttatcttgattacaatgaaaaacgtaaacaaggtgaatatattacgggttcatttgaaactaaagatttactaaatgcaaatcctaaatcaaatatcataccaaaaggtaaatacttagttttaggtgataacagagaagtcagtaaggatagtagggcgtttggtttaattgatagagatcaaattgttggtaaagtatcatttagattttggccattcagtgaatttaagtttaattttaatccagataatgaaaaataa

SEQ ID NO: 132 - protein, 190 aa, Staphylococcus haemolyticus
LKKEIIEWIVAIGGALLIVGIVLKFIGTSYTVSGSSMYPTFQDRNKVIVSKISKTLNHIDNGDVVVFHEDAQRDFIKRVIGTPGDKVEYEGDQLYVNDKKVSEPYLDYNKKHKQGKYLTGTFKTSQVNGANGKNKIPKDKYLVLGDNRQNSVDSRLAEVGLVDKDQLVGKVVLRYWPFNKWEAGFNPGTF

SEQ ID NO: 133 - DNA, 573 bp, Staphylococcus haemolyticus
ttgaaaaaagagataattgaatggattgtagccattggtggcgcactcttaattgtaggtattgtattaaagtttattggaacatcatacacagtatcaggttcatcgatgtatccaactttccaagatagaaataaagtgatagttagtaagatttcgaaaacattgaaccacattgataatggtgatgtcgttgtcttccatgaagatgcacaacgtgattttattaagcgtgtgattggtacgccaggtgataaagttgagtatgaaggtgatcaattatatgttaatgacaaaaaggtatcagagccttatttagattataataagaagcataaacaaggtaagtatttaacaggtacatttaaaacaagccaagtgaacggagcaaatggtaaaaataaaattcctaaagataagtatttagttttaggtgataacagacaaaatagtgtagatagccgtttggctgaagttggtttagtagataaagaccaacttgtaggtaaagttgttttaagatattggccatttaataaatgggaagcaggttttaacccaggcacattttag

SEQ ID NO: 134 - protein, 191 aa, Staphylococcus epidermidis
LKKEILEWIVAIAVAIALIAIITKFVGKSYSIKGDSMDPTLKDGERVVVNIIGYKLGGVEKGNVIVFHANKKDDYVKRVIGTPGDSVEYKNDTLYVNGKKQSEPYLNYNEKRKQTEYITGSFKTKNLPNANPQSNVIPKGKYLVLGDNREVSKDSRSFGLIDKDQIVGKVSLRYWPFSEFKSNFNPNNTKN

SEQ ID NO: 135 - DNA, 576 bp, Staphylococcus epidermidis; identical to SEQ ID NO: 2

SEQ ID NO: 136 - protein, 192 aa, Staphylococcus epidermidis
MKKEIIEWIVAIIVAIVIVTLVQKFLFASYTVKGASMHPTFENREKVIVSRIAKTLDHIDTGDVVIFHANAKQDYIKRLIGKPGDSVEYKKDQLYLNGKKVDEPYLSENKKHKVGEYLTENFKSKDLKGTNGNMKIPSGKYLVLGDNRQNSIDSRMDEVGLLDKNQVVGKVVLRYWPFNRWGGSFNPGTFPN

SEQ ID NO: 137 - DNA, 579 bp, Staphylococcus epidermidis
atgaagaaagaaataatagaatggattgtagccataatcgttgcaattgttatcgtcacacttgtgcaaaagtttttatttgcttcttatacagtcaaaggagcgtctatgcatccaacatttgaaaatagagaaaaagtgatagtaagtcgtatagcaaaaacacttgatcatattgatacaggagatgtagtgatttttcatgctaacgcgaagcaagattatattaagcgacttattggtaaaccaggtgattcagtagaatataaaaaagatcaactatatttaaacggtaaaaaagtagatgagccttatttaagtgaaaataaaaaacataaagttggagaatatctaacggaaaactttaagtctaaagatcttaagggtacgaatggcaatatgaaaattcctagtggtaaatacttggttttaggtgataatcgtcaaaacagtattgacagtcgcatggatgaagtaggtcttttagataaaaatcaagttgttggaaaagtagttttgagatactggccatttaatcggtggggcggtagttttaatcctggaacatttcctaactaa

SEQ ID NO: 138 - protein, 188 aa, Staphylococcus hominis
LKKEITEWIVAIAVGLLLVWLVVTFVAKSYTIKGDSMDPTLKDGQHVMVNILGYKVGNIKKGNVIVFHANQSDDYVKRVIGVPGDSVTYKKDQLYINGKKVNEPYLDYNEKHKQGEYITGSFETKDLLNAHPNSNVIPKNKYLVLGDNREVSKDSRAFGLIDKQQIVGKVSFRFWPLNNFKFNFNPDK

SEQ ID NO: 139 - DNA, 567 bp, Staphylococcus hominis
ttgaaaaaagaaataacagaatggattgttgcgatagctgtaggtttattgctcgtatggcttgtagtcacttttgttgccaaatcctatacaataaaaggtgactcaatggatccaacattaaaagatgggcaacatgtgatggttaacattttaggttataaggtaggaaacataaaaaaaggaaatgttattgtcttccatgctaatcaatctgatgactatgttaaaagagtaataggcgtaccaggagatagtgtgacatataaaaaagatcagctatatattaatgggaaaaaggtaaatgagccttacttagactataatgaaaaacataaacaaggagagtacattactggatcttttgaaactaaggatcttcttaatgctcatcctaactctaacgttattcctaaaaataaatacttagtattaggagataaccgtgaagttagtaaagatagtagagcgtttggattaatagataaacaacaaatcgtcggtaaagtatcatttagattttggccattaaataattttaaatttaattttaatccagataagtag

SEQ ID NO: 140 - protein, 189 aa, Staphylococcus lugdunensis
VKKELTEWLIAIAVGIILVILIINFVAKSYTIKGDSMNPTLKDGDHVLVNIIGYKVGTVKKGNVIVFHANQKDDYVKRVIGTPGDKVYYRDDQLIINGKKVKEPYLEYNMKRKQGEYITGSLDIKDLAGAKHNSNVIPQHKYLVLGDNREVSKDSRAFGLIDEKQIVGKVSLRFWPLTDFKFNFNPDMS

SEQ ID NO: 141 - DNA, 570 bp, Staphylococcus lugdunensis
gtgaaaaaggaattgacagaatggttaatagctatagcggtaggtattattttagtcatactaatcattaattttgtagcgaaatcatataccattaaaggagactcaatgaatccaacattaaaagatggcgatcatgttctggtcaatattatcggctataaagtaggcactgtgaaaaaggggaatgtcattgtcttccatgctaaccaaaaggatgattatgttaaacgcgttataggcacaccaggtgacaaagtatactatcgagatgatcaacttattataaacggaaaaaaagtaaaagaaccttatctcgaatacaatatgaaacgtaagcaaggagagtatattactggatctttagatataaaagatttggccggtgcaaaacataattctaatgtcatacctcaacataaatacctcgtgttaggagacaatcgtgaggtaagtaaagatagccgtgcttttggccttatcgatgaaaagcaaattgtcggtaaagtgtctttaagattttggccattaactgattttaaatttaattttaaccctgatatgagctaa

SEQ ID NO: 142 - protein, 192 aa, Staphylococcus lugdunensis
VKKEILEWIVSIAVALIIVGIVVKFIGVTYSVSGDSMYPTFKDREKVVVSKISKTLDHIDNGDIVVFKEDKDRDFIKRLIGKPGDKVEYKGDQLYVNNKKIDEPYLKYNKEHKNGKYLTGSFKSSDLQNANGETKIPKDKYLVLGDNRQNSLDSRFPQVGLIDKEQIVGKVVLRFWPFGEWTTKFNPGTFDK

SEQ ID NO: 143 - DNA, 579 bp, Staphylococcus lugdunensis
gtgaaaaaagagatcttagagtggattgtgtctatagcagttgcacttatcattgtaggtatagttgttaaatttattggagttacatattcagtttcgggagattcaatgtatccaacatttaaagatagagaaaaagtagtagtgagtaaaatttccaaaacgttagaccatattgataatggtgatatcgttgtctttaaagaagataaagatagagactttattaaacgtttaattggtaaacctggagacaaagttgagtataaaggtgaccaactatatgttaataataaaaaaattgatgagccttatttaaaatataacaaagagcataaaaatggtaagtatctgacaggttctttcaaatcgagtgatttgcaaaatgctaatggtgagacgaagattcctaaagacaaatatttagtgttaggtgataatcgtcaaaacagtttagatagtcgttttccacaggtagggcttattgataaagaacaaattgtaggtaaagttgtgttacgtttctggccatttggtgagtggacaacaaaatttaatcctggaacatttgataagtaa

SEQ ID NO: 144 - protein, 189 aa, Streptococcus agalactiae
MKRQISSDKLSQELDRVTYQKRFWSVIKNTIYILMAVASIAILIAVLWLPVLRIYGHSMNKTLSAGDVVFTVKGSNFKTGDVVAFYYNNKVLVKRVIAESGDWVNIDSQGDVYVNQHKLKEPYVIHKALGNSNIKYPYQVPDKKIFVLGDNRKTSIDSRSTSVGDVSEEQIVGKISFRIWPLGKISSIN

SEQ ID NO: 145 - DNA, 570 bp, Streptococcus agalactiae
atgaaaagacagattagttcagataaattatctcaagaactggatcgcgtaacttatcagaaacgcttttggagtgtcattaaaaataccatatacatcttgatggcggttgcctcaatagccattttaattgcggttttatggttgcctgtattaagaatctacggacattcaatgaataagactttaagtgcaggtgatgtagtctttacagtaaaaggttcaaattttaaaactggagacgttgtcgcgttttactacaataataaggtcctagtcaagcgggttattgcagagtcaggagactgggttaatattgattctcaaggggatgtttacgtgaatcaacataagttgaaagaaccatatgttattcataaagcactcggtaatagtaatataaaatacccatatcaagtacctgataaaaaaatttttgtattaggagacaaccgaaaaacttcaattgattctcgaagtacttctgtaggagatgtttcagaagaacaaattgtaggtaaaatttctttcagaatatggcctctaggtaagattagtagtatcaattaa

SEQ ID NO: 146 - protein, 197 aa, Streptococcus agalactiae
MKEFIKEWGVFILILSLFLLSRIFLWQFVKVDGHSMDPTLADKEQLVVLKQTKINRFDIVVANEEEGGQKKKIVKRVIGMPGDVIKYKNDTLTINNKKTEEPYLKEYTKLFKKDKLQEKYSYNPLFQDLAQSSTAFTTDSNGSSEFTTVVPKGHYYLVGDDRIVSKDSRAVGSFKKSTIVGEVKFRFWPIRRFGTIN

SEQ ID NO: 147 - DNA, 594 bp, Streptococcus agalactiae
atgaaagaatttattaaagaatggggtgtctttatcctcatcctctcactttttttactatcgcgtatctttttatggcaattcgttaaagttgacggacactccatggatccaactttagctgacaaggaacagctagtagttctcaaacaaacaaaaatcaatcgattcgatattgtagtggctaacgaagaagaaggcggccaaaagaaaaaaattgttaaacgtgtcattggtatgccaggtgatgtcatcaaatataaaaatgacaccttaactattaacaataaaaaaacagaagaaccttacctcaaggaatatactaaattatttaaaaaggataaattacaggaaaaatattcgtataacccacttttccaagacctagcacaaagctctaccgctttcaccactgacagcaatggcagcagcgaatttactactgtcgtgcctaaaggccactactatcttgttggtgatgaccgaattgtctctaaagatagtcgtgccgtcggttccttcaaaaaatcaacgattgtgggagaggttaaattccgcttctggccaattcgtcgttttggaactatcaactaa

SEQ ID NO: 148 - protein, 197 aa, Streptococcus dysgalactiae
MKHFIKEWGPFTLFLILFGLSRLFLWQAVKVDGHSMDPTLAHGERLIVLNQARIDRFDIVVAREEENGQKKEIVKRVVGMPGDTIAYNDDTLYINGKKTDEPYLVNYLKEFKKDKLQKTYAYNSLFQQLAETSDAFTTNAEGQTRFEISVPEGEYLLLGDDRIVSRDSREVGSFKKEKLIGEVKARFWPLNKMTLFK

SEQ ID NO: 149 - DNA, 594 bp, Streptococcus  dysgalactiae
atgaaacattttattaaagaatggggcccatttaccctctttctcatcctcttcggtttatctcgtcttttcttgtggcaagctgttaaagttgatggccactccatggaccctacgttagcccatggggaacgtctcattgttttaaaccaagctagaattgaccgtttcgatattgtcgttgcccgtgaggaagaaaatgggcagaaaaaagaaattgtcaaacgagttgtcggcatgccaggtgataccattgcctacaacgatgatacgctttacattaatggtaaaaaaacagatgagccttacctagttaactaccttaaagagttcaaaaaggacaagcttcaaaagacttacgcttacaatagtctatttcagcaattagctgaaacatcggatgccttcaccactaatgctgaaggtcaaacacgttttgaaatcagtgtaccagaaggtgaatacctccttcttggagatgaccgaattgtctcacgcgacagccgtgaagttggtagttttaaaaaagaaaaacttatcggtgaagtcaaggctcgcttctggccactcaataaaatgactctttttaagtaa

SEQ ID NO: 150 - protein, 204 aa, Streptococcus mitis
MNSFKNFLKEWGLFLLILSLLALSRIFFWSNVRVEGHSMDPTLADGEILFVVKHLPIDRFDIVVAHEEDGNKDIVKRVIGMPGDTIRYENDKLYINDKETDEPYLADYIKRFKDDKLQSTYSGKGFEGNKGTFFRSIAEKAQAFTVDVNYNTNFSFTVPEGEYLLLGDDRLVSSDSRHVGTFKAKDITGEAKFRFWPITRIGTF

SEQ ID NO: 151 - DNA, 615 bp, Streptococcus mitis
atgaattcatttaaaaatttcctaaaagagtggggattgttcctcctgattctgtcattactagctttgagccgtatctttttttggagtaatgtccgcgtagaagggcattccatggatccgaccctagcggatggcgaaattctcttcgttgtcaaacaccttcctattgaccgttttgatatcgtggtggcccatgaggaagatggcaataaggacatcgtcaagcgcgtgattggaatgcctggcgatactatccgttacgaaaacgataaactttacatcaatgataaagagacggacgaaccttacctagctgactatatcaaacgtttcaaggatgacaaactccaaagcacctactcaggcaagggctttgaaggaaataaaggaaccttctttagaagtattgcggaaaaagctcaagccttcacagttgatgtcaactataacaccaactttagctttactgttccagaaggagaataccttctcctcggagacgaccgcttggtttctagcgacagccgtcacgtaggtaccttcaaagcaaaagatatcacaggggaagctaaattccgcttctggccaatcacccgtatcggaacattttaa

SEQ ID NO: 152 - protein, 204 aa, Streptococcus oralis
MNSFKTFLKEWGVFFLIIALVGLSRIFLWSNVRVEGHSMDPTLADGEVLFVVKHLPIDRFDIVVAHEEDGNKDIVKRVIGMPGDTIRYENDKLFINGEETNEPYLAEYLNLFKTEKLQNTYTGKGFEGNKGVYFRELAQKAQAFTVDVNSNTRFSFTVPQGEYLLLGDDRLVSSDSRHVGTFKASDIKGEAKFRFWPLNRIGTF

SEQ ID NO: 153 - DNA, 615 bp, Streptococcus oralis
atgaattcgtttaaaacatttctaaaagaatggggagttttcttcctgattatcgcactggtcggtcttagccgcatctttctttggagcaatgtccgtgtggaaggacactctatggaccctaccctagctgacggagaagttctcttcgttgttaaacacctcccaattgaccgcttcgacatcgtggttgcgcatgaggaagacggaaataaagacattgtcaaaagggttatcggtatgcctggtgataccatccgctacgaaaatgacaaactctttatcaacggtgaagaaacgaatgaaccctacctagctgagtacctcaacttgttcaaaacagaaaagttgcaaaacacctatactggaaaaggatttgaaggcaataagggagtttactttagagaacttgctcaaaaagcacaagcctttacggtcgatgtcaattccaacaccagattcagctttactgtccctcaaggcgaataccttctccttggtgacgatcgtctagtctctagcgacagccgccatgtcggtaccttcaaggccagcgatatcaaaggcgaagcaaaattccgtttctggccacttaaccgtatcggaactttttaa

SEQ ID NO: 154 - protein, 204 aa, Streptococcus pneumoniae
MNLFKNFLKEWGLFLLILSLLALSRIFFWSNVRVEGHSMDPTLADGEILFVVKHLPIDRFDIVVAHEEDGNKDIVKRVIGMPGDTIRYENDKLYINDKETDEPYLADYIKRFKDDKLQSTYSGKGFEGNKGTFFRSIAQKAQAFTVDVNYNTNFSFTVPEGEYLLLGDDRLVSSDSRHVGTFKAKDITGEAKFRFWPITRIGTF

SEQ ID NO: 155 - DNA, 615 bp, Streptococcus pneumoniae
atgaatttatttaaaaatttcttaaaagagtggggattattcctcctgattctgtcattactagctttgagccgtatctttttttggagcaatgttcgcgtagaaggacattccatggatccgaccctagcggatggtgaaatcctctttgttgttaagcacctccctattgaccgttttgatatcgtggtggcccatgaggaagatggcaataaggacatcgtcaagcgcgtgattggaatgcctggcgacaccattcgttacgaaaatgataaactctacatcaatgacaaagaaacggacgagccttatctagcagactatatcaaacgcttcaaggatgacaaactccaaagcacttactcaggcaagggctttgaaggaaataaaggaactttctttagaagtatcgctcaaaaagcccaagccttcacagttgatgtcaactacaacaccaactttagctttactgttccagaaggagaataccttctcctcggagatgaccgcttggtttcgagcgacagccgccacgtaggtaccttcaaagcaaaagatatcacaggggaagctaaattccgcttctggccaatcacccgtatcggaacattttaa

SEQ ID NO: 156 - protein, 197 aa, Streptococcus pyogenes
MKQFIKEWGPFTLFLILFGLSRLFLWQAVKVDGHSMDPTLAHGERLIVFNQARIDRFDIVVAQEEENGQKKEIVKRVIGLPGDTISYNDDTLYINGKKTVEPYLAEYLKQFKNDKLQKTYAYNTLFQQLAETSDAFTTNSEGQTRFEMSVPKGEYLLLGDDRIVSRDSREVGSFKKENLIGEVKARFWPLNKMTVFN

SEQ ID NO: 157 - DNA, 594 bp, Streptococcus pyogenes
atgaaacagtttattaaagaatggggcccattcactctctttttaattctctttggtctatctcgtctttttttgtggcaggctgttaaagtagacggccattctatggacccaactctagctcatggcgaacgccttatcgtttttaatcaagctagaattgatcgctttgatattgtagttgctcaggaagaagaaaacggacaaaagaaagaaatcgtaaaaagagttattggattgccaggcgataccatttcttataatgatgacacactttatattaatggtaaaaaaacagttgagccgtatttggctgagtatctaaaacaatttaaaaacgataaactccaaaaaacttacgcctataataccctattccaacagttagcagaaacatctgatgcttttacaactaattctgagggacaaacacgctttgagatgagtgttccaaaaggagaataccttcttcttggtgatgatcgtattgtttccagggatagtcgcgaagttggtagtttcaaaaaagaaaaccttatcggtgaagtgaaagctcgtttttggccactcaataaaatgaccgtttttaattag

SEQ ID NO: 158 - protein, 324 aa, Escherichia coli
MANMFALILVIATLVTGILWCVDKFFFAPKRRERQAAAQAAAGDSLDKATLKKVAPKPGWLETGASVFPVLAIVLIVRSFIYEPFQIPSGSMMPTLLIGDFILVEKFAYGIKDPIYQKTLIETGHPKRGDIVVFKYPEDPKLDYIKRAVGLPGDKVTYDPVSKELTIQPGCSSGQACENALPVTYSNVEPSDFVQTFSRRNGGEATSGFFEVPKNETKENGIRLSERKETLGDVTHRILTVPIAQDQVGMYYQQPGQQLATWIVPPGQYFMMGDNRDNSADSRYWGFVPEANLVGRATAIWMSFDKQEGEWPTGLRLSRIGGIH

SEQ ID NO: 159 - DNA, 975 bp, Escherichia coli
atggcgaatatgtttgccctgattctggtgattgccacactggtgacgggcattttatggtgcgtggataaattctttttcgcacctaaacggcgggaacgtcaggcagcggcgcaggcggctgccggggactcactggataaagcaacgttgaaaaaggttgcgccgaagcctggctggctggaaaccggtgcttctgtttttccggtactggctatcgtattgattgtgcgttcgtttatttatgaaccgttccagatcccgtcaggttcgatgatgccgactctgttaattggtgattttattctggtagagaagtttgcttatggcattaaagatcctatctaccagaaaacgctgatcgaaaccggtcatccgaaacgcggcgatatcgtggtctttaaatatccggaagatccaaagcttgattacatcaagcgcgcggtgggtttaccgggcgataaagtcacttacgatccggtctcaaaagagctgacgattcaaccgggatgcagttccggccaggcgtgtgaaaacgcgctgccggtcacctactcaaacgtggaaccgagcgatttcgttcagaccttctcacgccgtaatggtggggaagcgaccagcggattctttgaagtgccgaaaaacgaaaccaaagaaaatggaattcgtctttccgagcgtaaagagacactgggtgatgtgacgcaccgcattctgacagtgccgattgcgcaggatcaggtggggatgtattaccagcagccagggcaacaactggcaacctggattgttcctccgggacaatacttcatgatgggcgacaaccgcgacaacagcgcggacagccgttactggggctttgtgccggaagcgaatctggtcggtcgggcaacggctatctggatgagcttcgataagcaagaaggcgaatggccgactggtctgcgcttaagtcgcattggcggcatccattaa

SEQ ID NO: 160 - protein, 324 aa, Salmonella enterica
MANMFALILVIATLVTGILWCVDKFVFAPKRRARQAAAQTASGDALDNATLNKVAPKPGWLETGASVFPVLAIVLIVRSFLYEPFQIPSGSMMPTLLIGDFILVEKFAYGIKDPIYQKTLIETGHPKRGDIVVFKYPEDPKLDYIKRAVGLPGDKITYDPVAKEVTIQPGCSSGQACENALPVTYSNVEPSDFVQTFARRNGGEATSGFFEVPLNETKENGIRLTERKETLGDVTHRILMVPIAQDQLGMYYQQPGQPLATWVVPPGQYFMMGDNRDNSADSRYWGFVPEANLVGKAVAIWMSFDKQEGEWPTGVRLSRIGGIH

SEQ ID NO: 161 - DNA, 975 bp, Salmonella enterica
atggcgaacatgtttgccctgattctggtgatagccacactggtgacgggcattttatggtgcgttgataagtttgttttcgcgccaaaacgtcgggcgcgccaggctgccgcgcaaacggcgtcgggagatgcgctggataacgctacgctcaataaagtggcgcctaagccgggctggctggagaccggggcgtcggttttcccggttctggcgatcgttctgatcgttcgttcatttctttatgaaccctttcagatcccgtcaggctcaatgatgccgacactgcttatcggcgattttattctggtggaaaaatttgcctacggcattaaagatccgatctaccagaaaaccctgattgaaaccggtcatccaaagcgcggggatattgtggtatttaaatatccggaagatcctaagttagattacatcaaacgcgccgtcggtttgccgggcgataaaatcacttatgatccggttgcgaaagaggtgacgattcagcctggctgtagctccggtcaggcgtgcgaaaatgcgctgccggttacctactctaacgttgagccgagcgattttgtacagacctttgcccgccgtaacggcggagaagcgaccagcggtttctttgaggttccgctaaacgagacaaaagaaaacggcattcgcctgaccgaacgtaaagagacgttaggcgatgtgacccaccgcatcctgatggtgccgatagcccaggatcagttgggcatgtattaccaacagccaggacaaccgctggcgacctgggttgtaccgccggggcaatatttcatgatgggcgacaaccgcgataacagcgcggatagtcgttactggggatttgttccggaagcgaatctggtcggtaaagcggtcgctatctggatgagctttgacaagcaggaaggggagtggccgacaggcgtacgcctgagtcgtatcggcggtattcactaa

SEQ ID NO: 162 - protein, 324 aa, Klebsiella pneumoniae
MANMFALILVIATLVTGVLWCLDKFIFAPKRRERQAAAQAATGEQLDKKTLKKVGPKPGWLETGASVFPVLAIVLVVRSFIYEPFQIPSGSMMPTLLIGDFILVEKFAYGIKDPIYQKTLIETGHPKRGDIVVFKYPEDPRLDYIKRAVGLPGDKVTYDPVAKQVTIQPGCSSGQACGNALPVTYSNVEPSDFVQTFSRSNGGEASSGFWQLPKGETKADGIRLTERQETLGDVTHRILMVPIAQDQVGMYYHQSGLPLATWIVPPGQYFMMGDNRDNSADSRYWGFVPEANLVGKATAIWMSFEKQEGEWPTGVRLSRIGGIH

SEQ ID NO: 163 - DNA, 975 bp, Klebsiella pneumoniae
atggcgaacatgtttgccctgatcctggtgattgcaaccctggtgacgggcgttttatggtgcctggacaagttcatttttgcaccgaaacgtcgtgaacgtcaggccgctgctcaggcagcgaccggcgagcaactggacaagaagacgctgaagaaagtcggcccgaaaccgggctggctggaaaccggcgcatcggttttcccggtgctggcgatcgttctggtggtacgttcatttatttatgagcctttccagatcccttcaggttcgatgatgccaacgctgctcatcggcgattttattctggtggagaaatttgcctacggcattaaagatcctatctaccagaaaacgctgatcgagaccggccatccgaagcgcggcgacatcgtggtatttaaatatccggaagacccgcgtctggactacattaagcgcgcggtggggttaccgggtgataaggtcacctacgatccggttgccaaacaggtcactattcagccgggctgcagttccggacaggcctgcggcaacgcgctgccggtgacctattccaacgtggagccgagcgattttgttcagaccttctcccgcagcaacggcggcgaagcgagcagcggtttctggcagttgccgaagggcgaaaccaaagccgacggcattcgtcttaccgagcgtcaggagacattgggcgacgtgacgcaccgaattctgatggtgccgattgcccaggatcaggttgggatgtactaccatcagtccggtctgccgctggccacctggattgtgccgcccggtcagtacttcatgatgggcgacaaccgggataacagcgccgacagccggtactggggctttgtgccggaagccaacctggtcggaaaagcaaccgctatctggatgagttttgaaaagcaggaaggtgaatggccgaccggcgtgcggttatcgcgcattggtggaattcattaa

SEQ ID NO: 164 - protein, 294 aa, Mycobacterium tuberculosis
VTETTDSPSERQPGPAEPELSSRDPDIAGQVFDAAPFDAAPDADSEGDSKAAKTDEPRPAKRSTLREFAVLAVIAVVLYYVMLTFVARPYLIPSESMEPTLHGCSTCVGDRIMVDKLSYRFGSPQPGDVIVFRGPPSWNVGYKSIRSHNVAVRWVQNALSFIGFVPPDENDLVKRVIAVGGQTVQCRSDTGLTVNGRPLKEPYLDPATMMADPSIYPCLGSEFGPVTVPPGRVWVMGDNRTHSADSRAHCPLLCTDDPLPGTVPVANVIGKARLIVWPPSRWGVVRSVNPQQGR

SEQ ID NO: 165 - DNA, 885 bp, Mycobacterium tuberculosis
gtgaccgaaaccacggactccccatcggagcgccagccgggtccggcagagccggagctctcctcccgggacccggacattgccggccaggtcttcgacgcagccccgttcgacgcagccccggatgcggactccgaaggcgactccaaggcggccaaaacggacgagccgcggcccgcgaagcgatcaacgctgcgggagttcgcggtgctggcggtgattgctgtggtgctctactacgtcatgctgacgtttgtcgcgcgcccttatctgattccgtcggaatcgatggaacccacgttgcacgggtgttcgacgtgcgtcggcgaccgcatcatggtggacaaactcagctaccgcttcggctcaccgcaacctggcgacgtcatcgtcttcaggggaccgccgtcgtggaacgttggttacaagtcgatccgttcgcacaacgtcgccgtgcgctgggtgcagaacgcgttgtcgttcatcggtttcgtgcctcccgacgagaacgacctggtcaagcgtgtcatcgcggtcggcggacagacggttcaatgccggtccgacaccggcctgacggtcaacggcaggccactgaaggagccatacctggatccggccaccatgatggccgacccgtcgatatacccgtgcctgggcagcgagttcgggccggtcaccgtcccgcccgggcgtgtctgggtgatgggcgacaaccgcacccattcggcggattcccgcgctcactgcccgttgctatgtactgacgatccgctaccggggaccgtgccggtggccaacgtcatcggtaaggccaggttgatcgtgtggccgccgtcgcgttggggtgttgtgcgttcggtgaatccccagcaaggtcggtag

SEQ ID NO: 166 - protein, 332 aa, Yersinia pestis
MANMFALILAIATLLTGIIWCFERFKWGPARQAKIAAVNAQTAEIKAQTGCAVDNKTLAQAAKQPGWIETCASIFPVLALVFIVRSFIYEPFQIPSGSMMPTLLIGDFILVEKFAYGIKDPITQTTLIPTGKPNRGDIAVFKYPLDPRLDYIKRVVGLPGDRVIYNPISKEVTVQPSCNTGTSCDSALAITYSTSEPSEFVQTFRYSGNGESSAGFFPIPLNQAVPDGGVRLRERTESLGPVAHHILTVPGRQDPLGSYYQQPDQPLGVWVVPEGHYFMMGDNRDNSADSRFWGFVPERNLVGKATAIWMSFEKQEGEWPTGVRLSRIGGIH

SEQ ID NO: 167 - DNA, 999 bp, Yersinia pestis
atggctaacatgtttgctttgattctggcaatagcaacgctgttgacggggattatctggtgcttcgagcggtttaaatgggggccagcccgtcaggcaaaaattgcggcagttaatgcacaaactgcggaaatcaaggcccaaaccgggtgtgccgtagataataaaaccttagcccaagctgcaaagcaaccgggttggatcgagacatgtgcctctatcttcccggtgctggccttggtctttatcgtgcgttcgtttatttacgagcctttccagatcccttctggttcgatgatgccaacgctgcttatcggtgattttattttggttgagaaatttgcttatgggattaaagatcccattactcagaccacattaattccaacaggtaagccaaaccgcggtgacattgcggtgtttaaatatccgttggatccacgtttggattatatcaagcgtgtggtggggctgccgggggatcgggtaatttataacccgataagtaaagaagtcacggtacaaccgtcatgtaataccggtacttcttgtgatagtgcgttggccatcacttacagcacgtctgagccaagtgagtttgtgcagacattccgttatagcggtaatggcgaaagctccgcagggttcttcccaatcccgctaaatcaggcagtacctgatggcggtgtccggttacgtgagcgtactgaaagcctcggcccggtagcgcatcacattctgaccgtcccagggcggcaggatccgttaggctcttattatcagcaacccgatcaaccgttaggggtttgggtggtaccggaaggccattactttatgatgggtgataaccgggataacagtgcagatagccgcttctggggttttgtaccagaacgtaatctggtaggtaaggctacggctatttggatgagttttgaaaagcaagaaggtgaatggccaacgggtgtgcgtttaagccgaattggtggaattcactaa

SEQ ID NO: 168 - protein, 194 aa, Staphylococcus aureus
VSKLKKEILEWIISIAVAFVILFIVGKFIVTPYTIKGESMDPTLKDGERVAVNIVGYKTGGLEKGNVVVFHANKNDDYVKRVIGVPGDKVEYKNDTLYVNGKKQDEPYLNYNLKHKQGDYITGTFQVKDLPNANPKSNVIPKGKYLVLGDNREVSKDSRAFGLIDEDQIVGKVSFRFWPFSEFKHNFNPENTKN

SEQ ID NO: 169 - DNA, 585 bp, Staphylococcus aureus
gtgtcaaaattgaaaaaagaaatattggaatggattatttcaattgcagtcgcttttgtcattttatttatagtaggtaaatttattgttacgccatatacaattaaaggtgaatcaatggatccaactttgaaagatggcgagcgagtagctgtaaacattgttggatataaaacaggtggtttggaaaaaggtaatgtagttgtcttccatgcaaacaaaaatgatgactatgttaaacgtgtcatcggtgttcctggtgataaagtagaatacaaaaatgatacattatatgtcaatggtaaaaaacaagatgaaccatatttaaactacaatttaaaacataaacaaggtgattacattactgggactttccaagttaaagatttaccgaatgcgaatcctaaatcaaatgtcattccaaaaggtaaatatttagtgcttggagataatcgtgaagtaagtaaagatagccgtgcgtttggcctcattgatgaagaccaaattgttggtaaagtttcatttaggttctggccatttagtgaatttaaacataatttcaatcctgaaaatactaaaaattaa

SEQ ID NO: 170 - protein, 298 aa, Vibrio cholerae
MANTFSLILVIVTLVTGIVWTLEKLVWAKKRQQKQAHLQAQTPDMPASALDKVVAQPWWIENSVSIFPVIAFVLVLRSFIYEPFQIPSGSMMPTLLVGDFILVEKYAYGLKDPVWRTQLVETGKPERGDIVVFKYPVNPEIDYIKRVVGMPGDTVRYSAGKELCIQHQGESECQAVKLSNVQESEFYQNEIPLIQLNEQLGKVEHNILVNPLSIDNVANYRPRSGVNEWVVPQGHYFVMGDNRDNSADSRFWGFVPEQNLVGKAVAIWISFEFERAEDSVLPRWIPTGVRFNRVGGIH

SEQ ID NO: 171 - DNA, 897 bp, Vibrio cholerae
atggcgaacacattctcactgattttggtgatcgtaactctggtcaccggtatcgtctggacactggaaaagctggtgtgggcgaaaaaacgccaacagaaacaagctcatttacaggcgcaaacgcccgatatgccagcctcagcgctggataaagtcgtggctcagccgtggtggattgaaaacagtgtctcgattttccctgttattgcttttgtgctggtactgcgctcgttcatttatgaaccgttccaaattccatccggttcgatgatgccgactctgctggtcggggattttattctggttgagaaatacgcttacggcttgaaagatcctgtatggcgcactcagttagtggaaacgggtaaacctgagcgtggtgatattgtggtgttcaaatacccagtgaaccctgagatcgactacatcaaacgtgtggtggggatgcccggagataccgtacgttacagcgcaggtaaagagctgtgtattcagcaccaaggcgagagcgaatgccaagcagttaaactctctaacgtgcaagagagcgagttttaccaaaatgagatccccctgatccagctgaacgaacagctaggtaaggttgagcacaatattttggttaacccattgagcattgataacgtggcgaattatcgcccacgcagtggcgtgaatgaatgggttgtaccacaagggcactattttgtgatgggtgataaccgtgacaacagtgctgacagccgtttctggggctttgtgccagagcagaatctggtcggaaaagctgtggctatctggatcagtttcgagtttgaacgcgctgaagacagcgtacttccacgctggattcctaccggagtacgattcaatcgtgttggtgggatccactaa

SEQ ID NO: 172 - protein, 349 aa, Haemophilus influenzae
MSNLFFVILLAVGFGVWKVLDYFQLPNTFSILLLILTALSGVLWCYHRFVVLPKRHRQVARAEQRSGKTLSEEEKAKIEPISEASEFLSSLFPVLAVVFLVRSFLFEPFQIPSGSMESTLRVGDFLVVNKYAYGVKDPIFQNTIIEGEKPQRGDVIVFKAPQQALIRTGLGATRAAFAENLALSSKDNMSGVDYIKRIVGKGGDRIIFDVEQKTLKIVYGKDGKPCEVDCETKAFEYTQNPTNPAFPNELELTEKGDVTHNVLIGEYRRYSDLEFFPQEGMQTAEWLVPEGQYFVMGDHRDHSDDSRFWGFVPEKNIVGKATYIWMSLEKEANEWPTGFRFDRFFTAIK

SEQ ID NO: 173 - DNA, 1050 bp, Haemophilus influenzae
atgtcaaatttattttttgtgattttattggctgtcggctttggtgtgtggaaagttttagattattttcagttgccaaatacttttagtattttgttactaattttgaccgcactttctggcgtattatggtgttatcatcgttttgtggtgctgccaaaacgtcatcgtcaagtggcacgtgcagaacaacgttctggtaaaaccttaagtgaggaagaaaaagccaaaattgaaccgatttctgaggcttcagaatttttgtcttcactttttcctgtgcttgcagtggtatttttggttcgttcttttttgtttgaaccgtttcaaattccctctggctcaatggagtccactttacgcgttggcgattttttagttgtgaataaatatgcttatggtgtgaaagatccgattttccaaaacaccattattgagggcgaaaaaccacaacgtggcgatgtgattgtgtttaaagcaccacaacaagcgttaattcgtactggtcttggggctactcgagcggcttttgcagaaaatttagcgttaagttcaaaagataatatgtctggtgtggattatattaagcgtattgttggaaagggcggagatcgcatcatttttgatgtggaacaaaaaacattaaaaattgtatatggcaaagatggtaaaccttgtgaagttgattgcgaaaccaaggcgtttgaatatacacaaaatccaacaaatcctgcttttccgaatgaattagaattgactgaaaaaggcgatgtaacacataacgtgttaattggtgagtatcgtcgttattcagaccttgaatttttcccacaagagggaatgcaaactgcagaatggcttgtgccagaggggcagtattttgtgatgggggatcatcgcgatcacagcgatgacagtcgtttttggggctttgtgcctgaaaaaaatattgtggggaaagccacttatatttggatgagcttagaaaaagaagcgaatgaatggccaacaggtttccgttttgatcgcttctttacagcaataaaataa

SEQ ID NO: 174 - protein, 284 aa, Pseudomonas aeruginosa
MTLNFPLLLVIAVAVCGALALVDLVLFAPRRRAAISSYEGQVNEPDPAVLEKLNKEPLLVEYGKSFFPVLFIVLVLRSFLVEPFQIPSGSMKPTLEVGDFILVNKFAYGIRLPVLDTKVIPIGDPQRGDVMVFRYPSEPNINYIKRVVGLPGDTVRYTKEKRLYVNGELVAEKLVGEEPGTLGSVTLYQEKLGQAEHLIRKEMSRYRIEPDRQWTIPAGHYFMMGDNRDNSNDSRYWNDPKIPKDLLGMVPDRNIVGKAFAVWMSWPDPKMSNLPNFSRVGVIH

SEQ ID NO: 175 - DNA, 855 bp, Pseudomonas aeruginosa
atgacactcaatttcccgttgttgctggtcatcgccgtggctgtatgcggcgccctggccctggtcgacctggtgctgttcgcgccgcgtcggcgggccgcgatctcttcctacgaagggcaggtgaacgagcccgatccggcagtgctggagaagctcaacaaggaaccgctgctggtggagtacggcaagtcgttcttcccggtgctgttcatcgtgctggtgctgcgttccttcctggtcgagccgttccagattccctcggggtcgatgaaacctaccctcgaggtcggcgatttcatcctggtcaacaagttcgcctacggtatccgcctgccggtgctggacaccaaggtgatcccgatcggtgatccgcagcgcggcgatgtcatggtgttccgctatcccagcgaaccgaacatcaactacatcaagcgcgtggtcggcctgcccggcgacaccgtgcgctacaccaaggaaaagcgcctgtacgtcaacggcgagctggtggcggagaaactggtcggcgaggaaccgggcaccctgggcagcgtgaccctgtaccaggagaagctgggccaggccgagcacctgatccgcaaggaaatgagccgctatcgcatcgagcccgaccgccagtggaccattcccgccggccactacttcatgatgggcgacaaccgcgacaactccaacgacagccgctactggaacgatccgaagatccccaaggatctgctgggcatggttccggaccgcaatatcgtcggcaaggccttcgccgtgtggatgagctggcccgatccgaagatgagcaacctgccgaacttctcccgggtcggcgtgattcactga

SEQ ID NO: 176 - protein, 275 aa, Acinetobacter baumannii
VDFDFNLILVPVTLILFAVWLLDKLVFKQRANKGRENENFVITWAYDFWPVLAVVLVLRSFLYEPFNIPSDSMVPTLETGDFILVNKFDYGVRLPIVNKKVIDVGEPKRGDVIVFRYPPQPTISYIKRVIGLPGDHIVYDHGQLIINGQKIPKVPTQFSREKDALDTPTSIYHKETIGDHTFTMRELEGVNVARQAPFINYVDNGKYANQDGLYWEVTVPKGHYFAMGDNRDQSADSRFWGFVPEENLTGRAFYVWMHKEPGFHLPSFNRNGKID

SEQ ID NO: 177 - DNA, 828 bp, Acinetobacter baumannii
gtggattttgattttaatttaattcttgttcctgttacgctgattttatttgcagtgtggttgctagataagcttgtttttaaacagcgtgcaaataaagggcgagagaacgaaaattttgttattacatgggcctatgacttttggccggttttagctgttgtgcttgtacttcgctcatttctttatgaaccatttaatattccatcagactctatggttccgaccttagagactggcgattttattttagttaataaatttgactatggtgtccgtttacctatcgtcaataaaaaagtgattgatgtcggtgaaccgaaacgtggtgatgtcattgtattccgttatccaccacaacctactattagttatattaaacgtgtaattggcttacctggtgaccatattgtttatgatcatggacaattgattattaatggtcaaaaaattcctaaagtaccaacacagtttagtcgcgaaaaagatgctttagatacaccaacttctatttatcataaagaaacaattggtgatcatacttttacgatgcgtgagcttgaaggcgtaaatgttgcgcgtcaggcgccatttatcaactatgttgataatggtaaatatgcaaaccaagacggtttatattgggaagtaacagttccgaaaggacattactttgcaatgggggataaccgtgatcaaagtgctgacagtcgtttctggggcttcgtacctgaagaaaatttaacaggccgagctttctatgtctggatgcataaagaacctggtttccacctgccaagctttaaccgaaatgggaaaatagattaa

SEQ ID NO: 178 - protein, 183 aa, Bacillus anthracis
MKENTKKELFSWAKTIGFTLVLIAIIRGVLFTPSLVQGESMMPTLENNERVLVNKIGYSISGLERFDIIVFHGKEGYDLVKRVIGLPGDTVEYKNDVLYVNGKAMEEPYLKEFKEKAAGRVLTPDFTLEQITGKTKVPEGQVFVLGDNREVSKDGRMFGFISEDEIVGKGQAVFWPLKQVRAL

SEQ ID NO: 179 - DNA, 552 bp, Bacillus anthracis
atgaaggaaaatacgaagaaagaattattctcatgggcgaaaacgataggatttacccttgtattaatcgcaattattcgcggtgttttatttacaccgtcattagtacaaggcgaatcgatgatgccgactttagaaaataacgaacgagttctcgtcaataagattggttatagtataagtggattagaacgctttgatattatcgttttccatggaaaagaaggatatgatttagtaaaacgagtaattggtttaccaggcgatacagttgagtataaaaatgatgttttatatgtaaacggcaaagcgatggaagaaccatatttaaaagagtttaaagaaaaagcagcaggtcgtgtattaactccagactttacgttagaacaaattacaggaaaaacgaaagtgccagaaggccaagtgtttgtattaggtgataatcgtgaagtttctaaagacggtcgtatgtttggatttatttcagaagatgaaattgtcggaaaaggacaagctgttttctggccgttgaaacaagtaagagcgctataa

SEQ ID NO: 180 - protein, 339 aa, Neisseria meningitidis
MNTMLMSGAAAALLAGIILYFKSDKKRQENGEWSSGLEYAYILTAVGVFAALSLFMSFTAVFLIFVVLCGTAWGVYKYRLKTHPEISESSHFGDYFGSFFPTVLVLFLIRSFIAEPFQIPSSSMRPGLIKGDFILVGKFSYGLRVPVLNNIFIPTGKIERGDVVVFNYPLQPEMTYIKRIVGIPGDVVEYRDKILTVNGKPTSDIPDGTYRYPDDTDPSEIHNTDMFRSGLDGKSFNILKKEGQPAVSLPVLGKYTSDIMSENGYSIEQSGLEHCQYADDGSGFVCKVPEGRYFAMGDNRDNSADSRYWGFVDDKLVVGKAMFILMNFGDFGRSGTAIR

SEQ ID NO: 181 - DNA, 1020 bp, Neisseria meningitidis
atgaacacaatgctaatgtcgggcgcggctgccgcgctgcttgccggcatcatcctttatttcaaaagcgacaagaagcggcaggaaaacggggaatggagttccggccttgaatacgcctatatcctgacagcggtcggcgtgtttgccgctttgtccctgtttatgagctttaccgccgttttcctgattttcgttgtattgtgcggtacggcttggggggtatataaataccgcctgaagactcatcccgaaatctcggaaagcagccacttcggcgattatttcggcagtttcttccctaccgttttggtattgttcctcatccggtcgtttatcgccgaaccgttccaaatcccgtccagctcgatgcgcccgggcctgatcaagggcgatttcattttggtcggcaaattttcctacggcctgcgcgtacccgttttaaacaatatatttattcctacaggcaaaatcgaacggggcgatgtcgttgtttttaattatcctctgcagccggagatgacctacatcaagcgtattgtcggcattccgggcgatgtggtcgaatatcgggataagattttgacggtaaatggcaaacccacttccgacattcctgacggcacataccgttatcccgacgacaccgacccttccgaaatccacaacacggatatgttccgcagcggtctagacggcaaatccttcaatattctgaaaaaagaaggacagcctgccgtttccctgcccgtattgggcaaatatacctccgatattatgtctgaaaacggatattccatagagcaaagcggtttggaacactgccaatatgccgacgacggcagcggtttcgtgtgcaaagttcccgaaggacgctatttcgctatgggcgacaaccgcgacaacagtgccgattcgcgctactggggatttgtggatgacaagctggttgtcggcaaggcaatgttcattttgatgaacttcggcgatttcggcaggtccggtacggcaatccgttag

SEQ ID NO: 182 - protein, 173 aa, Bacillus anthracis
MKQEIKRGWGKYILFVFVLVVAYHSFTLCKVEGKSMQPTLYEEDYVFVNKAAVHFSDLEHGEIVIIKEEDESKYYVKRVIGLPGDVINITNGSVYVNDKKQEEPYTNKDLFNNTQVFYNFQKTKIPPNKLFVMGDNRELSRDSRNGLGYIEEDNIIGKVEFVYYPFSKMKIIE

SEQ ID NO: 183 - DNA, 522 bp, Bacillus anthracis
atgaaacaggagattaaaagaggttgggggaaatatatactcttcgtgtttgttttggtagtagcttatcattcttttactttatgtaaagtggaagggaaatcaatgcaaccgactttatatgaagaagactacgtatttgtaaataaagcagcagtacatttttccgatttagagcatggagaaattgtcattataaaggaagaggatgaatcgaaatattatgtaaaacgagtaataggacttcctggtgacgtaattaacataacgaatggatctgtatatgtaaatgataaaaaacaagaagaaccgtatacaaataaagatttattcaataatacgcaagtgttttataactttcaaaagacaaaaatcccaccaaataaattatttgtaatgggagataatcgtgaacttagtagagatagtcgaaacggtttaggatatattgaagaagataatataataggcaaagtggaatttgtatattatcctttttcaaaaatgaagatcatagaataa

SEQ ID NO: 184 - protein, 195 aa, Streptococcus mutans
MKRFLKEWGLFLVIIFALLLPRLFIWFPVQVDGHSMDPTLANGEHLIVVRTTSIKHFDIVVAAEGNKNIVKRVIGMPGDTITYENDMLSINGKKVNETYLKQYKDKFAKDKLQKTYAYNQYFQELASQSTAFTTDEQGNASFTIKVPKGRYLLLGDDRIVSKDSRHVGTFAKNKIVGEVKFRFWPLNAIRFISNK

SEQ ID NO: 185 - DNA, 588 bp, Streptococcus mutans
atgaaaagatttttaaaagaatggggccttttcttggtcatcattttcgcattgctactcccgcgtctctttatctggtttcctgtccaagtagatggacattcaatggatcctaccttagccaatggggagcatctcattgtcgtcaggacaacttctatcaaacattttgacattgttgttgctgctgaaggcaataaaaatattgtcaaacgtgtgattggcatgcccggtgataccattacctatgaaaatgatatgctttctattaatgggaaaaaagtcaatgaaacttatctcaagcaatacaaggataaatttgccaaggacaaactccaaaagacttatgcctacaatcagtatttccaagaattagcctcacaatcaacagctttcacaacagacgaacaaggaaacgccagctttacgattaaagtaccaaaaggacgttacctgcttttaggtgatgatcgcattgtctctaaagacagccgccatgttggaacttttgctaagaataaaattgttggtgaagttaaattccgcttttggcctttaaacgctattcgtttcatttcaaataaataa

SEQ ID NO: 186 - protein, 324 aa, Shigella flexneri
MANMFALILVIATLVTGILWCVDKFFFAPKRRERQAAAQAAAGDSLDKATLKKVAPKPGWLETGASVFPVLAIVLIVRSFIYEPFQIPSGSMMPTLLIGDFILVEKFAYGIKDPIYQKTLIETGHPKRGDIVVFKYPEDPKLDYIKRAVGLPGDKVTYDPVSKELTIQPGCSSGQACENALPVTYSNVEPSDFVQTFSRRNGGEATSGFFEVPKNETKENGIRLSERKETLGDVTHRILTVPIAQDQVGMYYQQPGQQLATWIVPPGQYFMMGDNRDNSADSRYWGFVPEANLVGRATAIWMSFDKQEGEWPTGVRLSRIGGIH

SEQ ID NO: 187 - DNA, 975 bp, Shigella flexneri
atggcgaatatgtttgccctgattctggtgattgccacactggtgacgggcattttatggtgcgtggataaattctttttcgcacctaaacggcgggaacgtcaggcagcggcgcaggcggctgccggtgactcactggataaagcaacgttgaaaaaggttgcaccgaagcctggctggctggaaaccggagcttctgtttttccggtgctggctatcgtattgattgtacgttcgtttatttatgaaccgttccagatcccgtcaggttcgatgatgccgactctgttaatcggtgattttattctggtagagaagtttgcttatggcattaaagatcctatctaccagaaaacgctgatcgaaaccggtcatccgaaacgcggcgatatcgtggtctttaaatatccggaagatccaaagcttgattacatcaagcgcgcggtgggtttaccgggcgataaagtcacttacgatccggtctcaaaagagctgacgattcaaccgggatgcagttccggccaggcgtgtgaaaacgcgctgccggtcacctactcaaacgtggaaccgagcgatttcgttcagaccttctcacgccgtaatggtggggaagcgaccagcggattctttgaagtgccgaaaaacgaaaccaaagaaaatggaattcgtctttccgagcgtaaagagacactgggtgatgtgacgcaccgaattctgacagtgccgattgcgcaggaccaggtggggatgtattaccagcagccagggcaacaactggcaacctggattgttccgccgggacaatacttcatgatgggcgacaaccgcgacaacagcgcggacagccgttactggggctttgtgcctgaagcgaatctggtcggtcgggccacggctatctggatgagcttcgataagcaagaaggcgaatggccgactggtgtgcgcttaagtcgcattggcggcatccattaa

SEQ ID NO: 188 - protein, 324 aa, Citrobacter koseri
MANMFALILVIATLVTGILWCVDKFIFAPKRRERQAAAQAAAGDSLDKATLKKVAPKPGWLETGASVFPVLAIVLVVRSFIYEPFQIPSGSMMPTLLIGDFILVEKFAYGIKDPIYQKTLIETGHPKRGDIVVFKYPEDPRLDYIKRAVGLPGDKVTYDPVAKEVTVQPGCRSGQACENALPVTYSDVQPSDFVQTFARRNGGEASSGFFEVPLNETKDNGIRLAERKETLGDVTHRILTVPIAQDQAGMYYRQPGQQLATWIVPPGQYFMMGDNRDNSADSRYWGFVPEANLVGKATAIWMSFDKQEGEWPTGVRLSRIGGIH

SEQ ID NO: 189 - DNA, 975 bp, Citrobacter koseri
atggcgaatatgtttgccctgattctggtgattgccacactggtgacgggcattttatggtgcgttgataaatttatcttcgcgccaaaacgtcgggaacgtcaggcagcggcacaggccgctgcgggtgattcactggataaagccacgttgaaaaaagtggcgcctaagccgggctggctggaaacaggggcttcggtttttccggtactggcgattgtgctggtggtgcgctcatttatctatgaacctttccagatcccgtcgggttcgatgatgccgacgctgttaatcggtgactttattctggtggagaaattcgcctatggaattaaagatccgatttaccagaaaacgttgattgaaacgggtcatccgaaacgcggtgatatcgtggtctttaaatacccggaagatccgcgcctggactacattaaacgcgctgtcggcctgccgggtgacaaagtgacgtacgatccggtagccaaagaggttactgtacagccaggatgccgttccggtcaggcgtgtgaaaacgcgctgccggtgacttactctgacgttcagcccagcgatttcgtgcagacctttgcccgccgtaatgggggagaagccagcagtgggttcttcgaagtgccgttaaacgaaacgaaagataacggcattcgtctggcggagcgtaaagagacgctgggagacgtaacccaccgtattctgaccgtaccgatcgcgcaggatcaggcggggatgtattaccgtcagccggggcagcaactggcgacctggatcgtaccgccaggacaatacttcatgatgggtgataaccgcgataacagcgcggacagccgttactggggatttgtaccggaagcgaatctggttggtaaagcgaccgcgatctggatgagtttcgacaaacaggaaggtgaatggccgaccggcgtacgcttaagccgtattggtgggatccattaa

SEQ ID NO: 190 - protein, 294 aa, Bordetella pertussis
MSWNFALILFVLLVITGVIWGLDLALFRKRRERRAQAAAAQVDAAGITDAEQAGRERREAIDAARRAPWWIEYAVSFFPVILFVFVLRSFVVEPFHIPSGSMLPTLQSGDLILVNKFSYGIRLPIIDRKIIETGSLERGDVVVFRYPVDTDVDYIKRIVGLPGDQVAYLDKKLYINGKLVPHERDGDYFEPDRVSYIAQYKEKLGEVEHKILLDEQKIQDFGPIWKFPSIQNCQYARNGVRCTVPPGHYFAMGDNRDNSADSRYWGFVPDGNIVGKAFFVWMNFSDLSRIGRFH

SEQ ID NO: 191 - DNA, 885 bp, Bordetella pertussis
atgagttggaactttgccctgatactttttgtactgctggtgattaccggcgttatctggggattggatctggcgctgtttcgcaagcgacgcgaacggcgggcccaggcggcggccgcgcaagtggacgccgccggcatcacggatgccgagcaggccggccgcgagcggcgcgaggccatcgacgcggcgcgccgcgcgccctggtggatcgagtatgcggtcagcttcttcccggtgatcctgttcgtgttcgtgctgcgctcgttcgtggtcgagccgtttcacattccgtcggggtccatgctgcccacgctgcaatcgggcgacctgatcctggtgaacaagttcagctacggcatccgcctgcccatcatcgatcgcaagatcatcgagacgggctcgctggagcgtggcgacgtggtggtgttccgctacccggtcgatacggatgtcgactacatcaagcgcatcgtgggtctgccgggcgaccaggtggcctacctggacaagaagctgtacatcaacggaaaattggtgccgcatgaacgcgacggggattatttcgagcccgatcgcgtgtcctatattgcgcaatacaaggaaaaactgggcgaagtggagcataagatcctgcttgatgagcagaaaatacaggatttcggccccatctggaaatttcccagtatccagaactgccagtacgcccgcaacggcgtgcgctgtaccgtcccccccggccattatttcgccatgggagacaaccgtgacaatagtgcggacagccgctactggggattcgtgccagacggtaatatcgtggggaaggcattttttgtctggatgaacttcagcgatttgagccgcattggccgcttccattga

SEQ ID NO: 192 - protein, 176 aa, Clostridium difficile
MSVKKEIFDWIKSIAMAIVLAFVILQFIIPSIVSGESMYPTLDDKDYLILNRISYKVGKPEKGDIVVFKTNLVDGETGKKKDLIKRVIATEGDRIKISNSKVYVNGKLLNEPYIHNNYTSGDIDTVVPKGKLFAMGDNRENSNDSRFPDVGMVDEDEVLGKVMVRLLPLDNIGKVD

SEQ ID NO: 193 - DNA, 530 bp, Clostridium difficile
atgagtgttaaaaaagaaatatttgattggattaagtcaatagctatggctattgtacttgcatttgtaattctacaatttataataccttctattgtaagtggagaatcaatgtatcctactttagatgataaagattatctgattttaaataggatatcatacaaggttggtaaacctgaaaaaggcgatattgtagtttttaaaaccaatttagttgatggagaaacaggaaagaaaaaagacttaataaaaagagttatagctactgaaggtgacagaataaaaatatcaaattctaaagtgtatgtaaatggaaaattattaaatgaaccatatatacacaataactatacttctggagatatagatactgttgttccaaaaggtaaactatttgcaatgggagataatagagaaaatagtaatgatagtagattccctgatgtaggtatggttgatgaagatgaagttcttggtaaggttatggtgagactattacctcttgataatattgggaaagtagacta

SEQ ID NO: 194 - protein, 178 aa, Clostridium difficile
VGEAVKKEVVEWIKVIVIALVLAFAITRFIVPTIVKGESMYPTLVERDYLIVNRIAYKVGEPKYKDIIVFKTDLTEENGKKKDLVKRVIGVPGDHVKIQDSKVYVNDKLLDETSYIHNNRTDGDIDIVVPEGKLFAMGDNREKSLDSRYDEVGLVDEHTILGKVLVRLYPFSKIGTID

SEQ ID NO: 195 - DNA, 537 bp, Clostridium difficile
gtgggtgaagcagttaaaaaagaagttgtagaatggataaaagtgattgtcatagctcttgttttggcatttgcaataactcgttttatagtgccaacaatagtcaaaggagaatcaatgtatcctacattagttgaacgtgattatttgatagttaacagaattgcgtacaaggtaggagagccaaaatacaaagatataatagtattcaaaaccgacttaacagaggaaaatggaaagaaaaaagatttagtaaaaagagttatcggggttcctggtgaccatgtaaaaatacaagactccaaggtatatgtaaatgataagttgttagatgagacttcctatatacataataatcgtactgatggagatattgatatcgtagttccagaaggaaaattatttgcaatgggagataatagagaaaaaagtttagatagtagatacgatgaggttggattggtcgacgagcataccattttaggaaaggttctagtcagattgtatccattttctaagataggaactattgactaa

SEQ ID NO: 196 - protein, 178 aa, Clostridium difficile
MNETIKEEIVEWIKIIITALFFAFIITRFIKPTLVNGESMYPTLKSHDYLVANRMTYKLSEPKCGDIMIFKTDLLQENGRKKELVKRVIGVPGDHLKIKDSKVYINGKLLNEVSYIHDNYTEGDIDMVIPKGKVFAMGDNREVSLDSRYKEVGLVDEENIKGKVILRVFPFTDIGIFE

SEQ ID NO: 197 - DNA, 537 bp, Clostridium difficile
atgaatgaaactattaaagaagagattgtagagtggataaaaataattattactgcacttttttttgcatttattataactcgttttataaaaccaacattagtaaatggagaatcaatgtacccaacacttaaatcacatgattatttggtagcaaacaggatgacatataagttatcagaaccaaaatgtggagatataatgatatttaagactgatttattacaagagaatggaaggaaaaaagagcttgtaaaaagggttataggtgttcctggtgaccatctaaaaattaaggattctaaggtttatataaatggtaagttattaaatgaagtttcatatatacatgataattatactgaaggcgatattgatatggtgattcctaagggaaaagtatttgcgatgggagacaatagagaagttagtttagacagtagatataaagaagtgggattagtagatgaagaaaatattaaaggaaaagttattttaagagtatttccttttacagatataggtatttttgagtag

SEQ ID NO: 198 - protein, 178 aa, Enterococcus faecalis
MSSLLKRLVQLVLLVVAVLLIRHYVFSPAAVNGSSMEPTLHNNDRLWVTSIKKPQRFDIIAFPSPRNGQRVAKRLIGLPGETVEYRDDTLYINGVSLSEDYLASAKRNVSKNENYTQDFTLETLEATQSLTVPEGMYFVLGDNRPRSDDSRYFGFVKQASVEGVLTFRYYPLDKIGFP

SEQ ID NO: 199 - DNA, 537 bp, Enterococcus faecalis
atgtcctcattattaaaacgattggttcagttggttttgttagtcgtcgctgtcttgctgattcgacactatgttttctcccctgctgcggtgaacggctcttcaatggaaccaacacttcataacaacgaccgtttatgggtgacctcgattaaaaaaccacagcgctttgatattatcgctttccctagtcctcgcaacggccaacgagtagccaaacgtttaattggtttacctggcgaaacagtcgagtatcgcgatgataccctttatattaatggtgtatcactcagtgaagattacttagcaagtgctaaacgaaatgtctctaaaaatgaaaattatacccaagattttacgctagagaccttagaagccacccaatccctgaccgttccagaaggcatgtattttgtcttgggggataatcgcccgcgctcagacgacagtcgttattttggctttgttaaacaagcgagtgtggaaggtgttttgacttttcgttattatccattagataaaattggctttccataa

SEQ ID NO: 200 - protein, 241 aa, Enterococcus faecalis
MRTIRHIKRAFLKQKLPATYQLKKQKANTAMEYLLEQTDNHQSIRGPKRKMTAEEIKKKRQAYQKKQRVQVVKFFMPAILFAIFVFFFVLKTSSYPIAGQSMKPTLNAGERVLVQRTKQVARYDVIAFKAPLASKGTYVKRIIGVPGDRIWVNEGKLYLSEEPIASDNEALPENASRFDLSEEAAAQLRLFQKIPAGHYFVLGDNRTHSSDSRTFGFVEIQAIEGIVVFKMAPFKEIGKVK

SEQ ID NO: 201 - DNA, 726 bp, Enterococcus faecalis
atgcgaacaattcgccacattaagcgcgccttcttgaagcaaaagttgcctgcgacatatcagctaaaaaagcaaaaggctaacacagcaatggaatatttgcttgagcaaacagataaccatcaatcaataagaggaccgaaaagaaaaatgaccgctgaagagattaaaaaaaagcggcaagcctaccaaaagaaacaacgcgtccaagtcgttaaattttttatgccagctattcttttcgccatttttgtgttcttttttgtgttaaagacatctagctacccaattgctgggcaatccatgaagccgacacttaacgcaggggaacgagtcttagtacaacggacgaagcaagtagcaaggtacgatgtgattgcatttaaagcaccgctagctagcaaaggtacgtacgtcaagcgaatcatcggggttcctggtgatcgaatttgggtaaacgagggaaaactttatctttcagaagaacctatagcaagcgataatgaggcactgcctgagaatgccagtcgttttgacttatcagaagaagcggcagcccaacttcgcctgtttcagaagattccagctggtcattactttgtcttaggggacaatcgtacgcattcaagtgatagtcgtacgttcggctttgtcgagatacaagcgattgaaggaatcgtggtatttaaaatggcgccgtttaaggaaatagggaaagtaaaataa

SEQ ID NO: 202 - protein, 182 aa, Enterococcus faecalis
MSLKSKELIKTVVFFACLALGLFLLRQFVFTPVVVRGHSMDPTLADGERVITLKNTEINRFDIITFPAPDEPDKNYIKRVIGLPGDTIAYKDDTLYINGKEVDEPYLDEFKKALTDGQPLTGDFSLKEKVPADSYFVLGDNRRNSKDGRVIGFIHKKDILGEVKFVMWPFSRFGPIPEVSKQ

SEQ ID NO: 203 - DNA, 549 bp, Enterococcus faecalis
atgagtttgaaatcaaaagaattaattaaaacagtcgtcttttttgcctgtttagctttgggtctgtttttactgagacaatttgtatttacgcctgtcgtagtgagaggtcattcaatggatccaacgttagcagatggtgaacgggtaattacgttaaaaaacacagaaattaatcgtttcgatattattactttcccagcgccagatgaaccagataaaaattatattaaacgtgtgattggtttacctggagatacaattgcgtacaaggatgatacgttgtacatcaatggaaaagaagttgacgaaccctatttagatgaatttaaaaaagccttaacagatggtcaacctttgacaggcgatttttcattaaaagaaaaagtaccagcagatagctactttgttttaggtgataatcgacggaattcaaaagacggtcgtgtcattggttttattcataaaaaagatattttgggtgaagtgaaatttgtgatgtggccattctcacggtttggtccaataccagaagtgtcaaaacaataa

SEQ ID NO: 204 - protein, 184 aa, Enterococcus faecalis
LKKKRDYVGYLMYFLKILVPAIVAVFILRGFFLIPVRVDGHSMQKTLNQGDMIVMEKFSAIKRFDVVVFKTDTGSILIKRVIGLPGEAVRYENDQLYVNNQPIAEPYLTKNRKKDHETMPYTTNFDSKELLMQEKLPKDSYFVLGDNRRMSKDSRSFGAIHADQILGKAQFVYYPLTHMKIIPK

SEQ ID NO: 205 - DNA, 555 bp, Enterococcus faecalis
ttgaagaagaaacgtgattatgttgggtatttaatgtactttctgaaaattttagtaccagcaatcgtagccgtttttattttaagaggatttttcctgattcctgttcgggtggatggccattctatgcaaaaaaccttgaatcaaggagatatgattgtgatggaaaaattctccgccattaaacggtttgatgtggtggtctttaaaacagatacaggatcgattctgattaaacgtgtgattggtttaccaggagaagctgtgcgttacgaaaacgatcaattatatgtcaataatcagccaatcgctgaaccgtatttaactaaaaacagaaaaaaagatcatgaaacgatgccttacactacgaattttgattcaaaagaattgttaatgcaagaaaaattacctaaagatagctattttgtgcttggtgataatcgccgtatgtccaaagacagccgttcttttggtgcaatacatgcagatcaaatcttagggaaagcacaatttgtttattacccactcactcatatgaagatcattcctaaataa

SEQ ID NO: 206 - protein, 189 aa, Listeria monocytogenes
MTDQYDKKPKKKSGAHQLLSWVLVIVAALAIALVIRNFVVAPVKVEGTSMVPTYQDGDRIFIEKISKPDRFDIIVFDEPPMIGSGEHFIKRVIGLPGDKIAFKNGELYLNGKRKVENYLPEGTLTLWNPDPTQKPYIADYTLEDMTGESTVPKGKLFVLGDNRGGSSDSRVFGFIDDSMVNGTVIQFGK

SEQ ID NO: 207 - DNA, 570 bp, Listeria monocytogenes
atgacagatcaatatgacaaaaagcccaagaaaaaaagcggggcgcaccaattattaagctgggtgctagttatcgttgcagcgcttgcaattgcacttgtgattcgtaactttgtagttgcaccagtaaaagtagaaggaacatctatggttccaacatatcaagatggcgatagaattttcattgaaaaaatttccaagcctgatcgtttcgacattatcgtgtttgatgaacctccaatgattggttcaggagagcatttcatcaagcgagtgattggtttgccgggagataaaatagcatttaaaaacggtgaattatatttaaatggaaaacgaaaagtagaaaattacttgccagaaggaacattaaccctttggaatccagatccaacgcaaaaaccatacatagcggattatacgctggaggatatgacaggcgaaagtactgttccgaaagggaaactatttgtacttggagataatcgcggcgggagttcagatagtcgcgttttcggatttattgatgattccatggtaaacggtacagtgatacaatttggaaaataa

SEQ ID NO: 208 - protein, 188 aa, Listeria monocytogenes
MKSENKFFSGAFGWIKIILIALILAFGIRYFLISPVTVNGKSMDPTLHDGEHLFINKVSDPKRFDIIVFPAPDEENAEYIKRVIGLPGDKVEYKEDQLYINGKKYDEPYLDSEKEALKNGYLTTDAEGDPNFTMADIPNSDGSLTVPKGELFVLGDNRQVSKDSRYIGFISQDTVLGKVISFGKSLER

SEQ ID NO: 209 - DNA, 567 bp, Listeria monocytogenes
atgaaaagtgaaaacaaatttttttctggggcatttggatggataaaaataattctcatcgcgcttatacttgcttttggtattcgctattttttaatttctccagttactgttaatgggaaatcaatggacccaacacttcatgatggggaacatttatttattaacaaggtatcagatccgaagcgttttgacattattgtatttcctgcgcctgatgaggaaaatgcagagtacattaaacgcgtcattggccttccaggagataaagtggagtacaaagaagatcaactttatattaatggaaaaaaatatgatgaaccttatttagattcagaaaaagaagctctaaaaaacggttatttaaccactgatgcagaaggcgatcctaattttacgatggcagacattccaaactctgacggctctctcactgtccctaaaggagaactttttgttttaggagataatcgtcaagtaagtaaagatagtcgctacattggctttatatcacaggataccgtgcttggaaaagtaatttcatttggaaaatccttagaacgttaa

SEQ ID NO: 210 - protein, 180 aa, Listeria monocytogenes
LKEKNLKRLWSWIWAAVLAVLIAVIIRFYLFVPILVDGISMMPTLHSDDRVIINRFGNVDRFDVIVFRESDGKEYIKRVIGLPGDTVEYKEDQLYINGKKYNEPYLDTYKEKLKDGYLTDDYSSKDQLDGGKIPKDTYFVLGDNRRASKDSRIIGPIPFSKVLGTTPICYWPIEDAKLID

SEQ ID NO: 211 - DNA, 543 bp, Listeria monocytogenes
ttgaaggagaagaatttaaaacggttatggtcatggatttgggcggctgttctagcagtgttaatagctgttataatccgtttttatttatttgtccctattctcgtcgatgggatatcaatgatgcctacacttcatagcgatgaccgtgtaattataaatcgcttcggaaatgtagatcgtttcgatgtgattgttttccgagaatcagatggaaaagaatacatcaagcgagtgatcggtttgccgggtgatacagtagaatacaaagaagaccaactttacatcaatggtaaaaagtataatgaaccatatttggatacttacaaagaaaagttaaaagatggctatttaacagatgattacagttcgaaagatcaactagatggtggcaaaattccaaaagatacttattttgttttaggtgacaatcgaagagcaagcaaagacagtcggataattgggccaattccatttagcaaggtgttaggaacaacaccgatttgttactggccgattgaagatgccaaacttatagattag

(19) United States
(12) Patent Application Publication
(10) Pub. No.: US 2016/0297851 A1
(43) Pub. Date: Oct. 13, 2016

(54) BROAD SPECTRUM ANTIBIOTIC ARYLOMYCIN ANALOGS

(71) Applicants: Floyd E. Romesberg, La Jolla, CA (US); Peter A. Smith, San Francisco, CA (US); Tucker C. Roberts, San Diego, CA (US)

(72) Inventors: Floyd E. Romesberg, La Jolla, CA (US); Peter A. Smith, San Francisco, CA (US); Tucker C. Roberts, San Diego, CA (US)

(21) Appl. No.: 15/008,881

(22) Filed: Jan. 28, 2016

Related U.S. Application Data
(63) Continuation of application No. 14/384,638, filed on Sep. 11, 2014, now abandoned.
(60) Provisional application No. 61/610,922, filed on Mar. 14, 2012.

(51) Int. Cl. C07K 7/06; A61K 9/00; C12Q 1/18; G01N 33/569; C12Q 1/68; C12N 9/52; A61K 38/08; A61K 45/06

(52) U.S. Cl. CPC C07K 7/06; A61K 38/08; A61K 9/0014; A61K 45/06; G01N 33/56916; C12Q 1/689; C12N 9/52; C12Q 1/18; G01N 33/56938; G01N 33/56911; G01N 33/56927; G01N 2333/952; G01N 2469/10; C12Q 2600/156

(57) ABSTRACT

Arylomycin analogs are provided, wherein the analogs can have broad spectrum bioactivity. Resistance to the antibiotic bioactivity of natural product arylomycin in a range of pathogenic bacterial species has been found to depend upon single amino acid mutations at defined positions of bacterial Signal Peptidases (SPases), wherein the presence of a proline residue confers arylomycin resistance. Arylomycin analogs are provided herein that can overcome that resistance and provide for a broader spectrum of antibiotic bioactivity than can natural product arylomycins such as arylomycin A2. Methods for determining if a bacterial strain is susceptible to narrow spectrum arylomycin antibiotics, or if a broad spectrum analog is required for treatment, is provided. Pharmaceutical compositions and methods of treatment of bacterial infections, and methods of synthesis of arylomycin analogs, are provided.

### CROSS-REFERENCE TO RELATED APPLICATIONS

This application claims the priority of U.S. provisional application Ser. No. 61/610,922, filed Mar. 14, 2012, the disclosure of which is incorporated herein by reference in its entirety.

### STATEMENT OF GOVERNMENT SUPPORT

This invention was made with government support under Grant No. N00014-08-0478, awarded by the Office of Naval Research. The U.S. government has certain rights in the invention.

### BACKGROUND

The arylomycin class of natural product, which includes the arylomycin A and B series, was initially discovered by the group of Hans-Peter Frielder, and described in a 2002 publication in the Journal of Antibiotics (J. Schimana, et al., J. Antibiotics (2002), 55(6), 565-570 and 571-577). The arylomycins, as characterized in this publication, comprise a unique structural class of natural product composed of a hexapeptide with a unique biaryl bridge between N-methyl-4-hydroxyphenylglycine5 (MeHpg5) and tyrosine7, and N-terminal acyl tails of various lengths. See FIG. 1 showing the structures of natural products of the arylomycin A and B classes as determined by the Frielder group.

The initial report describing the arylomycins provided data describing the antibiotic activity of these compounds again the soil bacteria, Arthrobacter globiformis DSM20124, Arthrobacter oxygans DSM 6612, Arthrobacter pascens DSM 20545, Rhodococcus erythropolis DSM 1069 (currently characterized Rhodococcus opacus), Streptomyces viridochromogenes Tu 57, and Brevibacillus brevis DSM 30, and against the fungus Mucor hiemalis Tu 179/180. The data appear to suggest and the authors conclude that the arylomycins have limited to no activity against the examined bacteria with the exception of Rhodococcus opacus and Brevibacillus brevis. Furthermore while no data is provided, the authors state that no the arylomycin lack activity against the Gram-negative bacteria Escherichia coli K12, Proteus mirabilis ATCC 35501, Pseudomonas fluorescens DSM 50090 and against the eukaryotic organisms Saccharomyces cerevisiae ATCC 9080, Botrytis cinerea Tu 157 and against the green algae Chlorella fusca and against the duckweed Lemna minor.

In 2004 Kulanthaivel, et al., independently discovered a subclass of the arylomycins, the lipoglycopeptides, which differ from the arylomycin A and B series via glycosylation and aromatic hydroxylation of the hydroxyphenylglycine residue as well as in the length of the N-terminal acyl tail. Kinetic evidence was provided to support the conclusion that the lipoglycopepetides inhibit the essential bacterial enzyme type I signal peptidase in vitro. However, most active members of the lipoglycopeptides displayed only moderate whole cell activity against the human pathogen Streptococcus pnemoniae R6, and extremely little to no activity against the human pahogens Staphylococcus aureus ATCC 13709, Haemophilus influenzae ATCC49247, and Escherichia coli K12. Furthermore the intrinsic resistance of E. coli K12 to the lipoglycopeptide subclass of arylomycins was attributed to outer membrane penetrance of drug efflux based on genetic data, suggesting that the arylomycins are not suitable as antimicrobial agents against Gram-negative bacteria. See U.S. Pat. No. 6,951,840, issued Oct. 4, 2005; see also P. Kulanthaivel, et al., J. Biol. Chem. (2004), 279(35), 36250-36258.

The natural products as described by Frielder and Kulanthaivel are as shown in FIG. 1; the compounds termed “arylomycins” by Frielder's group, and the compounds termed “lipoglycopeptides” by the Lilly workers including Kulanthaivel.

A total synthesis of arylomycin A2 has been reported by the inventors herein, see T. Roberts, et al. (2007), J. Am. Chem. Soc. 129, 15830-15838.

### SUMMARY

The present invention is directed to the use of analogs of the natural product arylomycin for the treatment of microbial infections, such as for the treatment of bacterial infections. In various embodiments, the invention provides classes and subclasses of chemical compounds structurally related to arylomycin for the treatment of bacterial infections. In various embodiments, the bacterial infections are resistant to treatment with the natural product arylomycin, but are susceptible to treatment with an arylomycin analog of the invention.

In various embodiments, the invention provides a compound of formula (I)

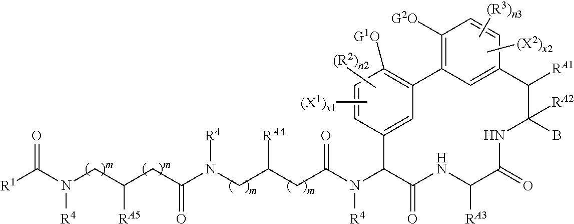

(I)
   wherein

B is CO₂H, CH₂CO₂H, C(═O)NHCH₂C(═O)H, CH₂C(═O)H, C(═O)NHCH₂B(OR^{B})₂ or C(═O)NHCH₂P(═O)(OR^{B})₂ wherein R^{B} is H, —(C₁-C₆)alkyl, or (C₆-C₁₀)aryl; or B is a group of formula

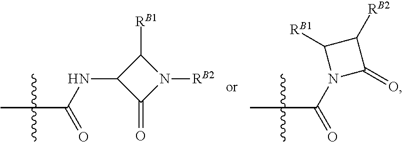

wherein R^{B1} and R^{B2} are each independently H, (C₁-C₆)alkyl, (C₃-C₆) cycloalkyl, OR^{C}, C(═O)NR^{C}₂, OC(═O)NR^{C}₂, C(═O)OR^{C}, OC(═O)OR^{C}, nitro, trifluoromethyl, trifluoromethoxy, (C₁-C₆)alkoxy, (C₁-C₆)thioalkoxy, NR^{C}₂, 5-7 membered heterocyclyl or 5-7 membered heteroaryl, or (C₆-C₁₀)aryl; R^{C} is independently at each occurrence H or (C₁-C₆)alkyl, and a wavy line indicates a point of attachment of B to a carbon of formula (I) bearing B;

R¹ comprises a group of formula (IIA), (IIB), or (IIC):

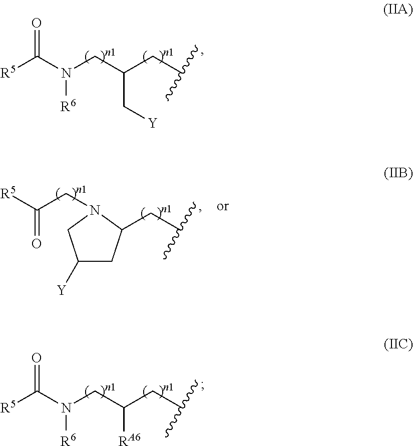

wherein each m is independently 0, 1, or 2, n1 is independently at each occurrence 0, 1, or 2; Y is (CH₂)₀₋₂H, (CH₂)₀₋₂OH, or (CH₂)₀₋₂OC(═O)(C₁-C₆)alkyl; R^{A6} is hydrogen, (C₁-C₆)alkyl, (C₃-C₇)cycloalkyl, 5- to 7-membered heteroaryl, 5- to 7-membered heterocyclyl, or (C₆-C₁₀)aryl, wherein any alkyl, cycloalkyl, heterocyclyl, aryl or heteroaryl may be substituted with 1 to 3 substituents, wherein each substituent is independently selected from the group consisting of halogen, amino, hydroxyl, aminocarbonyl, hydroxycarbonyl, nitro, cyano, trifluoromethyl, trifluoromethoxy, 5- to 7-membered heterocyclyl, (C₁-C₆)alkoxy, (C₁-C₆)alkyl, (C₃-C₇)cycloalkyl, (C₁-C₆)-mono- or di-alkylamino, (C₁-C₆)alkoxycarbonyl, (C₁-C₆)alkylhydroxycarbonyl, (C₁-C₆)alkylaminocarbonyl, (C₁-C₆)alkylsulfonylamino, and (C₆-C₁₀)-arylsulfonylamino; and a wavy line indicates a point of attachment of R¹ to an atom of formula (I) bearing R¹; and

R⁵ is a linear or branched alkyl chain of about 1-22 carbon atoms, bonded to the carbonyl carbon to which it is attached directly or by an O or NH, to provide an amide, carbamate, or urea linkage respectively; optionally comprising within the chain or at a chain terminus, any of the following groups:

(A)

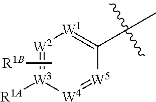

wherein W¹, W², W³, W⁴ and W⁵ are each independently C or N, provided that no more than two of W¹, W², W³, W⁴ and W⁵ are N; provided that when R^{1A} or R^{1B} is non-hydrogen, any W atom to which the R^{1A} or R^{1B} is respectively bonded is C, wherein there can be one or more R^{1B} bonded to the ring bearing the W atoms; R^{1A} is hydrogen, halogen, amino, hydroxyl, aminocarbonyl, hydroxycarbonyl, nitro, fluoroalkyl, cyano, (C₁-C₆)-thioether, fluoroalkoxy, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; R^{1B} is hydrogen, alkyl, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-alkylamino, (C₁-C₆)-alkyl, 5- to 7-membered heteroaryl, 5- to 7-membered heterocyclyl, or (C₆-C₁₀)aryl; wherein any R^{1A} or R^{1B} can be further substituted with one to three (C₁-C₁₂)-alkyl or -alkoxy groups, which can further bear halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-alkylamino, 5- to 7-membered heteroaryl, 5- to 7-membered heterocyclyl, or (C₆-C₁₀)aryl groups; wherein a wavy line indicates a point of attachment;

(B)

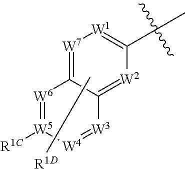

wherein W¹, W², W³, W⁴, W⁵, W⁶, and W⁷ are each independently C or N, provided that no more than three of W¹, W², W³, W⁴, W⁵, W⁶, and W⁷ are N; provided that when R^{1C} or R^{1D} is non-hydrogen, any W atom to which the R^{1C} or R^{1D} is respectively bonded is C, wherein either ring can bear one or more R^{1D}; R^{1C} is hydrogen, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; R^{1D} is hydrogen, alkyl, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein any R^{1C} or R^{1D} can be further substituted with one to three (C₁-C₁₂)-alkyl or -alkoxy groups, which can further bear halogen, amino, hydroxyl, aminocarbonyl, hydroxycarbonyl, nitro, fluoroalkyl, (C₁-C₆)thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein a wavy line indicates a point of attachment;

(C)

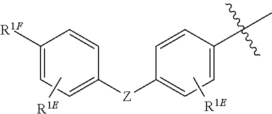

wherein Z is O, S, NH or CH₂; R^{1E} at each occurrence is independently hydrogen, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; R^{1F} is hydrogen or alkyl, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein any R^{1E} or R^{1F} can be further substituted with one to three (C₁-C₁₂)-alkyl or -alkoxy groups, which can further bear halogen, amino, hydroxyl, aminocarbonyl, hydroxycarbonyl, nitro, fluoroalkyl, (C₁-C₆)thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein a wavy line indicates a point of attachment; or

(D)

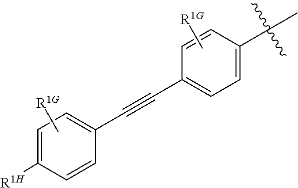

wherein R^{1G} at each occurrence is independently hydrogen, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; R^{1H} is hydrogen or alkyl, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein any R^{1G} or R^{1H} can be further substituted with one to three (C₁-C₁₂)-alkyl or -alkoxy groups, which can further bear halogen, amino, hydroxyl, aminocarbonyl, hydroxycarbonyl, nitro, fluoroalkyl, (C₁-C₆)thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein a wavy line indicates a point of attachment;

R² and R³ are each independently nitro, halo, cyano, hydroxy, glycosyloxy, amino, (C₁-C₄)alkoxy, (C₁-C₄)acyloxy, or (C₁-C₄)alkyl, wherein any carbon atom can be unsubstituted or substituted with J, wherein n² and n³ are independently 0, 1, 2, or 3; or wherein two R² groups taken together, and/or two R³ groups taken together, can comprise fused cycloalkyl, aryl, heterocyclyl, or heteroaryl ring or rings, any of which is substituted with 0-3 J;

R⁴ and R⁶ are each independently at every occurrence hydrogen, (C₁-C₆)alkyl, (C₃-C₇)cycloalkyl, 5- to 7-membered heteroaryl, 5- to 7-membered heterocyclyl, or (C₆-C₁₀)aryl, wherein any alkyl, cycloalkyl, heterocyclyl, aryl or heteroaryl can be substituted with 1 to 3 J;

R^{A1}, R^{A2}, R^{A3}, R^{A4}, R^{A5} are independently at each occurrence hydrogen, (C₁-C₆)alkyl, (C₃-C₇)cycloalkyl, 5- to 7-membered heteroaryl, 5- to 7-membered heterocyclyl, or (C₆-C₁₀)aryl, wherein any alkyl, cycloalkyl, heterocyclyl, aryl or heteroaryl can be substituted with 1 to 3 J;

J is halogen, R′, OR′, CN, CF₃, OCF₃, O, S, C(O), S(O), methylenedioxy, ethylenedioxy, (CH₂)₀₋ₚN(R¹)₂, (CH₂)₀₋ₚSR′, (CH₂)₀₋ₚS(O)R′, (CH₂)₀₋ₚS(O)₂R′, (CH₂)₀₋ₚS(O)₂N(R′)₂, (CH₂)₀₋ₚSO₃R′, (CH₂)₀₋ₚC(O)R′, (CH₂)₀₋ₚC(O)CH₂C(O)R′, (CH₂)₀₋ₚC(S)R′, (CH₂)₀₋ₚC(O)OR′, (CH₂)₀₋ₚOC(O)R′, (CH₂)₀₋ₚC(O)N(R′)₂, (CH₂)₀₋ₚOC(O)N(R′)₂, (CH₂)₀₋ₚC(S)N(R′)₂, (CH₂)₀₋ₚNH—C(O)R′, (CH₂)₀₋ₚN(R′)N(R′)C(O)R′, (CH₂)₀₋ₚN(R′)N(R′)C(O)OR′, (CH₂)₀₋ₚN(R′)N(R′)CON(R′)₂, (CH₂)₀₋ₚN(R′)SO₂R′, (CH₂)₀₋ₚN(R′)SO₂N(R′)₂, (CH₂)₀₋ₚN(R′)C(O)OR′, (CH₂)₀₋ₚN(R′)C(O)R′, (CH₂)₀₋ₚN(R′)C(S)R′, (CH₂)₀₋ₚN(R′)C(O)N(R′)₂, (CH₂)₀₋ₚN(R′)C(S)N(R′)₂, (CH₂)₀₋ₚN(COR′)COR′, (CH₂)₀₋ₚN(OR′)R′, (CH₂)₀₋ₚC(═NH)N(R′)₂, (CH₂)₀₋ₚC(O)N(OR′)R′, or (CH₂)₀₋ₚC(═NOR′)R′; wherein p is about 4,

each R′ is independently at each occurrence hydrogen, (C₁-C₁₂)-alkyl, (C₂-C₁₂)-alkenyl, (C₂-C₁₂)-alkynyl, (C₃-C₁₀)-cycloalkyl, (C₃-C₁₀)-cycloalkenyl, [(C₃-C₁₀)cycloalkyl or (C₃-C₁₀)-cycloalkenyl]-[(C₁-C₁₂)-alkyl or (C₂-C₁₂)-alkenyl or (C₂-C₁₂)-alkynyl], (C₆-C₁₀)-aryl, (C₆-C₁₀)-aryl-[(C₁-C₁₂)-alkyl or (C₂-C₁₂)-alkenyl or (C₂-C₁₂)-alkynyl], mono- or bicyclic 3-10 membered heterocyclyl, mono- or bicyclic 3-10 membered heterocyclyl-[(C₁-C₁₂)-alkyl or (C₂-C₁₂)-alkenyl or (C₂-C₁₂)-alkynyl], mono- or bicyclic 5-10 membered heteroaryl, or mono- or bicyclic 5-10 membered heteroaryl-[(C₁-C₁₂)-alkyl or (C₂-C₁₂)-alkenyl or (C₂-C₁₂)-alkynyl];

or, when two R′ are bound to a nitrogen atom or to two adjacent nitrogen atoms, the two R′ groups together with the nitrogen atom or atoms to which they are bound can form a 3- to 8-membered monocyclic heterocyclic ring, or an 8- to 20-membered, bicyclic or tricyclic, heterocyclic ring system, wherein any ring or ring system can further contain 1-3 additional heteroatoms selected from the group consisting of N, NR′, O, S, S(O) and S(O)₂;

wherein, in any bicyclic or tricyclic ring system, each ring is linearly fused, bridged, or spirocyclic, wherein each ring is either aromatic or nonaromatic, wherein each ring can be fused to a (C₆-C₁₀)aryl, mono- or bicyclic 5-10 membered heteroaryl, (C₃-C₁₀)cycloalkyl or mono- or bicyclic 3-10 membered heterocyclyl;

G¹ and G² are each independently a hydrogen or a glycosyl residue, or a group cleavable under physiological conditions to provide a compound of formula (I) wherein G¹ or G² respectively is hydrogen;

(X¹)_{X1} and (X²)_{X2} each signify that 0, 1, or 2 ring atoms of each respective ring can be nitrogen, provided that where a non-hydrogen substituent is bonded, X¹ or X², respectively, is C;

provided that when G¹ is a 6-deoxyhexopyranosyl residue, G² is H, R¹ is of formula (IIA), R² is hydrogen or hydroxy, R³ is hydrogen, R^{A1} and R^{A2} and R^{A4} are H, R^{A3} and R^{A5} are methyl, and B is CO₂H, or when G¹ and G² are H, R¹ is of formula (IIA), R² is hydrogen, R³ is hydrogen or nitro, R^{A1} and R^{A2} and R^{A4} are H, R^{A3} and R^{A5} are methyl, and B is CO₂H, then R⁵ is not unsubstituted (C₁₀-C₁₆)-alkyl;

or a salt thereof.

In various embodiments, the present invention relates to the use of arylomycins to treat a microbial or bacterial infection caused by a microbe (e.g., a bacterium) that encodes a type I signal peptidase bearing a specific amino acid sequence signature. In addition, in various embodiments, the present invention relates to the use of arylomycin to treat indications known to be caused predominantly by bacteria that encoded type I signal peptidases bearing a specific amino acid sequence signature.

In various embodiments, the invention is direct to the use of genetically modified organisms harboring mutations in the type I bacterial signal peptidase enzyme to screen for or to characterize the activity of type I signal peptidase inhibitors such as arylomycin analogs of the invention. In various embodiments, the invention provides methods for the use of genetically altered recombinant forms of bacterial signal peptidase protein, in which specific residues have been mutated, to screen for or characterize the activity of type I signal peptidase inhibitors, such as arylomycin analogs of the invention.

### BRIEF DESCRIPTION OF THE FIGURES

FIG. 1 shows the structures of arylomycins A and B, natural products, as characterized in J. Schimana, et al., J. Antibiotics (2002), 55(6), 565-570). R¹ refers to a substituent on the tyrosine residue, and R² refers to an acyl group of the indicated number of carbon atoms bonded to the N-methylserine nitrogen atom.

FIG. 2 graphically correlates the doubling time and the minimum inhibitory concentrations (MICs) of arylomycin C16 observed for E. coli strains harboring the indicated amino acid at SPase residue 84. Horizontal bars indicate standard deviation of doubling times from three independent experiments. MIC values varied less than 2-fold between experiments. The His (MIC 4 μg/ml) and Phe or His (MIC 2 μg/ml) variants have a temperature sensitive phenotype and therefore not shown. For Pro29, the MIC exceeded the detection limit of 256 μg/ml.

FIG. 3A shows the structure of crystalline E. coli SPase in complex with arylomycin A2 (PDB ID IT7D) (see, Paetzel et al., J. Biol. Chem. 279, 30781-30790 (2004)). Hydrogen-bonds observed in the crystal structure are shown in green, while the potential hydrogen bond prevented by Pro84 is shown in red. FIG. 3B-3C show the equilibrium binding affinities of arylomycin for Pro- and Ser-variants of E. coli (FIG. 3B) and S. aureus (FIG. 3C) SPases. Data points and bars represent average values and standard deviations within a single experiment. KD values shown are the average of three independent experiments. FIG. 3D shows the affinity of arylomycin C16 for the soluble N-terminally truncated E. coli SPase. The steady state binding affinities of arylomycin C16 for WT (open diamonds) and P84S (open square) variants of the soluble N-terminally truncated E. coli SPase are shown as a function of Arylomycin C concentration.

FIG. 4 shows a phylogenetic reconstruction of the evolution of Pro29 within Staphylococcaceae SPases. The left side shows the phylogeny of Staphylococcaceae based on 16S RNA sequences. For each species, color bars indicate the number of SPases and presence or absence of Pro at residue 29; the amino acid(s) at residue(s) 29 is/are parenthetically noted in single letter code following the species name. The right side shows the phylogenetic relationship of SPase sequences from the indicated species. For each gene, the amino acid at residue 29 is parenthetically noted in single letter code following the name of the species in which the gene is found. Red and Blue branches represent the evolution of two distinct SPase lineages present in the common ancestor of sequenced Staphylococcaceae. Branches with an alternative likelihood ratio test support of less than 50% are collapsed.

FIGS. 5A through 5H together provide a list of organisms that lack a proline residue at positions −5 and/or −7 (N-terminal) to the active site serine of the SPase encoded in their genome.

### DETAILED DESCRIPTION

### Definitions

As used in the specification and the appended claims, the singular forms “a,” “an” and “the” include plural referents unless the context clearly dictates otherwise.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

As used herein, “individual” (as in the subject of the treatment) means both mammals and non-mammals. Mammals include, for example, humans; non-human primates, e.g. apes and monkeys; and non-primates, e.g. dogs, cats, cattle, horses, sheep, and goats. Non-mammals include, for example, fish and birds.

The term “disease” or “disorder” or “malcondition” are used interchangeably, and are used to refer to diseases or conditions wherein a bacterial SPase plays a role in the biochemical mechanisms involved in the disease or malcondition such that a therapeutically beneficial effect can be achieved by acting on the enzyme. “Acting on” SPase can include binding to SPase and/or inhibiting the bioactivity of an SPase.

The expression “effective amount”, when used to describe therapy to an individual suffering from a disorder, refers to the amount of a compound of the invention that is effective to inhibit or otherwise act on SPase in the individual's tissues wherein SPase involved in the disorder is active, wherein such inhibition or other action occurs to an extent sufficient to produce a beneficial therapeutic effect.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

When a substituent is specified to be an atom or atoms of specified identity, “or a bond”, a configuration is referred to when the substituent is “a bond” that the groups that are immediately adjacent to the specified substituent are directly connected to each other in a chemically feasible bonding configuration.

All chiral, diastereomeric, racemic forms of a structure are intended, unless a particular stereochemistry or isomeric form is specifically indicated. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

The inclusion of an isotopic form of one or more atoms in a molecule that is different from the naturally occurring isotopic distribution of the atom in nature is referred to as an “isotopically labeled form” of the molecule. All isotopic forms of atoms are included as options in the composition of any molecule, unless a specific isotopic form of an atom is indicated. For example, any hydrogen atom or set thereof in a molecule can be any of the isotopic forms of hydrogen, i.e., protium (¹H), deuterium (²H), or tritium (³H) in any combination. Similarly, any carbon atom or set thereof in a molecule can be any of the isotopic form of carbons, such as ¹¹C, ¹²C, ¹³C, or ¹⁴C, or any nitrogen atom or set thereof in a molecule can be any of the isotopic forms of nitrogen, such as ¹³N, ¹⁴N, or ¹⁵N. A molecule can include any combination of isotopic forms in the component atoms making up the molecule, the isotopic form of every atom forming the molecule being independently selected. In a multi-molecular sample of a compound, not every individual molecule necessarily has the same isotopic composition. For example, a sample of a compound can include molecules containing various different isotopic compositions, such as in a tritium or ¹⁴C radiolabeled sample where only some fraction of the set of molecules making up the macroscopic sample contains a radioactive atom. It is also understood that many elements that are not artificially isotopically enriched themselves are mixtures of naturally occurring isotopic forms, such as ¹⁴N and ¹⁵N, ³²S and ³⁴S, and so forth. A molecule as recited herein is defined as including isotopic forms of all its constituent elements at each position in the molecule. As is well known in the art, isotopically labeled compounds can be prepared by the usual methods of chemical synthesis, except substituting an isotopically labeled precursor molecule. The isotopes, radiolabeled or stable, can be obtained by any method known in the art, such as generation by neutron absorption of a precursor nuclide in a nuclear reactor, by cyclotron reactions, or by isotopic separation such as by mass spectrometry. The isotopic forms are incorporated into precursors as required for use in any particular synthetic route. For example, ¹⁴C and ³H can be prepared using neutrons generated in a nuclear reactor. Following nuclear transformation, ¹⁴C and ³H are incorporated into precursor molecules, followed by further elaboration as needed.

The term “amino protecting group” or “N-protected” as used herein refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

The term “hydroxyl protecting group” or “O-protected” as used herein refers to those groups intended to protect an OH group against undesirable reactions during synthetic procedures and which can later be removed to reveal the amine. Commonly used hydroxyl protecting groups are disclosed in Protective Groups in Organic Synthesis, Greene, T. W.; Wuts, P. G. M., John Wiley & Sons, New York, N.Y., (3rd Edition, 1999). Hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. It is well within the skill of the ordinary artisan to select and use the appropriate hydroxyl protecting group for the synthetic task at hand.

In general, “substituted” refers to an organic group as defined herein in which one or more bonds to a hydrogen atom contained therein are replaced by one or more bonds to a non-hydrogen atom such as, but not limited to, a halogen (i.e., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOW, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted.

When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, which can also be written as “CO”, “C(O)”, or “C(═O)”, wherein the C and the O are double bonded. When a carbon atom is substituted with a double-bonded oxygen (═O) group, the oxygen substituent is termed an “oxo” group. When a divalent substituent such as NR is double-bonded to a carbon atom, the resulting C(═NR) group is termed an “imino” group. When a divalent substituent such as S is double-bonded to a carbon atom, the results C(═S) group is termed a “thiocarbonyl” group.

Alternatively, a divalent substituent such as O, S, C(O), S(O), or S(O)₂ can be connected by two single bonds to two different carbon atoms. For example, O, a divalent substituent, can be bonded to each of two adjacent carbon atoms to provide an epoxide group, or the O can form a bridging ether group, termed an “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR′₂)ₙ wherein n is 1, 2, 3, or more, and each R′ is independently selected.

C(O) and S(O)₂ groups can be bound to one or two heteroatoms, such as nitrogen, rather than to a carbon atom. For example, when a C(O) group is bound to one carbon and one nitrogen atom, the resulting group is called an “amide” or “carboxamide.” When a C(O) group is bound to two nitrogen atoms, the functional group is termed a urea. When a S(O)₂ group is bound to one carbon and one nitrogen atom, the resulting unit is termed a “sulfonamide.” When a S(O)₂ group is bound to two nitrogen atoms, the resulting unit is termed a “sulfamate.”

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups can also be substituted with alkyl, alkenyl, and alkynyl groups as defined herein.

By a “ring system” as the term is used herein is meant a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. By “spirocyclic” is meant the class of structures wherein two rings are fused at a single tetrahedral carbon atom, as is well known in the art.

As to any of the groups described herein, which contain one or more substituents, it is understood, of course, that such groups do not contain any substitution or substitution patterns which are sterically impractical and/or synthetically non-feasible. In addition, the compounds of this disclosed subject matter include all stereochemical isomers arising from the substitution of these compounds.

Selected substituents within the compounds described herein are present to a recursive degree. In this context, “recursive substituent” means that a substituent may recite another instance of itself or of another substituent that itself recites the first substituent. Because of the recursive nature of such substituents, theoretically, a large number may be present in any given claim. One of ordinary skill in the art of medicinal chemistry and organic chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

Recursive substituents are an intended aspect of the disclosed subject matter. One of ordinary skill in the art of medicinal and organic chemistry understands the versatility of such substituents. To the degree that recursive substituents are present in a claim of the disclosed subject matter, the total number should be determined as set forth above.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed above, for example, amino, hydroxy, cyano, carboxy, nitro, thio, alkoxy, and halogen groups. A description herein that a group is alkyl chain“optionally comprising within the chain or at a chain terminus” a moiety, the term signifies that the moiety can be disposed between two subunits of the alkyl chain, or can be disposed at an unsubstituted end of the chain, or can be disposed between the chain and a point of attachment of the chain, for example to a carbonyl, NR, or O group. For example, an alkylbenzoyl group is an alkyl chain with a phenyl group disposed between the alkyl and a carbonyl, fitting the above description; an N-alkylphenylcarboxamido is an alkyl chain with a phenyl group displosed between the alkyl and the aminocarbonyl group, filling within the above description.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic,” “carbocyclyl,” and “carbocycle” denote a ring structure wherein the atoms of the ring are carbon, such as a cycloalkyl group or an aryl group. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N−1 substituents wherein N is the size of the carbocyclic ring with, for example, alkyl, alkenyl, alkynyl, amino, aryl, hydroxy, cyano, carboxy, heteroaryl, heterocyclyl, nitro, thio, alkoxy, and halogen groups, or other groups as are listed above. A carbocyclyl ring can be a cycloalkyl ring, a cycloalkenyl ring, or an aryl ring. A carbocyclyl can be monocyclic or polycyclic, and if polycyclic each ring can be independently be a cycloalkyl ring, a cycloalkenyl ring, or an aryl ring.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like, provided they include at least one double bond within a ring. Cycloalkenyl groups also include rings that are substituted with straight or branched chain alkyl groups as defined above.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂CH₂—OH, —CH₂—CH₂—NH—CH₃, —CH₂—S—CH₂—CH₃, —CH₂CH₂—S(═O)—CH₃, and —CH₂CH₂—O—CH₂CH₂—O—CH₃. Up to two heteroatoms may be consecutive, such as, for example, —CH₂—NH—OCH₃, or —CH₂—CH₂—S—S—CH₃.

A “cycloheteroalkyl” ring is a cycloalkyl ring containing at least one heteroatom. A cycloheteroalkyl ring can also be termed a “heterocyclyl,” described below.

The term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, —CH₂—CH═CH—CH₂—SH, and —CH═CH—O—CH₂CH₂—O—CH₃.

Aryl groups are cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups or the term “heterocyclyl” includes aromatic and non-aromatic ring compounds containing 3 or more ring members, of which, one or more is a heteroatom such as, but not limited to, N, O, and S. Thus a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. A heterocyclyl ring can also include one or more double bonds. A heteroaryl ring is an embodiment of a heterocyclyl group. The phrase “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed above. Representative substituted heteroaryl groups can be substituted one or more times with groups such as those listed above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Heterocyclylalkyl groups are alkyl groups as defined above in which a hydrogen or carbon bond of an alkyl group as defined above is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroarylalkyl groups are alkyl groups as defined above in which a hydrogen or carbon bond of an alkyl group is replaced with a bond to a heteroaryl group as defined above.

The term “alkoxy” refers to an oxygen atom connected to an alkyl group, including a cycloalkyl group, as are defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structures are substituted therewith.

The terms “halo” or “halogen” or “halide” by themselves or as part of another substituent mean, unless otherwise stated, a fluorine, chlorine, bromine, or iodine atom, preferably, fluorine, chlorine, or bromine.

A “haloalkyl” group includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

A “haloalkoxy” group includes mono-halo alkoxy groups, poly-halo alkoxy groups wherein all halo atoms can be the same or different, and per-halo alkoxy groups, wherein all hydrogen atoms are replaced by halogen atoms, such as fluoro. Examples of haloalkoxy include trifluoromethoxy, 1,1-dichloroethoxy, 1,2-dichloroethoxy, 1,3-dibromo-3,3-difluoropropoxy, perfluorobutoxy, and the like.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is —CF₃.

The term “(Cₓ-C_{y})perfluoroalkylene,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkylene, more preferred is —(C₁-C₃)perfluoroalkylene, most preferred is —CF₂—.

The terms “aryloxy” and “arylalkoxy” refer to, respectively, an aryl group bonded to an oxygen atom and an aralkyl group bonded to the oxygen atom at the alkyl moiety. Examples include but are not limited to phenoxy, naphthyloxy, and benzyloxy.

An “acyl” group as the term is used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-20 additional carbon atoms bonded to the carbonyl group. An acyl group can include double or triple bonds within the meaning herein. An acryloyl group is an example of an acyl group. An acyl group can also include heteroatoms within the meaning here. A nicotinoyl group (pyridyl-3-carbonyl) group is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “amine” includes primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

An “amino” group is a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary or quaternary amino group. An “alkylamino” group includes a monoalkylamino, dialkylamino, and trialkylamino group.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions, and amines, within the meaning herein.

The term “amide” (or “amido”) includes C- and N-amide groups, i.e., —C(O)NR₂, and —NRC(O)R groups, respectively. Amide groups therefore include but are not limited to primary carboxamide groups (—C(O)NH₂) and formamide groups (—NHC(O)H). A “carboxamido” group is a group of the formula C(O)NR₂, wherein R can be H, alkyl, aryl, etc.

The term “azido” refers to an N₃ group. An “azide” can be an organic azide or can be a salt of the azide (N₃⁻) anion. The term “nitro” refers to an NO₂ group bonded to an organic moiety. The term “nitroso” refers to an NO group bonded to an organic moiety. The term nitrate refers to an ONO₂ group bonded to an organic moiety or to a salt of the nitrate (NO₃⁻) anion.

The term “urethane” (“carbamoyl” or “carbamyl”) includes N- and O-urethane groups, i.e., —NRC(O)OR and —OC(O)NR₂ groups, respectively.

The term “sulfonamide” (or “sulfonamido”) includes S- and N-sulfonamide groups, i.e., —SO₂NR₂ and —NRSO₂R groups, respectively. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

The term “amidine” or “amidino” includes groups of the formula —C(NR)NR₂. Typically, an amidino group is —C(NH)NH₂.

The term “guanidine” or “guanidino” includes groups of the formula —NRC(NR)NR₂. Typically, a guanidino group is —NHC(NH)NH₂.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula (I) compounds, for example in their purification by recrystallization. All of these salts may be prepared by conventional means from the corresponding compound according to Formula (I) by reacting, for example, the appropriate acid or base with the compound according to Formula (I). The term “pharmaceutically acceptable salts” refers to nontoxic inorganic or organic acid and/or base addition salts, see, for example, Lit et al., Salt Selection for Basic Drugs (1986), Int. J. Pharm., 33, 201-217, incorporated by reference herein.

A “hydrate” is a compound that exists in a composition with water molecules. The composition can include water in stoichiometric quantities, such as a monohydrate or a dihydrate, or can include water in random amounts. As the term is used herein a “hydrate” refers to a solid form, i.e., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometric or non-stoichiometric. As the term is used herein a “solvate” refers to a solid form, i.e., a compound in solution in a solvent, while it may be solvated, is not a solvate as the term is used herein.

A “prodrug” as is well known in the art is a substance that can be administered to a patient where the substance is converted in vivo by the action of biochemicals within the patients body, such as enzymes, to the active pharmaceutical ingredient. Examples of prodrugs include esters of carboxylic acid groups, which can be hydrolyzed by endogenous esterases as are found in the bloodstream of humans and other mammals. Conventional procedures for the selection and preparation of suitable prodrug derivatives are described, for example, in “Design of Prodrugs”, ed. H. Bundgaard, Elsevier, 1985.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

The present invention further embraces isolated compounds according to formula (I). The expression “isolated compound” refers to a preparation of a compound of formula (I), or a mixture of compounds according to formula (I), wherein the isolated compound has been separated from the reagents used, and/or byproducts formed, in the synthesis of the compound or compounds. “Isolated” does not mean that the preparation is technically pure (homogeneous), but it is sufficiently pure to compound in a form in which it can be used therapeutically. Preferably an “isolated compound” refers to a preparation of a compound of formula (I) or a mixture of compounds according to formula (I), which contains the named compound or mixture of compounds according to formula (I) in an amount of at least 10 percent by weight of the total weight. Preferably the preparation contains the named compound or mixture of compounds in an amount of at least 50 percent by weight of the total weight; more preferably at least 80 percent by weight of the total weight; and most preferably at least 90 percent, at least 95 percent or at least 98 percent by weight of the total weight of the preparation.

The compounds of the invention and intermediates may be isolated from their reaction mixtures and purified by standard techniques such as filtration, liquid-liquid extraction, solid phase extraction, distillation, recrystallization or chromatography, including flash column chromatography, or HPLC.

### Isomerism and Tautomerism in Compounds of the Invention

### Tautomerism

Within the present invention it is to be understood that a compound of the formula (I) or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the invention encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

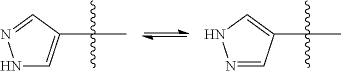 [SMILES: *c1cn[nH]c1 ; *c1cn[nH]c1]

Such tautomerism can also occur with substituted pyrazoles such as 3-methyl, 5-methyl, or 3,5-dimethylpyrazoles, and the like. Another example of tautomerism is amido-imido (lactam-lactim when cyclic) tautomerism, such as is seen in heterocyclic compounds bearing a ring oxygen atom adjacent to a ring nitrogen atom. For example, the equilibrium:

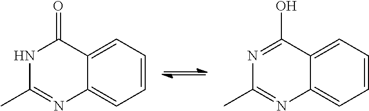

is an example of tautomerism. Accordingly, a structure depicted herein as one tautomer is intended to also include the other tautomer.

### Optical Isomerism

It will be understood that when compounds of the present invention contain one or more chiral centers, the compounds may exist in, and may be isolated as pure enantiomeric or diastereomeric forms or as racemic mixtures. The present invention therefore includes any possible enantiomers, diastereomers, racemates or mixtures thereof of the compounds of the invention.

The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 14, the Cahn-Ingold-Prelog ranking is A>B>C>D. The lowest ranking atom, D is oriented away from the viewer.

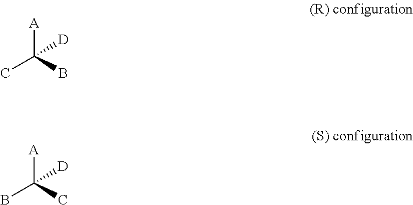

The present invention is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound of the invention, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

### Rotational Isomerism

It is understood that due to chemical properties (i.e., resonance lending some double bond character to the C—N bond) of restricted rotation about the amide bond linkage (as illustrated below) it is possible to observe separate rotamer species and even, under some circumstances, to isolate such species (see below). It is further understood that certain structural elements, including steric bulk or substituents on the amide nitrogen, may enhance the stability of a rotamer to the extent that a compound may be isolated as, and exist indefinitely, as a single stable rotamer. The present invention therefore includes any possible stable rotamers of formula (I) which are biologically active in the treatment of cancer or other proliferative disease states.

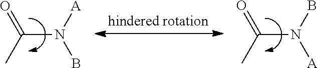

### Regioisomerism

The preferred compounds of the present invention have a particular spatial arrangement of substituents on the aromatic rings, which is related to the structure activity relationship demonstrated by the compound class. Often such substitution arrangement is denoted by a numbering system; however, numbering systems are often not consistent between different ring systems. In six-membered aromatic systems, the spatial arrangements are specified by the common nomenclature “para” for 1,4-substitution, “meta” for 1,3-substitution and “ortho” for 1,2-substitution as shown below.

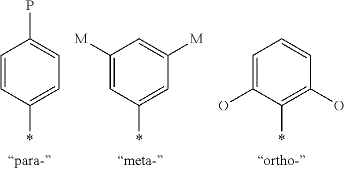

In various embodiments, the compound or set of compounds, such as are among the inventive compounds or are used in the inventive methods, can be any one of any of the combinations and/or sub-combinations of the above-listed embodiments.

### DETAILED DESCRIPTION

The present invention, in various embodiments is directed to analogs of arylomycins A and B. By arylomycins A and B are meant, respectively, the natural products of the following structures:

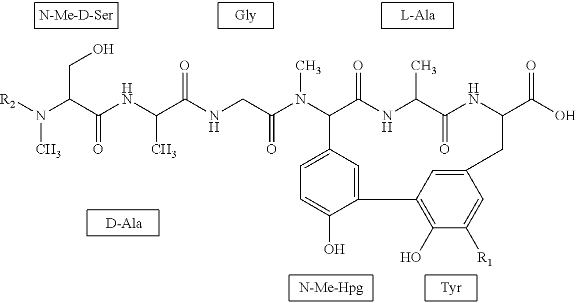

| Arylomycin | R₁ | R₂ |
|---|---|---|
| A₁ | H | iso-C₁₁ |
| A₂ | H | iso-C₁₂ |
| A₃ | H | n-C₁₂ |
| A₄ | H | anteiso-C₁₃ |
| A₅ | H | iso-C₁₄ |
| B₁ | NO₂ | iso-C₁₁ |
| B₂ | NO₂ | iso-C₁₂ |
| B₃ | NO₂ | n-C₁₂ |
| B₄ | NO₂ | anteiso-C₁₃ |
| B₅ | NO₂ | iso-C₁₃ |
| B₆ | NO₂ | iso-C₁₄ |
| B₇ | NO₂ | anteiso-C₁₅ |

The arylomycin A compounds bear a hydrogen atom in the R₁ position as defined in the above structure, and the arylomycin B compounds bear a nitro group in that position. The lipid tails, designated as group R₂ in the above structure, are n-alkyl, isoalkyl, and anteisoalkyl acyl groups with 11 to 15 total carbon atoms that form an amide bond with the N-Me-D-Ser residue. As used herein, the terms “arylomycins”, “arylomycin A”, “arylomycin B”, “arylomycin Aₓ”, “arylomycin natural products” and the like refer to these natural products, unless otherwise specified. The terms “arylomycin analogs”, “arylomycin derivatives”, “compounds of the invention”, and the like, refer to the compounds disclosed herein that do not fit within the herein-defined structural classes of arylomycin A or arylomycin B. Compounds of the invention are distinct from the natural products as specified above.

In various embodiments, the arylomycin analogs of the invention, i.e., the novel structures disclosed and claimed herein, exhibit a broader spectrum of antibiotic activity, i.e., against a wider variety of bacterial species, than do the natural products termed arylomycins A and B.

The invention, in various embodiments, also provides methods of treatment of bacterial infections using the analogs of the invention, and using arylomycins A and B, such as against bacterial species or strains that would not be expected, based upon ordinary knowledge, to be susceptible to treatment with arylomycins A and B. In this context, the invention includes the use of the inventive arylomycin analogs and of the arylomycin A and B natural products in carrying out an inventive method as disclosed and claimed herein.

### Compounds of the Invention

In various embodiments, the invention provides a compound of formula (I)

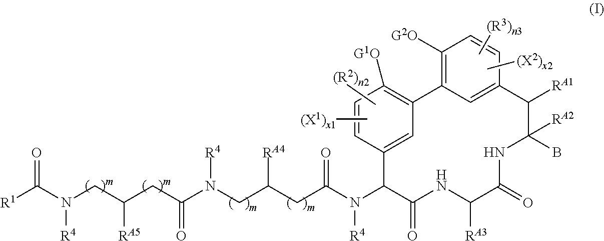

wherein

B is CO₂H, CH₂CO₂H, C(═O)NHCH₂C(═O)H, CH₂C(═O)H, C(═O)NHCH₂B(OR^{B})₂ or C(═O)NHCH₂P(═O)(OR^{B})₂ wherein R^{B} is H, (C₁-C₆)alkyl, or (C₆-C₁₀)aryl; or B is a group of formula

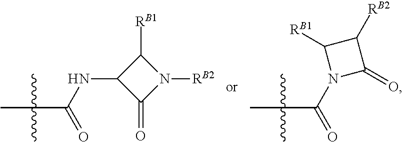

wherein R^{B1} and R^{B2} are each independently H, (C₁-C₆)alkyl, (C₃-C₆) cycloalkyl, OR^{C}, C(═O)NR^{C}₂, OC(═O)NR^{C}₂, C(═O)OR^{C}, OC(═O)OR^{C}, nitro, trifluoromethyl, trifluoromethoxy, (C₁-C₆)alkoxy, (C₁-C₆)thioalkoxy, NR^{C}₂, 5-7 membered heterocyclyl or 5-7 membered heteroaryl, or (C₆-C₁₀)aryl; R^{C} is independently at each occurrence H or (C₁-C₆)alkyl, and a wavy line indicates a point of attachment of B to a carbon of formula (I) bearing B;

R¹ comprises a group of formula (IIA), (IIB), or (IIC):

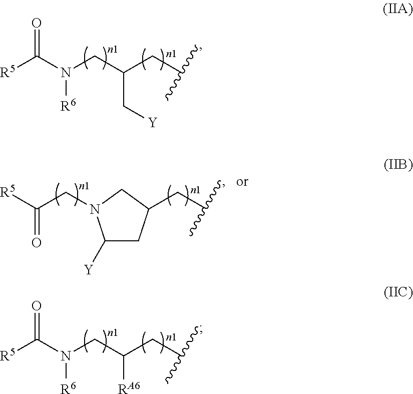

wherein each m is independently 0, 1, or 2, n1 is independently at each occurrence 0, 1, or 2; Y is (CH₂)₀₋₂H, (CH₂)₀₋₂OH, or (CH₂)₀₋₂OC(═O)(C₁-C₆)alkyl; R^{A6} is hydrogen, (C₁-C₆)alkyl, (C₃-C₇)cycloalkyl, 5- to 7-membered heteroaryl, 5- to 7-membered heterocyclyl, or (C₆-C₁₀)aryl, wherein any alkyl, cycloalkyl, heterocyclyl, aryl or heteroaryl may be substituted with 1 to 3 substituents, wherein each substituent is independently selected from the group consisting of halogen, amino, hydroxyl, aminocarbonyl, hydroxycarbonyl, nitro, cyano, trifluoromethyl, trifluoromethoxy, 5- to 7-membered heterocyclyl, (C₁-C₆)alkoxy, (C₁-C₆)alkyl, (C₃-C₇)cycloalkyl, (C₁-C₆)-mono- or di-alkylamino, (C₁-C₆)alkoxycarbonyl, (C₁-C₆)alkylhydroxycarbonyl, (C₁-C₆)alkylaminocarbonyl, (C₁-C₆)alkylsulfonylamino, and (C₆-C₁₀)-arylsulfonylamino; and a wavy line indicates a point of attachment of R¹ to an atom of formula (I) bearing R¹; and

R⁵ is a linear or branched alkyl chain of about 1-22 carbon atoms, bonded to the carbonyl carbon to which it is attached directly or by an O or NH, to provide an amide, carbamate, or urea linkage respectively; optionally comprising within the chain or at a chain terminus, any of the following groups:

(A)

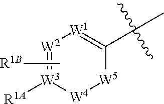

wherein W¹, W², W³, W⁴ and W⁵ are each independently C or N, provided that no more than two of W¹, W², W³, W⁴ and W⁵ are N; provided that when R^{1A} or R^{1B} is non-hydrogen, any W atom to which the R^{1A} or R^{1B} is respectively bonded is C, wherein there can be one or more R^{1B} bonded to the ring bearing the W atoms; R^{1A} is hydrogen, halogen, amino, hydroxyl, aminocarbonyl, hydroxycarbonyl, nitro, fluoroalkyl, cyano, (C₁-C₆)-thioether, fluoroalkoxy, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; R^{1B} is hydrogen, alkyl, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-alkylamino, (C₁-C₆)-alkyl, 5- to 7-membered heteroaryl, 5- to 7-membered heterocyclyl, or (C₆-C₁₀)aryl; wherein any R^{1A} or R^{1B} can be further substituted with one to three (C₁-C₁₂)-alkyl or -alkoxy groups, which can further bear halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-alkylamino, 5- to 7-membered heteroaryl, 5- to 7-membered heterocyclyl, or (C₆-C₁₀)aryl groups; wherein a wavy line indicates a point of attachment;

(B)

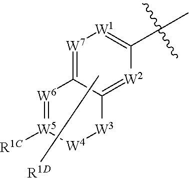

wherein W¹, W², W³, W⁴, W⁵, W⁶, and W⁷ are each independently C or N, provided that no more than three of W¹, W², W³, W⁴, W⁵, W⁶, and W⁷ are N; provided that when R^{1C} or R^{1D} is non-hydrogen, any W atom to which the R^{1C} or R^{1D} is respectively bonded is C, wherein either ring can bear one or more R^{1D}; R^{1C} is hydrogen, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; R^{1D} is hydrogen, alkyl, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein any R^{1C} or R^{1D} can be further substituted with one to three (C₁-C₁₂)-alkyl or -alkoxy groups, which can further bear halogen, amino, hydroxyl, aminocarbonyl, hydroxycarbonyl, nitro, fluoroalkyl, (C₁-C₆)thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein a wavy line indicates a point of attachment;

(C)

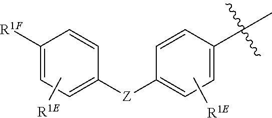

wherein Z is O, S, NH or CH₂; R^{1E} at each occurrence is independently hydrogen, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; R^{1F} is hydrogen or alkyl, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein any R^{1E} or R^{1F} can be further substituted with one to three (C₁-C₁₂)-alkyl or -alkoxy groups, which can further bear halogen, amino, hydroxyl, aminocarbonyl, hydroxycarbonyl, nitro, fluoroalkyl, (C₁-C₆)thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein a wavy line indicates a point of attachment; or

(D)

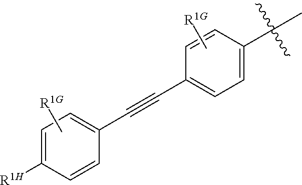

wherein R^{1G} at each occurrence is independently hydrogen, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; R^{1H} is hydrogen or alkyl, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein any R^{1G} or R^{1H} can be further substituted with one to three (C₁-C₁₂)-alkyl or -alkoxy groups, which can further bear halogen, amino, hydroxyl, aminocarbonyl, hydroxycarbonyl, nitro, fluoroalkyl, (C₁-C₆)thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein a wavy line indicates a point of attachment;

R² and R³ are each independently nitro, halo, cyano, hydroxy, glycosyloxy, amino, (C₁-C₄)alkoxy, (C₁-C₄)acyloxy, or (C₁-C₄)alkyl, wherein any carbon atom can be unsubstituted or substituted with J, wherein n² and n³ are independently 0, 1, 2, or 3; or wherein two R² groups taken together, and/or two R³ groups taken together, can comprise fused cycloalkyl, aryl, heterocyclyl, or heteroaryl ring or rings, any of which is substituted with 0-3 J;

R⁴ and R⁶ are each independently at every occurrence hydrogen, (C₁-C₆)alkyl, (C₃-C₇)cycloalkyl, 5- to 7-membered heteroaryl, 5- to 7-membered heterocyclyl, or (C₆-C₁₀)aryl, wherein any alkyl, cycloalkyl, heterocyclyl, aryl or heteroaryl can be substituted with 1 to 3 J;

R^{A1}, R^{A2}, R^{A3}, R^{A4}, R^{A5} are independently at each occurrence hydrogen, (C₁-C₆)alkyl, (C₃-C₇)cycloalkyl, 5- to 7-membered heteroaryl, 5- to 7-membered heterocyclyl, or (C₆-C₁₀)aryl, wherein any alkyl, cycloalkyl, heterocyclyl, aryl or heteroaryl can be substituted with 1 to 3 J;

J is halogen, R′, OR′, CN, CF₃, OCF₃, O, S, C(O), S(O), methylenedioxy, ethylenedioxy, (CH₂)₀₋ₚN(R′)₂, (CH₂)₀₋ₚSR′, (CH₂)₀₋ₚS(O)R′, (CH₂)₀₋ₚS(O)₂R′, (CH₂)₀₋ₚS(O)₂N(R′)₂, (CH₂)₀₋ₚSO₃R′(CH₂)₀₋ₚC(O)R′, (CH₂)₀₋ₚC(O)CH₂C(O)R′, (CH₂)₀₋ₚC(S)R′, (CH₂)₀₋ₚC(O)OR′, (CH₂)₀₋ₚOC(O)R′, (CH₂)₀₋ₚC(O)N(R′)₂, (CH₂)₀₋ₚOC(O)N(R′)₂, (CH₂)₀₋ₚC(S)N(R′)₂, (CH₂)₀₋ₚNH—C(O)R′, (CH₂)₀₋ₚN(R′)N(R′)C(O)R′, (CH₂)₀₋ₚN(R′)N(R′)C(O)OR′, (CH₂)₀₋ₚN(R′)N(R′)CON(R′)₂, (CH₂)₀₋ₚN(R′)SO₂R′, (CH₂)₀₋ₚN(R=′1)SO₂N(R′)₂, (CH₂)₀₋ₚN(R′)C(O)OR′, (CH₂)₀₋ₚN(R′)C(O)R′, (CH₂)₀₋ₚN(R′)C(S)R′, (CH₂)₀₋ₚN(R′)C(O)N(R′)₂, (CH₂)₀₋ₚN(R′)C(S)N(R′)₂, (CH₂)₀₋ₚN(COR′)COR′, (CH₂)₀₋ₚN(OR′)R′, (CH₂)₀₋ₚC(═NH)N(R′)₂, (CH₂)₀₋ₚC(O)N(OR′)R′, or (CH₂)₀₋ₚC(═NOR′)R′; wherein p is about 4,

each R′ is independently at each occurrence hydrogen, (C₁-C₁₂)-alkyl, (C₂-C₁₂)-alkenyl, (C₂-C₁₂)-alkynyl, (C₃-C₁₀)-cycloalkyl, (C₃-C₁₀)-cycloalkenyl, [(C₃-C₁₀)cycloalkyl or (C₃-C₁₀)-cycloalkenyl]-[(C₁-C₁₂)-alkyl or (C₂-C₁₂)-alkenyl or (C₂-C₁₂)-alkynyl], (C₆-C₁₀)-aryl, (C₆-C₁₀)-aryl-[(C₁-C₁₂)-alkyl or (C₂-C₁₂)-alkenyl or (C₂-C₁₂)-alkynyl], mono- or bicyclic 3-10 membered heterocyclyl, mono- or bicyclic 3-10 membered heterocyclyl-[(C₁-C₁₂)-alkyl or (C₂-C₁₂)-alkenyl or (C₂-C₁₂)-alkynyl], mono- or bicyclic 5-10 membered heteroaryl, or mono- or bicyclic 5-10 membered heteroaryl-[(C₁-C₁₂)-alkyl or (C₂-C₁₂)-alkenyl or (C₂-C₁₂)-alkynyl];

or, when two R′ are bound to a nitrogen atom or to two adjacent nitrogen atoms, the two R′ groups together with the nitrogen atom or atoms to which they are bound can form a 3- to 8-membered monocyclic heterocyclic ring, or an 8- to 20-membered, bicyclic or tricyclic, heterocyclic ring system, wherein any ring or ring system can further contain 1-3 additional heteroatoms selected from the group consisting of N, NR′, O, S, S(O) and S(O)₂;

wherein, in any bicyclic or tricyclic ring system, each ring is linearly fused, bridged, or spirocyclic, wherein each ring is either aromatic or nonaromatic, wherein each ring can be fused to a (C₆-C₁₀)aryl, mono- or bicyclic 5-10 membered heteroaryl, (C₃-C₁₀)cycloalkyl or mono- or bicyclic 3-10 membered heterocyclyl;

G¹ and G² are each independently a hydrogen or a glycosyl residue, or a group cleavable under physiological conditions to provide a compound of formula (I) wherein G¹ or G² respectively is hydrogen;

(X¹)_{X1} and (X²)_{X2} each signify that 0, 1, or 2 ring atoms of each respective ring can be nitrogen, provided that where a non-hydrogen substituent is bonded, X¹ or X², respectively, is C;

provided that when G¹ is a 6-deoxyhexopyranosyl residue, G² is H, R¹ is of formula (IIA), R² is hydrogen or hydroxy, R³ is hydrogen, R^{A1} and R^{A2} and R^{A4} are H, R^{A3} and R^{A5} are methyl, and B is CO₂H, or when G¹ and G² are H, R¹ is of formula (IIA), R² is hydrogen, R³ is hydrogen or nitro, R^{A1} and R^{A2} and R^{A4} are H, R^{A3} and R^{A5} are methyl, and B is CO₂H, then R⁵ is not unsubstituted (C₁₀-C₁₆)-alkyl;

or a salt thereof.

In various embodiments, compounds of the invention can include atropisomeric forms due to hindered rotation about the phenyl-phenyl bond of the biphenyl (or bi-aryl or aryl-heteroaryl or bi-heteroaryl) moiety. In various embodiments, a compound of the invention can comprise the Sa atropisomer. Applicants have found that when the amide bond connecting the macrocycle to the tail is not methylated both atropisomers can be present and interconvert freely.

In various embodiments, the invention provides a compound of formula I of the invention wherein when G¹ is a H or a 6-deoxyhexopyranosyl residue, G² is H, R¹ is of formula (IIA), R² is hydrogen or hydroxy, R³ is hydrogen or nitro, R^{A1} and R^{A2} and R^{A4} are H, R^{A3} and R^{A5} are methyl, and B is CO₂H, then R⁵ is not unsubstituted (C₁-C₂₂)alkyl.

In various embodiments, the invention provides a compound of the invention wherein the compound is of formula (IA)

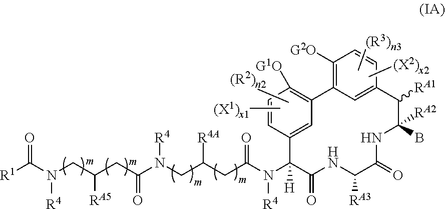

wherein R¹, R², R³, R⁴, R⁵, R⁶, R^{A1}, R^{A2}, R^{A3}, R^{A4}, R^{A5}, R^{A6}, R^{B}, R^{B1}, R^{B2}, and R^{C}, and m, n, n¹, n², n³, B, G¹, G², (X¹)_{X1}, (X²)_{X2}, and Y, are as defined herein;

or a salt thereof.

In various embodiments, the invention provides a compound of the invention wherein R¹ is a group of formula (IIAS), or (IIBS):

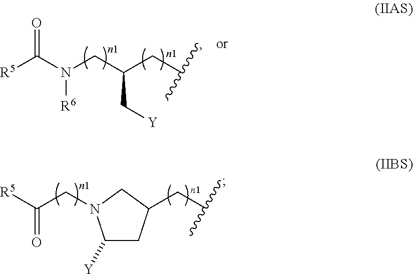

wherein n¹, R⁵, R⁶, and Y, are as defined herein and a wavy line indicates a point of attachment of R¹ to an atom bonded to R¹ in formula (I);

or a salt thereof.

In various embodiments, the invention provides a compound of the invention wherein R⁵ is a (C₁-C₂₂) linear or branched alkyl.

In various embodiments, the invention provides a compound of the invention wherein R⁵ is a (C₁-C₂₂) linear or branched alkyl comprising one or more of groups (A), (B), (C), or (D).

In various embodiments, the invention provides a compound of the invention wherein R⁵ is a (C₁-C₂₂) linear or branched alkyl.

In various embodiments, the invention provides a compound of the invention wherein R⁵ is a (C₁-C₂₂) linear or branched alkyl, comprising one or more of groups (A), (B), (C), or (D).

In various embodiments, the invention provides a compound of the invention wherein R⁵ is any of the following groups

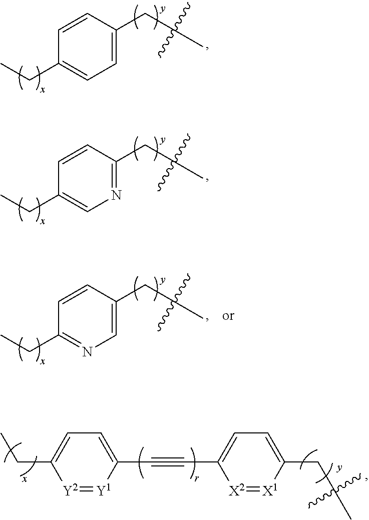

wherein x is 0-14, y is 0-14, provided that x+y≦22, r is 0 or 1, and X¹, X², Y¹ and Y² are each independently C or N, provided that no more than one of X¹ and X², and no more than one of Y¹ and Y², is N, wherein a wavy line indicates a point of attachment of R⁵ to an atom bonded to R⁵ in formula (IIA), (IIB), or (IIC).

In various embodiments, the invention provides a compound of the invention wherein R⁵ is any of the following: methyl, ethyl, (C₃-C₂₂)-n-alkyl, (C₃-C₂₂)-isoalkyl, (C₄-C₂₂)-anteisoalkyl, naphthyl, (C₂-C₁₀) naphthyl, naphthylmethyl, (C₂-C₁₀) naphthylmethyl, biphenyl, (C₂-C₁₀)alkylbiphenyl, biphenylmethyl, (C₂-C₁₀)alkylbiphenylmethyl, (C₄-C₁₂)phenyl, (C₄-C₁₂)benzyl, or (C₂-C₁₀)-1,2-diphenylethynyl, wherein a wavy line indicates a point of attachment of R⁵ to an atom bonded to R⁵ in formula (IIA), (IIB), or (IIC).

In various embodiments, the invention provides a compound of the invention wherein ring bearing one or more X¹ or X², respectively, is a phenyl, pyridyl, pyrazinyl, pyrimidyl, or pyridazinyl, optionally wherein R² and R³ are both hydrogen.

In various embodiments, the invention provides a compound of the invention wherein at least one of R² and R³ is hydrogen.

In various embodiments, the invention provides a compound of the invention wherein at least one of R² and R³ is nitro, halo, hydroxy, glycosyloxy, amino, (C₁-C₄)alkoxy, or (C₁-C₄)alkyl, and n² or n³ respectively, or both, is 1.

In various embodiments, the invention provides a compound of the invention wherein both G are hydrogen.

In various embodiments, the invention provides a compound of the invention wherein any of R^{A1}, R^{A2} and R^{A4} are hydrogen, any of R^{A3} and R^{A5} are methyl, or any combination thereof.

In various embodiments, the invention provides a compound of the invention wherein R^{A3} is hydrogen, methyl, ethyl, n-propyl, isopropyl, n-butyl, isobutyl, 3-hydroxypropyl, 4-hydroxybutyl, or 2,2,2-trifluoroethyl.

In various embodiments, the invention provides a compound of the invention wherein all of R⁴ and R⁶ are independently selected hydrogen or methyl.

In various embodiments, the invention provides a compound of the invention wherein the compound is any of the following compounds of formula (III)

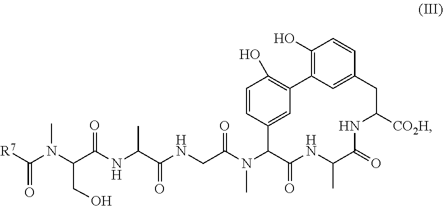

wherein R⁷ is (C₈-C₁₈)-n-alkyl, (C₈-C₁₈)-isoalkyl, (C₈-C₁₈)-anteisoalkyl, any of which includes a group (A), (B), (C), (D), or (E) of claim 1; or is 2-naphthyl, 6-(C₂-C₁₀)-2-naphthyl, 2-naphthylmethyl, 6-(C₂-C₁₀)-2-naphthylmethyl, 4-biphenyl, 4-biphenylmethyl, 4′-(C₂-C₁₀)alkyl-4-biphenyl, 4′-(C₂-C₁₀)alkyl-4-biphenylmethyl, p-(C₄-C₁₂)phenyl, p-(C₄-C₁₂)benzyl, or 4′-(C₂-C₁₀)-1,2-diphenylethynyl;

or a salt thereof.

In various embodiments, the invention provides a compound of the invention wherein the compound is any of the following compounds of formula (IV)

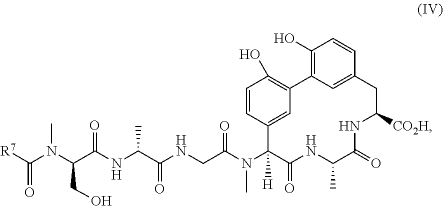

herein R⁷ is (C₈-C₁₈)-n-alkyl, (C₈-C₁₈)-isoalkyl, (C₈-C₁₈)-anteisoalkyl, any of which includes a group (A), (B), (C), (D), or (E) of claim 1; or is 2-naphthyl, 6-(C₂-C₁₀)-2-naphthyl, 2-naphthylmethyl, 6-(C₂-C₁₀)-2-naphthylmethyl, 4-biphenyl, 4-biphenylmethyl, 4′-(C₂-C₁₀)alkyl-4-biphenyl, 4′-(C₂-C₁₀)alkyl-4-biphenylmethyl, p-(C₄-C₁₂)phenyl, p-(C₄-C₁₂)benzyl, or 4′-(C₂-C₁₀)-1,2-diphenylethynyl;

or a salt thereof.

In various embodiments, the invention provides a compound comprising a hydrate, solvate, prodrug, or metabolite of a compound of the invention.

In various embodiments, the invention provides a pharmaceutical composition comprising the compound of the invention and a pharmaceutically acceptable excipient.

### Methods of Treatment of Bacterial Infections Using Compounds of the Invention.

The evolution of bacteria that are resistant to multiple antibiotics poses a serious threat to human health.^{1,2} Although much effort has been focused on the discovery and development of fully synthetic antibiotics,³ most of the antibiotics used in the clinic were derived from natural products, which evolved over eons of time to penetrate bacteria, avoid efflux, and inhibit essential and highly conserved biochemical processes.⁴ Unfortunately, broad-spectrum natural product antibiotics have become increasingly difficult to isolate and the more plentiful narrow-spectrum agents are limited either by unknown factors or by factors that are intrinsic to the compound, such as poor penetration or targeting proteins that are not sufficiently essential or conserved, and which are viewed as challenging to overcome by optimization.³ In contrast, there is much precedent for re-optimizing antibiotics after their spectrum has been compromised by specific resistance mechanisms acquired during clinical use, as evidenced by the development of many “next generation” antibiotics.⁵⁻⁸

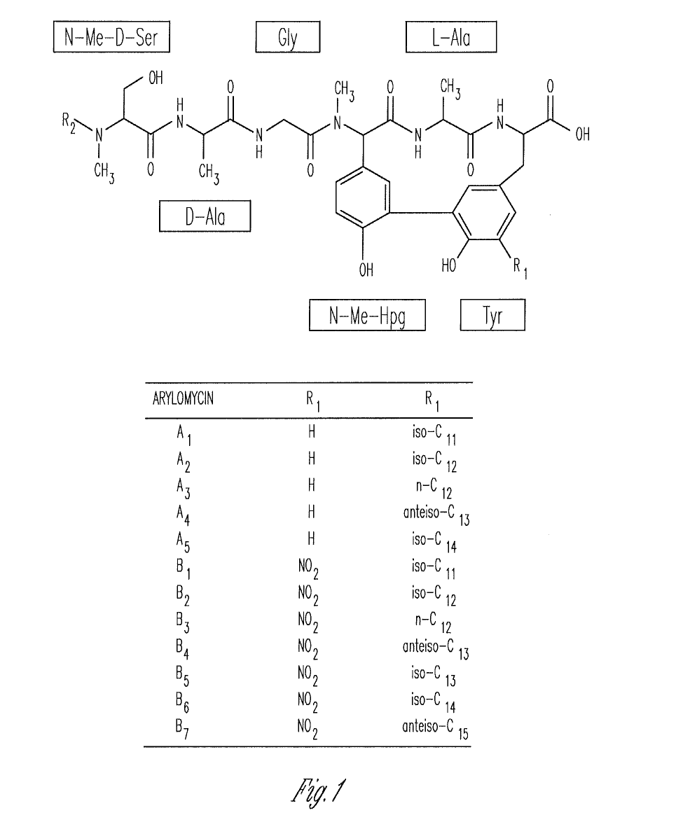

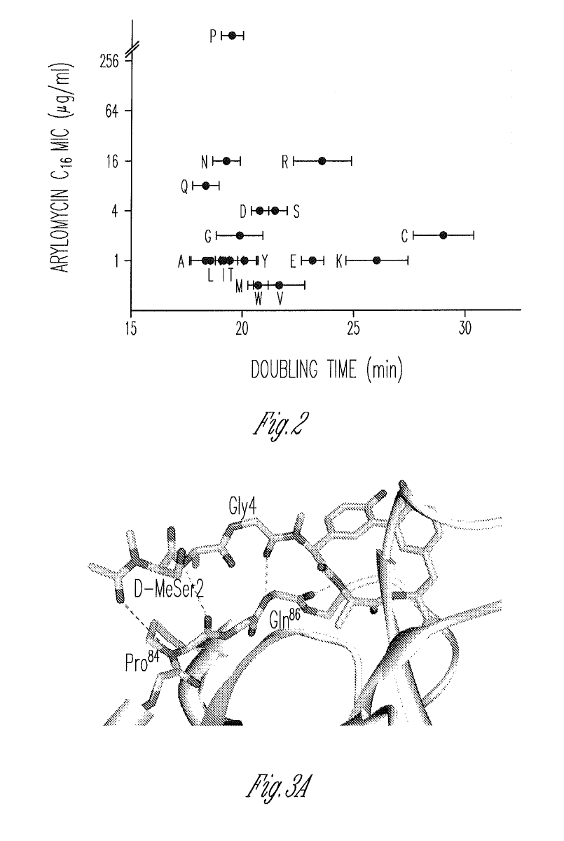

The arylomycins are a class of lipopeptide antibiotics that inhibit bacterial type I signal peptidase (SPase), an essential serine-lysine dyad protease that is anchored to the outer leaflet of the cytoplasmic membrane and that removes N-terminal signal peptides from proteins that are transported out of the cytoplasm.⁹⁻¹¹ Three related series of arylomycins have been identified, the arylomycins A and B and the lipoglycopeptides, which have similar core macrocycles, but different substituents and fatty acid tails (FIG. 1).^{12,13} Based on their novel mechanism of action, there was originally much enthusiasm for these compounds, but despite their ability to inhibit SPase in vitro, and their in vivo activity against the soil bacteria Rhodococcus opacus and Brevibacillus brevis and the human pathogen Streptococcus pneumoniae, they were found to have no activity against a variety of other important human pathogens.^{13,14} This apparently narrow spectrum is surprising considering that SPase is located on the outer leaflet of the cytoplasmic membrane and appears to be present and essential in all Eubacteria.^{10,15-17} To explore the origins of their narrow spectrum, we synthesized and evaluated arylomycin A₂, as well as several derivatives, including arylomycin C₁₆ (FIG. 1).¹⁸ Interestingly, we found that the arylomycins are as active against Staphylococcus epidermidis as the antibiotics used for its treatment, and importantly, we determined that S. epidermidis evolves resistance by introducing a Pro residue into SPase at position 29, which is located in the enzyme's P5 pocket. Remarkably, all bacteria that had been shown to be resistant to the arylomycins have a Pro at the corresponding position, and we identified a wide variety of bacteria that lack this residue and showed that the majority of them are sensitive to the arylomycins, including the Gram-positive pathogens Streptococcus pyogenes and Staphylococcus haemolyticus, and the Gram-negative pathogens Helicobacter pylori and Chlamydia trachomatis. Moreover, while the arylomycins slow the growth of Staphylococcus aureus strain 8325, they do not actually prevent it,¹⁸ even at concentrations as high as 128 μg/ml, however, they do prevent the growth of USA300, an epidemic MRSA isolate, with an MIC of 16 μg/ml. While this might result from unique features associated with methicillin resistance, it suggests that the arylomycin scaffold has the potential for broader spectrum S. aureus activity. Importantly, we showed that the Pro residue imparts resistance by reducing the affinity with which the arylomycin binds, and that removing it is sufficient to render resistant S. aureus, Escherichia coli, and Pseudomonas aeruginosa highly sensitive. This data suggests that if the arylomycins could be optimized to bind SPases regardless of the resistance-conferring Pro, they would have a remarkably broad spectrum of activity.

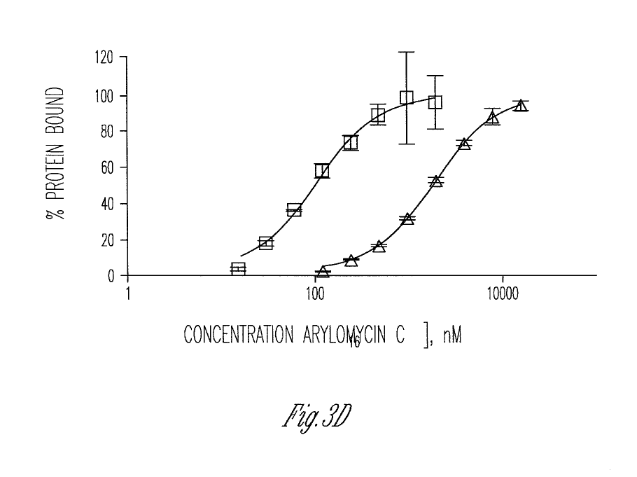

Despite not having activity against wild type E. coli, two crystal structures of arylomycin A₂ bound to a soluble fragment of E. coli SPase have been reported (FIG. 3A).^{19,20} The arylomycin is seen to bind in an extended β-sheet conformation that likely mimics the binding of membrane bound pre-protein substrates.¹⁹ The C-terminal macrocycle of the arylomycin binds in a deep hydrophobic cleft and makes multiple hydrogen-bonds and hydrophobic interactions with the protein, while the C-terminal carboxyl group forms a critical salt bridge with the catalytic residues. The peptide tail extends down a shallow cleft in the surface of SPase and forms two hydrogen-bonds with backbone residues of the protein. The critical resistance-conferring residue, Pro⁸⁴, interacts with the N-terminal end of the peptidic tail and appears to preclude the formation of a hydrogen-bond to a carbonyl oxygen of the arylomycin and possibly to alter the trajectory of the lipid moiety as it enters the membrane (FIG. 3A). While the crystal structures are likely to reveal little information about the biologically relevant structure of the lipid tail due to the use of a soluble truncated fragment of SPase and the absence of a membrane bilayer, it most likely adopts an extended conformation to maximize packing within the outer leaflet of the cytoplasmic membrane.

As with a variety of other antibiotics with membrane-associated targets,²¹⁻²⁴ the lipopeptide tail of the arylomycins has been shown to play an important role in their activity.^{13,14,21-24} Here, we report the first structure-activity relationship study of synthetic arylomycins, focusing on derivatives with altered lipopeptide tails. The activities of the arylomycin derivatives were evaluated with S. epidermidis, S. aureus, E. coli, and P. aeruginosa. With each pathogen, the derivatives were evaluated in the context of SPases with and without the critical resistance-conferring Pro, to identify the changes in activity that result from altered interactions with this resistance-conferring residue and to identify the types of modifications that might be pursued to overcome resistance and thereby instill the arylomycin scaffold with broad-spectrum antibacterial activity.

SPase and the N-terminal portion of its natural substrates are embedded within the bacterial cell membrane,^{26,27} suggesting that some part of the inhibitor's lipopeptide tail must also be accommodated within the membrane. To probe this interaction, and also to determine whether a positive charge at the lipid-peptide junction of arylomycin is capable of interacting with the negatively charged head groups of the phospholipid bilayer, we synthesized a derivative of the formula shown (Compound 25 of Table 2B), wherein R¹ is H and R² (of FIG. 1) is a C16-n-alkyl, not an alkanoyl, chain, which serves to replace the lipid tail amide with a charged tertiary amine. This derivative has significantly reduced activity against S. epidermidis and sensitized S. aureus (MICS of 32 and 64 μg/ml, respectively), and no activity against any of the wild type or mutant Gram-negative strains examined. The decreased activity likely results from the inability of a hydrophobic environment to accommodate the charge, suggesting that this portion of the tail is embedded in the membrane or within the interface between the membrane and SPase.

Accordingly, a series of hydrophobic tail analogs of arylomycin A2, adapted to accommodate the proline residues of the resistant forms of SPase as described in the Specification and documents cited herein, were designed. Certain compounds were synthesized and tested, as shown below in the bioactivity Tables 1-4, below.

To explore the minimal tail length required for activity and to determine whether there is a limit to the tail length that can be accommodated within the cytoplasmic membranes of the different bacteria, we synthesized and characterized derivatives 2-5 (Table 1). None of these derivatives gained activity against any of the resistant bacteria relative to arylomycin C₁₆, but significant differences were apparent with S. epidermidis and the genetically sensitized strains. With the sensitive strains, the C₈ derivative 2 has no activity, but the C₁₀ derivative 3 has activity against S. epidermidis, S. aureus, and E. coli, while only 4 and 5 show activity against P. aeruginosa, revealing that at minimum a C₁₂ tail is required. In each case, activity increased with increasing tail length until it plateaued with the C₁₆ fatty acid tail (i.e. arylomycin C₁₆), and activity decreased slightly with the C₁₈ derivative 5 with all but P. aeruginosa.

To further explore the effects of increased hydrophobicity, we synthesized and characterized derivatives with tails that contain one or more aromatic rings (Table 1). We first examined the series of napthyl and biphenyl derivatives 6-8. The napthyl derivative 6 shows no activity against any of the bacteria tested, while the biphenyl derivative 7 retains some activity against wild type S. epidermidis. We found that compound 8, which lacks the methylene spacer between the fatty acid carbonyl and the biphenyl moiety also retains some activity against S. epidermidis, suggesting that flexibility of the biphenyl moiety is not essential. To further explore this biphenyl architecture, we synthesized the p-alkyl substituted biphenyl derivatives 9-12. We observed an increase in activity with increasing alkyl substituent length against wild type S. epidermidis that plateaued with the C₆ and C₈ derivatives 11 and 12, which are also active against resistant S. epidermidis. Interestingly, several of the compounds in this series are also active against both sensitized and wild type S. aureus, with relative activities similar to those observed with S. epidermidis, but with absolute activities that were somewhat lower. None of the biphenyl derivatives have activity against the wild type or sensitized strains of P. aeruginosa, but they do maintain activity against sensitized E. coli, again showing trends that were similar to those observed with S. epidermidis and S. aureus. Overall, the data reveal that relative to the straight chain derivatives, the biphenyl derivatives show similar activities against S. epidermidis and E. coli, less activity against P. aeruginosa, but greater activity against S. aureus.

We next examined the series of phenyl substituted tail mimetics 13-15 (Table 1). With S. epidermidis and the genetically sensitized strains, we again observed an increase in activity with increasing alkyl chain length. Moreover, the decylphenyl derivative 15 has activity against wild type S. aureus. Because the number of carbon atoms in this derivative is similar to that of arylomycin C₁₆, which has no activity against wild type S. aureus, the data suggest that at least some of the activity is mediated by the interaction of the polarizable aromatic moiety with the membrane or with SPase.

To explore the effects of lipopeptide methylation, and to begin a more focused exploration of modifications that might overcome the deleterious effects of the resistance-conferring Pro, we synthesized and characterized derivatives with altered N-methylation at d-MeSer2 and d-Ala3 (Table 2), arylomycin residues that are proximal to this critical residue when bound to SPase (FIG. 3A). The absence of the d-MeSer2 N-methyl group in 16 results in a slight decrease in activity against both the wild type and resistant S. epidermidis strains, and a more pronounced loss of activity against each of the other strains that is most pronounced with P. aeruginosa. It is unlikely that this reduced activity results from specific deleterious interactions with the lipid membrane due to membrane fluidity, nor with SPase, as the structure of the E. coli SPase-arylomycin A₂ complex suggests that this region of the lipopeptide tail is either disordered or oriented away from the protein (although as discussed above, the N-terminally truncated form of SPase used in the structural studies renders this conclusion somewhat speculative).¹⁹ Thus, the observed decrease in activity is likely the result of decreased hydrophobicity, outer membrane penetration, or protease resistance. The loss in activity is even more pronounced with compound 17 where methylation of d-Ala3 ablates activity against all organisms tested, likely resulting from replacement of a stabilizing H-bond with a destabilizing steric clash.

To explore the effects of lipopeptide tail rigidity, and to further explore modifications that might directly compensate for the resistance conferred by Pro29/84 of SPase, we designed the hydroxyproline derivative 18 (Table 2). In this compound, the side chain of d-MeSer2, which interacts with the sidechain and backbone of the SPase residue at position 29/84,¹⁹ is homologated by a methylene unit and fused with the methyl group of the neighboring N-methyl amide bond. We found that this modification results in a complete loss of activity against the Gram-negative organisms but only little to moderate loss in activity against the Gram-positive organisms. Interestingly, because 18 retained full activity against resistant S. epidermidis, the disparity in activities against the wild type and resistant variants is greatly decreased, suggesting that at least for this organism, 18 recognizes both the Ser- and the Pro-variant SPases similarly.

To explore the effects of decreased rigidity, we synthesized derivative 19 (Table 2), which lacks the peptide bond between the serine and the fatty acid tail (and thus should impart the tail with greater rotational freedom). This molecule has significantly less activity compared to arylomycin C₁₆ against all organisms tested, with no observable activity against E. coli, S. aureus, or P. aeruginosa, and only moderate activity against S. epidermidis.

To increase flexibility without introducing or deleting other peptidic functionalities that might contribute to binding SPase, we synthesized and evaluated derivatives with one or two methylene units inserted immediately N-terminal or C-terminal to the amide bond linking d-MeSer2 to d-Ala3 (20-23, Table 2). These compounds did not gain activity against either of the wild type Gram-negative bacteria. With the sensitized Gram-negative strains, activity was observed only with 20, which relative to the parent compound is 16-fold less active against E. coli, but only 2-fold less active against P. aeruginosa. The effects of methylene addition were significantly different with the Gram-positive bacteria. Relative to arylomycin C₁₆, derivatives 20-23 lost 8- to 16-fold activity against sensitive S. epidermidis, but retained activity against the resistant strain. This demonstrates that once the hydrogen-bond donor of the protein is removed (by mutation to Pro), perturbing the H-bond acceptor does not further decrease activity. The results were somewhat more complicated with S. aureus. As expected, relative to arylomycin C₁₆, addition of the methylene units decreased activity against the sensitized strain of S. aureus, 2- to 8-fold for 21-23, and at least 64-fold for 20. In the case of wild type S. aureus, however, no activity is observed with 20 or 21, but interestingly, 22 and especially 23 gain activity.

TABLE 1
MICs (μM) of selected compounds.

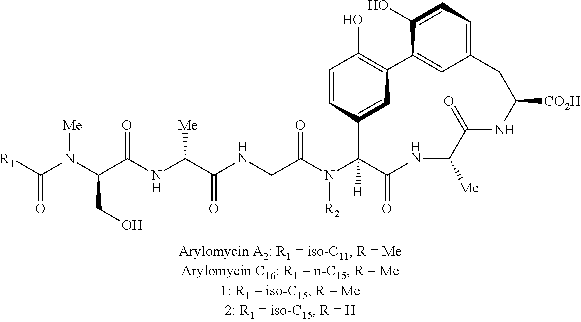

| Strain | Arylomycin A₂ | Compound 1 | Compound 2 |
|---|---|---|---|
| E. coli MG1655 | >128 | >128 | >128 |
| S. aureus 8325 | >128 | >128 | >128 |
| S. epidermidis ATCC 35984 | 1 | 0.5 | 1 |
| B. anthracis Sterne | n.d. | 32 | n.d. |
| E. faecium AEFA001^{a} | n.d. | >64 | n.d. |
| E. faecalis ATCC 29212 | n.d. | >64 | n.d. |
| E. faecalis ATCC 51299 | n.d. | >64 | n.d. |

^{a}Part of the Achaogen, Inc. strain collection.

TABLE 2A
Activity (MICs (μg/ml)) of arylomycin derivatives with altered fatty acid tails
against strains of S. epidermidis, S. aureus, E. coli, and P. aeruginosa harboring SPase
without (Sensitive) and with (Resistant) the arylomycin-resistance conferring Pro residue.

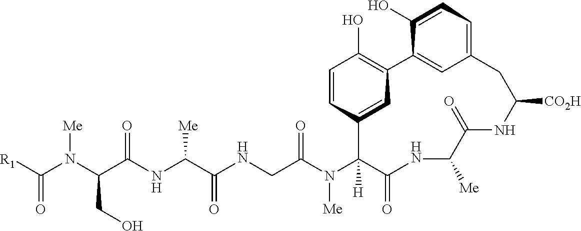

| | | Sensitive^{b} | | | | Resistant^{c} | | | |
|---|---|---|---|---|---|---|---|---|---|
| | R | Se | Sa | Ec | Pa | Se | Sa | Ec | Pa |
| 3 | 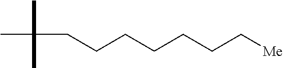 | >64 | >64 | >64 | >64 | >64 | >64 | >64 | >64 |
| 4 | 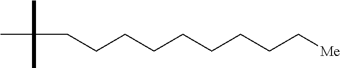 | 16 | 64 | 32 | >64 | >64 | >64 | >64 | >64 |
| 5 | 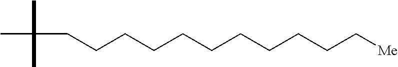 | 0.5 | 16 | 8 | 64 | >64 | >64 | >64 | >64 |
| —^{a} | 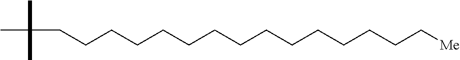 | 0.5 | 2 | 0.5 | 8 | 8 | >64 | >64 | >64 |
| 6 | 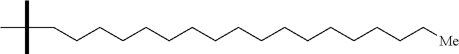 | 1 | 4 | 2 | 8 | 16 | >64 | >64 | >64 |
| 7 | 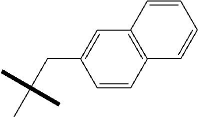 | >64 | >64 | >64 | >64 | >64 | >64 | >64 | >64 |
| 8 | 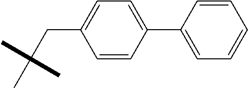 | 32 | >64 | >64 | >64 | >64 | >64 | >64 | >64 |
| 9 | 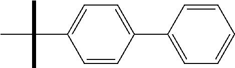 | 64 | >64 | >64 | >64 | >64 | >64 | >64 | >64 |
| 10 | 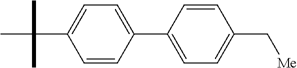 | 8 | 64 | 16 | >64 | >64 | >64 | >64 | >64 |
| 11 | 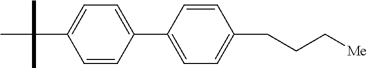 | 1 | 16 | 4 | >64 | >64 | >64 | >64 | >64 |
| 12 | 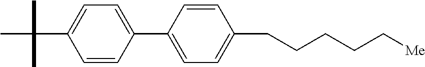 | 0.5 | 8 | 1 | >64 | 32 | 64 | >64 | >64 |
| 13 | 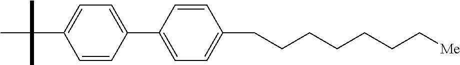 | 1 | 8 | 1 | >64 | 16 | 16 | >64 | >64 |
| 14 | 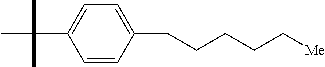 | 8 | 64 | 32 | >64 | >64 | >64 | >64 | >64 |
| 15 | 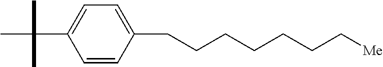 | 1 | 8 | 8 | 64 | >64 | >64 | >64 | >64 |
| 16 | 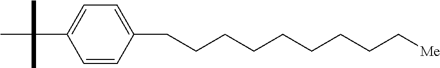 | 0.5 | 4 | 2 | 16 | 16 | 32 | >64 | >64 |

Se = S. epidermidis, Sa = S. aureus, Ec = E. coli, Pa = P. aeruginosa
^{a}Arylomycin C₁₆ (fourth entry) included for reference.
^{b}Sensitive strains include wild type S. epidermidis RP62A, lepB(P29S) S. aureus 8325, lepB(P84L) E. coli MG1655, and lepB(P84L) P. aeruginosa PAO1.
^{c}Resistant strains include spsB(S29P) S. epidermidis RP62A, and wild type S. aureus 8325, E. coli MG1655, and P. aeruginosa PAO1.

TABLE 2B
Activity (MICs (μg/ml)) of arylomycin derivatives with altered lipopeptide tails
against strains of S. epidermidis, S. aureus, E. coli, and P. aeruginosa harboring SPase
without (Sensitive) and with (Resistant) the arylomycin-resistance conferring Pro residue.

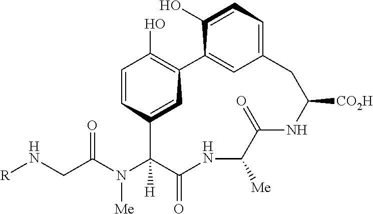

| | | Sensitive^{b} | | | | Resistant^{c} | | | |
|---|---|---|---|---|---|---|---|---|---|
| | R | Se | Sa | Ec | Pa | Se | Sa | Ec | Pa |
| —^{a} | 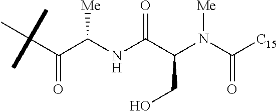 | 0.5 | 2 | 0.5 | 8 | 8 | >64 | >64 | >64 |
| 17 | 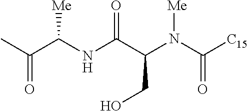 | 1 | 16 | 4 | >64 | 32 | >64 | >64 | >64 |
| 18 | 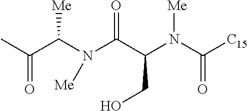 | >64 | >64 | >64 | >64 | >64 | >64 | >64 | >64 |
| 19 | 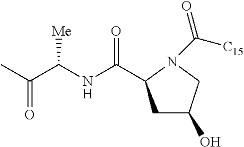 | 4 | 4 | >64 | >64 | 8 | >64 | >64 | >64 |
| 20 | 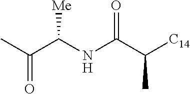 | 8 | >64 | >64 | >64 | >64 | >64 | >64 | >64 |
| 21 | 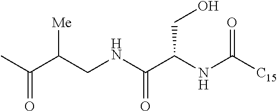 | 8 | >64 | 8 | 16 | 16 | >64 | >64 | >64 |
| 22 | 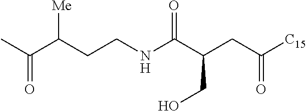 | 8 | 8 | >64 | >64 | 8 | >64 | >64 | >64 |
| 23 | 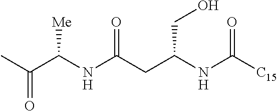 | 4 | 16 | >64 | >64 | 8 | 64 | >64 | >64 |
| 24 | 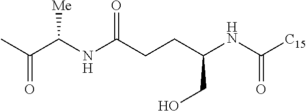 | 8 | 4 | >64 | >64 | 16 | 16 | >64 | >64 |

Se = S. epidermidis, Sa = S. aureus, Ec = E. coli, Pa = P. aeruginosa
^{a}Arylomycin C16 (fourth entry) included for reference.
^{b}Sensitive strains include wild type S. epidermidis RP62A, /epB(P29S) S. aureus 8325, lepB(P84L) E. coli MG1655, and lepB(P84L) P. aeruginosa PAO1. See text for details.
^{c}Resistant strains include spsB(S29P) S. epidermidis RP62A, and wild type S. aureus 8325, E. coli MG1655, and P. aeruginosa PAO1.

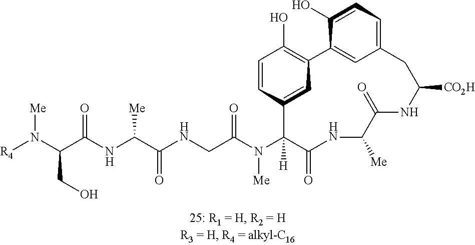

TABLE 3
Arylomycin P3 Position Derivatives

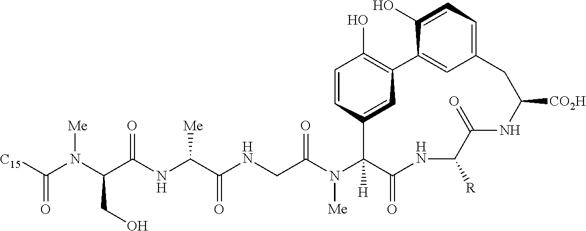

| | R | S. epidermidis^{b} | S. aureus^{c} | E. coli^{d} | P. aeruginosa^{e} |
|---|---|---|---|---|---|
| 26 | 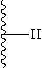 [SMILES: *[H]] | 8 | >64 | 16 | >64 |
| ^{a} | 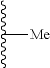 | 0.25 | 4 | 2 | 4 |
| 27 | 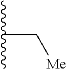 [SMILES: *CC] | 0.5 | 8 | 1 | 4 |
| 28 | 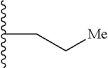 [SMILES: *CCC] | 1 | 8 | 2 | 8 |
| 29 | 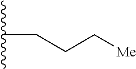 [SMILES: *CCCC] | 4 | 16 | >128 | >128 |
| 30 | 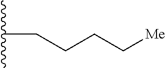 [SMILES: *CCCCC] | 4 | 16 | >128 | >128 |
| 31 | 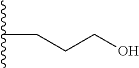 [SMILES: *CCCO] | >64 | >64 | >64 | >64 |
| 32 | 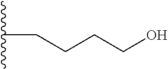 [SMILES: *CCCCO] | >64 | >64 | >64 | >64 |
| 33 | 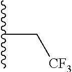 [SMILES: *CC(F)(F)F] | 8 | >64 | 16 | >64 |
| 34 | 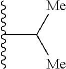 [SMILES: *C(C)C] | 4 | 32 | 4 | 16 |
| 35 | 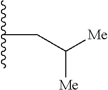 [SMILES: *CC(C)C] | 2 | 32 | 4 | >64 |

^{a}Arylomycin C₁₆ (fourth entry) included for reference.
^{b}wild type S. epidermidis RP62A.
^{c}lepB(P29S) S. aureus 8325,
^{d}lepB(P84L) E coli MG1655, and lepB(P84L)
^{e}P. aeruginosa PAO1

TABLE 4
MICs of Arylomycin Derivatives (μg/mL)

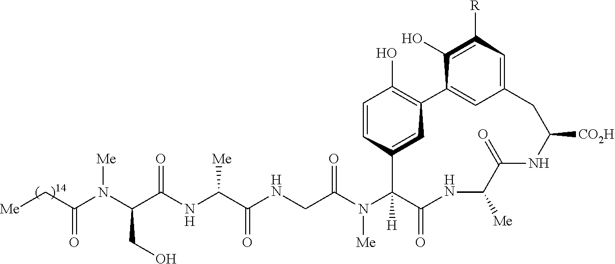

| | Arylomycin C₁₆ | Arylomycin B-C₁₆ | 36 |
|---|---|---|---|
| Strain | (R = H) | (R = NO₂) | (R = NH₂) |
| S. epidermidis | 0.25 | 0.13 | 8 |
| S. aureus P29S | 8 | 8 | 64 |
| E. coli P84L | 2 | 2 | 16 |
| E. coli | >64 | >64 | >64 |
| P. aeruginosa P84L | 4 | 4 | 32 |
| B. brevis | >64 | >64 | >64 |
| R. equi | 16 | 32 | nd |
| R opacus | 1 | 4 | nd |
| S. agalactiae | >128 | 8 | nd |
| S. pyogenes | 8 | 4 | nd |
| S. pneumoniae | 8 | 16 | nd |
| C. efficiens | 16 | 16 | nd |
| C. glutamicum | 2 | 2 | nd |
| L. lactis | 16 | 32 | nd |

The phenyl- and biphenyl-fatty acid tail series showed similar activities against S. epidermidis, S. aureus, and E. coli, with the longer p-alkyl derivatives having activity against both sensitive and resistant strains of S. aureus. Interestingly, P. aeruginosa again shows unique behavior as it is not inhibited by any of the biphenyl-modified derivatives. This is particularly noteworthy considering that it is inhibited by the C₈- and C₁₀-substituted phenyl analogs, which in some cases are less hydrophobic. While some of the differences may result from altered outer membrane penetration or in vivo stability, the data likely reflect suboptimal insertion of the arylomycin into the plasma membrane of P. aeruginosa. It is interesting to speculate that this might result from unique aspects of the phospholipids that comprise the plasma membrane of P. aeruginosa, such as the presence of phosphatidylcholine,⁴⁶⁻⁵⁰ or from different constituent fatty acids.⁵¹⁻⁵⁴ For example, P. aeruginosa appears to employ a higher percentage of cis-vaccenic acid (a C₁₈ fatty acid) relative to palmitic and palmitoleic acids (which are C₁₆ fatty acids),⁵¹⁻⁵⁴ possibly resulting in a slightly thicker plasma membrane and possibly accounting for the generally longer fatty acid tail lengths that were observed to be required for P. aeruginosa inhibition. Overall, the data collected with the different tail derivatives suggest that the phenyl-modified derivatives are likely better scaffolds for arylomycin optimization than the natural, saturated fatty acid chains. This is most clearly highlighted by compound 15, which retains all of the activities of the parent compound arylomycin C₁₆ but also gains activity against S. aureus.

N-methylation is common with non-ribosomally synthesized peptides such as the arylomycins, and is generally thought to optimize hydrophobicity, hydrogen-bonding potential, conformation, and/or resistance to proteases.⁵⁵⁻⁵⁸ The peptide portion of the arylomycin lipopeptide tail is backbone methylated at D-MeSer2 and MeHpg5, but not at D-Ala3 or Gly4. Previously, we showed that the methyl group at MeHpg5 pre-organizes the biaryl ring system for recognition of SPase.¹⁸ When we altered the backbone methylation state of D-MeSer2 and D-Ala3, which are both proximal to the critical resistance-conferring Pro in the E. coli SPase-arylomycin A₂ complex,¹⁹ significant activity was lost against both Gram-positive and Gram-negative bacteria. Although the specific origins of the decreased activity may differ at the two sites examined, the data suggests that natural lipopeptide tail methylation pattern is already optimized for activity.

In an effort to more directly compensate for the negative interactions introduced by the resistance-conferring Pro, we synthesized several derivatives with increased or decreased flexibility around D-MeSer2 and D-Ala3. None of the derivatives gained activity against either of the wild type Gram-negative bacteria, and while 20 retained activity against sensitized P. aeruginosa, it and the other derivatives lost activity against sensitized E. coli. The results were significantly different with the Gram-positive pathogens. Relative to arylomycin C₁₆, derivatives 20-23 each lost 8 to 16-fold activity against sensitive S. epidermidis and 2- to >32-fold activity against the genetically sensitized S. aureus. This trend is consistent with the modifications shifting the register of the β-sheet formed between SPase and the inhibitor such that a stabilizing H-bond is lost. However, and more importantly, 22, and especially 23, gain activity against wild type S. aureus. As mentioned above, the structure of the E. coli SPase-arylomycin A₂ complex suggests that Pro84 (and by inference the homologous Pro in the other bacteria) disrupts arylomycin binding by physically occluding the lipopeptide tail from a hydrophobic groove and by disrupting an otherwise stabilizing hydrogen-bond with the carbonyl group of the lipid tail. While the precise mechanism by which these derivatives gain activity against S. aureus remains to be determined, the data nonetheless support the possibility that the spectrum of the arylomycins may be optimized by derivatization.

Accordingly, a series of derivatives with residues of varied side chains at the position of the macrocycle of arylomycin C16 where Ala is present, adapted to compensate for binding energy lost due to the existence of the proline residues of the resistant forms of SPase as described in the specification and documents cited herein, were designed. Certain compounds were synthesized and tested, as shown below in the bioactivity Tables 3, below. The derivatives were evaluated by characterizing their minimal inhibitory concentration (MIC) against wild type S. epidermidis (strain RP62A), as well as mutant strains of S. aureus 8325, E. coli MG1655, and P. aeruginosa PAO1 that are rendered sensitive to the arylomycins via mutation of the resistance-conferring SPase Pro residue to Ser (S. aureus) or Leu (E. coli, and P. aeruginosa).

We initially explored the effects of removing the side chain at P3 by incorporation of Gly (26, Table 3). This compound lost significant activity against S. epidermidis (32-fold), S. aureus (>16 fold), E. coli (32-fold) and P. aeruginosa (>16-fold), suggesting that α-branching is required at this position either for packing within the hydrophobic environment of the S3 pocket and/or to help orient the backbone of the arylomycin, and thereby decrease the internal entropy of the macrocycle lost upon binding.

Having established the requirement for an α-substituent at the P3 position to maintain activity, we systematically explored the effect of increased side chain length with arylomycin derivatives 27-30. Interestingly, the data reveal that arylomycin C₁₆, which bears a methyl group at this position, and analogs with ethyl, or n-propyl side chain results all have indistinguishable activities against each of the bacteria tested (Table 5-1). In contrast, the derivatives and 29 with n-butyl and n-pentyl side chains, respectively, had significant effects that were somewhat different with the different bacteria examined. Relative to arylomycin C₁₆, both compounds 29 and 30 lost 8- to 32-fold activity against S. epidermidis, 32-fold against S. aureus, and at least 32- and 64-fold against P. aeruginosa and E. coli, respectively. The data suggest that the different SPases do not discriminate against shorter side chains at this position, but have varying abilities to tolerate increasingly longer P3 side chains.

While an inability to accommodate the longer P3 side chains is a simple explanation for the structure activity-relationships revealed with 27-30, the crystal structure of the inhibitor-bound E. coli peptidase reveals the presence of several crystallographically observable water molecules near the P3 pocket. Thus, it is also possible that the n-butyl and n-pentyl derivatives may have lost activity by forcing a hydrophobic methyl group proximal to these water molecules, or by forcing desolvation of the protein without compensating with any stabilizing interactions. To address these possibilities, we explored the effects of increased hydrophilicity and/or hydrogen-bonding via derivatives 31 and 32 which bear n-propanol and n-butanol side chains, respectively. All activity was lost with these compounds against all bacteria tested, suggesting that the longer alkyl or hydroxyalkyl are not accommodated due to size constraints with the S3 pocket.

To further explore how changes in the hydrophobicity of the P3 side chain impact activity we examined trifluoroethyl derivative 33. Such fluorinated derivatives are thought to be ‘superhydrophobic’²⁴ and are often used in drug optimization.²⁵ We found that compound 33 is 8-fold less active against E. coli, at least 16-fold less active against P. aeruginosa and S. aureus, and 32-fold less active against S. epidermidis.

Having explored the effect of altered length and hydrophobicity within a linear side chain, we turned our attention to the Val and Leu derivatives 34 and 35, which possess β- and γ-branched side chains, respectively. While these compounds are also less active than the parent compound, the loss in activity was again organism dependent. Both 34 and 35 lost 8- to 16-fold activity against both Gram-positive bacteria, but they maintained activity against E. coli. Interestingly, P. aeruginosa more strongly differentiated between the branched derivatives, with 34 losing 4-fold activity, but 35 losing >16-fold activity. This data suggests that the S3 pocket of the E. coli SPase is relatively tolerant toward branching in general, that the Gram-positive SPase are less so, and that the S3 pocket of P. aeruginosa SPase is relatively tolerant of β-branching but intolerant of γ-branching.

In summary, we have synthesized the first series of arylomycin derivatives with substitution in the backbone of the macrocycle. We found that the side chain at the P3 position of arylomycin contributes significantly to binding. In addition, we found that the S3 pocket of SPase can accommodate up to three linear saturated carbons from the P3 position of arylomycin however, increasing the size of the substituent filling this pocket does not increase overall activity. Arylomycin derivatives with side-chains larger than three linear saturated carbons either linearly or laterally at the P3 position lose activity against most strains with larger losses of activity seen when larger substituents were present. While these derivatives did not make arylomycin more potent, they defined the limits of the S3 pocket for the inhibitor and they suggest a binding model where the arylomycins, and perhaps the recognition sequence of preproteins, are not free in solution but are bound in the lipophilic membrane before binding SPase.

To explore the effect of substituents on the aromatic ring affect binding of the arylomycin we synthesized and arylomycin B derivative arylomycin B C₁₆ and its amino derivative 36. They were adapted to compensate for binding energy lost due to the existence of the proline residues of the resistant forms of SPase as described in the specification and documents cited herein, were designed. Certain compounds were synthesized and tested, as shown below in the bioactivity Table 4, below.

The activity of arylomycin B—C₁₆ and its derivative 36 was characterized by determining the minimal inhibitory concentration (MIC) required to inhibit the growth of wild type S. epidermidis (strain RP62A), and E. coli (MG1655). The compounds were also tested against strains of S. aureus (8325), E. coli (MG1655), and P. aeruginosa (PAO1) that were rendered sensitive to the arylomycins by mutation of the resistance-conferring Pro to a residue that does not confer resistance (P29S in the S. aureus protein, and P84L in the E. coli, and P. aeruginosa proteins).

Like arylomycin A₂ and its derivative arylomycin C₁₆, arylomycin B—C₁₆ has potent activity against S. epidermidis (Table 4) and no activity against wild-type E. coli. In addition, similar to the arylomycin A compounds, arylomycin B—C₁₆ has activity against the mutant strains of S. aureus, E. coli, and P. aeruginosa. Thus, the activity of the B series compound is limited against natural isolates via the same mechanism as the A series compounds. In fact, the level of arylomycin B—C₁₆ activity against all strains tested is indistinguishable from that of arylomycin C₁₆ (Table 4). Surprisingly, our data contradicts the previously reported conclusion that the arylomycins have activity against B. brevis and that nitro substitution increases the activity of the arylomycin scaffold.¹⁸ In our experiments, using both MHBII broth and nutrient broth (as was used in the previously reported studies) both compounds showed no activity against B. brevis.

Relative to the two natural products, we found that the amino derivative 36 is significantly less active against all bacteria tested and the loss in activity is slightly larger against the Gram-positive bacteria (32-fold) than against the Gram-negative pathogens (8-fold). The amino group is expected to be protonated and thus charged at physiological pH, and the inability of the SPase binding site to accommodate this charge likely explains the decreased activity.

To generate a broader assessment of the activity of the A- and B-series arylomycins, we examined representatives from a broad range of bacteria that are sensitive to arylomycin C₁₆²⁰ including Rhodococcus equi, Rhodococcus opacus, Streptococcus agalactiae, Streptococcus pneumonia, Streptococcus pyogenes, Corynebacterium efficiens, Corynebacterium glutamicum and Lactococcus lactis (Table 4). For almost all of the bacteria tested the inhibitory concentrations for arylomycin C₁₆ and arylomycin B—C₁₆ were identical or within the error of the experiment. However, Streptococcus agalactiae showed significant differences in MICs between the compounds with arylomycin B—C₁₆ having greater than 16-fold more activity than its non-nitrosylated analogue arylomycin C₁₆. This result is especially interesting given that the in neither of the two SPases of S. agalactiae is the resistance conferring Pro present and that identical activities were observed between the two compounds against the closely related organisms Streptococcus pneumoniae, and Streptococcus pyogenes. It is interesting to speculate that in the context of an arms race this species may have found an alternative method for mediating resistance to the arylomycin A series that was overcome by the installation of a nitro group in the arylomycin B series.

In conclusion, we have shown that aminated arylomycin loses significant activity relative to the nitrosylated variant. In addition, we have showed that arylomycin B—C₁₆ has no difference in activity against S. epidermidis, mutant strains of S. aureus, E. coli and P. aeruginosa and multiple other strains of bacteria when compared to the analogous arylomycin C₁₆ of the A series. Importantly, it was found that S. agalactiae is uniquely sensitive to the arylomycin B—C₁₆. In addition, closely related strains of bacteria showed no difference between the A and B series arylomycins. These results support the idea that nitrosylation of the arylomycin is important for biological activity against some bacteria and that its inclusion in the repertoire of arylomycins was possibly the result of selection on the producing organism.

Reports by other workers indicate that arylomycins (e.g., those shown in FIG. 1) have little whole cell activity against most bacterial pathogens except possibly for Staphylococcus epidermidis, and Rhodococcus opacus. See, e.g., Kulanthaivel et al., J. Biol. Chem. 279: 36250-58 (2004); Schimana et al., J. Antibiotics 55:565-70 (2002). For example, currently available reports indicate that arylomycins A and B lack activity against the Gram-negative bacteria Escherichia coli K12, Proteus mirabilis ATCC 35501, Pseudomonas fluorescens DSM 50090 and against the eukaryotic organisms Saccharomyces cerevisiae ATCC 9080, Botrytis cinerea Tu 157 and against the green algae Chlorella fusca and against the duckweed Lemna minor.

However, according to the invention, arylomycins actually do have activity against a variety of bacterial species. For example, the following bacterial species are susceptible to arylomycins: Rhodococcus equi, Corynebacterium diphtheriae, Lactococcus lactis subsp. cremoris, Corynebacterium glutamicum, Francisella tularensis, Campylobacter jejuni, Helicobacter pylori, Propionibacterium acnes, Chlamydia trachomatis, Chlamydophila pneumoniae, Staphylococcus carnosus, Staphylococcus haemolyticus, Staphylococcus hominis, Staphylococcus lugdunensis, Streptococcus agalactiae, Streptococcus dysgalactiae, Streptococcus mitis, Streptococcus oralis, and Streptococcus pyogenes, as illustrated in the following Table 5.

TABLE 5
Bacteria Predicted by sequence to be Sensitive and Confirmed Sensitive.
Predicted by sequence to be Sensitive and Confirmed Sensitive.

| | Aminio acid −7 from | |
|---|---|---|
| Species | catalytic Ser^{a} | MIC (μg/ml) |
| Staphylococcus epidermidis | S,S | 0.25 |
| Staphylococcus haemolyticus | S,S | 2 |
| Staphylococcus hominis | S,S | 0.25 |
| Staphylococcus lugdunensis | S,T | 0.25 |
| Staphylococcus simulans | S,? | 0.25 |
| Staphylococcus cohnii | S,? | 8 |
| Streptococcus pneumoniae | N | 16 |
| Streptococcus pyogenes | A | 16 |
| Corynebacterium glutamicum | M | 2 |
| Rhodococcus opacus | V | 2 |
| Lactococcus lactis | L | 16, >128^{b} |
| Rhodococcus equi | V,I | 16 |
| Helicobacter pylori | A | 4 |
| Chlamydia trachomatis | L | 6 |
| Francisella tularensis | N | 4-16, >64^{b} |

^{a}Multiple amino acids indicate where organisms express multiple SPases
^{b}Range of MICs across different isolates

Accordingly, another aspect of the invention is a method of treating a bacterial infection in an animal that includes administering any one or any combination of the arylomycin compounds (e.g., compounds of Formula I) to the animal, wherein the bacterial infection comprises an infection by Rhodococcus equi, Corynebacterium diphtheriae, Lactococcus lactis subsp. cremoris, Corynebacterium glutamicum, Francisella tularensis, Campylobacter jejuni, Helicobacter pylori, Propionibacterium acnes, Chlamydia trachomatis, Chlamydophila pneumoniae, Staphylococcus carnosus, Staphylococcus haemolyticus, Staphylococcus hominis, Staphylococcus lugdunensis, Streptococcus agalactiae, Streptococcus dysgalactiae, Streptococcus mitis, Streptococcus oralis, and Streptococcus pyogenes. The arylomycin compounds, including those of Formula I can be administered in a therapeutically effective amount.

As further described below, arylomycin antibiotics inhibit the bacterial type I signal peptidase (SPase) enzyme, particularly when the bacteria's SPase enzyme does not have a proline within about 10 amino acids N-terminal to the SPase catalytic serine, and especially when the bacteria's SPase enzyme does not have a proline at 5 to 7 amino acids N-terminal to the SPase catalytic serine. Thus, another aspect of the invention is a method of treating a bacterial infection in an animal that includes administering any one or any combination of arylomycin A, arylomycin B or the arylomycin compounds of Formula I to the animal, wherein the bacterial infection comprises an infection by a bacteria that encodes or expresses an SPase enzyme that does not have a proline within about 10 amino acids N-terminal to the SPase catalytic serine, or is an infection by Yersinia pestis. In some embodiments, the bacteria encodes or expresses an SPase enzyme that does not have a proline at 5 to 7 amino acids N-terminal to the SPase catalytic serine. The arylomycin A, arylomycin B and/or the compounds of Formula I can be administered in a therapeutically effective amount. Examples of organism with SPase genes that encode amino acids other than proline at residues −5 and −7 from the catalytic serine include but are not limited to Staphylococcus epidermidis, Staphylococcus haemolyticus, Staphylococcus luganensis, Staphylococcus hominis subsp. hominis, Staphylococcus hominis subsp. novobiosepticus, Staphylococcus cohnii, Streptococcus pnemoniae, Streptococcus pyogenes, Streptococcus agalactiae, Lactococcus lactis subsp. lactis, Rhodococcus opacus, Rhodococcus equi, Corynebacterium glutamicum Helicobacter pylori, Chlamydia trachomatis, and Francisella tularensis.

The following Table 6 provides a representative listing of bacterial species that do not have a proline at 5 to 7 amino acids N-terminal to the SPase catalytic serine.

TABLE 6
Bacteria Expressing a SPase enzyme that does not have a proline
5 to 7 amino acids N-terminal to the SPase catalytic serine

| | 8 residues N-terminal to and |
|---|---|
| Species | including catalytic Ser |
| Rhodococcus equi | VYLIPSES |
| Rhodococcus opacus | VYLIPSES |
| Corynebacterium diphtheriae | VYMIPSQS |
| Lactococcus lactis subsp. cremoris | LVVVDGHS |
| Corynebacterium glutamicum | MYMIPSGS |
| Francisella tularensis | NFLIPTAS |
| Campylobacter jejuni | AFVIPSGS |
| Helicobacter pylori | AFIIPSRS |
| Propionibacterium acnes | MFVIPSKS |
| Chlamydia trachomatis | LYEVPTGS |
| Chlamydophila pneumoniae | LYEVPTGS |
| Staphylococcus carnosus | SYTVRGDS |
| Staphylococcus haemolyticus | SYTIKGDS |
| | SYTVSGSS |
| Staphylococcus epidermidis | SYSIKGDS |
| | SYTVKGAS |
| Staphylococcus hominis | SYTIKGDS |
| | SYTVSGSS |
| Staphylococcus lugdunensis | SYTIKGDS |
| | TYSVSGDS |
| Streptococcus pneumoniae | NVRVEGHS |
| Streptococcus agalactiae | VLRIYGHS |
| | FVKVDGHS |
| Streptococcus dysgalactiae | AVKVDGHS |
| Streptococcus mitis | NVRVEGHS |
| Streptococcus oralis | NVRVEGHS |
| Streptococcus pyogenes | AVKVDGHS |

While almost all bacteria that do not have a proline within about 10 amino acids N-terminal to the SPase catalytic serine are susceptible to arylomycins (including arylomycin A, arylomycin B and the compounds of Formula I), there are a few exceptions. For example, some strains of Staphylococcus aureus, Staphylococcus capitis, Staphylococcus caprae and Yersinia pestis are still susceptible to arylomycins even though they do have a proline residue within 10 amino acids N-terminal to the catalytic serine (see Table 7).

TABLE 7
Bacterial Species Susceptible to Arylomycins
but with a Proline N-terminal to the SPase Catalytic Serine

| | Aminio acid −7 from | |
|---|---|---|
| Species | catalytic Ser^{a} | MIC (μg/ml) |
| Yersinia pestis | P | 4 |
| Staphylococcus capitis | P,S | 8 |
| Staphylococcus caprae | P,S | 8 |

^{a}Multiple amino acids indicate where organisms express multiple SPases

Thus, another aspect of the invention is a method of treating a bacterial infection in an animal that includes administering arylomycin A and/or arylomycin B and/or a compound of Formula I to the animal, wherein the infection is an infection involving a bacterial species that is susceptible to arylomycin but where the bacterial species has a proline residue within 10 amino acids N-terminal to the catalytic serine (e.g., at positions −5 and/or −7 from the catalytic serine). Such organisms include certain strains of Staphylococcus aureus, Staphylococcus capitis, Staphylococcus caprae and Yersinia pestis.

For example, Yersinia pestis has a single SPase with a proline at position 29, but as demonstrated herein, Yersinia pestis, is sensitive to arylomycin compounds. Yersinia pestis is a significant pathogen that can infect humans and other animals—it is the causative agent of the plague. Thus, new methods of treating Yersinia pestis infections are highly desirable. Therefore, another aspect of the invention is a method of treating a Yersinia pestis infection in an animal that includes administering arylomycin A and/or arylomycin B and/or a compound of Formula I to the animal. The arylomycin A and/or arylomycin B and/or the compound of Formula I can be administered in a therapeutically effective amount.

However, according to the invention, bacterial species with a proline 5-7 amino acids N-terminal to the catalytic serine are resistance to arylomycins. Such species include those shown in Table 8.

TABLE 8
Bacterial Species with a Proline
within 10 amino acids N-terminal to the
SPase catalytic Serine

| | 8 residues N-terminal to |
|---|---|
| Species | and including catalytic Ser |
| Escherichia coli | PFQIPSGS |
| Klebsiella pneumoniae | PFQIPSGS |
| Salmonella entericia | PFQIPSGS |
| Vibrio cholerae | PFQIPSGS |
| Pseudomonas aeruginosa | PFQIPSGS |
| Acinetobacter baumanii | PFNIPSDS |
| Neiserria meningitidis | PFQIPSSS |
| Haemophilus influenzae | PFQIPSGS |
| Citrobacter koseri | PFQIPSGS |
| Shigella flexneri | PFQIPSGS |
| Bordetella pertussis | PFHIPSGS |
| Mycobacterium | PYLIPSES |
| tuberculosis | |
| Staphylococcus aurues | PYTIKGES |
| Bacillus anthracis | PSLVQGES |
| | LCKVEGKS |
| Streptococcus mutans | PVQVDGHS |
| Clostridium difficile | PSIVSGES |
| | PTIVKGES |
| | PTLVNGES |
| Enterococcus faecalis | PAAVNGSS |
| | SYPIAGQS |
| | PVVVRGHS |
| | PVRVDGHS |
| Listeria monocytogenes | PVKVEGTS |
| | PVTVNGKS |
| | PILVDGIS |

Infections of particular interest that can be treated using arylomycins include those commonly detected in humans and/or those infections that are frequently inadequately treated by other antibiotics. Examples of infections that are susceptible to arylomycin treatment include those involving Staphylococcus epidermidis, Staphylococcus haemolyticus, Staphylococcus luganensis, Staphylococcus hominis subsp. hominis, Staphylococcus hominis subsp. novobiosepticus, Staphylococcus cohnii, Streptococcus pnemoniae, Streptococcus pyogenes, Streptococcus agalactiae, Lactococcus lactis subsp. lactis, Rhodococcus opacus, Rhodococcus equi, Corynebacterium glutamicum Helicobacter pylori, Chlamydia trachomatis, and Francisella tularensis, Rhodococcus equi, Corynebacterium diphtheriae, Lactococcus lactis subsp. cremoris, Corynebacterium glutamicum, Francisella tularensis, Campylobacter jejuni, Helicobacter pylori, Propionibacterium acnes, Chlamydia trachomatis, Chlamydophila pneumoniae, Staphylococcus carnosus, Staphylococcus haemolyticus, Staphylococcus hominis, Staphylococcus lugdunensis, Streptococcus agalactiae, Streptococcus dysgalactiae, Streptococcus mitis, Streptococcus oralis, and Streptococcus pyogenes.

In one embodiment, the infection involves Helicobacter pylori (whose SPase has Ala29), which infection is readily identified by available procedures. Hence, when such infections are identified the infections can be treated by administering to the affected animal any one or any combination of arylomin A, arylomycin B and/or any of the arylomycin compounds of Formula I. The arylomycin A and/or arylomycin B and/or the compound of Formula I can be administered in a therapeutically effective amount.

In other embodiments, the bacterial infection does not involve Staphylococcus epidermidis and/or Rhodococcus opacus.

The animal can be any animal suspected of suffering from a bacterial infection. For example, the animal can be a human, a domesticated animal, a zoo animal or an animal under the treatment of a doctor, nurse or veterinarian. Examples of animals that can be treated include humans, dogs, cats, horses, cattle, pigs, goats, sheep, chickens, geese, turkeys, rats, mice, hamsters, ferrets, parrots, lizards and the like.

Thus, the compounds of the invention can be administered to an animal (e.g., a mammal), especially a human in need of such treatment, prevention, elimination, alleviation or amelioration of a malcondition.

The compounds of the invention are effective over a wide dosage range. For example, in the treatment of adult humans, dosages from about 0.05 to about 5000 mg, preferably from about 1 to about 2000 mg, and more preferably between about 2 and about 2000 mg per day can be used. A typical dosage is about 10 mg to about 1000 mg per day. In choosing a regimen for patients it can frequently be necessary to begin with a higher dosage and when the condition is under control to reduce the dosage. The exact dosage will depend upon the activity of the compound, mode of administration, on the therapy desired, form in which administered, the subject to be treated and the body weight of the subject to be treated, and the preference and experience of the physician or veterinarian in charge.

Generally, the compounds of the invention are dispensed in unit dosage form including from about 0.05 mg to about 1000 mg of active ingredient together with a pharmaceutically acceptable carrier per unit dosage.

Usually, dosage forms suitable for oral, nasal, pulmonal or transdermal administration include from about 125 μg to about 1250 mg, preferably from about 250 μg to about 500 mg, and more preferably from about 2.5 mg to about 250 mg, of the compounds admixed with a pharmaceutically acceptable carrier or diluent.

Dosage forms can be administered daily, or more than once a day, such as twice or thrice daily. Alternatively dosage forms can be administered less frequently than daily, such as every other day, or weekly, if found to be advisable by a prescribing physician.

It is within ordinary skill to evaluate any compound disclosed and claimed herein for effectiveness in inhibition of susceptible and resistant variants of bacterial SPases, and in the various in vivo assays, using the procedures described above or found in the scientific literature. Accordingly, the person of ordinary skill can prepare and evaluate any of the claimed compounds without undue experimentation.

Any compound found to be an effective inhibitor of a bacterial SPase can likewise be tested in animal models and in human clinical studies using the skill and experience of the investigator to guide the selection of dosages and treatment regimens.

### DOCUMENTS CITED

(1) Miller, L. G.; Kaplan, S. L. Infect. Dis. Clin. North Am, 2009, 23, 35-52.
(2) Pearson, A. J Hosp Infect 2009, 73, 296-304.
(3) Payne, D. J.; Gwynn, M. N.; Holmes, D. J.; Pompliano, D. Nat. Rev. Drug Discov. 2007, 6, 29-40.
(4) Clardy, J.; Fischbach, M. A.; Walsh, C. T. Nat Biotechnol 2006, 24, 1541-50.
(5) Gringauz, A. Introduction to Medicinal Chemistry: How Drugs Act and Why; Wiley-VCH: Weinheim, Germany, 1996.
(6) Schneider, P.; Hawser, S.; Islam, K. Bioorg. Med. Chem. Lett. 2003, 13, 4217-21.
(7) von Nussbaum, F.; Brands, M.; Hinzen, B.; Weigand, S.; Habich, D. Angew. Chem. Int. Ed. Engl. 2006, 45, 5072-129.
(8) Sharma, P. C.; Jain, A.; Jain, S. Acta Pol Pharm 2009, 66, 587-604.
(9) Carlos, J. L.; Paetzel, M.; Brubaker, G.; Karla, A.; Ashwell, C. M.; Lively, M. O.; Cao, G.; Bullinger, P.; Dalbey, R. E. J. Biol. Chem. 2000, 275, 38813-38822.
(10) Paetzel, M.; Dalbey, R. E.; Strynadka, N. C. Pharmacol. Ther. 2000, 87, 27-49.
(11) Paetzel, M.; Karla, A.; Strynadka, N. C. J.; Dalbey, R. E. Chem. Rev, 2002, 102, 4549-4580.
(12) Höltzel, A.; Schmid, D. G.; Nicholson, G. J.; Stevanovic, S.; Schimana, J.; Gebhardt, K.; Fiedler, H. P.; Jung, G. The Journal of antibiotics 2002, 55, 571-577.
(13) Kulanthaivel, P., et al. J. Biol. Chem. 2004, 279, 36250-8.
(14) Schimana, J.; Gebhardt, K.; Holtzel, A.; Schmid, D. G.; Sussmuth, R.; Muller, J.; Pukall, R.; Fiedler, H. P. The Journal of antibiotics 2002, 55, 565-570.
(15) Cragg, G. M.; Newman, D. J.; Snader, K. M. J Nat Prod 1997, 60, 52-60.
(16) Dalbey, R. E.; Lively, M. O.; Bron, S.; van Dijl, J. M. Protein Sci 1997, 6, 1129-38.
(17) Date, T. J. Bacteriol. 1983, 154, 76-83.
(18) Roberts, T. C.; Smith, P. A.; Cirz, R. T.; Romesberg, F. E. J Am Chem Soc 2007, 129, 15830-8.
(19) Paetzel, M.; Goodall, J. J.; Kania, M.; Dalbey, R. E.; Page, M. G. J. Biol. Chem. 2004, 279, 30781-90.
(20) Luo, C.; Roussel, P.; Dreier, J.; Page, M. G.; Paetzel, M. Biochemistry 2009, 48, 8976-84.
(21) Beauregard, D. A.; Williams, D. H.; Gwynn, M. N.; Knowles, D. J. Antimicrob Agents Chemother 1995, 39, 781-5.
(22) Breukink, E.; de Kruijff, B. Nat Rev Drug Discov 2006, 5, 321-32.
(23) Kim, S. J.; Schaefer, J. Biochemistry 2008, 47, 10155-61.
(24) Nagarajan, R. J Antibiot (Tokyo) 1993, 46, 1181-95.
(25) Dufour, J.; Neuville, L.; Zhu, J. P. Synlett 2008, 2355-2359.
(26) Heller, H.; Schaefer, M.; Schulten, K. The Journal of Physical Chemistry 1993, 97, 8343-8360.
(27) Wang, Y.; Bruckner, R.; Stein, R. L. Biochemistry 2004, 43, 265-70.
(28) Baltz, R. H. Journal of Industrial Microbiology & Biotechnology 2006, 33, 507-13.
(29) Martinez, J. L. Proc. Biol. Sci. 2009, 276, 2521-30.

(30) D'Costa, V. M.; Griffiths, E.; Wright, G. D. Curr Opin Microbiol 2007, 10, 481-9.
(31) Allen, H. K.; Donato, J.; Wang, H. H.; Cloud-Hansen, K. A.; Davies, J.; Handelsman, J. Nat. Rev. Microbiol. 2010, 8, 251-9.
(32) Laskaris, P.; Tolba, S.; Calvo-Bado, L.; Wellington, L. Environ, Microbiol. 2010, 12, 783-96.
(33) Czaran, T. L.; Hoekstra, R. F.; Pagie, L. Proc. Natl. Acad. Sci. U.S.A. 2002, 99, 786-90.
(34) Lynch, M. Proc. Natl. Acad. Sci. U.S.A. 2007, 104 Suppl 1, 8597-604.
(35) Allen, N. E.; LeTourneau, D. L.; Hobbs, J. N., Jr. J Antibiot (Tokyo) 1997, 50, 677-84.
(36) Chen, L.; Yuan, Y.; Helm, J. S.; Hu, Y.; Rew, Y.; Shin, D.; Boger, D. L.; Walker, S. JAm Chem Soc 2004, 126, 7462-3.
(37) Cooper, M. A.; Williams, D. H. Chem Biol 1999, 6, 891-9.
(38) Dong, S. D.; Oberthur, M.; Losey, H. C.; Anderson, J. W.; Eggert, U. S.; Peczuh, M. W.; Walsh, C. T.; Kahne, D. J Am Chem Soc 2002, 124, 9064-5.
(39) Kerns, R.; Dong, S. D.; Fukuzawa, S.; Carbeck, J.; Kohler, J.; Silver, L.; Kahne, D. Journal of the American Chemical Society 2000, 122, 12608-12609.
(40) Mackay, J. P.; Gerhard, U.; Beauregard, D. A.; Maplestone, R. A.; Williams, D. H. Journal of the American Chemical Society 1994, 116, 4573-4580.
(41) Maffioli, S. I.; Ciabatti, R.; Romano, G.; Marzorati, E.; Preobrazhenskaya, M.; Pavlov, A. Bioorg. Med. Chem. Lett. 2005, 15, 3801-5.
(42) Nagarajan, R.; Schabel, A. A.; Occolowitz, J. L.; Counter, F. T.; Ott, J. L. J Antibiot (Tokyo) 1988, 41, 1430-8.
(43) Nagarajan, R.; Schabel, A. A.; Occolowitz, J. L.; Counter, F. T.; Ott, J. L.; Felty-Duckworth, A. M. J Antibiot (Tokyo) 1989, 42, 63-72.
(44) Rodriguez, M. J.; Snyder, N. J.; Zweifel, M. J.; Wilkie, S. C.; Stack, D. R.; Cooper, R. D.; Nicas, T. I.; Mullen, D. L.; Butler, T. F.; Thompson, R. C. J Antibiot (Tokyo) 1998, 51, 560-9.
(45) Sharman, G. J.; Try, A. C.; Dancer, R. J.; Cho, Y. R.; Staroske, T.; Bardsley, B.; Maguire, A. J.; Cooper, M. A.; O'Brie, D. P.; Williams, D. H. Journal of the American Chemical Society 1997, 119, 12041-12047.
(46) Albelo, S. T.; Domenech, C. E. FEMS Microbiol. Lett. 1997, 156, 271-4.
(47) Wilderman, P. J.; Vasil, A. I.; Martin, W. E.; Murphy, R. C.; Vasil, M. L. J. Bacteriol. 2002, 184, 4792-9.
(48) Sohlenkamp, C.; Lopez-Lara, I. M.; Geiger, O. Prog. Lipid Res. 2003, 42, 115-62.
(49) Cronan, J. E.; Vagelos, P. R. Biochim. Biophys. Acta 1972, 265, 25-60.
(50) Brundish, D. E.; Shaw, N.; Baddiley, J. Biochem. J. 1967, 104, 205-11.
(51) Mechin, L.; Dubois-Brissonnet, F.; Heyd, B.; Leveau, J. Y. J. Appl. Microbiol. 1999, 86, 859-66.
(52) Oliver, J. D.; Colwell, R. R. Int J Syst Bacteriol 1973, 23, 442-458.
(53) Nielsen, L. E.; Kadavy, D. R.; Rajagopal, S.; Drijber, R.; Nickerson, K. W. Appl, Environ, Microbiol. 2005, 71, 5171-6.
(54) De Siervo, A. J. J. Bacteriol. 1969, 100, 1342-9.
(55) Chatterjee, J.; Gilon, C.; Hoffman, A.; Kessler, H. Acc Chem Res 2008, 41, 1331-42.
(56) Conradi, R. A.; Hilgers, A. R.; Ho, N. F.; Burton, P. S. Pharm Res 1992, 9, 435-9.
(57) De Zotti, M.; Biondi, B.; Formaggio, F.; Toniolo, C.; Stella, L.; Park, Y.; Hahm, K. S. J Pept Sci 2009, 15, 615-9.
(58) Walsh, C. T.; Chen, H.; Keating, T. A.; Hubbard, B. K.; Losey, H. C.; Luo, L.; Marshall, C. G.; Miller, D. A.; Patel, H. M. Curr Opin Chem Biol 2001, 5, 525-34.

### Methods of Preparation

Compounds of the invention can be prepared either by semi-synthesis starting with an arylomycin compound isolated from a fermentation procedure, or by total chemical synthesis. Procedures are provided herein that, in combination with the knowledge of the synthetic organic chemist of ordinary skill in the art, can be used to prepare the full range of compounds of the invention as disclosed and claimed herein.

### Total Chemical Synthesis

For total synthesis, a retrosynthetic analysis was carried out, as shown in Scheme 1.

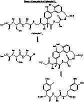

Arylomycin derivatives can be synthesized synthesized by synthesizing tripeptides by solution phase peptide couplings and then cyclization via Suzuki-Miyaura macrocyclization (the final step shown in the above retrosynthetic analysis).

Alternatively, natural product arylomycins can provide a core for further synthetic elaboration in some cases, depending upon the desired substituent pattern.

Starting with a cyclic core, for example by a total synthesis approach as shown above and exemplified in the Examples section, below, the exocyclic peptide/peptidomimetic domain, and the lipophilic tail domain, can be elaborated using approaches and methods described herein and those within the knowledge of the person of ordinary skill. See, for example, T. Roberts, et al. (2007), J. Am. Chem. Soc. 129, 15830-15838; Dufour, J.; Neuville, L.; Zhu, J. P. Synlett 2008, 2355-2359.

The various lipopeptide tails can be assembled via solution phase peptide couplings and then coupling to the macrocyclic core. The molecule can be considered to include three major domains: the cyclic core, an exocyclic peptide or peptidomimetic moiety, and a lipophilic tail moiety. In the natural product arylomycins, such as arylomycin A2, the lipophilic tail is an n-alkanoyl, isoalkanoyl, or anteisoalkanoyl acyl group; in compounds of the invention groups are introduced into the lipophilic tail that are adapted to provide a more favorable binding interaction of the inventive arylomycin analog with an SPase including a proline residue at the −5 and −7 position relative to the catalytic SPase serine residue, as shown in the X-ray crystal structure of arylomycin bound to a fragment of a resistant form of SPase, shown in FIG. 3A. As discussed above, the presence of a proline residue at one of these positions has been found by the inventors herein to provide resistance of the SPase to inhibition by natural product arylomycins such as arylomycin A2. The inventive compounds can overcome this resistance by designing the lipophilic tail to bind more effectively to SPase forms having the proline residue(s).

The R⁵ group can be bonded to the exocyclic peptide moiety via acyl, carbamate, or urea linkages, which can be formed as described below, for the three classes of linkages.

For compounds where the R⁵ linkage to the peptide is an amide bond, and wherein the aromatic ring is connected directly to the carbonyl group of the amide, these compounds can be synthesized by coupling of commercially available benzoic acids or heterocyclic acids that had been substituted by electrophilic or nucleophilic aromatic substitution or palladium catalyzed processes (and appropriately protected using standard protecting groups^{S1}) to the N-terminus of the peptide chain. Heterocycles where the commercially available acids are not available will be synthesized via any one of a number of methods for synthesizing pyridines, pyrazines, pyrimidines or pyradizines^{S2}.

For compounds where the linkage of R⁵ to the exocyclic peptide is an amide bond, and where the aromatic ring is not connected directly to the carbonyl group of the amide, these compounds can be synthesized via the scheme:

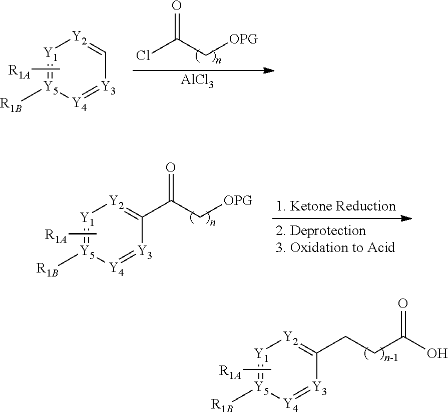

Appropriately functionalized or unfunctionalized aryl rings (appropriately protected using standard protecting groups^{S1}) will be subjected to Friedel-Crafts acylation conditions with an alkyl chain bearing an acid chloride and a protected hydroxyl group. The ketone is then reduced, the protected hydroxyl group is deprotected, the hydroxyl is oxidized to an acid and the resulting acid is coupled to the N-terminus of the peptide.

For compounds where the R⁵ linkage to the peptide is a carbamate and the aryl ring is not attached directly to the carbamate, functionalized phenols (appropriately protected using standard protecting groups^{S1}) can be treated with phosgene to create the aryl carbamoyl chloride which can then be used to acylate the N-terminus of the peptide. Functionalized or unfunctionalized aryl rings will be subjected to Friedel-Crafts acylation conditions with an alkyl chain bearing an acid chloride and a protected hydroxyl group. The ketone of the resulting compound will be reduced and the protecting group will be removed. The compound will then be treated with phosgene to form the carbamoyl chloride^{S3} and this compound will be used to acylate the N-terminus of the peptide as shown in the scheme:

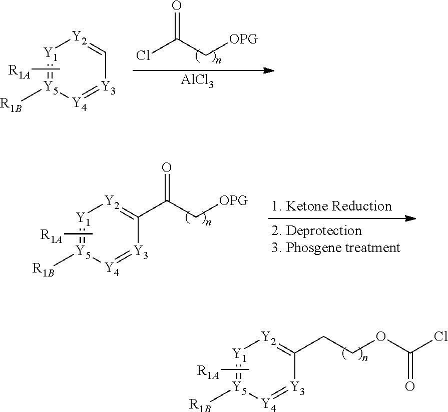

Heterocycles where Friedel-Crafts acylations are not possible will be halogenated (and appropriately protected using standard protecting groups^{S1}) and the appropriate length hydrocarbon chain terminated on one end with a protected alcohol and the other end with a halogen or boronic acid/ester will be attached via palladium mediated coupling.

For compounds where the R⁵ linkage to the peptide is a urea and the aryl ring is attached directly to the nitrogen atom, functionalized aryl amines will be treated with phosgene to create the aryl ureayl chloride which will then be used to acylate the N-terminus of the peptide.

For compounds where the R⁵ linkage to the peptide is a urea and the aryl ring is not attached directly to the carbamate, the compounds will be synthesized via the scheme:

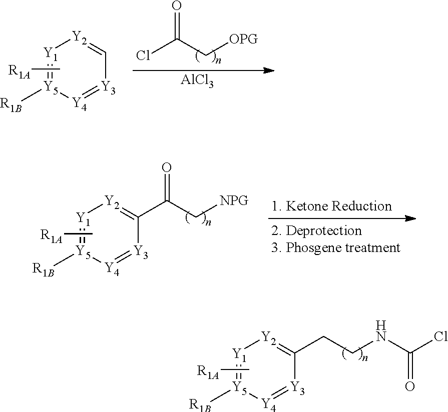

Appropriately functionalized or unfunctionalized aryl rings (and appropriately protected using standard protecting groups^{S1}) will be subjected to Friedel-Crafts acylation conditions with an alkyl chain bearing an acid chloride and a protected amine. The ketone of the resulting compound will be reduced and the protecting group will be removed. The compound will then be treated with phosgene to form the ureayl chloride^{S4} and this compound will be used to acylate the N-terminus of the peptide. Heterocycles where Friedel-Crafts acylations are not possible are halogenated (and appropriately protected using standard protecting groups^{S1}) and an appropriate length hydrocarbon chain terminated on one end with a protected amine and the other end with a halogen or boronic acid/ester will be attached via palladium mediated coupling.

The various embodiments of compounds of the invention with the variants of the R⁵ group can be synthesized using the above approaches, in conjunction with ordinary knowledge concerning the use of any protecting or blocking groups that may be necessary. See, for example, Protective Groups in Organic Synthesis, Greene, T. W.; Wuts, P. G. M., John Wiley & Sons, New York, N.Y., (3rd Edition, 1999).

In various embodiments of the invention, R⁵ can be straight chain or branched chain alkyl, wherein the chain can include any of the following groups (A)-(E). Synthetic approaches appropriate for each class of R⁵ group are provided.

(A)

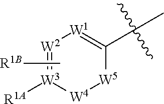

wherein W¹, W², W³, W⁴ and W⁵ are each independently C or N, provided that no more than two of W¹, W², W³, W⁴ and W⁵ are N; provided that when R^{1A} or R^{1B} is non-hydrogen, any W atom to which the R^{1A} or R^{1B} is respectively bonded is C, wherein there can be one or more R^{1B} bonded to the ring bearing the W atoms; R^{1A} is hydrogen, halogen, amino, hydroxyl, aminocarbonyl, hydroxycarbonyl, nitro, fluoroalkyl, cyano, (C₁-C₆)-thioether, fluoroalkoxy, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; R^{1B} is hydrogen, alkyl, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-alkylamino, (C₁-C₆)-alkyl, 5- to 7-membered heteroaryl, 5- to 7-membered heterocyclyl, or (C₆-C₁₀)aryl; wherein any R^{1A} or R^{1B} can be further substituted with one to three (C₁-C₁₂)-alkyl or -alkoxy groups, which can further bear halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-alkylamino, 5- to 7-membered heteroaryl, 5- to 7-membered heterocyclyl, or (C₆-C₁₀)aryl groups; wherein a wavy line indicates a point of attachment.

For compounds where the R⁵ linkage to the peptide is an acyl group and where the aromatic ring is connected directly to the acyl group these compounds can be synthesized by peptide coupling of commercially available benzoic or heterocyclic acids that had been substituted by electrophilic aromatic substitution, nucleophilic aromatic substitution or palladium catalyzed processes (and appropriately protected using standard protecting groups^{S1}) to the N-terminus of the peptide chain. Heterocycles where the commercially available acids are not available can be synthesized via any one of a number of methods for synthesizing pyridines, pyrazines, pyrimidines or pyradizines^{S2}.

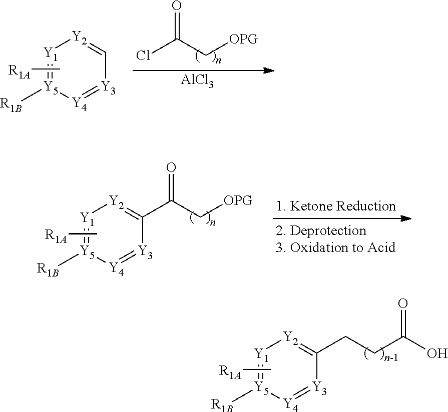

For compounds where the R⁵ linkage to the peptide is an acyl group and where the aromatic ring is not connected directly to the acyl group, these compounds can be synthesized via the above scheme. Appropriately functionalized or unfunctionalized aryl rings (appropriately protected using standard protecting groups^{S1}) can be subjected to Friedel-Crafts acylation conditions with an alkyl chain bearing an acid chloride and a protected hydroxyl group. The ketone can then be reduced, the protected hydroxyl group deprotected, the hydroxyl oxidized to an acid, and the resulting acid coupled to the N-terminus of the peptide.

For compounds where the R⁵ linkage to the peptide is a carbamate and the aryl ring is attached directly to the carbamate, functionalized phenols (appropriately protected using standard protecting groups^{S1}) can be treated with phosgene to create the aryl carbamoyl chloride which can then be used to acylate the N-terminus of the peptide.

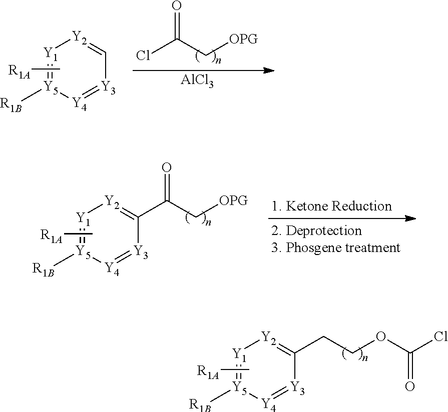

For compounds where the R⁵ linkage to the peptide is a carbamate and the aryl ring is not attached directly to the carbamate, the compounds can be synthesized via the route shown in the above scheme. Appropriately functionalized benzenes (appropriately protected using standard protecting groups^{S1}) can be subjected to Friedel-Crafts acylation conditions with an alkyl chain bearing an acid chloride and a protected hydroxyl group. The ketone of the resulting compound can be reduced and the protecting group removed. The compound can then be treated with phosgene to form the carbamoyl chloride^{S3} and this compound can be used to acylate the N-terminus of the peptide. Heterocycles where Friedel-Crafts acylations are not possible can be halogenated (and appropriately protected using standard protecting groups^{S1}) and the appropriate length hydrocarbon chain terminated on one end with a protected alcohol and the other end with a halogen or boronic acid/ester can be attached via palladium mediated coupling.

For compounds where the R⁵ linkage to the peptide is a urea and the aryl ring is attached directly to the carbamate, functionalized aryl amines can be treated with phosgene to create the aryl ureayl chloride which can then be used to acylate the N-terminus of the peptide.

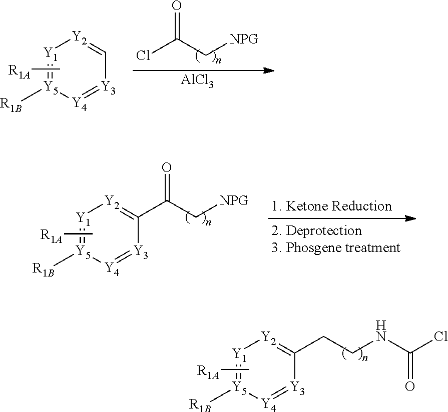

For compounds where the R⁵ linkage to the peptide is a urea and the aryl ring is not attached directly to the carbamate, the compounds can be synthesized via the route shown in the above scheme. Appropriately functionalized can be subjected to Friedel-Crafts acylation conditions with an alkyl chain bearing an acid chloride and a protected amine. The ketone of the resulting compound can be reduced and the protecting group be removed. The compound can then be treated with phosgene to form the ureayl chloride^{S4} and this compound can be used to acylate the N-terminus of the peptide. Heterocycles where Friedel-Crafts acylations are not possible can be halogenated (and appropriately protected using standard protecting groups^{S1}) and an appropriate length hydrocarbon chain terminated on one end with a protected amine and the other end with a halogen or boronic acid/ester attached via palladium mediated coupling.

(B)

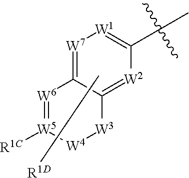

wherein W¹, W², W³, W⁴, W⁵, W⁶, and W⁷ are each independently C or N, provided than no more than three of W¹, W², W³, W⁴, W⁵, W⁶, and W⁷ are N; provided that when R^{1C} or R^{1D} is non-hydrogen, any W atom to which the R^{1C} or R^{1D} is respectively bonded is C, wherein either ring can bear one or more R^{1D}; R^{1C} is hydrogen, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; R^{1D} is hydrogen, alkyl, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein any R^{1C} or R^{1D} can be further substituted with one to three (C₁-C₁₂)-alkyl or -alkoxy groups, which can further bear halogen, amino, hydroxyl, aminocarbonyl, hydroxycarbonyl, nitro, fluoroalkyl, (C₁-C₆)thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein a wavy line indicates a point of attachment.

For compounds where the R⁵ linkage to the peptide is an acyl group and where the aromatic rings are connected directly to the acyl group these compounds can be synthesized by peptide coupling of commercially available heterocyclic acids that are substituted by electrophilic aromatic substitution, nucleophilic aromatic substitution, heteroaryl lithium formation or palladium catalyzed processes (and appropriately protected using standard protecting groups^{S1}) to the N-terminus of the peptide chain. Heterocycles where the commercially available acids are not available can be synthesized via any one of a number of methods for synthesizing quinolines, isoquinolines, quinazolines, quinoxalines or 1,8-napthyridines^{S2}.

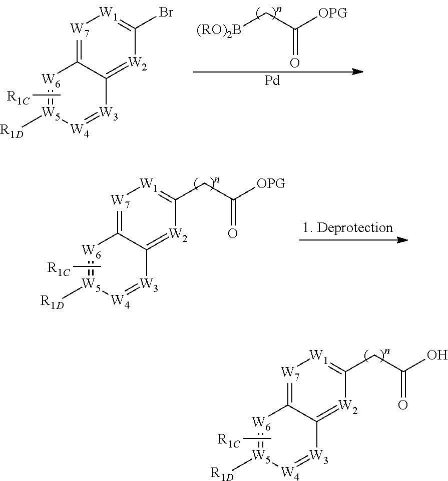

For compounds where the R⁵ linkage to the peptide is an acyl group and where the aromatic ring is not connected directly to the acyl group, these compounds can be synthesized via the above scheme. Halogenated and appropriately functionalized or unfunctionalized aryl rings (appropriately protected using standard protecting groups^{S1}) and an appropriate length hydrocarbon chain terminated on one end with a protected carboxylate and the other end with a halogen or boronic acid/ester can be attached via palladium mediated coupling

For compounds where the R⁵ linkage to the peptide is a carbamate and the aryl rings are attached directly to the carbamate, these compounds can be by peptide coupling of commercially available heterocyclic alcohols that are substituted by electrophilic aromatic substitution, nucleophilic aromatic substitution, heteroaryllithium formation or palladium catalyzed processes (and appropriately protected using standard protecting groups^{S1}) to the N-terminus of the peptide chain. Heterocycles where the commercially available alcohols are not available can be synthesized via any one of a number of methods for synthesizing quinolines, isoquinolines, quinazolines, quinoxalines or 1,8-napthyridines^{S2}.

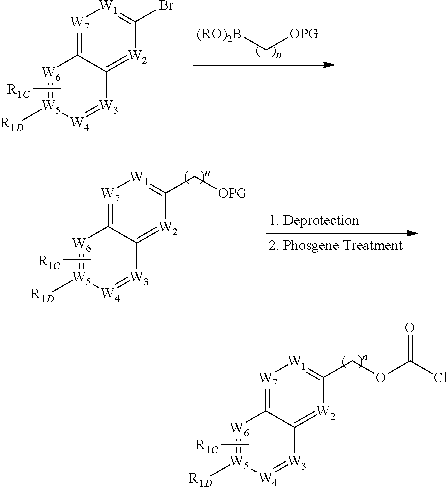

For compounds where the R⁵ linkage to the peptide is a carbamate and the aryl rings are not attached directly to the carbamate, these compounds an be synthesized via the above scheme. Halogenated and appropriately functionalized or unfunctionalized aryl rings (appropriately protected using standard protecting groups^{S1}) and an appropriate length hydrocarbon chain terminated on one end with a protected alcohol and the other end with a halogen or boronic acid/ester can be attached via palladium mediated coupling. The alcohol can then be deprotected, the compound treated with phosgene and the resulting carbamoyl chloride used to acylate the N-terminus of the peptide.

For compounds where the R⁵ linkage to the peptide is a urea and the aryl rings are attached directly to the urea, these compounds can be synthesized by peptide coupling of commercially available heterocyclic amines that are substituted by electrophilic aromatic substitution, nucleophilic aromatic substitution, heteroaryllithium formation or palladium catalyzed processes (and appropriately protected using standard protecting groups^{S1}) then treated with phosgene^{S4} to the N-terminus of the peptide chain. Heterocycles where the commercially available amines are not available can be synthesized via any one of a number of methods for synthesizing quinolines, isoquinolines, quinazolines, quinoxalines or 1,8-napthyridines^{S2}.

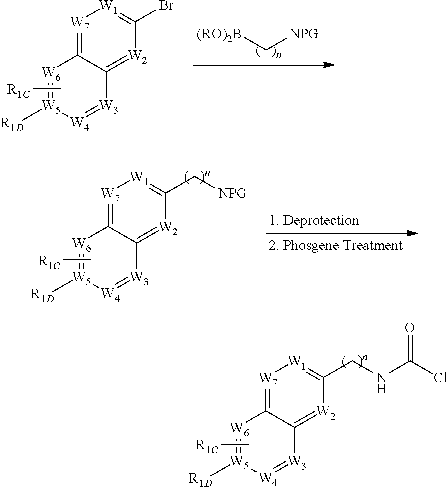

For compounds where the R⁵ linkage to the peptide is a urea and the aryl rings are not attached directly to the urea, these compounds can be synthesized via the above scheme. Halogenated and appropriately functionalized or unfunctionalized aryl rings (appropriately protected using standard protecting groups^{S1}) and an appropriate length hydrocarbon chain terminated on one end with a protected amine and the other end with a halogen or boronic acid/ester can be attached via palladium mediated coupling. The amine can then be deprotected, the compound will be treated with phosgene and the resulting carbamoyl chloride used to acylate the N-terminus of the peptide.

(C)

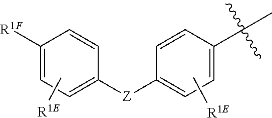

wherein Z is O, S, NH or CH₂; R^{1E} at each occurrence is independently hydrogen, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; R^{1F} is hydrogen or alkyl, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein any R^{1E} or R^{1F} can be further substituted with one to three (C₁-C₁₂)-alkyl or -alkoxy groups, which can further bear halogen, amino, hydroxyl, aminocarbonyl, hydroxycarbonyl, nitro, fluoroalkyl, (C₁-C₆)thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₆)aryl; wherein a wavy line indicates a point of attachment.

In addition to the procedures outlined above for attachment to the peptide, compounds of this functionality are synthesized by employment of the Buchwald-Hartwig coupling conditions^{S5} when Z═O or N. Where a para-halogen substituted protected benzoic acid, homologated benzoic acid or precursor is coupled with a phenol functionalized by electrophilic or nucleophilic aromatic substitution or palladium catalyzed processes (and appropriately protected using standard protecting groups^{S1}). When Z═S these compounds can be formed using transition metal catalyzed couplings of a para-halogen substituted protected benzoic acid, homologated benzoic acid or precursor combined with an appropriately functionalized thiophenol.

(D)

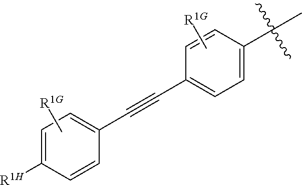

wherein R^{1G} at each occurrence is independently hydrogen, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; R^{1H} is hydrogen or alkyl, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein any R^{1G} or R^{1H} can be further substituted with one to three (C₁-C₁₂)-alkyl or -alkoxy groups, which can further bear halogen, amino, hydroxyl, aminocarbonyl, hydroxycarbonyl, nitro, fluoroalkyl, (C₁-C₆)thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein a wavy line indicates a point of attachment.

In addition to the procedures outlined above for attachment to the peptide, compounds of this functionality can be synthesized by employment of Sonagashira reaction conditions^{S6} on a para-halogen substituted protected benzoic acid, homologated benzoic acid or precursor combined with the appropriately functionalized by electrophilic or nucleophilic aromatic substitution or palladium catalyzed processes (and appropriately protected using standard protecting groups^{S1}) aryl acetylene as shown in the below scheme.

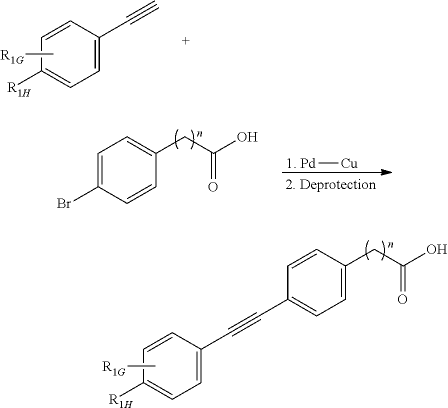

The peptidic tail can be assembled analogously to procedures described herein using standard solution or solid phase peptide couplings. Constituent amino acids containing substituents at the R^{A3}, R^{A4}, and R^{A5} positions, and the groups of formulas (IIA), (IIB), and (IIC), can either be purchased commercially or synthesized via amino acid synthesis procedures described in the literature^{S7-S9}.

Peptidic tails where any R⁴ or R⁶ are not hydrogen can be assembled using literature protocols for peptide-peptoid conjugates^{S10}. The monomers can be synthesized using amine alkylation protocols^{S11} for example an amino acid with a protected carboxylate is protected at the amine with a nosyl group, the nosylated amine is selectively alkylated with base and an electrophile and the nosyl group is deprotected by thiolate anion.

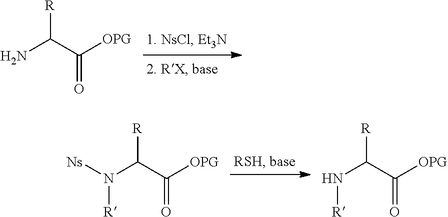

Where m, n1, or n2 are either 0 or 1, amino acids building blocks where m, n1, and n2 are equal to 1 are commercially available or can be synthesized via methods found in the literature^{S12}, for example from succinates where one acid is protected with a carboxyl protecting group and the other attached to a chiral auxiliary which then allows asymmetric monoalkylation. The protected carboxyl can then be deprotected and transformed into an amine via a Curtius rearrangement followed by cleavage of the chiral auxiliary with peroxide.

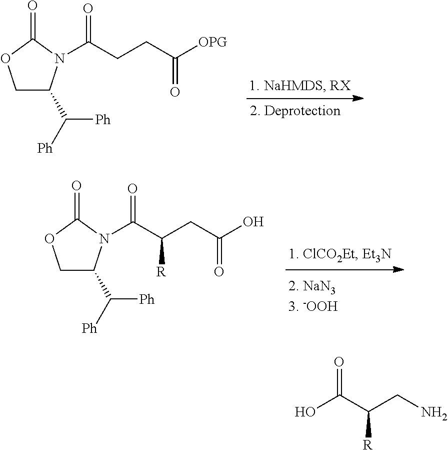

Where m, n1, and n2 are 0, 1 or 2, amino acids building blocks where m, n1, and n2 are equal to 1 or 2 can be synthesized analogously wherein the differentially protected aspartic or glutamic acid is functionalized at the free carboxylate attached to the alpha carbon by any number of strategies including but not limited to peptide coupling, reduction whereby the acid can be converted to a functionalized ketone via a Weinreb amide or reduction whereby the acid is converted to an alcohol that is subsequently converted to a tosylate and either displaced by a nucleophile or coupled to another aryl or alkyl group via a palladium mediated process:

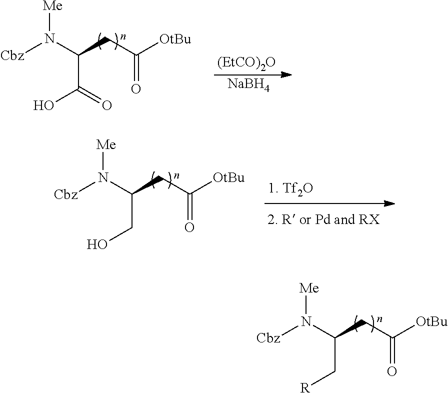

These amino acids can be synthesized via protocols found in the literature^{S12-S13} for example Arndt Eistert homologation(s) as shown in the below scheme.

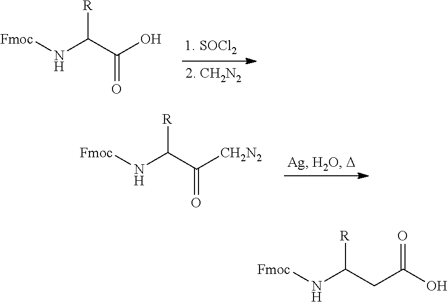

Amino acids building blocks for the synthesis of compounds where R² and R³ are each independently not hydrogen can either be purchased commercially or can be synthesized via amino acid synthesis procedures described in the literatures^{S7-S9,S14} and appropriately protected using standard protecting groups^{S1}.

Where OG¹ and OG² hydroxyl, O-alkyl, or O-glycosyl, compounds can be synthesized by protocols developed for synthesis of the arylomycin natural product^{S15}.

Where R^{A1} is not hydrogen can be synthesized by the methods described for the synthesis of the arylomycin macrocycle. The tyrosine derivatives required as building blocks for that synthesis can be synthesized as described by Michaux et. al.^{S16} and the references described therein. A Homer Wadsworth Emmons reaction can be used, followed by halogenations of the alkene Suzuki coupling of the desired substituent and asymmetric catalytic hydrogenation to the desired tyrosine derivative.

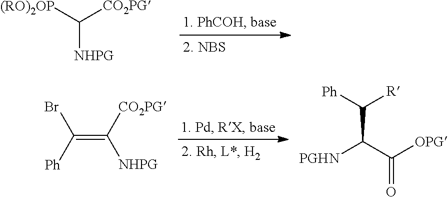

Where R^{A2} is not hydrogen, compounds can be synthesized using protocols for the synthesis of the natural product and protocols for peptide coupling of disubstituted amino acids^{S17}. The amino acid building blocks can be synthesized by literature protocols^{S18}. For example the amino and carboxyl groups of an appropriately protected tyrosine can be condensed with benzaldehyde to form an oxazolidinone which can then be asymmetrically alkylated with strong base and an electrophile and hydrolyzed to yield the substituted tyrosine derivative^{S19}.

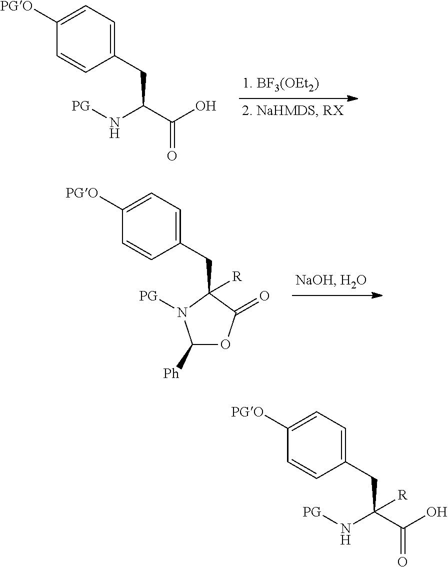

Compounds where a carbonyl group is directly attached to the scaffold at B can be synthesized from the fully deprotected arylomycin. Peptide coupling to an amino acid where the carboxylate is replaced by a protected or unprotected electrophilic moiety can install the aldehydes^{S20}, boronic acids/esters^{S21} and phosphonates^{S22}. Azetidinones that are attached to the arylomycin through an amine at the 3-position of the azetidinone ring can be synthesized via peptide coupling of the amine of the azetidinone to the carboxylate of arylomycin^{S23} Azetidinones that are attached to the arylomycin through the cyclic nitrogen can be synthesized by peptide coupling of the cyclic NH to the arylomycin carboxylate^{S24}. The azetidinone building blocks can be synthesized via literature protocols^{S25-S26}.
(S1) Wuts, P. G. M.; Greene, T. W. Greene's protective groups in organic synthesis; 4th ed.; Wiley-Interscience: Hoboken, N.J., 2007.
(S2) Joule, J. A.; Mills, K. Heterocyclic chemistry; 4th ed.; Blackwell Science: Oxford; Malden, M A, 2000.
(S3) Shin, D.-S.; Lee, Y.-S. Synlett 2009, 2009, 3307.
(S4) Musser, J. H.; Chakraborty, U.; Bailey, K.; Sciortino, S.; Whyzmuzis, C.; Amin, D.; Sutherland, C. A. Journal of Medicinal Chemistry 1987, 30, 62.
(S5) Hartwig, J. F. Angew Chem Int Edit 1998, 37, 2047.
(S6) Sonogashira, K. J Organomet Chem 2002, 653, 46.
(S7) Nájera, C.; Sansano, J. M. Chemical Reviews 2007, 107, 4584.
(S8) Maruoka, K.; Ooi, T. Chemical Reviews 2003, 103, 3013.
(S9) Easton, C. J. Chemical Reviews 1997, 97, 53.
(S10) Olsen, C. A. Chem Bio Chem 2010, 11, 152.
(S11) Kan, T.; Fukuyama, T. Chemical Communications 2004, 353.
(S12) Liu, M.; Sibi, M. P. Tetrahedron 2002, 58, 7991.
(S13) Lelais, G.; Seebach, D. Peptide Science 2004, 76, 206.
(S14) Williams, R. M.; Hendrix, J. A. Chemical Reviews 1992, 92, 889.
(S15) Roberts, T. C.; Smith, P. A.; Cirz, R. T.; Romesberg, F. E. J Am Chem Soc 2007, 129, 15830.
(S16) Michaux, J.; Niel, G.; Campagne, J.-M. Chemical Society Reviews 2009, 38, 2093.
(S17) Humphrey, J. M.; Chamberlin, A. R. Chemical Reviews 1997, 97, 2243.
(S18) Ohfune, Y.; Shinada, T. European Journal of Organic Chemistry 2005, 2005, 5127.
(S19) Aberle, N.; Ovenden, S. P. B.; Lessene, G.; Watson, K. G.; Smith, B. J. Tetrahedron Letters 2007, 48, 2199.
(S20) Zhang, X.; Rodrigues, J.; Evans, L.; Hinkle, B.; Ballantyne, L.; Pena, M. The Journal of Organic Chemistry 1997, 62, 6420.
(S21) Zhu, Y.; Yao, S.; Xu, B.; Ge, Z.; Cui, J.; Cheng, T.; Li, R. Bioorganic & Medicinal Chemistry 2009, 17, 6851.
(S22) Sienczyk, M.; Lesner, A.; Wysocka, M.; Legowska, A.; Pietrusewicz, E.; Rolka, K.; Oleksyszyn, J. Bioorganic & Medicinal Chemistry 2008, 16, 8863.
(S23) Setti, E. L.; Davis, D.; Janc, J. W.; Jeffery, D. A.; Cheung, H.; Yu, W. Bioorganic & Medicinal Chemistry Letters 2005, 15, 1529.
(S24) Vidya, R.; Eggen, M.; Nair, S. K.; Georg, G. I.; Himes, R. H. The Journal of Organic Chemistry 2003, 68, 9687.
(S25) Brandi, A.; Cicchi, S.; Cordero, F. M. Chemical Reviews 2008, 108, 3988.
(S26) Magriotis, P. A. Angewandte Chemie International Edition 2001, 40, 4377.

### Semisynthesis

Compounds of the invention can also be prepared by semisynthesis, that is, through synthetic conversions applied to arylomycin compounds isolated from natural sources such as fermentation broths or in vitro biosynthesis systems.

It is known in the art that the natural product arylomycin, such as arylomycin A2, can be isolated and purified from its microbial source.^{N1-N2} The natural product can then be treated with a 50:50 mixture of trifluoroacetic acid and CH₂Cl₂ to cleave off the n-terminal lipid and N-Me serine residue leaving a free amine, as shown below. When the arylomycin starting material is in the A series, X is hydrogen, and when the arylomycin starting material is in the B series, X is nitro.

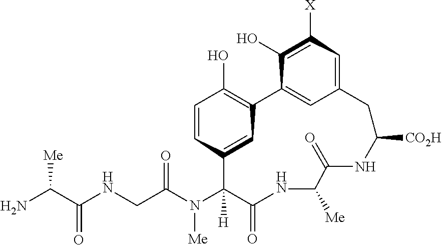

The resulting free amine can then either be coupled to a new N-alkyl amino acid and lipid tail or protected with a nosyl group, selectively methylated, denosylated^{N3}, acetylated and treated again with a 50:50 mixture of trifluoroacetic acid and CH₂Cl₂ to cleave off the N-terminal alanine.

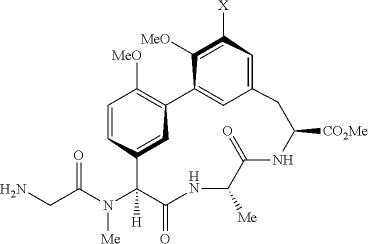

The free amine of the alanine cleaved compound can then be alkylated, if desired, then coupled to an appropriately functionalized and protected lipodipeptide tail and globally deprotected^{N4} (scheme 2, below).

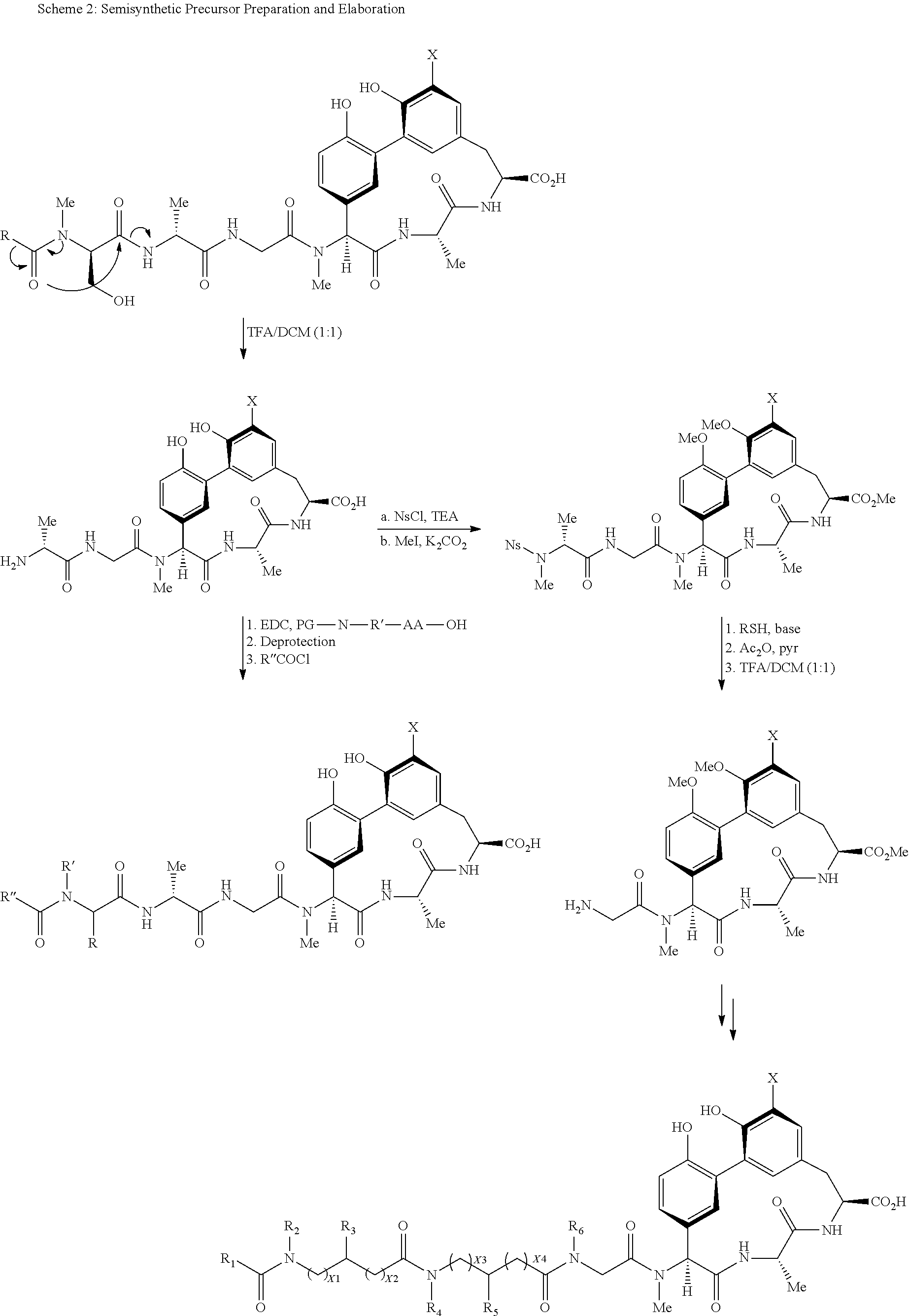

The isolated natural product of the arylomycin B series bears a nitro group on the tyrosine^{N1-N2}, so the free amine that is the result of TFA mediated cleavage of the lipid tail and N-terminal serine of this compound, or the free amine that is the result of cleavage of the lipid tail and the N-terminal serine and alanine residues of this compound, contain a nitro functionality that can then be used to prepare other modifications of the tyrosine ring and, due to the nitro deactivation of the tyrosine ring, of the hydroxyphenylglycine ring as well. After protection or deprotection with the appropriate protecting groups^{N5} the nitro compounds of the arylomycin B derivatives can be selectively iodinated ortho to the hydroxyl of the hydroxyphenylglycine residue. This compound can then be functionalized to install numerous chemical groups selectively on the hydroxyphenylglycine ring using, for example, palladium mediated coupling^{N6}. For further functionalization of the tyrosine ring, after appropriate protection or deprotection, the nitro group can be reduced to an amine and converted into a diazo salt^{N7}. This compound can be subsequently functionalized via the Sandmeyer reaction^{N8} to yield a variety of different functional groups. The resulting compound can then be attached to a lipopeptide tail via peptide coupling, then globally deprotected^{N4} (scheme 3, below).

### DOCUMENTS CITED

(N1) Schimana, J.; Gebhardt, K.; Holtzel, A.; Schmid, D. G.; Sussmuth, R.; Muller, J.; Pukall, R.; Fiedler, H. P. J Antibiot 2002, 55, 565.
(N2) Holtzel, A.; Schmid, D. G.; Nicholson, G. J.; Stevanovic, S.; Schimana, J.; Gebhardt, K.; Fiedler, H. P.; Jung, G. J Antibiot 2002, 55, 571.
(N3) Kan, T.; Fukuyama, T. Chemical Communications 2004, 353.
(N4) Roberts, T. C.; Smith, P. A.; Cirz, R. T.; Romesberg, F. E. J Am Chem Soc 2007, 129, 15830.
(N5) Wuts, P. G. M.; Greene, T. W. Greene's protective groups in organic synthesis; 4th ed.; Wiley-Interscience: Hoboken, N.J., 2007.
(N6) Miyaura, N.; Suzuki, A. Chem Rev 1995, 95, 2457.
(N7) Evans, D. A.; Katz, J. L.; Peterson, G. S.; Hintermann, T. Journal of the American Chemical Society 2001, 123, 12411.
(N8) Galli, C. Chem Rev 1988, 88, 765.

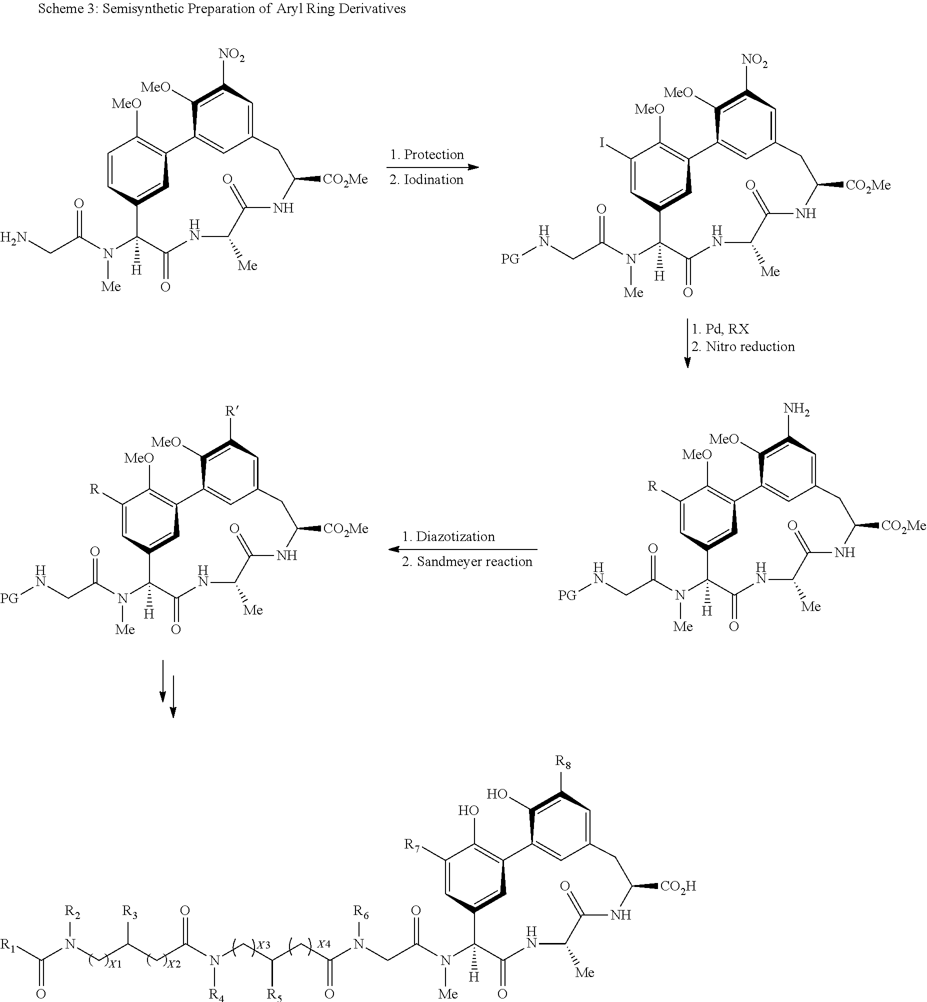

### Signal Peptidase (SPase)

As described and illustrated herein, arylomycin antibiotics can inhibit the bacterial type I signal peptidase (SPase) enzyme, an essential serine-lysine dyad protease that is anchored to the outer leaflet of the bacterial cytoplasmic membrane and that removes N-terminal signal peptides from proteins that are transported out of the cytoplasm. Bacterial SPase proteases appear to be present and essential in all Eubacteria. However, not all Eubacteria are susceptible to arylomycin antibiotics.

According to the invention, the sequence of a bacterial SPase is correlated with susceptibility or resistance to arylomycin antibiotics. In particular, the presence of one or more proline residues near the catalytic serine gives rise to an arylomycin-resistant phenotype. The term “resistant” or “resistance” is used to refer to the reduced sensitivity of a strain harboring a resistance-conferring Pro relative to the isogenic strain without the Pro, and not to the absolute sensitivities of the different bacteria (e.g. resistant S. epidermidis is inhibited with an MIC of 8 μg/ml and is significantly more sensitive to arylomycin C₁₆ than the resistant mutants of the other pathogens, which are not inhibited at concentrations as high as 128 μg/ml).

Sequences for bacterial SPase nucleic acids and polypeptides are publicly available, for example, in the database maintained by the National Center for Biotechnology (ncbi.nlm.nih.gov) and can be used to make probes, primers and antigens useful for detecting arylomycin-susceptible or arylomycin-resistant bacteria.

For example, the Staphylococcus epidermidis RP62A signal peptidase IB has the following sequence (SEQ ID NO:1; see NCBI accession no. YP_188144.1, gi:57866486).

| | |
|---|---|
| 1 | MKKEILEWIV AIAVAIALIA IITKFVGKSY SIKGDSMDPT |
| 41 | LKDGERVVVN IIGYKLGGVE KGNVIVFHAN KKDDYVKRVI |
| 81 | GTPGDSVEYK NDTLYVNGKK QSEPYLNYNE KRKQTEYITG |
| 121 | SFKTKNLPNA NPQSNVIPKG KYLVLGDNRE VSKDSRSFGL |
| 161 | IDKDQIVGKV SLRYWPFSEF KSNFNPNNTK N |

The underlining shows the region of the catalytic serine, which is at position 36 in this SPase sequence. A nucleic acid sequence for the SEQ ID NO:1 Staphylococcus epidermidis RP62A signal peptidase IB can have the following sequence (SEQ ID NO:2, see NCBI accession no. NC_002976.3, GI:57865352).

| | |
|---|---|
| 1 | TTGAAAAAAG AAATTTTAGA GTGGATTGTT GCCATAGCCG |
| 41 | TTGCCATTGC ACTTATTGCC ATAATCACTA AATTTGTCGG |
| 81 | AAAATCATAT TCTATTAAAG GTGATTCAAT GGATCCTACA |
| 121 | TTAAAAGATG GGGAGCGTGT AGTGGTAAAT ATTATTGGCT |
| 161 | ATAAATTAGG TGGCGTTGAA AAAGGAAATG TCATTGTATT |
| 201 | TCATGCTAAT AAAAAAGATG ATTATGTTAA AAGAGTTATT |
| 241 | GGAACTCCAG GAGATAGTGT TGAATATAAA AATGATACAC |
| 281 | TCTATGTTAA TGGTAAAAAG CAATCAGAAC CATACTTGAA |
| 321 | CTATAATGAA AAACGTAAGC AAACTGAGTA TATCACAGGT |
| 361 | AGTTTCAAAA CAAAAAATTT ACCAAATGCT AATCCTCAAT |
| 401 | CTAATGTTAT TCCTAAAGGT AAATATTTAG TTTTGGGGGA |
| 441 | TAACCGTGAG GTAAGTAAAG ATAGTCGTTC ATTCGGTTTA |
| 481 | ATTGACAAAG ACCAAATTGT TGGAAAGGTA TCGCTCAGAT |
| 521 | ATTGGCCTTT CAGTGAATTT AAATCTAACT TTAATCCAAA |
| 561 | TAACACTAAA AATTAA |

According to the invention, a bacterium having a mutant SPase gene is resistant to arylomycin antibiotics when one or more proline residues are present near the catalytic serine (e.g., at position 36 in SEQ ID NO:1) of the SPase polypeptide, for example, within about 10 amino acid positions on the N-terminal side of the catalytic serine of the SPase polypeptide. Thus, as illustrated herein, a bacterium with a proline between about amino acid position 28 to about amino acid position 35 of the SEQ ID NO:1 SPase amino acid sequence tends to exhibit resistance to arylomycin antibiotics. In some embodiments, the proline residue that gives rise to arylomycin resistance is present at about position −7 relative to catalytic serine. In other embodiments, the proline residue that gives rise to arylomycin resistance is present at about position −5 relative to catalytic serine. For example, in the SEQ ID NO:1 SPase sequence, a proline at position 29 or a proline at position 31 gives rise to arylomycin resistance.

One aspect of the invention therefore includes a method for detecting whether bacterial cells susceptible to arylomycin compounds are present in a test sample. Such susceptibility can be detected by detecting whether a SPase polypeptide is present that does not have a proline near the catalytic serine, for example, within about 10 amino positions on the N-terminal side of the catalytic serine and about two positions on the C-terminal side of the bacterial SPase polypeptide (e.g., within about positions 26-38 of SEQ ID NO:1). The absence of such a proline within the SPase polypeptide sequence indicates that bacterial cells within the sample are susceptible to arylomycin treatment.

In some embodiments, the method may also include detecting whether bacteria are present a test sample. The presence or absence of a proline in the Spase polypeptides can be simultaneously or subsequently detected to ascertain whether bacterial cells in the test sample are resistant or susceptible, respectively, to arylomycin compounds.

The presence of bacterial resistance to arylomycin compounds can be detected by determining whether a proline is present within about 10-12 amino acids of the catalytic serine in a SPase polypeptide. For example, an antibody that is specific for the proline-containing SPase polypeptide can be employed. Such a specific antibody binds with detectably greater affinity to a SPase polypeptide having one or more prolines than to a corresponding SPase polypeptide that does not have any such proline residues.

Conversely, the presence of susceptibility to arylomycin compounds can be detected by determining whether no proline residues are present within about 10-12 amino acids of the catalytic serine in a SPase polypeptide. An antibody specific for a SPase polypeptide that does not have a proline within about 10-12 amino acids of the catalytic serine in a SPase polypeptide can, for example, be used to detect whether a bacteria cell in a test sample is susceptible to arylomycin treatment.

Alternatively, such antibiotic resistance or susceptibility can be detected by detecting a nucleic acid encoding a SPase protein with such a proline residue. Thus, for example, nucleic acids in a test sample can be isolated using available procedures and the presence of a nucleic acid can be detected that encodes a SPase protein with or without such a proline residue. These isolated nucleic acids can be tested by available hybridization and/or nucleic acid amplification procedures to ascertain whether proline-encoding or non-proline-encoding SPase nucleic acids are present in the test sample.

Probes, primers and antigenic peptides useful for detecting the presence or absence of a proline within a SPase nucleic acid or polypeptide can readily be designed by one of ordinary skill in the art. For example, the following description illustrates how the SEQ ID NO:1 and 2 sequences can be used to design such probes, primers and/or antigenic peptides.

When the SEQ ID NO:1 and 2 sequences are aligned as illustrated below, the nucleic acid and amino acid sequences near the catalytic serine become apparent, as well as the sequences and codons that can become proline residue(s) in arylomycin resistant SPases (e.g., the underlined sequences).

| DNA: | ATGAAAAAAGAAATTTTAGAGTGGATTGTTGCCATAGCCGTTGCCATTGCA |
|---|---|
| 1: | M K K E I L E W I V A I A V A I A |
| DNA: | CTTATTGCCATAATCACTAAATTTGTCGGAAAATCATATTCTATTAAAGGT |
| 18: | L I A I I T K F V G K S Y S I K G |
| DNA: | GATTCAATGGATCCTACATTAAAAGATGGGGAGCGTGTAGTGGTAAATATT |
| 35: | D S M D P T L K D G E R V V V N I |
| DNA: | ATTGGCTATAAATTAGGTGGCGTTGAAAAAGGAAATGTCATTGTATTTCAT |
| +1: | I G Y K L G G V E K G N V I V F H |
| DNA: | GCTAATAAAAAAGATGATTATGTTAAAAGAGTTATTGGAACTCCAGGAGAT |
| +1: | A N K K D D Y V K R V I G T P G D |
| DNA: | AGTGTTGAATATAAAAATGATACACTCTATGTTAATGGTAAAAAGCAATCA |
| +1: | S V E Y K N D T L Y V N G K K Q S |
| DNA: | GAACCATACTTGAACTATAATGAAAAACGTAAGCAAACTGAGTATATCACA |
| +1: | E P Y L N Y N E K R K Q T E Y I T |
| DNA: | GGTAGTTTCAAAACAAAAAATTTACCAAATGCTAATCCTCAATCTAATGTT |
| +1: | G S F K T K N L P N A N P Q S N V |
| DNA: | ATTCCTAAAGGTAAATATTTAGTTTTGGGGGATAACCGTGAGGTAAGTAAA |
| +1: | I P K G K Y L V L G D N R E V S K |
| DNA: | GATAGTCGTTCATTCGGTTTAATTGACAAAGACCAAATTGTTGGAAAGGTA |
| +1: | D S R S F G L I D K D Q I V G K V |
| DNA: | TCGCTCAGATATTGGCCTTTCAGTGAATTTAAATCTAACTTTAATCCAAAT |
| +1: | S L R Y W P F S E F K S N F N P N |
| DNA: | AACACTAAAAATTAA |
| +1: | N T K N * |

As described herein, when position −7 or position −5 from the catalytic serine is occupied by a proline rather than a serine, bacteria containing such a SPase enzyme are arylomycin resistant. When no such proline is present in the SPase enzyme, the bacteria are arylomycin susceptible. In the SEQ ID NO:1 SPase amino acid sequence, the −7 position is at position 29 and the −5 position is at position 31—serine is typically present in both positions within the wild type, arylomycin-susceptible SEQ ID NO:1 sequence.

Examples of shorter SPase peptide and nucleotide sequences from SEQ ID NO:1 and 2 that are correlated with arylomycin resistance or susceptibility include the following, where the position of the mutation in the nucleotide sequence is noted with a small arrow.

| DNA: | GTCGGAAAATCATATTCTATTAAAGGTGATTCA | Arylomycin Susceptible |
|---|---|---|
| 28: | V G K S Y S I K G D S | SEQ ID NOs: 3 and 4 |
| | ↓ | |
| DNA: | GTCGGAAAACCATATTCTATTAAAGGTGATTCA | Arylomycin Resistant |
| 28: | V G K P Y S I K G D S | SEQ ID NOs: 5 and 6 |
| | ↓ ↓ | |
| DNA: | GTCGGAAAACCGTATTCTATTAAAGGTGATTCA | Arylomycin Resistant |
| 28: | V G K P Y S I K G D S | SEQ ID NOs: 7 and 6 |
| | ↓ ↓ | |
| DNA: | GTCGGAAAACCCTATTCTATTAAAGGTGATTCA | Arylomycin Resistant |
| 28: | V G K P Y S I K G D S | SEQ ID NOs: 8 and 6 |
| | ↓ ↓ | |
| DNA: | GTCGGAAAACCTTATTCTATTAAAGGTGATTCA | Arylomycin Resistant |
| 28: | V G K P Y S I K G D S | SEQ ID NOs: 9 and 6 |
| | ↓ | |
| DNA: | GTCGGAAAATCATATCCTATTAAAGGTGATTCA | Arylomycin Resistant |
| 28: | V G K S Y P I K G D S | SEQ ID NOs: 10 and 11 |
| | ↓ ↓ | |
| DNA: | GTCGGAAAATCATATCCCATTAAAGGTGATTCA | Arylomycin Resistant |
| 28: | V G K S Y P I K G D S | SEQ ID NOs: 12 and 11 |
| | ↓ ↓ | |
| DNA: | GTCGGAAAATCATATCCAATTAAAGGTGATTCA | Arylomycin Resistant |
| 28: | V G K S Y P I K G D S | SEQ ID NOs: 13 and 11 |
| | ↓ ↓ | |
| DNA: | GTCGGAAAATCATATCCGATTAAAGGTGATTCA | Arylomycin Resistant |
| 28: | V G K S Y P I K G D S | SEQ ID NOs: 14 and 11 |

The first set of nucleotide and peptide sequences are just short sequences taken from the wild type, arylomycin susceptible SEQ ID NO:1 and 2 SPase sequences. Note that proline is encoded by four different codons (CCT, CCC, CCA, CCG), so four different SPase nucleotide sequences (e.g., SEQ ID NOs: 5, 7, 8 and 9) can exist for each proline-containing SPase polypeptide (e.g., a SPase polypeptide containing SEQ ID NO:6).

Primers and probes can readily be designed that are complementarity to nucleic acids encoding SPase-susceptible (non-proline containing) or SPase-resistant (proline-encoding) polypeptides. Such primers and probes can be designed to have sufficient sequence identity and/or sufficient complementary sequence identity to selectively hybridize with bacterial nucleic acids that encode SPase-susceptible (non-proline containing) or SPase-resistant (proline-encoding) polypeptides, and thereby permit detection of whether bacteria are arylomycin susceptible or arylomycin resistant. For example, to detect whether S. epidermidis bacteria in a test sample are arylomycin susceptible or arylomycin resistant primers or probes are designed to selectively hydridize to regions of nucleic acids that include any of SEQ ID NOs:3, 5, 7-10, 12, 13 or 14. Further information is provided below on selective hybridization and on the selection of probes and primers to detect bacterial nucleic acids that encode SPase-susceptible (non-proline containing) or SPase-resistant (proline-encoding) polypeptides, and thereby permit detection of whether bacteria are arylomycin susceptible or arylomycin resistant. For example, the Examples provide specific primer sequences that can be used to detect an/or isolate SPase nucleic acids.

One of skill in the art can also readily generate antibodies that selectively bind to bacterial SPase-susceptible (non-proline containing) polypeptides or SPase-resistant (proline-encoding) polypeptides, and to use those antibodies to detect whether bacteria are arylomycin susceptible or arylomycin resistant. For example, peptides or polypeptides that contain any of SEQ ID NOs:4, 6 or 11 can be used to generate such antibodies. These antibodies can be screened to identify antibody preparations that selectively bind to bacterial SPase-susceptible (non-proline containing) polypeptides or SPase-resistant (proline-encoding) polypeptides. Further information is provided below on making and using antibodies for detecting SPase-susceptible (non-proline containing) or SPase-resistant (proline-encoding) polypeptides, to thereby detect whether bacteria with such polypeptides are arylomycin susceptible or arylomycin resistant.

Primers and/or probes can be made from other bacterial SPase polypeptide and nucleic acid sequences, for example, any of those described herein or available in sequence databases. For example, the Staphylococcus epidermidis RP62A signal peptidase I has the following sequence, where the catalytic serine is identified in bold and with underlining (SEQ ID NO:15; see NCBI accession no. YP_187624.1, gi:57865986).

| | |
|---|---|
| 1 | MKKEIIEWIV AIIVAIVIVT LVQKFLFASY TVKGASMHPT |
| 41 | FENREKVIVS RIAKTLDHID TGDVVIFHAN AKQDYIKRLI |
| 81 | GKPGDSVEYK KDQLYLNGKK VDEPYLSENK KHKVGEYLTE |
| 121 | NFKSRDLKGT NGNMKIPSGK YLVLGDNRQN SIDSRMDEVG |
| 161 | LLDKNQVVGK VVLRYWPFNR WGGSFNPGTF PN |

The nucleotide sequence for the SEQ ID NO:15 Staphylococcus epidermidis RP62A signal peptidase I has the following sequence (SEQ ID NO:16; see NCBI accession no. NC_002976.3 GI:57865352).

| | |
|---|---|
| 1 | ATGAAGAAAG AAATAATAGA ATGGATTGTA GCCATAATCG |
| 41 | TTGCAATTGT TATCGTCACA CTTGTGCAAA AGTTTTTATT |
| 81 | TGCTTCTTAT ACAGTCAAAG GAGCATCTAT GCATCCAACA |
| 121 | TTTGAAAATC GAGAAAAAGT GATAGTAAGT CGTATAGCAA |
| 161 | AAACGCTTGA TCATATTGAT ACAGGAGATG TAGTGATTTT |
| 201 | TCATGCTAAC GCGAAGCAAG ATTATATTAA GCGACTTATT |
| 241 | GGTAAACCAG GTGATTCAGT AGAATATAAA AAAGATCAAC |
| 281 | TATATTTAAA CGGTAAAAAA GTAGATGAGC CTTATTTAAG |
| 321 | TGAAAATAAA AAACATAAAG TTGGAGAATA TCTAACGGAA |
| 361 | AACTTTAAGT CTAGAGATCT TAAGGGTACG AATGGCAATA |
| 401 | TGAAAATTCC TAGTGGTAAA TACTTGGTTT TAGGTGATAA |
| 441 | TCGTCAAAAC AGTATTGACA GTCGCATGGA TGAAGTAGGT |
| 481 | CTTTTAGATA AAAATCAAGT TGTTGGAAAA GTAGTTTTGA |
| 521 | GATACTGGCC ATTTAATCGG TGGGGCGGTA GTTTTAATCC |
| 561 | TGGAACATTT CCTAACTAA |

Additional bacterial SPase sequences are available, for example, in the NCBI sequence database.

### Genetically Modified Bacterial Signal Peptidases

Another aspect of the invention is a modified bacterial SPase and/or a bacterial host cell that includes a modified bacterial SPase. Such modified SPases are useful for identifying arylomycin compounds that can inhibit the activity of modified and/or naturally occurring SPases. For example, when a proline is present within about 10 amino acids N-terminal to the catalytic serine, the SPase does not effectively bind an arylomycin and the arylomycin does not effectively inhibit the activity of the SPase. The presence of the proline reduces arylomycin binding to such an extent that the effects of structural changes in the arylomycin compound structure cannot readily be detected.

According to the invention, one way to detect improved arylomycin structures during structure-activity studies is to observe the binding of a test arylomycin compound to an SPase that has been modified by replacement of the proline at position 5 to 7 N-terminal to the catalytic serine with another amino acid (e.g., a serine or other amino acid). Alternatively, improved arylomycin structures can be identified by observing the binding of a test arylomycin compound to an SPase that has been modified by replacement of a naturally occurring amino acid at position 5 to 7 N-terminal to the catalytic serine with a proline (thereby converting an arylomycin-susceptible SPase into an arylomycin-resistant SPase). These types of modified SPase enzymes therefore permit evaluation of test compounds against non-proline containing “arylomycn resistant” SPase sequences so that secondary sites of potential (minor) resistance can be identified and the arylomycin structure can be modified to address any such secondary sites of resistance. Similarly, SPases that are naturally resistant to arylomycin, due to the absence of a proline within about 10 amino acids of the catalytic serine, are modified by substitution to place a proline at −5 to −7 positions N-terminal to the catalytic serine so that the test compounds can be identified that effectively bind and inhibit such an SPase even though there is a proline in what is otherwise an“arylomycin-susceptible” SPase structure.

Therefore, another aspect of the invention is a method of identifying a compound that can bind to and/or inhibit the activity of a bacterial SPase that involves contacting a modified SPase with a test compound and observing whether the test compound binds to and/or inhibits the activity of the modified SPase, wherein the modified SPase has a natural bacterial SPase amino acid sequence that has been modified at position −5 to −7 relative to the catalytic serine by substitution or replacement of a proline that position. In some embodiments, a proline at position −5 and/or at position −7 is replaced with another amino acid (e.g., a serine). In other embodiments, the amino acid that is naturally present at position −5 and/or at position −7 is replaced with a proline. Test compounds that bind and/or inhibit the activity of the modified SPase are compounds of interest.

In another embodiment, test compounds that have antibiotic activity against bacteria are identified by contacting a culture of bacteria with the test compound and identifying whether the test compound inhibits the growth of the bacteria, wherein the bacteria express a modified SPase that has a natural bacterial SPase amino acid sequence that has been modified at position −5 to −7 relative to the catalytic serine by substitution or replacement of a proline that position. In some embodiments, a proline at position −5 and/or at position −7 is replaced with another amino acid (e.g., a serine). In other embodiments, the amino acid that is naturally present at position −5 and/or at position −7 is replaced with a proline. Test compounds that inhibit the growth of a bacterium expressing such modified SPase have antibiotic activity.

The bacteria can be modified by recombinant techniques available to those of skill in the art so that the bacteria express a modified SPase. Such techniques can include removal, replacement or mutation of an endogenous SPase gene so that endogenous SPase gene is not expressed, thereby allowing expression of only the modified SPase enzyme. Such “knockout” procedures for removal, replacement and/or mutation of an endogenous gene in a bacterium are available in the art, and can readily be employed to generate bacterial populations that express a selected modified SPase enzyme.

Examples of procedures for generating such modified SPase enzymes and modified bacterial populations are provided, for example, in the Examples and in the art. A “knockout cassette” can be employed. Such a knockout cassette refers to a fragment of native chromosomal DNA having a foreign DNA piece that may provide a selectable marker. In one embodiment “knock-out mutation cassettes” are created by interrupting a fragment of genomic DNA with a foreign piece of DNA, and replacing the wild-type chromosomal copy of the sequence with the knock-out cassette. In this embodiment, the knock-out protocol involves cloning a modified SPase DNA segment into a target DNA such that “tails” comprising the target site DNA remain at the 5′ and 3′ ends of the knock-out cassette. The tails may be at least 50 base pairs and preferably greater than 200 to 500 base pairs for efficient recombination and/or gene conversion. For convenience, the foreign DNA cloned into the target DNA also provides a selectable marker, for example, an antibiotic resistance gene. Where the target DNA is disrupted with a marker antibiotic resistance gene, selection of transformants is carried out on agar plates containing suitable levels of an appropriate antibiotic. Following transformation, a fraction of cells that have taken up the knockout cassette will have undergone homologous recombination or gene conversion across the genomic DNA tails of the cassette, resulting in replacement of the wild-type genomic sequence by the knock-out cassette. Knock-out recombination events are easily confirmed by, for example, Southern blot hybridization, or by PCR.

Detection and/or Amplification of SPase Nucleic Acids

The presence of bacterial species susceptible or resistant to arylomycin antibiotics can be detected by detecting SPase nucleic acids in a test sample suspected of containing bacteria. As described and illustrated herein arylomycin antibiotic resistance or susceptibility can be detected by detecting a nucleic acid encoding a SPase protein with a proline residue near the catalytic serine residue.

Nucleic acids in a test sample can be isolated using available procedures. For example, bacterial nucleic acids can be isolated from a test sample by lysing the bacterial cells using detergents, heat, proteases and/or phenol extraction and alcohol precipitation.

The presence of a nucleic acid can be detected that encodes a SPase protein with or without such a proline residue by employing available hybridization, single nucleotide polymorphism and/or nucleic acid amplification procedures to ascertain whether proline-encoding or non-proline-encoding SPase nucleic acids are present in the test sample. In general, selective hybridization conditions are employed to facilitate detection of the proline-encoding and/or non-proline-encoding SPase nucleic acids and these procedures.

The term “selectively hybridize” includes hybridization, under stringent hybridization conditions, of a nucleic acid sequence to a specified nucleic acid target sequence (e.g., SEQ ID NO:2 or any SPase nucleic acid) to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, or 60-90% sequence identity, or 90-95% sequence identity, or 90-99% sequence identity, or 95-97% sequence identity, or 98-99% sequence identity, or 100% sequence identity (or complementarity) with each other. In some embodiments, a selectively hybridizing sequence has about at least about 70% sequence identity with SEQ ID NO:2. Note that if a value of a variable that is necessarily an integer, e.g., the number of nucleotides or amino acids in a nucleic acid or protein, is described as a range, e.g., or 90-99% sequence identity, or 100% sequence identity, what is meant is that the value can be any integer between 90 and 99 inclusive, i.e., 90, 91, 92, 93, 94, 95, 96, 97, 98 or 99.

The probes and primers of the invention include those with about 10-100 of the same nucleotides as either strand of a bacterial SPase DNA or RNA (e.g., SEQ ID NOs:2), or about 12-50, or about 13-40, or about 14-30 of the same nucleotides as either strand of a bacterial SPase DNA or RNA (e.g., SEQ ID NO:2). The probes and primers of the invention also include those with about 10-30 of the same nucleotides as either strand of any of SEQ ID NOs: 2, 5, 7, 8, 9 or the other SPase nucleic acids disclosed herein or available in a public data. The identical nucleotides or amino acids can be distributed throughout the nucleic acid or the protein, and need not be contiguous. Based on such methodologies, a person skilled in the art can readily design primers in suitable regions 5′ and 3′ to the signal peptidase segment that may encode the proline.

The terms “stringent conditions” or “stringent hybridization conditions” include conditions under which a probe will hybridize to its target sequence to a detectably greater degree than other sequences (e.g., at least 2-fold over background). Stringent conditions are somewhat sequence-dependent and can vary in different circumstances. By controlling the stringency of the hybridization and/or washing conditions, target sequences can be identified which can be up to 100% complementarity to the probe (homologous probing). Alternatively, stringency conditions can be adjusted to allow some mismatching in sequences so that lower degrees of similarity are detected (heterologous probing).

A probe or primer can vary in length. For example, a probe can be approximately 20-500 nucleotides in length, but can vary greatly in length from about 15, or about 16, or about 17, or about 18 nucleotides to equal to the entire length of the target sequence. In some embodiments, the probe is about 10-50 nucleotides in length, or about 15-40 nucleotides in length, or about 18-50 nucleotides in length, or about 18-100 nucleotides in length.

In some embodiments, primers are shorter than probes. For example, a primer may be about 12 to 50 nucleotides in length, or about 13 to 40 nucleotides in length, or about 14 to 35 nucleotides in length.

In some embodiments, stringent hybridization conditions and procedures are employed. Typically, stringent conditions will be those where the salt concentration is less than about 1.5 M Na ion, typically about 0.01 to 1.0 M Na ion concentration (or other salts) at pH 7.0 to 8.3 and the temperature is at least about 30° C. for short probes or primers (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1M NaCl, 1% SDS (sodium dodecyl sulphate) at 37° C., and a wash in 1×SSC to 2×SSC (where 20×SSC is 3.0 M NaCl, 0.3 M trisodium citrate) at 50 to 55° C. Exemplary moderate stringency conditions include hybridization in 40 to 45% formamide, 1M NaCl, 1% SDS at 37° C., and a wash in 0.5×SSC to 1×SSC at 55 to 60° C. Exemplary high stringency conditions include hybridization in 50% formamide, 1M NaCl, 1% SDS at 37° C., and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl (Anal. Biochem. 138:267-84 (1984)):

Tₘ=81.5° C.+16.6 (log M)+0.41 (% GC)−0.61 (% formamide)−500/L where M is the molarity of monovalent cations; % GC is the percentage of guanosine and cytosine nucleotides in the DNA, % formamide is the percentage of formamide in the hybridization solution, and L is the length of the hybrid in base pairs. The Tₘ is the temperature (under defined ionic strength and pH) at which 50% of a complementary target sequence hybridizes to a perfectly matched probe. The Tₘ is reduced by about 1° C. for each 1% of mismatching. Thus, the Tₘ, hybridization and/or wash conditions can be adjusted to hybridize to sequences of the desired identity. For example, if sequences with greater than or equal to 90% sequence identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or a wash at 1, 2, 3 or 4° C. lower than the thermal melting point (Tₘ). Moderately stringent conditions can utilize a hybridization and/or a wash at 6, 7, 8, 9 or 10° C. lower than the thermal melting point (Tₘ). Low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill can identify and isolate nucleic acids with sequences related to SEQ ID NO:1. Those of skill in the art also understand how to vary the hybridization and/or wash solutions. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, part 1, chapter 2, “Overview of principles of hybridization and the strategy of nucleic acid probe assays,” Elsevier, N.Y. (1993); and Current Protocols in Molecular Biology, chapter 2, Ausubel, et al., eds, Greene Publishing and Wiley-Interscience, New York (1995). Unless otherwise stated, in the present application high stringency is defined as hybridization in 4×SSC, 5×Denhardt's (5 g Ficoll, 5 g polyvinylpyrrolidone, 5 g bovine serum albumin in 500 ml of water), 0.1 mg/ml boiled salmon sperm DNA, and 25 mM Na phosphate at 65° C., and a wash in 0.1×SSC, 0.1% SDS at 65° C.

Therefore hybridization procedures can be used to detect the presence or absence of an encoded proline in bacterial signal peptidase nucleic acids. In addition, the presence or absence of such an encoded proline can be detected by nucleic acid amplification, single nucleotide polymorphism (SNP), sequencing and other procedures available to one of skill in the art.

Amplification methods available in the art can be utilized, including polymerase chain reaction (PCR) (U.S. Pat. Nos. 4,683,195; and 4,683,202; PCR Technology: Principles and Applications for DNA Amplification, ed. H. A. Erlich, Freeman Press, NY, N.Y., 1992), ligase chain reaction (LCR) (Wu and Wallace, Genomics 4:560, 1989; Landegren et al., Science 241:1077, 1988), strand displacement amplification (SDA) (U.S. Pat. Nos. 5,270,184; and 5,422,252), transcription-mediated amplification (TMA) (U.S. Pat. No. 5,399,491), linked linear amplification (LLA) (U.S. Pat. No. 6,027,923), and the like, and isothermal amplification methods such as nucleic acid sequence based amplification (NASBA), and self-sustained sequence replication (Guatelli et al., Proc. Natl. Acad. Sci. USA 87: 1874, 1990).

A variety of single nucleotide polymorphism (SNP) genotyping methods are available including those described in Chen et al., “Single nucleotide polymorphism genotyping: biochemistry, protocol, cost and throughput”, Pharmacogenomics J. 2003; 3(2):77-96; Kwok et al., “Detection of single nucleotide polymorphisms”, Curr Issues Mol. Biol. 2003 April; 5(2):43-60; Shi, “Technologies for individual genotyping: detection of genetic polymorphisms in drug targets and disease genes”, Am J. Pharmacogenomics. 2002; 2(3):197-205; and Kwok, “Methods for genotyping single nucleotide polymorphisms”, Annu Rev Genomics Hum Genet 2001; 2:235-58; see also, U.S. Patent Application Publication No. 20100216154, contents of which publications are incorporated by reference in their entireties. Exemplary techniques for high-throughput SNP genotyping are described in Marnellos, “High-throughput SNP analysis for genetic association studies”, Curr Opin Drug Discov Devel. 2003 May; 6(3):317-21, which is incorporated herein by reference in its entirety. Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, OLA (U.S. Pat. No. 4,988,167, which is incorporated herein by reference in its entirety), multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

Various methods for detecting polymorphisms include, but are not limited to, methods in which protection from cleavage agents is used to detect mismatched bases in RNA/RNA or RNA/DNA duplexes (Myers et al., Science 230:1242 (1985); Cotton et al., PNAS 85:4397 (1988); and Saleeba et al., Meth. Enzymol. 217:286-295 (1992)), comparison of the electrophoretic mobility of variant and wild type nucleic acid molecules (Orita et al., PNAS 86:2766 (1989); Cotton et al., Mutat. Res. 285:125-144 (1993); and Hayashi et al, Genet. Anal. Tech. Appl. 9:73-79 (1992)), and assaying the movement of polymorphic or wild-type fragments in polyacrylamide gels containing a gradient of denaturant using denaturing gradient gel electrophoresis (DGGE) (Myers et al., Nature 313:495 (1985)); the contents of which publications are incorporated herein by reference in their entirety. Sequence variations at specific locations can also be assessed by nuclease protection assays such as RNase and S1 protection or chemical cleavage methods.

For example, in some embodiments, SNP genotyping is performed using the TaqMan assay, which is also known as the 5′ nuclease assay (U.S. Pat. Nos. 5,210,015 and 5,538,848, which are incorporated herein by reference in their entirety). The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

In another aspect of the invention, the signal peptidase sequences of various bacterial species are determined and compared to generate bacterial phylogenetic profiles of drug resistance useful for identifying drugs that can readily be modified to overcome such drug resistance.

To generate such drug resistance phylogenetic profiles, the degree of sequence similarity and difference is determined. The following terms are used to describe the sequence relationships between two or more nucleic acids or nucleic acids or polypeptides: (a) “reference sequence,” (b) “comparison window,” (c) “sequence identity,” (d) “percentage of sequence identity” and (e) “substantial identity.” As used herein, “reference sequence” is a defined sequence used as a basis for sequence comparison. The reference sequence can be a nucleic acid sequence (e.g., SEQ ID NO:2) or an amino acid sequence (e.g., SEQ ID NO:1). A reference sequence may be a subset or the entirety of a specified sequence. For example, the reference sequence can be an entire SPase DNA, RNA or polypeptide sequence, or a segment of a full-length SPase DNA, RNA or polypeptide sequence, or a peptide, DNA or RNA including/encoding just the region of the catalytic serine and/or a region that is N-terminal to catalytic serine (e.g., from about amino acid position −10 to about amino acid +2 relative to the catalytic serine).

As used herein, “comparison window” means includes reference to a contiguous and specified segment of a nucleic acid or an amino acid sequence, wherein the nucleic acid/amino acid sequence may be compared to a reference sequence and wherein the portion of the nucleic acid/amino acid sequence in the comparison window may comprise additions or deletions (i.e., gaps) compared to the reference sequence (which does not comprise additions or deletions) for optimal alignment of the two sequences. The comparison window can vary for nucleic acid and polypeptide sequences. Generally, for nucleic acids, the comparison window is at least 20 contiguous nucleotides in length, and optionally can be 22, 25, 30, 35, 40, 50, 100 or more nucleotides. For amino acid sequences, the comparison window is at least about 10 to 15 amino acids, and can optionally be 20, 22, 25, 30, 35, 40, 50, 100 or more amino acids. Those of skill in the art understand that to avoid a high similarity to a reference sequence due to inclusion of gaps in the nucleic acid or amino acid sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package, Version 8 (available from Genetics Computer Group (GCG™ programs (Accelrys, Inc., San Diego, Calif.)). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65 and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., eds., Greene Publishing and Wiley-Interscience, New York (1995).

GAP uses the algorithm of Needleman and Wunsch, (1970) J. Mol. Biol. 48:443-53, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402). As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (C.sub.1-ayerie and States, (1993) Comput. Chem. 17:191-201) low-complexity filters can be employed alone or in combination.

### Anti-SPase Antibodies

Another aspect of the invention is an antibody that can distinguish between a SPase that contains or does not contain a proline within about 10-12 amino acids of the catalytic serine in the SPase polypeptide. Thus, in some embodiments, the antibody binds with specificity to a bacterial SPase epitope that contains a proline within about 10-12 amino acids of the catalytic serine in the SPase polypeptide. In other embodiments, the antibody binds with specificity to a bacterial SPase epitope that does not contain a proline within about 10-12 amino acids of the catalytic serine in the SPase polypeptide.

Antibodies that selectively bind to a SPase polypeptide can be isolated using conventional methods. Such antibodies can be polyclonal or monoclonal antibodies. In some embodiments, the anti-SPase antibodies are monoclonal antibodies.

For example, antibodies of the invention can be obtained from the blood or spleen of a animal that has been immunized with an SPase peptide or polypeptide that contains a selected amino acid sequence (e.g., SPase that contains or does not contain a proline within about 10-12 amino acids of the catalytic serine in the SPase polypeptide). The SPase polypeptide can be obtained using conventional methods, for example, as described in the Examples. Peptides from SPase polypeptides can be obtained by proteolytic cleavage of a SPase polypeptide or by recombinant expression of the SPase peptide. The animal can be, for example, a rabbit, goat, rat, horse or mouse. At the appropriate time after immunization, antibody molecules can be isolated from the animal, e.g. from the blood, spleen or other fluid of the animal, and further purified using standard techniques that include, without limitation, precipitation using ammonium sulfate, gel filtration chromatography, ion exchange chromatography or affinity chromatography using protein A. Antibodies that bind to SPase-specific antigens, can be identified using ELISA. Antibodies that bind to proline-containing epitopes on SPase proteins, but do not bind to non-proline-containing epitopes (or vice cersa) can be identified by screening methods available in the art.

Antibodies specific for proline-containing and non-proline-containing SPase polypeptides can also be obtained using various methods. Non-limiting examples include: (1) the generation of an antibody from an antibody-producing cell of a animal that has been immunized with a SPase polypeptide or peptide using single human B cell RT-PCR and expression vector cloning; (2) isolation from immortalized antibody-secreting B cells; and (3) isolation from an antibody-producing hybridoma generated by fusion of an antibody-producing cell with a myeloma cell. These techniques are known in the art. See, for example, Kohler & Milstein, Nature 256:495-97 (1975); Kozbor et al. Immunol Today 4: 72 (1983); Tiller et al., J Immunol Methods 329:112-124 (2008) and Traggiai et al., Nat Med 10:871-875(2004).

Antibodies specific for proline-containing and non-proline-containing SPase polypeptides can also be prepared using other methods known in the art, such as, for example, screening of a recombinant combinatorial immunoglobulin library such as an antibody phage display library using antigenic epitope of the SPase polypeptide. See, for example, Barbas, C. F. et al., PHAGE DISPLAY—A LABORATORY MANUAL (2001) Cold Spring Harbor, NewYork: Cold Spring Harbor Laboratory Press; and Kontermann & Dubel, ANTIBODY ENGINEERING (2001) Berlin, Heidelberg: Springer-Verlag.

Nucleic acids encoding antibodies specific for the SPase polypeptides of the invention can be derived from an animal immunized with the SPase polypeptide or a peptide fragment thereof by generating an expression library using the RNA of the animal's B cells or plasma cells and then screening for antibody-coding sequences. See, for example, in Antibodies, A Laboratory Manual, by Harlow and Lane, Cold Spring Harbor Laboratory Press, Cold Spring Harbor, N.Y., 1988, and in Molecular Cloning, A Laboratory Manual by Sambrook, et al., Cold Spring Harbor Laboratory Press, Cold Spring Harbor, N.Y. 1989, the disclosures of which are incorporated herein by reference.

For example, antibodies can be used that specifically detect any of the following peptides, where the proline(s) that give rise to arylomycin-resistance are shown as a P within a box (i.e., [symbol] ) and the catalytic serine is shown at the C-terminus.

TABLE 9
Peptide Epitopes

| | Mutant | | |
|---|---|---|---|
| | or | SPase | SEQ |
| | Wild | peptide | ID |
| Bacterial Strain | type | sequence | NO: |
| S. epidermidis RP62A | WT | VGKSYSIKGDS | |
| S. epidermidis PAS9001 | S29P | 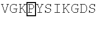 | |
| S. epidermidis PAS9002 | S31P | 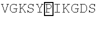 | |
| S. aureus NTCT 8325 | WT | VAKPYTVKGDS | |
| S. aureus PAS8001 | P29S | 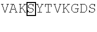 | |
| E. coli MG1655 | WT | IYEPFQIPSGS | |
| E. coli PAS0232 | P84S | 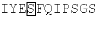 | |
| P. aeruginosa PAO1 | WT | LFEPFQIPSGS | |
| P. aeruginosa PAS2006 | P84S | 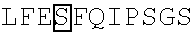 | |

Compositions and Combinations Another aspect of an embodiment of the invention provides compositions of the compounds of the invention, alone or in combination with another medicament. As set forth herein, compounds of the invention include stereoisomers, tautomers, solvates, prodrugs, pharmaceutically acceptable salts and mixtures thereof. Compositions containing a compound of the invention can be prepared by conventional techniques, e.g. as described in Remington: The Science and Practice of Pharmacy, 19th Ed., 1995, or later versions thereof, incorporated by reference herein. The compositions can appear in conventional forms, for example capsules, tablets, aerosols, solutions, suspensions or topical applications.

Typical compositions include a compound of the invention and a pharmaceutically acceptable excipient which can be a carrier or a diluent. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents that do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

Moreover, the compositions can include other therapeutic agents such as analgesics, other antibiotics, antihistamines, anti-inflammatory agents and the like. In some embodiments, the compositions include a second type of antibiotic, for example, a non-arylomycin antibiotic. Examples of non-arylomycin antibiotics that can be incorporated in the compositions (and used in the therapeutic methods described herein) include [LIST].

The route of administration can be any route which effectively transports the active compound of the invention to the appropriate or desired site of action, such as oral, nasal, pulmonary, buccal, subdermal, intradermal, transdermal, topical or parenteral, e.g., rectal, depot, subcutaneous, intravenous, intraurethral, intramuscular, intranasal, ophthalmic solution or an ointment, the oral route being preferred. In some embodiments, the compositions containing any of the compounds described herein are administered topically. For example, compositions containing arylomycon A and/or arylomycin B (with or without some of the compounds of formula I may advantageously be administered topically.

If a solid carrier is used for oral administration, the preparation can be tableted, placed in a hard gelatin capsule in powder or pellet form or it can be in the form of a troche or lozenge. If a liquid carrier is used, the preparation can be in the form of a syrup, emulsion, soft gelatin capsule or sterile injectable liquid such as an aqueous or non-aqueous liquid suspension or solution.

Injectable dosage forms generally include aqueous suspensions or oil suspensions which can be prepared using a suitable dispersant or wetting agent and a suspending agent Injectable forms can be in solution phase or in the form of a suspension, which is prepared with a solvent or diluent. Acceptable solvents or vehicles include sterilized water, Ringer's solution, or an isotonic aqueous saline solution. Alternatively, sterile oils can be employed as solvents or suspending agents. Preferably, the oil or fatty acid is non-volatile, including natural or synthetic oils, fatty acids, mono-, di- or tri-glycerides.

For injection, the formulation can also be a powder suitable for reconstitution with an appropriate solution as described above. Examples of these include, but are not limited to, freeze dried, rotary dried or spray dried powders, amorphous powders, granules, precipitates, or particulates. For injection, the formulations can optionally contain stabilizers, pH modifiers, surfactants, bioavailability modifiers and combinations of these. The compounds can be formulated for parenteral administration by injection such as by bolus injection or continuous infusion. A unit dosage form for injection can be in ampoules or in multi-dose containers.

The formulations of the invention can be designed to provide quick, sustained, or delayed release of the active ingredient after administration to the patient by employing procedures well known in the art. Thus, the formulations can also be formulated for controlled release or for slow release.

Compositions contemplated by the present invention can include, for example, micelles or liposomes, or some other encapsulated form, or can be administered in an extended release form to provide a prolonged storage and/or delivery effect. Therefore, the formulations can be compressed into pellets or cylinders and implanted intramuscularly or subcutaneously as depot injections. Such implants can employ known inert materials such as silicones and biodegradable polymers, e.g., polylactide-polyglycolide. Examples of other biodegradable polymers include poly(orthoesters) and poly(anhydrides).

For nasal administration, the preparation can contain a compound of the invention, dissolved or suspended in a liquid carrier, preferably an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet that can be prepared by conventional tabletting techniques can contain:

Core:

| | | |
|---|---|---|
| Active compound (as free compound or salt thereof) | 250 | mg |
| Colloidal silicon dioxide (Aerosil) ® | 1.5 | mg |
| Cellulose, microcryst. (Avicel) ® | 70 | mg |
| Modified cellulose gum (Ac-Di-Sol) ® | 7.5 | mg |

| | |
|---|---|
| Magnesium stearate | Ad. |

Coating:

| | | |
|---|---|---|
| HPMC approx. | 9 | mg |
| *Mywacett 9-40 T approx. | 0.9 | mg |

*Acylated monoglyceride used as plasticizer for film coating.

A typical capsule for oral administration contains compounds of the invention (250 mg), lactose (75 mg) and magnesium stearate (15 mg). The mixture is passed through a 60 mesh sieve and packed into a No. 1 gelatin capsule. A typical injectable preparation is produced by aseptically placing 250 mg of compounds of the invention into a vial, aseptically freeze-drying and sealing. For use, the contents of the vial are mixed with 2 mL of sterile physiological saline, to produce an injectable preparation.

The following non-limiting Examples illustrate aspects of the invention.

### EXAMPLES

### Example 1

### General Chemical Methods

¹H and ¹³C NMR spectra were recorded on Bruker AMX 400, Bruker DRX 500, or Bruker DRX 600 spectrometers. Chemical shifts are reported relative to either chloroform (δ 7.26), methanol (δ 3.31), or dimethylsulfoxide (DMSO) (δ 2.50) for ¹H NMR and either chloroform (δ 77.16), methanol (δ 49.00), or DMSO (δ 39.52) for ¹³C NMR. IR measurements were taken using a Nicolet 6700 ATR FT-IR. High resolution mass spectra were measured at the Scripps Center for Mass Spectrometry. All assigned structures are consistent with spectral data obtained.

Optical rotations were measured on a Perkin Elmer model 341 polarimeter. Yields refer to chromatographically and spectroscopically pure compounds unless otherwise stated. Reactions were magnetically stirred, and monitored by thin layer chromatography (TLC) with 0.25 mm Whatman pre-coated silica gel (with fluorescence indicator) plates. Flash chromatography was performed with silica gel (particle size 40-63 μm, EMD chemicals). Acetone was dried over anhydrous potassium carbonate, and all other dry solvents were purchased from Acros. H-D-Ser(Bzl)-OH and H-Ala-OBzl HCl were purchased from Bachem.

4-Nitrobenzenesulfonylchloride, and 1,1′-bis(diphenylphosphino)ferrocene palladium(II) chloride complex with dichloromethane (PdCl₂(dppf)) were purchased from Alfa Aesar and Strem Chemicals, respectively. Boc-Gly-OH was purchased from Novabiochem. Anhydrous 1-hydroxybenzotriazole (HOBT) was purchased from Chem-Impex. Diazomethane was prepared according to Arndt, F. Org. Synth. 1934, 2:165. All other chemicals were purchased from Fisher/Acros or Aldrich. Abbreviations: THF, tetrahydrofuran; EtOH, ethanol; MeOH, methanol; AcOH, acetic acid; DCM, dichloromethane; DMF, N,N-dimethylformamide; EDC, 1-(3-dimethylaminopropyl)-3-ethylcarbodiimide hydrochloride; EtOAc, ethyl acetate; Ar, argon; DBU, 1,8-diazabicyclo[5.4.0]undec-7-ene; TFA, trifluoroacetic acid.

All preparative reverse phase chromatography was performed using Dynamax SD-200 pumps connected to a Dynamax UV-D II detector (monitoring at 220 nm). The column used was a Phenomenex Jupiter C₁₈ (10 □m, 2.12×25 cm, 300 Å pore size). All solvents contained 0.1% TFA; Solvent A, H₂O; Solvent B, acetonitrile with 10% H₂O. All samples were loaded onto the column at 0% B and the column was allowed to equilibrate for ˜10 min. before a linear gradient was started. Retention values are reported according to the linear gradient used and the % B at the time the sample eluted.

### Procedures and Characterization

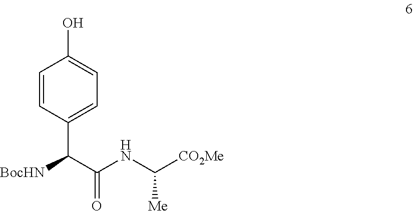 [SMILES: COC(=O)[C@H](C)NC(=O)[C@@H](NC(=O)OC(C)(C)C)c1ccc(O)cc1]

To a solution of 4-hydroxyphenylglycine (12 g, 71.8 mmol) in a 1:1 mixture of acetone and water was added di-tert-butyldicarbonate (16.5 mL, 71.8 mmol, 1 eq) and sodium bicarbonate (6.03 g, 0.11 mol, 1.5 eq). The solution was allowed to stir overnight, and then was quenched with the addition of citric acid (pH 3) to pH 4. The aqueous layer was then extracted 2× with EtOAc and the combined organic layers were washed with brine, dried over Na₂SO₄ and concentrated to a white foam. The crude material (18.43 g, 69 mmol (assumed)) was used without further purification by dissolving it in anhydrous DMF and treating sequentially with triethylamine (12.6 mL, 75.9 mmol, 1.3 eq), HOBT (9.32 g, 69 mmol, 1 eq) and Ala-OMe HCl (9.63 g, 69 mmol, 1 eq). The solution was then cooled to 0° C. and EDC (19.55 g, 0.1 mol, 1.5 eq) was added in one portion. The reaction was allowed to warm to room temperature and stirred overnight. Water and EtOAc were added, the aqueous layer was extracted 3×, and the combined organic layers were washed with brine, dried over Na₂SO₄ and concentrated. The residue was purified by flash chromatography (6% MeOH, 0.6% AcOH in DCM) to give a clear residue (17.82 g, 71% yield). R_{f}=0.39 (7% MeOH in DCM). ¹H NMR (CDCl₃, 600 MHz) δ (ppm) 7.11 (d, J=8.4 Hz, 2H), 6.64 (d, J=8.4 Hz, 2H), 6.51 (br d, J=6.6 Hz, 1H), 5.71 (br s, 1H), 5.07 (br s, 1H), 4.57-4.52 (m, 1H), 3.69 (s, 3H), 1.42-1.40 (m, 12H). ¹³C NMR (CDCl₃, 600 MHz) δ (ppm) 173.2, 170.5, 156.6, 155.4, 129.0, 128.7 (2C), 116.1 (2C), 80.5, 58.2, 52.7, 48.5, 28.4 (3C), 18.4. IR (film) vₘₐₓ=1655, 1512, 1450, 1365, 1215, 1157, 1049 cm⁻¹. ESI HRMS calcd for [(M+Na)⁺] C₁₇H₂₄N₂O₆: 375.1526. found: 375.1532.

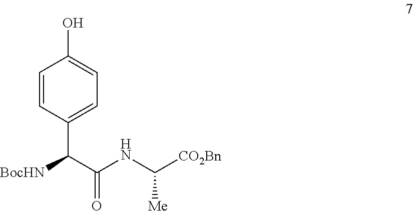 [SMILES: C[C@H](NC(=O)[C@@H](NC(=O)OC(C)(C)C)c1ccc(O)cc1)C(=O)OCc1ccccc1]

Compound 7 was synthesized in the same manner as compound 6. R_{f}=0.3 (4.5% MeOH in DCM). ¹H NMR (CDCl₃, 600 MHz) δ (ppm) 7.38-7.31 (m, 3H), 7.29-7.26 (m, 2H), 7.11 (d, J=8.5 Hz, 2H), 6.63 (d, J=8.3 Hz, 2H), 6.44-6.38 (m, 2H), 5.69 (br s, 1H), 5.15-4.99 (m, 3H), 4.59 (p, J=7.2 Hz, 1H), 1.44-1.39 (m, 12H). ¹³C NMR (CDCl₃, 600 MHz) δ (ppm) 172.5, 170.4, 162.8, 156.4, 135.2, 128.8 (2C), 128.7, 128.6 (2C), 128.3 (2 C), 116.1 (2C), 99.7, 67.5, 48.7, 36.7, 31.7, 28.5 (3C), 18.5. IR (film) vₘₐₓ=1655, 1510, 1209, 1153, 1045, 696 cm⁻¹. ESI HRMS calcd for C₂₃H₂₈H₂O₆ [(M+H)⁺] 429.2020. found 429.2025.

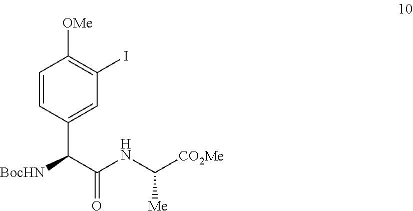 [SMILES: COC(=O)[C@H](C)NC(=O)[C@@H](NC(=O)OC(C)(C)C)c1ccc(OC)c(I)c1]

A mixture of 6 (20.6 g, 58 mmol) dissolved in dry acetone (390 mL) and potassium carbonate (40.4 g, 0.29 mol, 5 eq) under Ar was treated with iodomethane (25.4 mL, 0.41 mol, 7 eq) and heated to reflux. After 17 h, the solution was cooled, filtered and concentrated to the crude product (R_{f}−0.35 (2% MeOH in DCM)). To a solution of the crude compound (16.68 g, 45.6 mmol (assumed)) in MeOH (607 mL) was added AgSO₄ (14.9 g, 47.8 mmol, 1.05 eq) and I₂ (12.1 g, 47.8 mmol, 1.05 eq) consecutively. The reaction was stirred vigorously until TLC analysis (starting material stains with cerium(IV)sulfate; product does not) showed no remaining starting material (˜30 min) solid Na₂S₂O₃ (large excess) was then added. The solids were filtered, the filtrate was concentrated, and the crude residue was purified via flash column chromatography (2% MeOH in DCM). The product was a white solid (21.19 g, 76% yield). R_{f}=0.50 (3% MeOH in DCM). ¹H NMR (CDCl₃, 600 MHz) δ (ppm) 7.76 (d, J=1.8 Hz, 1H), 7.33 (d, J=7.8 Hz, 1H), 6.78 (d, J=8.4 Hz, 1H), 6.31 (d, J=7.2 Hz, 1H), 5.69 (br s, 1H), 5.07 (br s, 1H), 4.56-4.51 (m, 1H), 3.86 (s, 3H), 3.70 (s, 3H), 1.41-1.40 (m, 12H). ¹³C NMR (CDCl₃, 600 MHz) δ (ppm) 172.9 (2C), 169.5, 158.3, 138.3, 132.2, 128.9, 111.1, 86.6, 80.4, 57.4, 56.5, 52.7, 48.6, 28.4 (3C), 18.4. IR (film) vₘₐₓ=1655, 1489, 1363, 1248, 1155, 1047, 1016, 548 cm⁻¹. ESI HRMS calcd for C₁₈H₂₅IN₂O₆ [(M+H)⁺] 493.0830. found 493.0829.

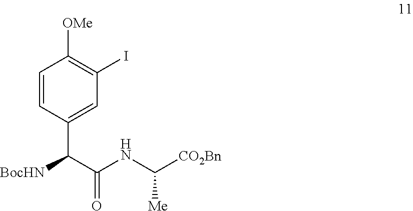 [SMILES: COc1ccc([C@H](NC(=O)OC(C)(C)C)C(=O)N[C@@H](C)C(=O)OCc2ccccc2)cc1I]

Compound 11 was synthesized in the same manner as compound 10. R_{f}=0.59 (2% MeOH in DCM). ¹H NMR (CDCl₃, 600 MHz) δ (ppm) 7.76 (s, 1H), 7.38-7.23 (m, 6H), 6.72 (d, J=8.4 Hz, 1H), 6.32 (d, J=6.6 Hz, 1H), 5.68 (s, 1H), 5.16-5.02 (m, 3H), 4.61-4.55 (m, 1H), 3.84 (s, 3H), 1.45-1.35 (m, 12H). ¹³C NMR (MeOD, 600 MHz) δ (ppm) 173.5, 172.4, 159.5, 157.3, 139.6, 137.1, 133.1, 129.9, 129.5 (2C), 129.1, 129.0 (2 C), 114.9, 111.9, 86.3, 80.9, 67.8, 58.1, 56.8, 28.7 (3C), 17.3. IR (film) vₘₐₓ=1655, 1489, 1246, 1153, 1045, 735, 696 cm⁻¹. ESI HRMS calcd for C₂₄H₂₉IN₂O₆ [(M+H)⁺] 569.1143. found 569.1149.

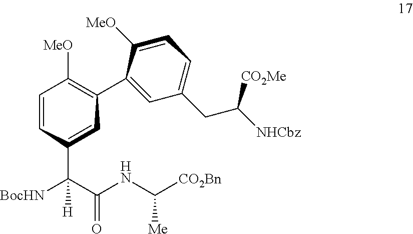

To a mixture of compound 11 (200 mg, 350 μmol), compound 15 (198 mg, 420 μmol, 1.2 eq) and K₂CO₃ (243 mg, 1.35 mmol, 5 eq) under Ar was added an Ar sparged suspension of PdCl₂(dppf) (57.5 mg, 70 μmol, 0.2 eq) in DMSO (3.5 mL) via cannula. The reaction was allowed to stir at 80° C. for 36 hrs. then was cooled and dilute NH₄Cl_{(aq)} and EtOAc were added. The aqueous layer was extracted 2× with EtOAc and the combined organic fractions were dried over Na₂SO₄ and concentrated. Purification by flash column chromatography (30% EtOAc in hexanes) yielded compound 17 (99.9 mg, 36% yield). R_{f}=0.14 (35% EtOAc in hexanes). ¹H NMR (CDCl₃, 600 MHz) multiple isomers. ¹³C NMR (CDCl₃, 600 MHz) multiple isomers. IR (film) vₘₐₓ=1707, 1666, 1500, 1452, 1242, 1209, 1151, 1049, 1022, 742, 696 cm⁻¹. ESI HRMS calcd for C₄₃H₄₉N₃O₁₁ [(M+H)⁺] 783.3440. found 7833444.

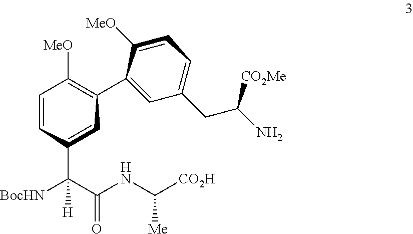

A solution of compound 17 (51.3 mg, 66 μmol) in 95% EtOH (16 mL) was treated with 10% Pd/C (17 mg) and allowed to stir until the presence of starting material could no longer be detected by TLC (˜3 hrs). The reaction mixture was filtered through Celite, washed with ethyl acetate, and concentrated. The product was taken forward with no further purification. ¹H NMR (MeOD, 600 MHz) multiple isomers. IR (film) vₘₐₓ=1497, 1246, 1155, 1132, 1045, 1024 cm⁻¹. ESI HRMS calcd for C₂₈H₃₇N₃O₉ [(M+H)⁺] 560.2602. found 560.2597.

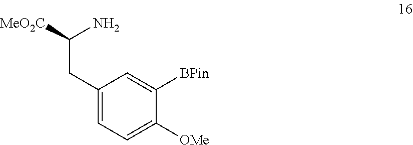

Compound 15 could not be purified completely due to instability on silica gel. A solution of semi-pure compound 15 (646 mg, 1.38 mmol) in 95% EtOH (70 mL) was treated with 10% Pd/C (215 mg) and allowed to stir until the presence of starting material could no longer be detected by TLC (35% EtOAc in Hex) (˜4 hrs). The reaction mixture was filtered through Celite, washed with ethyl acetate, and concentrated. The product, a yellow oil, was taken forward with no further purification (431 mg, 93% yield). ¹H NMR (CDCl₃, 400 MHz) δ (ppm) 7.48 (d, J=2.3 Hz, 1H), 7.21 (dd, J=8.4, 2.3 Hz, 1H), 6.80 (d, J=8.5 Hz, 1H), 3.81 (s, 3H), 3.76-3.68 (m, 4H), 3.03 (dd, J=13.7, 4.9 Hz, 1H), 2.78 (dd, J=13.6, 8.1 Hz, 1H), 1.39-1.32 (m, 12H) ¹³C NMR (CDCl₃, 600 MHz) δ (ppm) 175.6, 163.4, 137.6, 133.4, 128.5, 110.8, 83.6 (2C), 83.2, 56.1, 56.0, 52.1, 40.2, 25.0 (3 C). IR (film) vₘₐₓ=1605, 1493, 1417, 1335, 1248, 1142, 1070, 1022, 852, 796, 673, 538 cm⁻¹. ESI HRMS calcd for C₁₇H₂₆BNO₅ [(M+H)⁺] 336.1977. found 336.1973.

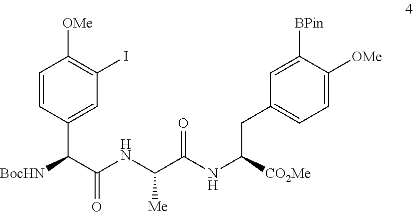

To a solution of compound 10 (1 g, 2.0 mmol) in THF (100 mL) was added 17 mL of 0.2 M LiOH_{(aq)} (3.4 mmol, 1.7 eq). The reaction was allowed to stir until TLC indicated all starting material had been consumed. The reaction was then quenched by the addition of 5% NH₄Cl and most of the THF was blown off under a stream of nitrogen. Water and EtOAc were added and the aqueous phase was extracted 2× with EtOAc. The combined organic layers were then washed with brine, dried over Na₂SO₄, and concentrated. Compound 19 (1.9 g, 4.36 mmol) was dissolved in a 2.2:1 mixture of acetonitrile and DMF (25.7 mL). The compound was then treated sequentially with NaHCO₃ (catalytic), HOBT (1.5 g, 10.9 mmol, 2.5 eq), compound 16 (1.61 g, 4.8 mmol, 1.1 eq), and EDC (1.8 g, 9.59 mmol, 2.2 eq) and stirred under Ar overnight. The volatiles were evaporated, the residue was taken up in ethyl acetate and washed with 5% NaHCO_{3(aq)}, citric acid_{(aq)}, water, and brine. The organic layer was dried over Na₂SO₄ and concentrated. Abbreviated column chromatography (2.5% MeOH in DCM) yielded a semi-pure product as a yellow foam (1.22 g, 79% yield). The product could not be purified further due to instability upon prolonged exposure to silica gel or a C₁₈ HPLC column. Crude spectra were used to characterize the compound. R_{f}=0.32 (3% MeOH in DCM). ¹H NMR (CDCl₃, 600 MHz) δ (ppm) 7.85 (s, 1H), 7.30 (d, J=10.8 Hz, 1H), 7.11 (d, J=8.2 Hz, 1H), 6.81-6.71 (m, 3H), 6.23 (br s, 1H), 5.81 (br s, 1H), 5.15 (br s, 1H), 4.74-4.68 (m, 1H), 4.58-4.51 (m, 1H), 3.87 (s, 3H), 3.85 (s, 3H), 3.73 (s, 3H), 3.05-3.02 (m, 1H), 2.87-2.80 (m, 1H), 1.52-1.36 (m, 24H) ¹³C NMR (CDCl₃, 600 MHz) δ (ppm) 171.5, 171.0, 169.9, 163.5, 158.2, 155.1, 138.1, 137.7, 133.4, 132.3, 128.5, 127.0, 111.3, 110.8, 86.7, 83.9 (2C), 80.3, 57.4, 56.5, 56.0, 53.5, 52.4, 49.0, 36.8, 28.4 (3C), 25.2 (4 C), 18.2 (no signal was observed for the carbon attached to the boron). IR (film) vₘₐₓ=1645, 1489, 1344, 1248, 1144, 1072, 1047, 1018, 854, 656, 550 cm⁻¹. ESI HRMS calcd for [(M+H)⁺] C₃₄H₄₇BIN₃O₁₀: 796.2472. found: 796.2465.

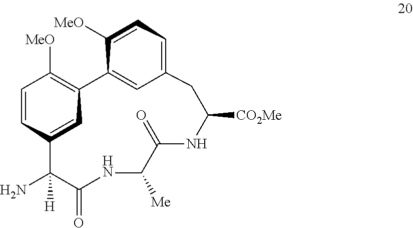

A solution of compound 4 (100 mg, 126 μmol) and K₂CO₃ (174 mg, 1.26 mmol, 10 eq) in acetonitrile (1.9 mL, 20 mM final) was stirred in a sealed vial and purged extensively with Ar. The mixture was then treated via syringe with a suspension of PdCl₂(dppf) (10.3 mg, 12.6 μmol, 10 mol %) in acetonitrile (1.3 mL, 5 mM) that had been sparged with Ar. The vial was then heated to 80° C., and allowed to stir for 21 hrs. The reaction mixture was cooled, EtOAc and dilute NH₄Cl_{(aq)} were added. The aqueous phase was extracted 2× with EtOAc and the combined organic phases were washed with brine, dried over Na₂SO₄, and concentrated. The crude residue was then filtered through an abbreviated silica column (4% MeOH in DCM) to remove palladium species yielding a semipure residue. A solution of crude compound 18 (R_{f}=0.36 (4% MeOH in DCM)) was then taken up in anhydrous CH₂Cl₂ (2 mL) under Ar and treated dropwise with trifluoroacetic acid (0.5 mL). The reaction was allowed to stir until TLC indicated complete disappearance of starting material (˜50 min). The volatiles were then evaporated under a stream of nitrogen, the crude material was purified via column chromatography (9.5% MeOH in DCM with trace triethylamine) and the purified material was filtered through basic alumina to give compound 20 (26 mg, 48% yield). R_{f}=0.44 (10% MeOH in DCM with 1 drop of TEA/10 mL). ¹H NMR (MeOD, 600 MHz) multiple isomers. ¹³C NMR (MeOD, 600 MHz) multiple isomers. IR (film) vₘₐₓ=1624, 1508, 1269, 1246, 1176, 1022, 795, 582 cm⁻¹. ESI HRMS calcd for C₂₃H₂₇N₃O₆ [(M+H)⁺] 442.1973. found 442.1966.

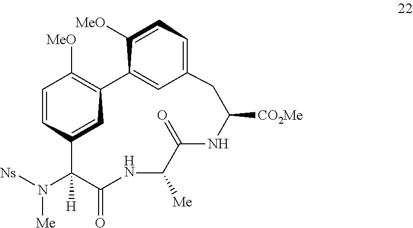

A solution of compound 20 (94 mg, 223 μmol) in acetonitrile (3 mL) was treated with nosyl chloride (76 mg, 335 μmol, 1.5 eq) and triethylamine (91 μL, 669 μmol, 3 eq). After stirring for 2 hrs all starting material was consumed as determined by TLC and a white precipitate had formed. The solvent was then evaporated, purged with Ar, and crude compound 21 (R_{f}−0.36 (7% MeOH in DCM)) was taken up in dry acetone (4 mL). The dissolved material was treated with K₂CO₃ (314 mg, 2.3 mmol 10 eq), the reaction tube was sealed, and iodomethane (129 μL, 2.3 mmol, 10 eq) was added. The mixture was stirred at 65° C. overnight then cooled, filtered, and concentrated. The residue was then subjected to flash column chromatography (2.5% MeOH in DCM) yielding compound 22 (54 mg, 37% yield). R_{f}=0.32 (2.5% MeOH in DCM). ¹H NMR (CDCl₃, 600 MHz) δ (ppm) 8.34 (d, J=7.2 Hz, 1H), 8.02 (d, J=7.2 Hz, 1H), 7.38 (d, J=8.3 Hz, 1H), 6.94 (d, J=8.2 Hz, 1H), 6.82 (d, J=8.2 Hz, 1H), 6.67-6.59 (m, 2H), 6.56 (s, 1H), 6.24 (d, J=4.8 Hz, 1H), 5.81 (s, 1H), 4.85-4.80 (m, 1H), 4.52-4.44 (m, 1H), 3.84-3.76 (m, 9H), 3.44 (d, J=15.3 Hz, 1H), 2.99 (dd, J=15.5, 5.7 Hz, 1H), 2.83 (s, 3H), 1.30 (d, J=5.3 Hz, 3H) ¹³C NMR (CDCl₃, 600 MHz) δ (ppm) 172.0, 171.8, 169.0, 157.4, 156.1, 150.1, 145.1, 134.5, 134.5, 129.7, 128.9, 128.7 (2C), 128.6, 128.3, 127.0, 125.1, 124.2 (2C), 112.3, 111.8, 62.6, 56.1 (2C), 53.1, 53.0, 49.8, 34.5, 32.0, 19.9. IR (film) vₘₐₓ=1645, 1527, 1504, 1348, 1269, 1173, 1146, 735, 606 cm⁻¹. ESI HRMS calcd for C₃₀H₃₂N₄O₁₀S [(M+Na)⁺]: 663.1731. found: 663.1724.

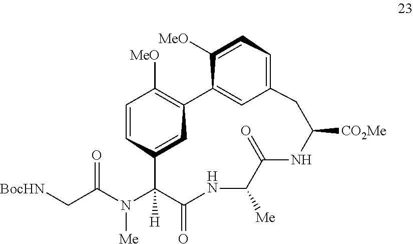

To a solution of compound 22 (20 mg, 31.3 μmol) in acetonitrile (1 mL) under Ar was added sequentially 2-mercaptoacetic acid (6.5 μL, 94 μmol, 3 eq) and DBU (23 μL, 157 μmol, 5 eq). The reaction was monitored by TLC for the disappearance of starting material and the volatiles were evaporated under a stream of nitrogen when the starting material had been completely consumed (˜30 min). The residue was taken up in EtOAc and 1 N HCl was added. The organic layer was extracted 2× with 1 N HCl and the combined aqueous layers were basified with saturated NaHCO₃. The aqueous layer was then extracted 2× with EtOAc and the combined organic layers were dried over sodium sulfate and concentrated. The product (R_{f}=0.29 (8% MeOH in DCM with 1 drop of TEA/10 mL)) was not further purified and was taken directly to the next reaction. A solution of the crude material in CH₂Cl₂:DMF (3:1, 1 mL) was treated sequentially with HoBT (11.1 mg, 82.5 μmol, 3.3 eq), Boc-Gly-OH (4.8 mg, 27.5 μmol, 1.1 eq), and EDC (14.4 mg, 75 μmol, 3 eq). The reaction was stirred overnight, then the volatiles were evaporated and EtOAc and water were added. The aqueous layer was extracted (2×) with EtOAc and the combined organic layers were washed with brine, dried over Na₂SO₄ and concentrated. The crude material was purified via column chromatography (4% MeOH in DCM) to yield a white foam (14.1 mg, 74% yield). R_{f}=0.32 (5% MeOH in DCM). ¹H NMR (CDCl₃, 600 MHz) δ (ppm) 7.15 (d, J=7.9 Hz, 1H), 6.99 (d, J=7.1 Hz, 1H), 6.93 (d, J=8.3 Hz, 1H), 6.86 (d, J=8.3 Hz, 1H), 6.80 (s, 1H), 6.71 (s, 1H), 6.44-6.26 (m, 3H), 5.57 (s, 1H), 4.96-4.89 (m, 1H), 4.74-4.66 (m, 1H), 4.13 (d, J=16.3 Hz, 1H), 4.00 (d, J=16.7 Hz, 1H), 4.86-4.80 (m, 9H), 4.13 (d, J=16.3 Hz, 1H), 4.00 (d, J=16.7 Hz, 1H), 3.54 (d, J=15.9 Hz, 1H), 3.04 (dd, J=15.7, 7.0 Hz, 1H), 2.79 (s, 3H), 1.46 (s, 9H), 1.38 (d, J=6.53 Hz, 3H) ¹³C NMR (CDCl₃, 600 MHz) δ (ppm) 172.3, 172.0, 170.2, 170.0, 157.2, 156.1, 156.0, 135.3, 1343, 129.9, 129.0, 129.0, 128.4, 127.2, 125.9, 112.1, 111.8, 79.7, 60.3, 56.1, 53.0, 52.7, 49.9, 46.0, 42.9, 34.5, 31.7, 28.5 (3C), 19.7. IR (film) vₘₐₓ=1639, 1508, 1269, 1246, 1163, 1024 cm⁻¹. ESI HRMS calcd for C₃₁H₄₀N₄O₉ [(M+H)⁺]: 613.2868. found: 613.2860.

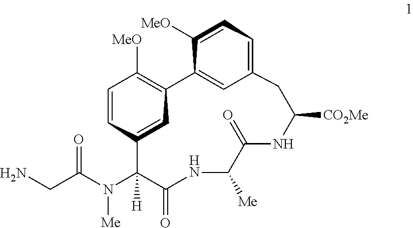

A solution of compound 23 (29.2 mg, 47.7 μmol) in CH₂Cl₂ (0.66 mL) was treated with TFA (0.33 mL) while stirring at 0° C. in a sealed vial. After 30 min, the reaction was found to be complete by TLC analysis, the vial was warmed to room temperature, a small amount of ethyl acetate was added, and the volatiles were evaporated under a stream of dry N₂ gas and dried in vacuo. The residue was then taken up in ethyl acetate and K₂CO₃ solid was added. The mixture was then filtered, concentrated, and purified by column chromatography (10% MeOH in DCM w/trace TEA). The product was obtained as a white residue (69 mg, 96% yield). R_{f}=0.33 (14% MeOH in DCM with 1 drop of TEA/10 mL). ¹H NMR (MeOD, 400 MHz) multiple isomers, see page S34. ¹³C NMR (CDCl₃, 600 MHz) δ (ppm) 172.3, 1710, 170.5, 157.1, 156.1, 135.3, 134.4, 129.8, 128.9, 128.9, 128.4, 127.3, 126.3, 112.1, 111.8, 60.4, 56.1, 56.1, 53.0, 52.7, 49.8, 43.6, 34.5, 31.6, 19.6. IR (film) vₘₐₓ=1639, 1506, 1269, 1202, 1175, 1128, 1022, 800, 719 cm⁻¹. ESI HRMS calcd for C₂₆H₃₂N₄O₇ [(M+H)⁺]: 513.2344. found: 513.2340.

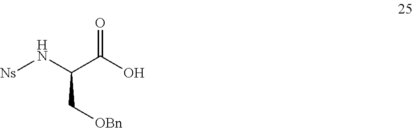

A solution of D-Ser-(OBzl)-OH (1.47 g, 15.4 mmol) in 1 N NaOH (14.7 mL) was treated dropwise with 4-nitrobenzenesulfonyl chloride (1.85 g, 7.9 mmol, 1.05 eq) in THF (1.4 mL). The solution was allowed to stir overnight then neutralized with citric acid (pH-3.5) and extracted with ethyl acetate (3×). The organic layers were combined, dried over Na₂SO₄, and concentrated. Flash column chromatography (6% MeOH in DCM w/trace AcOH) gave compound 25 as an off-white powder (62% yield). R_{f}=0.42 (7% MeOH in DCM w/1 drop/10 mL AcOH). ¹H NMR (CDCl₃, 600 MHz) δ (ppm) 8.24 (d, J=11.0 Hz, 2H), 7.98 (d, J=11.0 Hz, 2H), 7.32-7.19 (m, 5H), 5.82 (d, J=11.0 Hz, 1H), 4.50-4.43 (m, 2H), 4.23 (m, 1H), 3.85 (dd, J=14.4 Hz, J=4.5 Hz, 1H), 3.65 (dd, J=14.4 Hz, J=4.5 Hz, 1H). ¹³C NMR (CDCl₃, 600 MHz) δ (ppm) 173.3, 150.2, 146.0, 136.7, 128.7 (2C), 128.4, 128.4 (2C), 128.0 (2C), 124.4 (2C), 73.8, 70.1, 56.0. IR (film) vₘₐₓ=1740, 1529, 1350, 1173, 1086, 856, 737, 656, 609, 554 cm⁻¹. ESI HRMS calcd for C₁₆H₁₆N₂O₇S [(M+Na)⁺]: 403.0570. found: 403.0566.

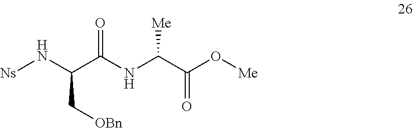

A solution of D-Ala-OMe HCl (147 mg, 1.05 mmol) in a 3:1 mixture of dichloromethane and DMF (30 mL) under Ar at 0° C. was treated sequentially with NaHCO₃ (88 mg, 1.05 mmol, 1 eq), anhydrous HoBT (468 mg, 3.5 mmol, 3.3 eq.), compound 25 (400 mg, 1.05 mmol, 1 eq.), and EDC (604 mg, 3.15 mmol, 3 eq.). The solution was allowed to warm to room temperature and stirred overnight. All volatiles were then evaporated, and the mixture was diluted with EtOAc and water. The aqueous layer was extracted (2×) and the combined organic layers were dried over Na₂SO₄ and concentrated. Flash column chromatography (1% MeOH in DCM) afforded the product in 88% yield. R_{f}=0.20 (1% MeOH in DCM with 1 drop of AcOH/10 mL). ¹H NMR (CDCl₃, 500 MHz) δ (ppm) 8.27 (d, J=8.9 Hz, 2H), 7.99 (d, J=8.9 Hz, 2H), 7.37-7.30 (m, 3H), 7.28-7.23 (m, 2H), 7.18 (d, J=7.3 Hz, 1H), 5.85 (d, J=5.7 Hz, 1H), 4.51 (s, 1H), 4.47-4.40 (m, 1H), 3.89-3.83 (m, 2H), 3.72 (s, 3H), 3.54-3.49 (m, 1H), 1.29 (d, J=7.2 Hz, 1H) ¹³C NMR (CDCl₃, 600 MHz) δ (ppm) 172.7, 168.1, 150.3, 145.1, 136.8, 128.8 (2C), 128.7 (2C), 128.5, 128.2 (2C), 124.5 (2C), 73.9, 70.1, 55.4, 52.7, 48.6, 18.2. IR (film) vₘₐₓ=1645, 1525, 1450, 1348, 1310, 1165, 1119, 1092, 852, 733, 617, 546, 525 cm⁻¹. ESI HRMS calcd for C₂₀H₂₃N₃O₈S [(M+Na)⁺]: 488.1098. found: 488.1095.

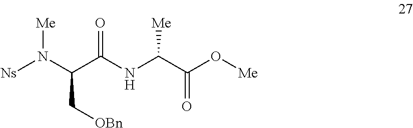

A solution of compound 26 (417 mg, 0.90 mmol) in dry CH₂Cl₂ (15 mL) under Ar was treated with diazomethane (0.66 M in ether, 16.3 mL, 12 eq, 11 mmol). The solution was monitored by TLC and when all starting material had been consumed, the volatiles were evaporated under a stream of dry nitrogen. Flash column chromatography (1% MeOH in DCM) yielded the product (86 mg, 90% yield). R_{f}=0.57 (2% MeOH in DCM). ¹H NMR (CDCl₃, 400 MHz) δ (ppm) 8.05 (d, J=9 Hz, 2H), 7.92 (d, J=9 Hz, 2H), 7.30-7.26 (m, 3H), 7.12-7.02 (m, 3H), 4.50 (p, J=7 Hz, 1H), 4.29 (dd, J=40.0 Hz, 11.0 Hz, 2H), 3.90-3.84 (m, 1H), 3.78-3.70 (m, 4H), 2.89 (s, 3H), 1.41 (d, J=7.2 Hz, 3H). ¹³C NMR (CDCl₃, 600 MHz) δ (ppm) 172.8, 167.6, 149.9, 144.4, 136.7, 128.9 (2C), 128.6 (2 C), 128.5, 128.3 (2C), 123.9 (2C), 73.9, 67.1, 59.7, 52.8, 48.4, 30.7, 18.2. IR (film) vₘₐₓ=1740, 1670, 1525, 1346, 1153, 1107, 1086, 854, 741, 604 cm⁻¹. ESI HRMS calcd for C₂₁H₂₅N₃O₈S [(M+H)⁺]: 480.1435. found 480.1445.

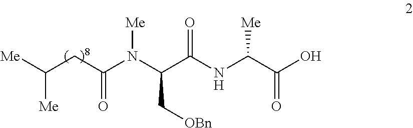

A solution of compound 27 (86.2 mg, 180 μmol) in anhydrous acetonitrile under Ar was treated sequentially with 2-mercaptoacetic acid (38 μL, 540 μmol, 3 eq) and DBU (135 μL, 900 μmol, 5 eq). The compound was allowed to stir until TLC indicated all of the starting material had been consumed. At this point, HCl (0.5 N) and EtOAc were added, the aqueous layer was extracted and then basified with saturated NaHCO₃. The aqueous layer was then extracted again with EtOAc (2×) and the combined organic layers from this extraction were pooled, dried over Na₂SO₄, and concentrated yielding compound 28. A 9% NaHCO₃ solution was then added to the crude material and stirred. In a separate reaction, isolauric acid (34 mg, 170 mmol) in anhydrous CH₂Cl₂ (1.5 mL) was treated with SOCl₂ (148 μL, 2.0 mmol, 12 eq) and refluxed for ˜45 min. The volatiles were then blown off under a stream of N₂ and pumped off in vacuo. The resulting white film was taken up in 2 mL of anhydrous CH₂Cl₂ and this solution was added to the freshly deprotected compound 28 in 9% NaHCO₃. This mixture was stirred vigorously for ˜5 hrs. The organic layer was removed and the water layer was extracted 2× with additional CH₂Cl₂ The combined organic layers were then dried over Na₂SO₄ and concentrated. This crude material was then dissolved in THF (2 mL), treated with 0.2 N LiOH (0.98 ml, 1.15 eq) and allowed to stir for 3 hrs. Then 5% NH₄Cl_{(aq)} and EtOAc were added the aqueous phase was extracted 2× with EtOAc, the combined organic layers were dried over Na₂SO₄, and concentrated. The crude material was purified using column chromatography (8% MeOH in DCM w/trace AcOH) yielding compound 2 (15.2 mg, 19% yield). R_{f}=0.35 (8% MeOH in DCM with 1 drop of AcOH/10 mL). ¹H NMR (CDCl₃, 600 MHz) multiple isomers, see page S42. IR (film) vₘₐₓ=2924, 2850, 1726, 1624, 1529, 1454, 1402, 1205, 1105, 733, 696 cm⁻¹. ESI HRMS calcd for C₂₆H₄₂N₂O₅ [(M+H)⁺] 463.3166. found 463.3158.

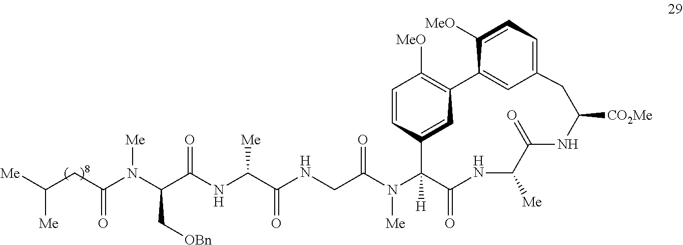

To a solution of compound 1 (16.5 mg, 0.03 mmol) in a 2.2:1 mixture of AcCN and DMF (1.5 mL) was added sequentially HOBT (13.5 mg, 0.1 mmol, 3.1 eq), compound 2 (14.9 mg, 0.03 mmol, 1 eq) and EDC (18.5 mg, 0.97 mmol, 3 eq) under Ar. The reaction was allowed to stir overnight. Water and EtOAc were then added and the aqueous layer was extracted 2× with EtOAc. The combined organic layers were then washed with brine, dried over Na₂SO₃, and concentrated. Flash column chromatography (4.5% MeOH in DCM) afforded the product (19.5 mg, 63% yield). R_{f}=0.25 (3% MeOH in DCM). ¹H NMR (CDCl₃, 600 MHz) multiple isomers. ¹³C NMR (CDCl₃, 600 MHz) multiple isomers. IR (film) vₘₐₓ=1630, 1506, 1265, 1103, 1026, 798, 696 cm⁻¹. ESI HRMS calcd for C₅₂H₇₂N₆O₁₁ [(M+H)⁺] 957.5332. found 957.5334.

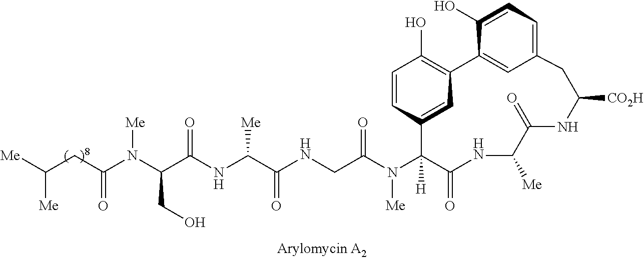

To a solution of compound 29 (8 mg, 8.4 μmol) in ethanethiol (0.4 mL) under Ar was added AlBr₃ in a 1.0 M solution of CH₂Br₂ (0.21 mL, 25 eq) via syringe. The reaction was then heated to 50° C. for 4 hrs. Upon cooling, water and EtOAc were added and the aqueous layer was extracted (2×) with EtOAc. The aqueous layer was then extracted (2×) with acetonitrile and the combined organic layers were dried over Na₂SO₄ and concentrated. The residue was then taken up in 100% B (1 mL) and this solution and the aqueous layer were purified by HPLC column (linear gradient, 0.5% B per minute, product eluted at 58.3% B) to yield a white powder after lyophilization (7.2 mg, 46% yield). ¹H NMR (MeOD, 600 MHz) δ (ppm) multiple isomers, see page S45. ¹³C NMR (MeOD, 600 MHz) multiple isomers. IR (film) vₘₐₓ=1630, 1506, 1408, 1230, 810 cm⁻¹. ESI HRMS calcd for C₄₂H₆₀N₆O₁₁ [(M+H)⁺] 825.4393. found 825.4397. [α]²⁰_{D} (c 3.4, MeOH): +85.1°.

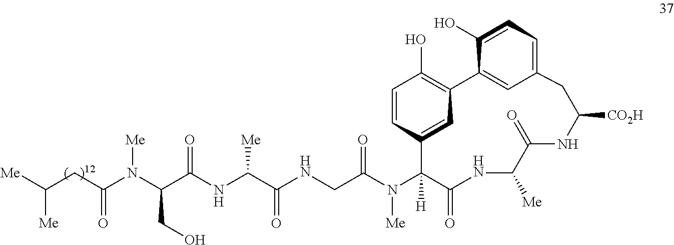

Compound 37 was synthesized in the same manner as arylomycin A₂. ¹H NMR (MeOD, 600 MHz) δ (ppm) multiple isomers. ¹³C NMR (MeOD, 600 MHz) δ (ppm) multiple isomers. IR (film) vₘₐₓ=3275, 2922, 2852, 1632, 1506, 1410, 1230, 810 cm⁻¹. ESI HRMS calcd for C₄₆H₆₈N₆O₁₁ [(M+H)⁺] 881.5019. found 881.5025. [α]²⁰_{D} (c 1.8, MeOH): +64.9°.

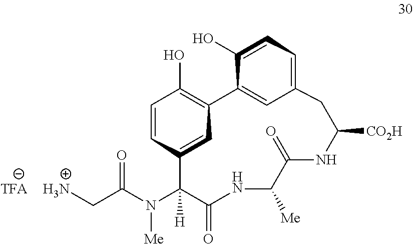

To a solution of compound 1 (49 mg, 0.08 mmol) in ethanethiol (2 mL) under Ar in a sealed tube was added AlBr₃ in CH₂Br₂ (1.0 M, 2 mL, 25 eq). The reaction was heated to 50° C. and allowed to stir for five hours. The reaction was then cooled to room temperature, a small amount of water was added, and the volatiles were evaporated under a stream of nitrogen. Additional water was added, followed by EtOAc, and the aqueous layer was extracted, filtered through a 0.22 μm syringe filter and purified by HPLC (linear gradient, 0.5% B per minute, product eluted at 17.9% B). Lyophilization afforded the product as the TFA salt, (30 mg, 63% yield). ¹H NMR (DMSO, 500 MHz) multiple isomers. ¹³C NMR (DMSO, 600 MHz) multiple isomers. IR (film) vₘₐₓ=1639, 1509, 1416, 1184, 1134, 798, 721, 511 cm⁻¹. ESI HRMS calcd for C₂₃H₂₆N₄O₇ [(M+H)⁺] 471.1874. found 471.1880.

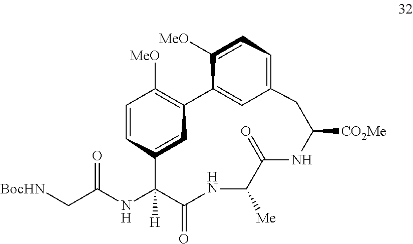

To a solution of compound 17 (30 mg, 0.073 mmol) in AcCN:DMF (2:1, 1.5 mL) under Ar was added consecutively HOBT (30 mg, 0.22 mmol, 3 eq), Boc-Gly-OH (14 mg, 0.08 mmol, 1.1 eq), and EDC (42 mg, 0.22 mmol, 3 eq). The reaction was stirred overnight then water and EtOAc were added. The water layer was extracted (2×), and the combined organic layers were washed with brine, dried over Na₂SO₄, and concentrated. Flash column chromatography (5.5% MeOH in DCM) yielded the product as a white flaky solid (30 mg, 68% yield). R_{f}=0.37 (7% MeOH in DCM). ¹H NMR (DMSO, 500 MHz) multiple isomers. ¹³C NMR (DMSO, 600 MHz) multiple isomers. IR (film) vₘₐₓ=1633, 1506, 1267, 1246, 1163, 1026, 580 cm⁻¹. ESI HRMS calcd for C₃₀H₃₈N₄O₉ [(M+H)⁺] 599.2711. found 599.2701.

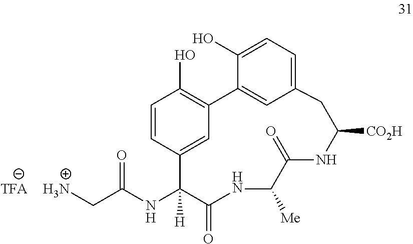 [SMILES: C[C@@H]1NC(=O)[C@@H](NC(=O)C[NH3+])c2ccc(O)c(c2)-c2cc(ccc2O)C[C@@H](C(=O)O)NC1=O]

To a solution of compound 32 (15 mg, 0.025 mmol) in ethanethiol (0.6 mL) under Ar in a sealed tube was added AlBr₃ in CH₂Br₂ (1.0 M, 0.6 mL, 25 eq). The reaction was heated to 50° C. and allowed to stir for five hours. The reaction was then cooled to room temperature, a small amount of water was added and the volatiles were blown off under a stream of nitrogen. Additional water was added and the aqueous layer was extracted with EtOAc, the aqueous layer was filtered through a 0.22 μm syringe filter and purified by HPLC (linear gradient=0.67% B per minute, product eluted at 17.9% B). Lyophilization afforded the product as the TFA salt. (5.8 mg, 52% yield). ¹H NMR (MeOD, 600 MHz) δ (ppm) 7.24 (dd, J=8.4, 2.2 Hz, 1H), 7.15-7.10 (m, 2H), 7.06 (d, J=2.00 Hz, 1H), 6.92 (d, J=8.41 Hz, 1H), 6.86 (d, J=8.26 Hz, 1H), 5.80 (s, 1H), 4.97-4.92 (m, 1H), 4.74 (dd, J=10.96, 2.27 Hz, 1H), 3.84-3.76 (m, 2H), 3.35-3.30 (m, 1H), 3.02 (dd, J=15.82, 11.05 Hz, 1H), 1.37 (d, J=6.83 Hz, 3H) (¹³C NMR (DMSO, 600 MHz) δ (ppm) 182.8, 181.1, 179.0, 175.9, 163.2, 162.4, 141.3, 140.9, 139.1, 138.7, 138.3, 136.0, 135.9, 135.8, 125.6, 63.9, 62.6, 58.2, 57.8, 50.1, 43.9, 28.8. IR (film) vₘₐₓ=3271 (br), 1633, 1543, 1500, 1236, 1188, 1136, 798, 683, 563 cm⁻¹. ESI HRMS calcd for C₂₂H₂₄N₄O₇ [(M+H)⁺] 457.1718. found 457.1721.

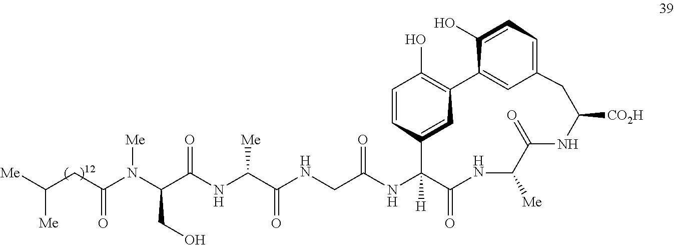

Compound 29 was synthesized in the same manner as arylomycin A₂. ¹H NMR (MeOD, 600 MHz) δ (ppm) multiple isomers. ¹³C NMR (MeOD, 600 MHz) δ (ppm) multiple isomers. ESI HRMS calcd for C₄₅H₆₆N₆O₁₁ [(M+H)⁺] 867.4862. found 867.4853.

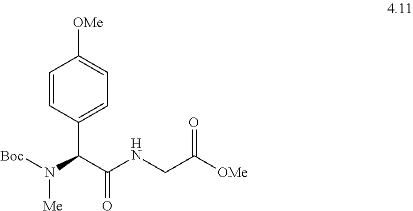 [SMILES: COC(=O)CNC(=O)[C@H](c1ccc(OC)cc1)N(C)C(=O)OC(C)(C)C]

To a solution of H-Gly-OMe HCl (40 mg, 0.32 mmol, 1 eq) in DMF (3 mL) was added sequentially Boc-N-Me-Hydroxyphenylglycine (122 mg, 1.35 eq), HOBT (43 mg, 1 eq), EDC (67 mg, 1.1 eq) and TEA (49 μL, 1.1 eq). The solution was allowed to stir overnight then dilute NaHCO_{3(aq)} and EtOAc, the aqueous phase was extracted 3× with EtOAc and the combined organic fractions were washed with 5% citric acid (pH—3), water and brine. The organics were dried over sodium sulfate, concentrated then the crude was taken on directly without further purification. The crude residue (113 mg) was taken up in acetone (3.5 mL), treated with MeI (139 μL, 7 eq) and K₂CO₃ (221 mg, 5 eq) and heated at reflux in a sealed vial overnight. The reaction was then allowed to warm to room temperature, the volatiles were blown off and water and EtOAc were added. The aqueous layer was extracted 3× with EtOAc and the combined organic layers were dried over sodium sulfate and concentrated. Column chromatography (3% MeOH in DCM) yielded the product (64.3 mg, 55% yield over two steps). ¹H NMR (CDCl₃, 500 MHz) δ (ppm) 7.27 (d, J=8.5 Hz, 2H), 6.89 (d, J=8.5 Hz, 2H), 6.49 (br s, 1H), 5.84 (br s, 1H), 4.09-4.08 (m, 2H), 3.80 (s, 3H), 3.75 (s, 3H), 2.71 (s, 3H), 1.47 (s, 9H) ¹³C NMR (CDCl₃, 500 MHz) δ (ppm) 170.6, 1702, 159.6, 130.6, 127.2, 114.1, 80.6, 55.3, 52.4, 41.3, 31.5, 32.1, 28.4. MS (ESI) m/z 389.2 (M+Na⁺).

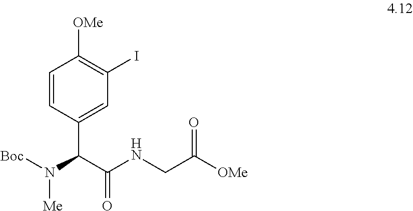 [SMILES: COC(=O)CNC(=O)[C@H](c1ccc(OC)c(I)c1)N(C)C(=O)OC(C)(C)C]

To a solution of Compound 4.11 (63 mg, 0.17 mmol, 1 eq) in MeOH (2 mL) was added sequentially AgSO₄ (56 mg, 1.05 eq) and I₂ (46 mg, 1.05 eq). The reaction mixture was allowed to stir for 2 hrs then an excess of solid sodium thiosulfate was added, the reaction was filtered through glass wool and concentrated by rotary evaporation. The crude material was purified via column chromatography (2% MeOH in DCM) to yield the product (74 mg, 88% yield). ¹H NMR (CDCl₃, 500 MHz) δ (ppm) 7.72 (d, J=2.0 Hz, 1H), 7.30 (dd, J=8.5 Hz, J=1.5 Hz, 1H), 6.78 (d, J=8.5 Hz, 1H), 6.54 (br s, 1H), 5.78 (br s, 1H), 4.06-4.05 (m, 2H), 3.85 (s, 3H), 3.74 (s, 3H), 2.70 (s, 3H), 1.45 (s, 9H). ¹³C NMR (CDCl₃, 500 MHz) δ (ppm) 170.1, 170.0, 158.1, 140.2, 130.6, 129.3, 110.7, 86.1, 80.9, 56.5, 52.5, 41.3, 31.7, 28.4. MS (ESI) m/z XX (M+H⁺).

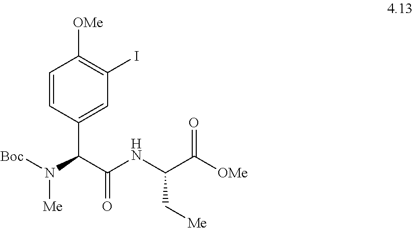 [SMILES: CC[C@H](NC(=O)[C@H](c1ccc(OC)c(I)c1)N(C)C(=O)OC(C)(C)C)C(=O)OC]

To a solution of Boc-N-Me-3-Iodo-Hydroxyphenylglycine (150 mg, 0.37 mmol, 1 eq) in DMF (3.5 mL) was added sequentially L-2-aminobutyric acid HCl (48 mg, 1.1 eq), HOBT (50 mg, 1 eq), EDC (78 mg, 1.1 eq) and NaHCO₃ (34 mg, 1.1 eq). The solution was allowed to stir overnight then was diluted with NaHCO_{3(aq)} and EtOAc. The aqueous phase was extracted 3× with EtOAc and the combined organic layers were washed with 5% citric acid (pH—3), water and brine. The organics were then dried over sodium sulfate then concentrated. TLC analysis of the crude mixture showed one major product so the crude material (105 mg, 0.20 mmol (assumed), 1 eq) was taken up in acetone (2.5 mL) and treated with MeI (63 μL, 5 eq) and K₂CO₃ (138 mg, 5 eq). The reaction was stirred for 1.5 h at reflux in a sealed tube then the acetone was blown off under a stream of nitrogen. To the residue was added water and EtOAc and the aqueous was extracted 3× with EtOAc. The combined organic layers were then dried over sodium sulfate, concentrated and the crude material was purified via column chromatography (1% MeOH in DCM) to give the product (58 mg, 29% yield over 2 steps). ¹H NMR (CDCl₃, 500 MHz) δ (ppm) 7.73-7.71 (m, 1H), 7.32-7.28 (m, 1H), 6.45-6.37 (m, 1H), 5.76 (br s, 1H), 4.61-4.55 (m, 1H), 3.87 (s, 3H), 3.74 (s, 3H), 2.71-2.70 (m, 3H), 1.93-1.88 (m, 1H), 1.74-1.69 (m, 1H), 1.47-1.46 (m, 9H), 0.93-0.87 (m, 3H) ¹³C NMR (CDCl₃, 500 MHz) δ (ppm) 172.5, 169.5, 158.1, 140.2, 130.5, 129.3, 110.8, 86.1, 80.9, 56.5, 53.6, 52.5, 31.6, 28.5, 25.5, 9.8. MS (ESI) m/z 543.1 (M+Na⁺).

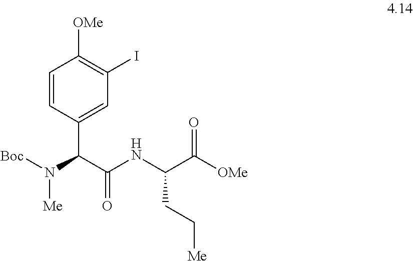 [SMILES: CCC[C@H](NC(=O)[C@H](c1ccc(OC)c(I)c1)N(C)C(=O)OC(C)(C)C)C(=O)OC]

Compound 4.14 was synthesized identically to compound 4.13 giving the product (47 mg, 15% yield over 2 steps). ¹H NMR (MeOD, 600 MHz) δ (ppm) 7.72 (s, 1H), 7.33 (d, J=8.4 Hz, 1H), 6.98 (d, J=8.4 Hz, 1H), 5.77 (br s, 1H), 4.45-4.42 (m, 1H), 3.88 (s, 3H), 3.75 (s, 3H), 2.64 (s, 3H), 1.80-1.78 (m, 1H), 1.69-1.67 (m, 1H), 1.53-1.29 (m, 11H), 0.95 (t, J=7.2 Hz, 3H) ¹³C NMR (MeOD, 600 MHz) δ (ppm) 174.0, 172.7, 159.7, 157.6, 141.2, 131.9, 130.7, 111.9, 86.3, 81.8, 62.5, 56.9, 53.7, 52.8, 34.2, 32.1, 28.7, 20.2, 13.9. MS (ESI) m/z 557.1 (M+Na⁺).

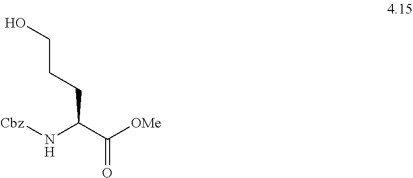 [SMILES: COC(=O)[C@H](CCCO)NC(=O)OCc1ccccc1]

To a solution of Z-Glu-OMe (268 mg, 0.91 mmol, 1 eq) in THF (3 mL) at 0° C. was added ethyl chloroformate (174 μL, 2 eq), and TEA (253 μL, 2 eq). The mixture was allowed to stir for 45 min. then filtered through glass wool (washing with THF) and the filtrate was treated with NaBH₄ (151 mg, 4.4 eq) in H₂O (1.5 mL) at 0° C. This reaction was allowed to stir for 45 min then it was warmed to rt. Next, saturated NaHCO₃, water and EtOAc were added and the aqueous phase was extracted 3× with EtOAc. The combined organic layers were washed with 5% citric acid and brine then dried over sodium sulfate and concentrated. Column chromatography (4% MeOH in DCM) yielded the product (106 mg, 41% yield). ¹H NMR (CDCl₃, 600 MHz) δ (ppm) 7.36-7.28 (m, 5H), 5.08 (s, 2H), 4.21-4.19 (m, 1H), 3.70 (s, 3H), 3.56-3.54 (m, 2H), 1.93-1.87 (m, 1H), 1.74-1.68 (m, 1H), 1.65-1.55 (m, 2H).). ¹³C NMR (CDCl₃, 500 MHz) δ (ppm) 174.6, 158.6, 138.1, 129.4, 129.0, 128.8, 67.6, 62.1, 55.3, 52.6, 29.8, 29.1. MS (ESI) m/z 304.1 (M+Na⁺).

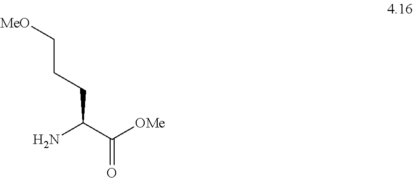 [SMILES: COCCC[C@H](N)C(=O)OC]

To a solution of compound 4.15 (104 mg, 0.37 mmol, 1 eq) in DCM (2.0 mL) was added 50% HBF_{4(aq)} (23 μL, 1 eq) and 2M TMSCH₂N₂ in diethyl ether (370 μL, 2 eq) dropwise over 15 min at 0° C. The reaction was monitored by TLC for disappearance of starting material. TMSCH₂N₂ (190 μL, 1 eq) was added every (˜45 min) four times over the course of 3 hrs. along with 50% HBF_{4(aq)} (12 μL, 0.5 eq). The reaction was allowed to stir for an additional 30 min. then the volatiles were evaporated, water was added and the aqueous layer was washed with EtOAc 3×. The combined organic layers were dried over sodium sulfate concentrated and purified via column chromatography (35% EtOAc in Hex). This material (55 mg, R_{f}−0.35 in 35% EtOAc in Hex) was dissolved in MeOH, to it was added 10% Pd/C (20 mg, ⅓ by weight) and the mixture was placed under an atmosphere of H₂ (1 atm). When TLC indicated a complete consumption of starting material the mixture was filtered through Celite and concentrated. The resulting Cbz deprotected material (30 mg, 50% yield) was used directly in the next step of the synthesis.

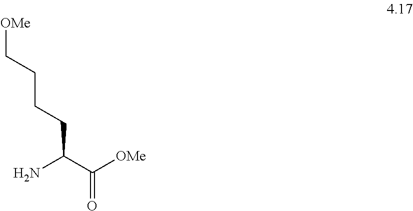 [SMILES: COCCCC[C@H](N)C(=O)OC]

To a solution of Boc-L-6-hydroxynorleucine (500 mg, 2 mmol, 1 eq) in a 9:1 mixture of toluene:MeOH (5 mL) was added TMSCH₂N₂ (1.15 mL, 1.15 eq) and the solution was allowed to stir for 1.5 hrs. The volatiles were then evaporated and the crude was purified via column chromatography (4% MeOH in DCM) to yield the product (472 mg). This material (418 mg, 1.6 mmol) was then dissolved in DCM (7 mL) and treated with 50% HBF_{4(aq)} (200 μL, 1 eq) and TMSCH₂N₂ in hexanes (1.6 mL, 2 eq) at 0° C. After 0.5 hr the solution was treated with TMSCH₂N₂ in Hexanes (0.5 eq, 0.4 mL) and after a further 15 min the solution was treated with 50% HBF_{4(aq)} (50 μL, 1 eq) and TMSCH₂N₂ in Hexanes (0.5 eq, 0.4 mL). The reaction was allowed to stir for another 40 min then dilute NaHCO₃ was added and the aqueous layer was extracted 3× with DCM. The combined organic layers were dried over sodium sulfate and concentrated. The crude material was purified via column chromatography (1.5% MeOH in DCM) to give the product (255 mg, 52% over 2 steps). ¹H NMR (CDCl₃, 400 MHz) δ (ppm) 5.05 (d, J=8.0 Hz, 1H), 4.27-4.21 (m, 1H), 3.68 (s, 3H), 3.31 (t, J=6.4 Hz, 2H), 3.26 (s, 3H), 1.81-1.72 (m, 1H), 1.64-1.34 (m, 14H). ¹³C NMR (CDCl₃, 400 MHz) δ (ppm) 173.4, 155.4, 79.8, 72.4, 58.6, 53.4, 52.2, 32.5, 29.2, 28.4, 22.1. MS (ESI) m/z 176.2 (M+H⁺). The preceding compound (136 mg, 0.49 mmol) was dissolved in DCM (3 mL) and treated with TFA (0.75 mL). The reaction was allowed to run for 1.5 hrs then the volatiles were evaporated under a stream of nitrogen and the crude was taken up in EtOAc and treated with dilute NaHCO₃. The aqueous phase was extracted 9× with EtOAc then the combined organic layers were dried over sodium sulfate and concentrated to give compound 4.17 (75 mg, 87%) which was used in the following step without purification.

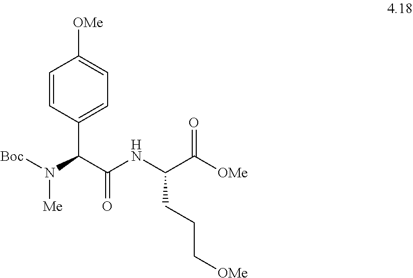 [SMILES: COCCC[C@H](NC(=O)[C@H](c1ccc(OC)cc1)N(C)C(=O)OC(C)(C)C)C(=O)OC]

Boc-N-Me-Hydroxyphenylglycine (71 mg, 0.19 mmol, 1 eq) was dissolved in DMF (1.0 mL) and treated sequentially with compound 4.16 (30 mg, 1 eq), HOBT (26 mg, 1 eq), EDC (37 mg, 1 eq) and TEA (catalytic). The reaction was allowed to stir overnight under Ar then water and EtOAc were added. The aqueous layer was extracted 3× with EtOAc then the combined organic layers were washed with dilute NaHCO_{3(aq)} and brine. The organics were dried over sodium sulfate, concentrated and the crude was used without further purification. This material (75 mg, 0.18 mmol (assumed), 1 eq) was dissolved in acetone (2 mL), treated with MeI (77 μL, 7 eq) and K₂CO₃ (124 mg, 5 eq) and heated to reflux in a sealed vial overnight. The reaction mixture was then cooled to room temperature and the acetone was evaporated under a stream on nitrogen. Water and EtOAc were added, the aqueous phase was extracted 3× with EtOAc, and the combined organic layers were dried over sodium sulfate. After concentrating, the crude material was purified via column chromatography (3% MeOH in DCM) to yield the product (68 mg, 81% over two steps). ¹H NMR (CDCl₃, 400 MHz) δ (ppm) 7.28 (d, J=8.4 Hz, 2H), 6.88 (d, J=8.8 Hz, 2H), 6.56 (d, J=7.2 Hz, 1H), 5.82 (br s, 1H), 4.63-4.58 (m, 1H), 3.79 (s, 3H), 3.72 (s, 3H), 3.34 (t, J=6.0 Hz, 2H) 3.22 (s, 3H), 2.67 (s, 3H) 1.94-1.79 (m, 2H), 1.63-1.42 (m, 11H) ¹³C NMR (CDCl₃, 400 MHz) δ (ppm) 172.7, 170.2, 159.6, 130.8, 127.4, 114.1, 80.5, 71.9, 58.6, 55.4, 52.4, 52.2, 31.4, 29.0, 28.5, 28.5, 25.6. MS (ESI) m/z 461.5 (M+Na⁺).

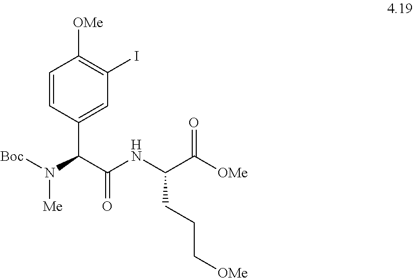 [SMILES: COCCC[C@H](NC(=O)[C@H](c1ccc(OC)c(I)c1)N(C)C(=O)OC(C)(C)C)C(=O)OC]

To a solution of compound 4.18 (67 mg, 0.15 mmol, 1 eq) in MeOH (3.5 mL) was added sequentially AgSO₄ (50 mg, 1.05 eq) and I₂ (41 mg, 1.05 eq). The reaction mixture was allowed to stir for 2.5 hrs then the proportion of product to starting material was checked by mass spectrometry. Seeing that starting material remained AgSO₄ (5.0 mg, 0.1 eq) and I₂ (4.0 mg, 0.1 eq). This process was repeated every 2-3 hrs for 6 hrs then 10% sodium thiosulfate was added and some of the volatiles were blown off under a stream of nitrogen. EtOAc was added, the aqueous layer was extracted 3× with EtOAc then the combined organic layers were washed with brine, dried over sodium sulfate and concentrated by rotary evaporation. The crude material was purified via column chromatography (2% MeOH in DCM) to yield the product (44 mg, 52% yield). Some NMR resonances appeared broadened or doubled (with one isomer in large excess to the other) due to slow rotation about the N-Me amide. ¹H NMR (CDCl₃, 600 MHz) δ (ppm) 7.74 (s, 1H), 7.33 (d, J=7.8, 1H), 6.80 (d, J=8.4 Hz, 1H), 6.67-6.66 (m, 1H), 5.77 (br s, 1H), 4.60-4.57 (m, 1H), 3.87 (s, 3H), 3.72 (s, 3H), 3.39 (t, J=6.0 Hz, 2H), 3.23 (s, 3H), 2.68 (s, 3H), 1.96-1.77 (m, 2H), 1.61-1.47 (m, 11H). ¹³C NMR (CDCl₃, 600 MHz) δ (ppm) 172.5, 169.6, 158.1, 140.3, 140.2, 130.7, 129.5, 129.4, 121.6, 110.7, 86.0, 80.8, 71.9, 58.6, 56.5, 52.5, 52.3, 31.5, 28.9, 28.5, 27.8, 25.5. MS (ESI) m/z 587.2 (M+Na⁺).

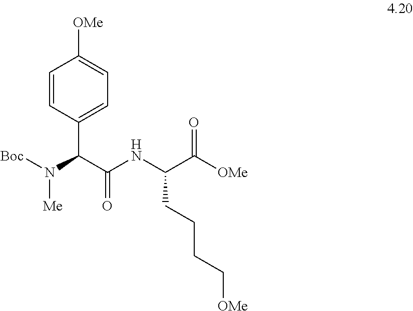 [SMILES: COCCCC[C@H](NC(=O)[C@H](c1ccc(OC)cc1)N(C)C(=O)OC(C)(C)C)C(=O)OC]

Boc-N-Me-Hydroxyphenylglycine (39 mg, 0.14 mmol, 1 eq) was dissolved in DMF (1.5 mL) and treated sequentially with compound 4.17 (25 mg, 1 eq), HOBT (19 mg, 1 eq), EDC (30 mg, 1 eq) and TEA (catalytic). The reaction was allowed to stir overnight under Ar then water and EtOAc were added. The aqueous layer was extracted 3× with EtOAc then the combined organic layers were washed with dilute NaHCO_{3(aq)} and brine. The organics were dried over sodium sulfate, concentrated and the crude was used without further purification. This material (61 mg, 0.14 mmol (assumed), 1 eq) was dissolved in acetone (1.5 mL), treated with MeI (88 μL, 10 eq) and K₂CO₃ (58 mg, 3 eq) and heated to reflux in a sealed vial overnight. The reaction mixture was then cooled to room temperature and the acetone was evaporated under a stream on nitrogen. Water and EtOAc were added, the aqueous phase was extracted 3× with EtOAc, and the combined organic layers were dried over sodium sulfate. After concentrating, the crude material was purified via column chromatography (2.5% MeOH in DCM) to yield the product (46 mg, 72% over two steps). ¹H NMR (CDCl₃, 500 MHz) δ (ppm) 7.27 (d, J=8.5 Hz, 2H), 6.89 (d, J=8.5 Hz, 2H), 6.33 (d, J=8.0 Hz, 1H), 5.81 (br s, 1H), 4.64-4.60 (m, 1H), 3.80 (s, 3H), 3.73 (s, 3H), 3.34 (t, J=6.5 Hz, 2H) 3.29 (s, 3H), 2.69 (s, 3H) 1.89-1.84 (m, 1H), 1.72-1.65 (m, 1H), 1.61-1.34 (m, 13H) ¹³C NMR (CDCl₃, 500 MHz) δ (ppm) 172.7, 170.1, 159.6, 130.7, 127.2, 114.1, 80.5, 72.4, 58.6, 55.4, 52.4, 52.4, 32.0, 31.4, 29.1, 28.5, 22.3. MS (ESI) m/z 475.2 (M+Na⁺).

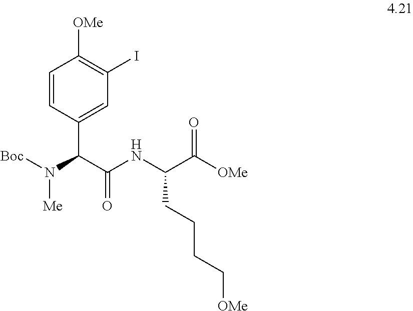 [SMILES: COCCCC[C@H](NC(=O)[C@H](c1ccc(OC)c(I)c1)N(C)C(=O)OC(C)(C)C)C(=O)OC]

To a solution of compound 4.20 (44 mg, 0.097 mmol, 1 eq) in MeOH (1.5 mL) was added sequentially AgSO₄ (32 mg, 1.05 eq) and I₂ (26 mg, 1.05 eq). The reaction mixture was allowed to stir for 2.5 hrs then the proportion of product to starting material was checked by mass spectrometry. Seeing that starting material remained AgSO₄ (3.0 mg, 0.1 eq) and I₂ (3.0 mg, 0.1 eq). This process was repeated every 2-3 hrs for 6 hrs then 10% sodium thiosulfate was added and some of the volatiles were blown off under a stream of nitrogen. EtOAc was added, the aqueous layer was extracted 3× with EtOAc then the combined organic layers were washed with brine, dried over sodium sulfate and concentrated by rotary evaporation. The crude material was purified via column chromatography (2.5% MeOH in DCM) to yield the product (46 mg, 81% yield). Some NMR resonances appeared broadened or doubled (with one isomer in large excess to the other) due to slow rotation about the N-Me amide. ¹H NMR (CDCl₃, 500 MHz) δ (ppm) 7.73 (d, J=1.5 Hz, 1H), 7.31 (d, J=8.5, 1H), 6.79 (d, J=8.5 Hz, 1H), 6.38 (d, J=7.5 Hz, 1H), 5.74 (br s, 1H), 4.61-4.57 (m, 1H), 3.86 (s, 3H), 3.73 (s, 3H), 3.33 (t, J=6.5 Hz, 2H), 3.27 (s, 3H), 2.69 (s, 3H), 1.90-1.85 (m, 1H), 1.72-1.65 (m, 1H), 1.58-1.34 (m, 13H). ¹³C NMR (CDCl₃, 500 MHz) δ (ppm) 172.6, 169.5, 158.1, 140.3, 130.6, 129.3, 110.8, 86.1, 80.8, 72.4, 58.6, 56.5, 52.5, 52.5, 32.0, 31.6, 29.1, 28.5, 22.3. MS (ESI) m/z 601.2 (M+Na⁺).

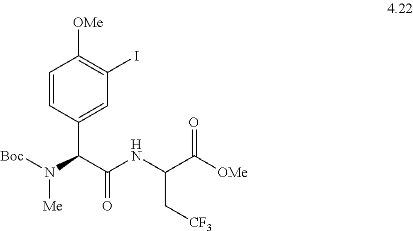 [SMILES: COC(=O)C(CC(F)(F)F)NC(=O)[C@H](c1ccc(OC)c(I)c1)N(C)C(=O)OC(C)(C)C]

Boc-N-Me-3-Iodo-Hydroxyphenylglycine (116 mg, 0.28 mmol, 1 eq) was dissolved in a 3:1 mixture of DCM:DMF (2.8 mL) and treated sequentially with racemic 2-amino-4,4,4-trifluoro-butyric acid hydrochloride (59 mg, 1 eq), HOBT (38 mg, 1 eq), DIC (49 μL, 1.1 eq) and TEA (40 μL, 1 eq). The reaction was allowed to stir overnight under Ar then the DCM was blown off and dilute NaHCO_{3(aq)} and EtOAc were added. The aqueous layer was extracted 3× with EtOAc then the combined organic layers were washed with 5% citric acid (pH—3), water and brine. The organics were dried over sodium sulfate, concentrated and purified via column chromatography (4% MeOH in DCM) to yield an oil. This material (81 mg, 0.145 mmol (assumed), 1 eq) was dissolved in acetone (2 mL), treated with MeI (45 μL, 5 eq) and K₂CO₃ (100 mg, 5 eq) and heated to reflux in a sealed vial overnight. The reaction mixture was then cooled to room temperature and the acetone was evaporated under a stream on nitrogen. Water and EtOAc were added, the aqueous phase was extracted 3× with EtOAc, and the combined organic layers were dried over sodium sulfate. After concentrating, the crude material was purified via column chromatography (1.8% MeOH in DCM) to yield the product as a foam (47 mg, 15% over two steps). ¹H NMR (MeOD, 600 MHz) δ (ppm) 7.70 (s, 1H), 7.32-7.29 (m, 1H), 6.99-6.97 (m, 1H), 5.78 (br s, 1H), 4.82-4.68 (m, 1H), 3.88 (s, 3H), 3.80-3.79 (m, 3H), 2.94-2.86 (m, 1H), 2.74-2.66 (m, 5H), 1.49 (s, 1H) ¹³C NMR (MeOD, 600 MHz) δ (ppm) 172.4, 172.2, 171.4, 171.3, 159.8, 141.2, 132.0, 131.9, 130.4, 128.3, 128.3, 126.5, 126.4, 112.0, 112.0, 86.3, 86.3, 82.0, 62.8, 56.9, 53.5, 53.4, 35.6, 35.4, 35.4, 35.2, 31.9, 28.6. MS (ESI) m/z 597.1 (M+Na⁺).

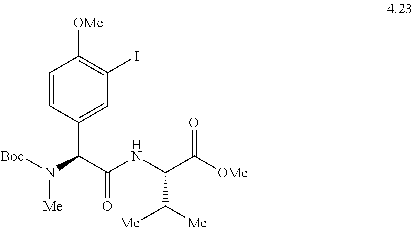 [SMILES: COC(=O)[C@@H](NC(=O)[C@H](c1ccc(OC)c(I)c1)N(C)C(=O)OC(C)(C)C)C(C)C]

Compound 4.23 was synthesized in a manner identical to compound 4.13 (53% yield).

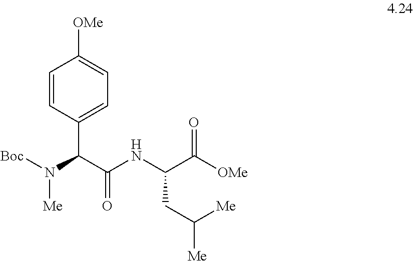 [SMILES: COC(=O)[C@H](CC(C)C)NC(=O)[C@H](c1ccc(OC)cc1)N(C)C(=O)OC(C)(C)C]

Boc-N-Me-Hydroxyphenylglycine (124 mg, 0.44 mmol, 1 eq) dissolved in DMF (2.0 mL) was added sequentially H-Leu-OMe (80 mg, 1 eq), HOBT (59 mg, 1 eq), EDC (93 mg, 1.1 eq) and TEA (67 μL, 1.1 eq). The reaction was allowed to stir overnight then dilute NaHCO_{3(aq)} and EtOAc, the aqueous phase was extracted 3× with EtOAc and the combined organic fractions were washed with 5% citric acid (pH—3), water and brine. The organics were then dried over sodium sulfate, concentrated and the crude was used without further purification. The crude material (170 mg, 0.42 mmol (assumed), 1 eq) was taken up in acetone (2 mL) and treated with MeI (260 μL, 10 eq) and K₂CO₃ (172 mg, 3 eq). The reaction was stirred for overnight at reflux in a sealed tube then the acetone was blown off under a stream of nitrogen. To the residue was added water and EtOAc and the aqueous was extracted 3× with EtOAc. The combined organic layers were then dried over sodium sulfate, concentrated and the crude material was purified via column chromatography (1.5% MeOH in DCM) to give the product (130 mg, 70% yield over 2 steps). ¹H NMR (CDCl₃, 500 MHz) δ (ppm) 7.26 (d, J=8.5 Hz, 2H), 6.88 (d, J=8.5 Hz, 2H), 6.49 (d, J=8.0 Hz 1H), 5.81 (br s, 1H), 4.68-4.63 (m, 2H), 3.79 (s, 3H), 3.72 (s, 3H), 1.66-1.63 (s, 2H), 1.54-1.46 (m, 10H), 0.93 (t, J=6.0 Hz, 3H) ¹³C NMR (CDCl₃, 500 MHz) δ (ppm) 173.3, 170.1, 159.6, 130.7, 127.2, 114.2, 80.5, 55.4, 52.4, 51.0, 41.5, 31.4, 28.5, 25.0, 22.9, 22.0. MS (ESI) m/z 445.2 (M+Na⁺).

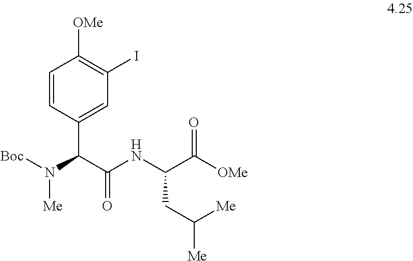 [SMILES: COC(=O)[C@H](CC(C)C)NC(=O)[C@H](c1ccc(OC)c(I)c1)N(C)C(=O)OC(C)(C)C]

To a solution of compound 4.24 (129 mg, 0.31 mmol, 1 eq) in MeOH (2 mL) was added sequentially AgSO₄ (101 mg, 1.05 eq) and I₂ (81 mg, 1.05 eq). The reaction mixture was allowed to stir for 2.5 hrs then 10% sodium thiosulfate and some of the volatiles were blown off under a stream of nitrogen. EtOAc was added, the aqueous layer was extracted 3× with EtOAc then the combined organic layers were washed with brine, dried over sodium sulfate and concentrated by rotary evaporation. The crude material was purified via column chromatography (1% MeOH in DCM) to yield the product (135 mg, 80% yield). ¹H NMR (CDCl₃, 500 MHz) δ (ppm) 7.72 (d, J=2.0 Hz, 1H), 7.30 (dd, J=8.5 Hz, J=1.5 Hz, 1H), 6.78 (d, J=8.5 Hz, 1H), 6.23 (br s, 1H), 5.74 (br s, 1H), 4.65-4.60 (m, 2H), 3.85 (s, 3H), 3.72 (s, 3H), 2.68 (s, 3H), 1.65-1.63 (m, 2H), 1.53-1.45 (m, 10H), 0.92 (t, J=6 Hz, 6H). ¹³C NMR (CDCl₃, 500 MHz) δ (ppm) 173.1, 169.5, 158.1, 140.2, 130.5, 129.2, 110.7, 86.0, 80.8, 56.5, 52.4, 41.3, 31.6, 28.4, 24.9, 22.9, 21.9. MS (ESI) m/z XX (M+H⁺).

### General Procedure A

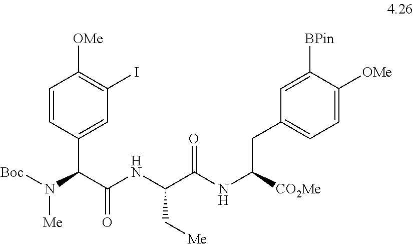

To a solution of compound 4.13 (57 mg, 0.11 mmol, 1 eq) in THF (1.3 mL) was added a 0.2 M LiOH solution (1.1 mL, 2 eq). The solution was allowed to stir until all starting material had been consumed by TLC analysis then the reaction was quenched by the addition of citric acid (pH—3) and THF was blown off under a stream of nitrogen. The aqueous layer was extracted 3× with EtOAc and the combined organic layers were washed with brine, dried over sodium sulfate and concentrated. The resulting crude material (55 mg) was used without further purification. To a solution of this crude material (52 mg, 0.1 mmol, 1 eq) and compound XX in a 1:1 mixture of AcCN:DMF (2.6 mL) was added sequentially HOBT (24 mg, 2.5 eq) and EDC (42 mg, 2.2 eq). The reaction was allowed to stir overnight then dilute NaHCO_{3(aq)} was added and the aqueous phase was extracted 3× with EtOAc. The combined organic layers were washed with 5% citric acid (pH—3), water and brine then dried over sodium sulfate and concentrated. The crude material was purified via abbreviated column chromatography (2.5% MeOH in DCM) due to its instability in the presence of silica gel. The purification yielded a semi-pure residue (65 mg, 77% yield). ¹H NMR (CDCl₃, 600 MHz) δ (ppm) ¹³C NMR (CDCl₃, 600 MHz) δ (ppm) 171.8, 171.5, 171.0, 170.9, 170.8, 169.7, 163.7, 163.5, 163.4, 158.2, 158.1, 158.0, 140.4, 140.2, 137.8, 137.6, 133.4, 133.3, 130.7, 130.3, 130.2, 130.0, 129.2, 127.8, 127.0, 114.2, 114.1, 110.9, 110.8, 110.8, 110.6, 110.0, 86.2, 86.1, 83.7, 83.7, 81.1, 80.9, 62.2, 56.5, 56.5, 56.4, 55.9 (2C), 55.7, 55.3, 54.5 (2C), 54.4, 53.8, 53.6 (2C), 53.2, 52.5 (2C), 52.4, 37.0, 36.8, 36.6, 32.1, 28.5, 25.5, 25.0, 24.9 (2C), 10.1, 9.9 (2C), 9.8. MS (ESI) m/z 846.3 (M+Na⁺).

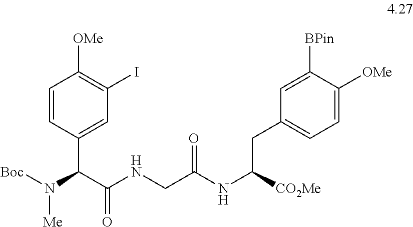

Compound 4.27 was synthesized via general procedure A starting from compound 4.12 (66% yield). R_{f}−0.28 (4% MeOH in DCM). ¹H NMR (CDCl₃, 600 MHz) δ (ppm) 7.70-7.68 (m, 1H), 7.45-7.33 (m, 1H), 7.23-7.22 (m, 1H), 7.15-7.10 (m, 1H), 6.75-6.66 (m, 4H), 5.61 (br s, 1H), 4.79-4.73 (m, 1H), 4.03-3.96 (m, 1H) 3.84-3.67 (m, 9H), 3.11-2.95 (m, 2H), 2.70-2.69 (m, 3H), 1.45-1.44 (m, 9H) 1.31-1.30 (m, 10H). ¹³C NMR (CDCl₃, 600 MHz) δ (ppm) 171.8, 171.8, 171.6, 170.1, 168.4, 163.6, 163.4, 163.3, 158.1, 158.1, 140.3, 140.3, 140.2, 137.7, 137.6, 133.2, 134.8, 130.5, 129.2, 127.0, 110.8, 110.7, 110.6, 86.1, 83.6, 80.9, 62.4, 56.5, 56.4, 56.4, 55.9, 55.8, 55.6, 53.7, 53.5, 52.5, 52.4, 52.4, 43.0, 43.0, 36.7, 36.6, 32.2, 28.4, 27.7, 25.0, 24.9, 24.9, 24.8. MS (ESI) m/z 818.3 (M+H⁺).

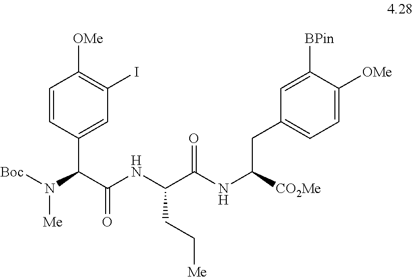

Compound 4.28 was synthesized via general procedure A using compound 4.14 (81% yield). ¹H NMR (MeOD, 600 MHz) δ (ppm) 7.69-7.64 (m, 1H), 7.45 (s, 1H), 7.29-7.26 (m, 1H), 7.16-7.07 (m, 1H), 6.97-6.81 (m, 2H), 5.68-5.64 (m, 1H), 4.62-4.59 (m, 1H), 4.45-4.43 (m, 1H) 3.87-3.67 (m, 9H), 3.09-3.06 (m, 1H), 2.89-2.93 (m, 1H), 2.66-2.64 (m, 3H), 1.75-1.69 (m, 1H) 1.62-1.27 (m, 24H), 0.95-0.88 (s, 3H). ¹³C NMR (MeOD, 600 MHz) δ (ppm) 174.0, 173.3, 172.2, 164.8, 164.8, 141.3, 141.2, 138.7, 138.6, 134.8, 134.8, 132.0, 131.6, 131.3, 130.6, 129.3, 114.9, 112.1, 112.0, 111.7, 111.6, 86.5, 86.3, 84.8, 81.9, 57.0, 56.9, 55.9, 55.8, 55.7, 55.6, 54.3, 52.7, 52.6, 37.6, 37.4, 35.2, 32.4, 28.7, 28.7, 25.2, 25.1, 20.0, 19.9, 14.1. MS (ESI) m/z 860.3 (M+Na⁺).

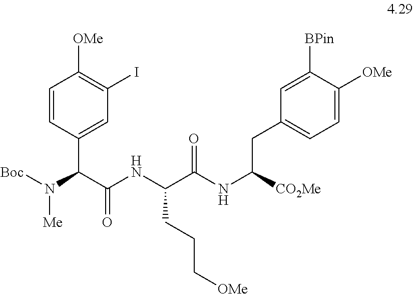

Compound 4.29 was synthesized via general procedure A using compound 4.19 (72% yield). ¹H NMR (CDCl₃, 400 MHz) δ (ppm) 7.70-7.69 (s, 1H), 7.43-7.37 (m, 1H), 7.24-7.07 (m, 2H), 7.00-6.98 (m, 1H), 6.78-6.67 (m, 3H), 5.66-5.62 (m, 1H), 4.76-4.71 (m, 1H), 4.57-4.51 (m, 1H) 3.86-3.68 (m, 9H), 3.44-3.39 (m, 2H), 3.28-3.18 (m, 3H), 3.06-2.95 (m, 2H), 2.75-2.68 (m, 3H), 1.85-1.71 (m, 2H), 1.62-1.41 (m, 11H) 1.37-1.23 (m, 12H). ¹³C NMR (CDCl₃, 400 MHz) δ (ppm) 171.9, 171.0, 169.3, 163.4, 158.0, 140.2, 137.6, 133.2, 130.3, 129.4, 127.2, 110.8, 110.7, 86.1, 83.6, 80.8, 72.8, 62.2, 58.7, 56.5, 55.9, 53.8, 52.7, 52.3, 36.9, 32.0, 30.5, 28.5, 25.3, 25.0, 24.9. MS (ESI) m/z 890.3 (M+Na⁺).

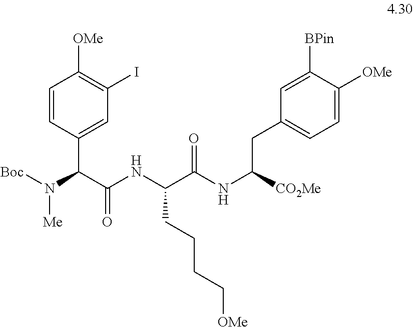

Compound 4.30 was synthesized via general procedure A using compound 4.21 (65% yield). R_{f}−0.26 (4% MeOH in DCM). ¹H NMR (CDCl₃, 600 MHz) δ (ppm) 7.74-7.70 (s, 1H), 7.42-7.33 (m, 1H), 7.22-7.16 (m, 1H), 7.11-7.10 (m, 1H), 6.78-6.69 (m, 2H), 6.63-6.53 (m, 1H), 6.42-6.40 (m, 1H), 5.66-5.61 (m, 1H), 4.77-4.74 (m, 1H), 4.49-4.40 (m, 1H) 3.87-3.69 (m, 9H), 3.34-3.26 (m, 5H), 3.06-2.98 (m, 2H), 2.76-2.68 (m, 3H), 2.05-2.03 (m, 1H), 1.91-1.86 (m, 1H), 1.61-1.24 (m, 25H). ¹³C NMR (CDCl₃, 600 MHz) δ (ppm) 171.8, 171.0, 169.7, 163.4, 158.1, 140.2, 137.6, 133.3, 130.3, 129.2, 127.0, 110.9, 110.6, 86.2, 83.7, 83.6, 80.9, 72.5, 62.2, 58.6, 56.5, 55.9, 53.7, 53.2, 52.3, 36.9, 32.1, 32.0, 29.0, 28.5, 25.0, 24.9, 24.8, 22.2. MS (ESI) m/z 904.3 (M+Na⁺).

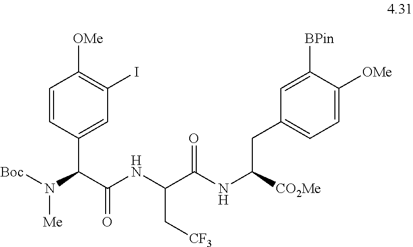

Compound 4.31 was synthesized via general procedure A using compound 4.22 (68% yield). ¹H NMR (MeOD, 600 MHz) δ (ppm) 7.69-7.64 (m, 1H), 7.49-7.46 (m, 1H), 7.31-7.06 (m, 2H), 6.95-6.80 (m, 2H), 5.66-5.56 (m, 1H), 4.87-4.83 (m, 1H), 4.66-4.56 (m, 1H) 3.87-3.68 (m, 9H), 3.14-3.08 (m, 1H), 2.99-2.96 (m, 1H), 2.66-2.43 (m, 5H), 1.49-1.47 (m, 9H), 1.33 (s, 11H). ¹³C NMR (MeOD, 600 MHz) δ (ppm) 173.1 (2C), 173.0, 172.3, 172.1, 171.9, 171.5, 171.3, 171.1, 171.0, 164.9, 164.8, 164.8, 164.7, 159.9, 159.8, 159.7, 141.5, 141.4, 141.3, 141.2, 138.8, 138.7, 138.6, 134.8, 134.8, 132.0, 131.7, 131.7, 130.4, 130.0, 129.3, 129.2, 129.1, 128.3, 126.4, 118.5, 115.0, 112.1 (3C), 111.9, 111.8, 111.7, 111.7, 86.5, 86.4, 86.3, 84.8, 82.0, 63.7, 63.7, 63.2, 57.0, 56.9, 56.0, 55.9, 55.9, 55.9, 52.9, 52.8, 52.8, 52.7, 37.6, 37.5, 28.7, 28.7, 28.6, 25.2, 25.1, 25.1. MS (ESI) m/z 900.3 (M+Na⁺).

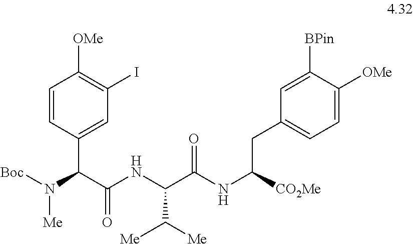

Compound 4.32 was synthesized via general procedure A using compound 4.23 (68% yield). ¹H NMR (CDCl₃, 600 MHz) δ (ppm) ¹³C NMR (CDCl₃, 600 MHz) δ (ppm) 171.8, 171.8, 170.6, 170.5, 170.5, 169.9, 169.6, 163.5, 163.4, 158.8, 158.1, 158.0, 156.1, 140.4, 140.0, 137.7, 137.6, 137.5, 133.4, 133.3, 130.7, 130.3, 130.2, 130.1, 129.2, 129.1, 127.1, 118.0, 114.1, 114.1, 110.8, 110.8, 110.8, 110.6, 86.1, 86.0 (2C), 83.6, 83.6, 83.5, 80.9, 62.5, 58.4, 58.3, 58.3, 56.5 (2C), 55.9, 55.9, 53.7, 53.6, 53.5, 52.4, 52.4, 52.4, 52.3, 36.9, 36.9, 36.8, 30.8, 28.4, 24.9 (3C), 24.8, 19.3 (3C), 19.2, 17.8. MS (ESI) m/z 860.3 (M+Na⁺).

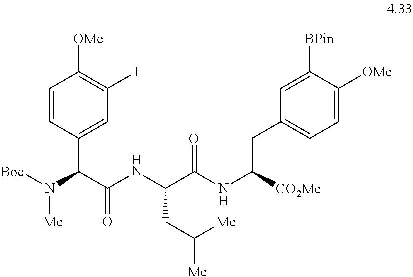

Compound 4.33 was synthesized via general procedure A starting from compound 4.25 (69% yield). R_{f}−0.41 (4% MeOH in DCM). ¹H NMR (CDCl₃, 600 MHz) δ (ppm) 7.68 (s, 1H), 7.33-7.31 (m, 1H), 7.20-7.17 (m, 1H), 7.11-7.06 (m, 1H), 6.77-6.59 (m, 3H), 6.30 (d, J=7.8 Hz, 1H), 5.64-5.56 (m, 1H), 4.76-4.72 (m, 1H), 4.53-4.50 (m, 1H) 3.86-3.68 (m, 9H), 3.06-2.97 (m, 2H), 2.72-2.67 (m, 3H), 1.69-1.60 (m, 2H), 1.46-1.45 (m, 10H) 1.32-1.27 (m, 11H) 0.90-0.85 (m, 3H). ¹³C NMR (CDCl₃, 600 MHz) δ (ppm) 171.8, 171.4, 169.7, 163.3, 158.0, 140.1, 137.6, 133.3, 1303, 129.1, 128.6, 127.0, 110.8, 110.5, 86.2, 83.6, 81.0, 80.8, 56.4, 55.8, 53.6, 52.3, 51.7, 40.8, 36.8, 28.4, 25.0, 24.9, 24.8, 24.8, 23.1, 21.8. MS (ESI) m/z 874.3 (M+Na⁺).

### General Procedure B

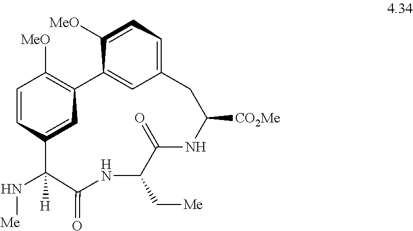

A solution of compound 4.26 (50 mg, 61 μmol, 1 eq) and NaHCO₃ (49 mg, 10 eq) in DMF (1.75 mL) was purged several times via cycling with vacuum and Ar and sealed with a crimped septa. To this solution was added a solution of PdCl₂(dppf) (9.9 mg, 0.2 eq) in DMF (1.2 mL) that had been sparged with Ar for ˜15 minutes via syringe. The resulting mixture was submitted to several more cycles of vacuum and Ar then heated to 80° C. The mixture was cooled to room temperature and water was added. The aqueous phase was extracted with EtOAc 3× then washed with water and brine, dried over sodium sulfate and concentrated. The crude material was subjected to abbreviated column chromatography (4% MeOH in DCM) to remove most of the Pd species then used without further purification. The resulting semi-pure material (16 mg) was taken up in DCM (1.5 mL) and treated with TFA (0.3 mL). The reaction was monitored via TLC and when starting material was no longer present the volatiles were blown off under a stream of nitrogen. DCM was added and blown off under nitrogen twice more and the crude residue was dissolved EtOAc. The organic layer was washed with saturated NaHCO₃, dried over sodium sulfate and concentrated. The crude material was purified via pipette column chromatography (7.5% MeOH in DCM) to give the product (7.5 mg, 26% yield). MS (ESI) m/z 470.2 (M+H⁺).

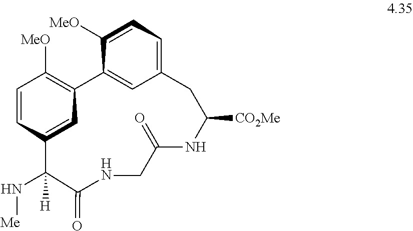

Compound 4.35 was synthesized via general procedure B starting from compound 4.27 (22% yield). MS (ESI) m/z 442.2 (M+H⁺).

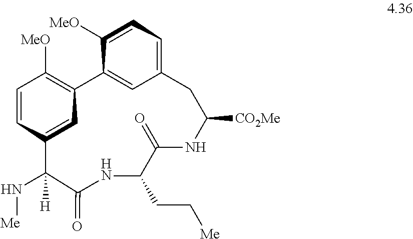

Compound 4.36 was synthesized via general procedure B starting from compound 4.28 (29% yield). MS (ESI) m/z 484.2 (M+H⁺).

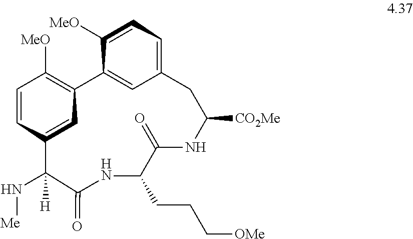

Compound 4.37 was synthesized via general procedure B starting from compound 4.29 (44% yield).

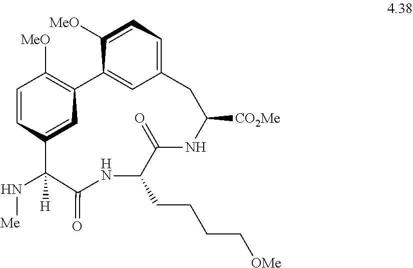

Compound 4.38 was synthesized via general procedure B starting from compound 4.30 (32% yield). MS (ESI) m/z 528.3 (M+H⁺).

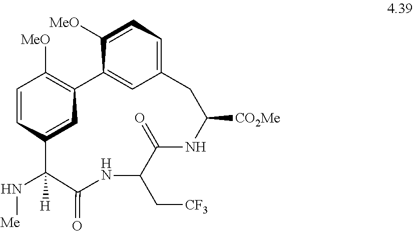

Compound 4.39 was synthesized via general procedure B starting from compound 4.31 (26% yield). MS (ESI) m/z 524.2 (M+H⁺).

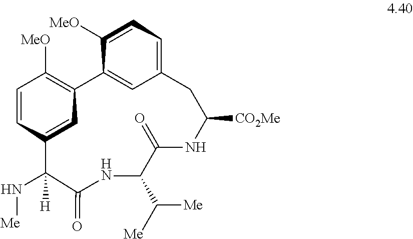

Compound 4.40 was synthesized via general procedure B starting from compound 4.32 (32% yield). MS (ESI) m/z 484.2 (M+H⁺).

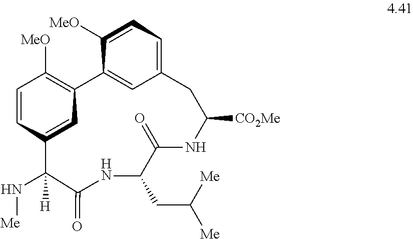

Compound 4.41 was synthesized via general procedure B starting from compound 4.33 (39% yield). MS (ESI) m/z 498.2 (M+H⁺).

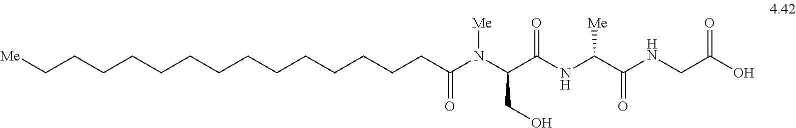 [SMILES: CCCCCCCCCCCCCCCC(=O)N(C)[C@H](CO)C(=O)N[C@H](C)C(=O)NCC(=O)O]

Compound 4.42 was synthesized via standard Fmoc/piperidine solid phase peptide synthesis. Fmoc-Gly-OH was loaded onto chlorotrityl chloride resin with DIEA, then the constituent amino acids, Fmoc-d-Ala-OH and Fmoc-N-Me-d-Ser-OH were coupled to the resin using HCTU/HOBT/DIEA in DMF followed by palmitic acid coupling with HCTU/HOBT/DIEA in DMF and enough DCM to completely dissolve the acid. Cleavage from the resin was achieved using 1% TFA in DCM using protocols detailed in the Novabiochem catalogue. The product was purified via HPLC (linear gradient, 0.66% B per minute, product eluted at 97% B).

### General Procedure C

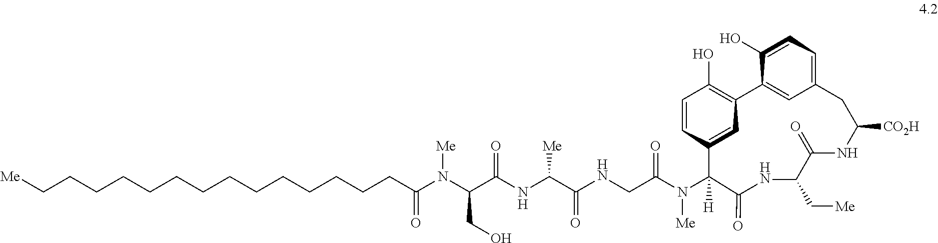

To a solution of Compound 4.34 (7.0 mg, 14.9 μmol) and compound 4.42 (12.8 mg, 1.5 eq) in THF (0.5 mL) at 0° C. was added DEPBT (7.0 mg, 1.6 eq) and NaHCO₃ (1.3 mg, 1 eq). The reaction was then allowed to warm to room temperature and stirred overnight. The THF was blown off under a stream of nitrogen and the reaction was dried under vacuum. The crude reaction mixture was taken up in EtOAc, washed 2× with saturated NaHCO₃, then brine, dried over sodium sulfate and concentrated. The crude was purified via pipette column chromatography (3% MeOH in DCM then 4.5% MeOH in DCM) to give the intermediate product. The intermediate product (6.6 mg, 6.4 μmol, 1 eq) was dissolved in ethanethiol (300 μL), treated with 1.0 M AlBr₃ in CH₂Br₂ (128 μL, 20 eq) and heated to 50° C. for 5 hrs. The reaction was allowed to cool to room temperature, quenched by the addition of MeOH and the volatiles were blown off under a stream of nitrogen. The crude was taken up in MeOH and dried twice more to remove lingering ethanethiol then it was dissolved in MeOH centrifuged and purified via HPLC (linear gradient, 0.67% B per minute, product eluted at 82% B) to give the final product (1.8 mg, 13% yield). ESI HRMS calcd for C₄₇H₇₀N₆O₁₁ [(M+H)⁺] 895.5175. found 895.5165.

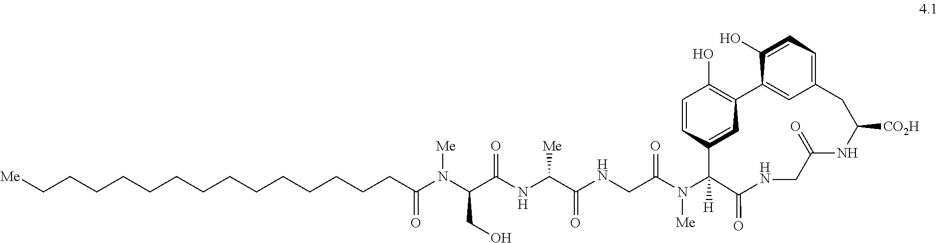

Compound 4.1 was synthesized via general procedure C starting from compound 4.35 (31% yield). ESI HRMS calcd for C₄₅H₆₆N₆O₁₁ [(M+H)⁺] 867.4862. found 867.4860.

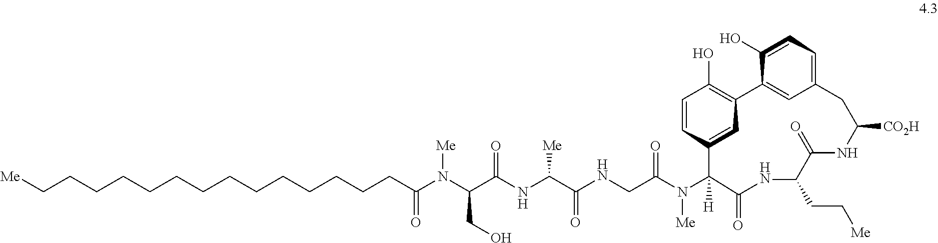

Compound 4.3 was synthesized via general procedure C starting from compound 4.36 (27% yield). ESI HRMS calcd for C₄₈H₇₂N₆O₁₁ [(M+H)⁺] 909.5332. found 909.5336.

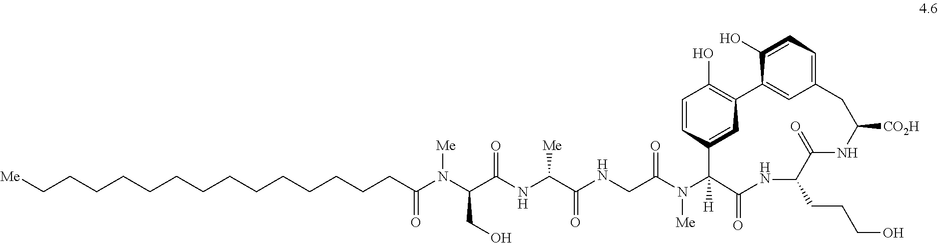

Compound 4.6 was synthesized via general procedure C starting from compound 4.37 (31% yield). ESI HRMS calcd for C₄₈H₇₂N₆O₁₂ [(M+H)⁺] 925.5281. found 925.5275.

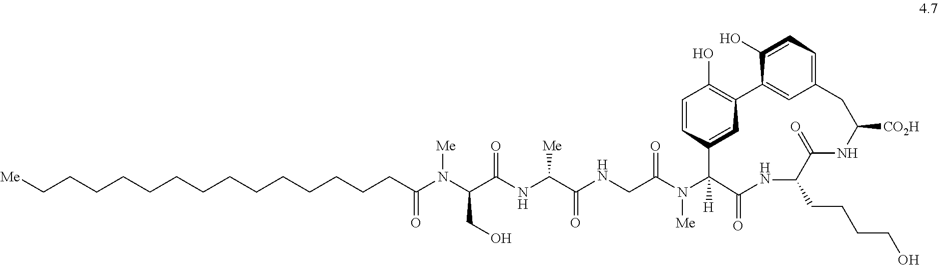

Compound 4.7 was synthesized via general procedure C starting from compound 4.38 (35% yield). ESI HRMS calcd for C₄₉H₇₄N₆O₁₂ [(M+H)⁺] 939.5437. found 939.5459.

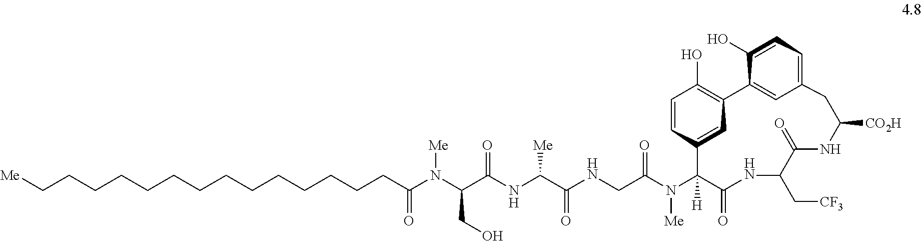

Compound 4.8 was synthesized via general procedure C starting from compound 4.39 (24% yield). ESI HRMS calcd for C₄₇H₆₇N₆O₁₁ [(M+H)⁺] 949.4892. found 949.4886.

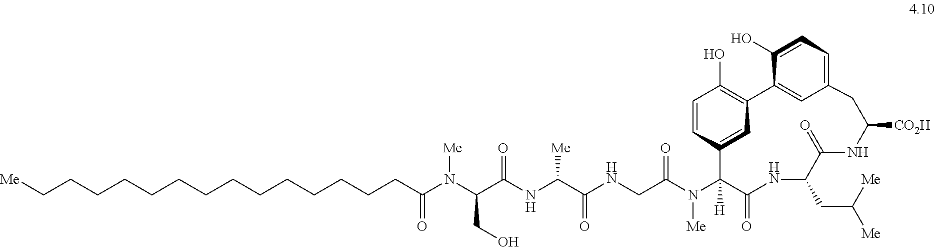

Compound 4.10 was synthesized via general procedure C starting from compound 4.40 (36% yield). ESI HRMS calcd for C₄₉H₇₄N₆O₁₁ [(M+Na)⁺] 945.5307. found 945.5306.

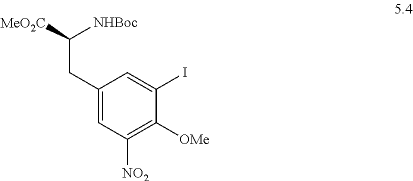 [SMILES: COC(=O)[C@H](Cc1cc(I)c(OC)c([N+](=O)[O-])c1)NC(=O)OC(C)(C)C]

A solution of 3-nitro-tyrosine (1 g, 4.4 mmol, 1 eq) was dissolved in acetone:H₂O (1:1, 10 mL) and treated with NaHCO₃ (554 mg, 1.5 eq) and Boc₂O (946 μL, 1 eq) and allowed to stir overnight. The reaction was acidified with 5% citric acid (pH—3) and extracted 3× with EtOAc then the combined organic fractions were washed with brine, dried over sodium sulfate and concentrated. The crude (1.37 g, 4.2 mmol, 1 eq) was taken up in a 5:2 mixture of DCM:MeOH (56 mL), treated with BTMA-ICl₂ (1.6 g, 1.1 eq) and NaHCO₃ (2.47 g, 7 eq) and allowed to stir overnight. The solid NaHCO₃ was then filtered, the filtrate was concentrated and acidified with 5% citric acid (pH—3). The aqueous layer was extracted 3× with EtOAc and the combined organic layers were dried over sodium sulfate and concentrated. The crude material (1.89 g, 4.19 mmol, 1 eq) was dissolved in acetone, and treated with K₂CO₃ (2.9 g, 5 eq) and MeI (1.3 mL, 5 eq) and heated to reflux over two days. The reaction mixture was then allowed to cool to room temperature, the reaction was quenched with a small amount of water and the volatiles were evaporated. 5% citric acid (pH—3) and EtOAc were added then separated and the aqueous layer was extracted 2× with EtOAc. The combined organic layers were washed with brine, dried over sodium sulfate and concentrated. The crude material was purified via column chromatography (0-0.5% MeOH in DCM) to yield compound 5.4 (1.67 g, 82% yield over 3 steps). ¹H NMR (CDCl₃, 500 MHz) δ (ppm) 7.80 (d, J=1.5 Hz, 1H), 7.56 (d, J=1.5 Hz, 1H), 5.12 (d, J=6.5 Hz, 1H), 4.54-4.53 (m, 1H), 3.94 (s, 3H), 3.76 (s, 3H), 3.18 (dd, J=5.0 Hz, J=14.0 Hz, 1H) 2.98 (dd, J=6.5 Hz, J=14.0 Hz, 1H) 1.41 (s, 9H) ¹³C NMR (CDCl₃, 500 MHz) δ (ppm) 171.5, 155.0, 152.1, 144.9, 143.8, 135.1, 126.4, 94.3, 80.5, 62.8, 54.2, 52.8, 37.0, 28.4. MS (ESI) m/z 503.0 (M+Na⁺). Compound 5.4 (127 mg, 0.27 mmol, 1 eq) was then dissolved in DCM (2.5 mL) and treated with TFA (0.5 mL). When TLC analysis indicated the complete consumption of starting material the volatiles were blown off and the residue was dried under vacuum. The residue was then taken up in EtOAc and saturated NaHCO₃, the aqueous layer was extracted 3× with EtOAc, the combined organic layers were dried over sodium sulfate and concentrated. The resulting compound 5.6 (101 mg) was used without further purification.

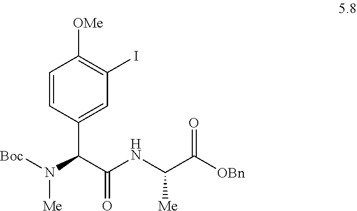 [SMILES: COc1ccc([C@@H](C(=O)N[C@@H](C)C(=O)OCc2ccccc2)N(C)C(=O)OC(C)(C)C)cc1I]

To a solution of compound 5.7 (300 mg, 0.74 mmol, 1 eq) in DMF (7.4 mL) was added sequentially H-Ala-OBn HCl (160 mg, 1 eq), EDC (170 mg, 1.2 eq), HOBt (100 mg, 1 eq) and NaHCO₃ (71 mg, 1.15 eq) and the reaction was allowed to stir overnight. Dilute NaHCO₃ was added and the aqueous phase was extracted 3× with EtOAc. The combined organic layers were washed with 5% citric acid (pH—3), water and brine then dried over sodium sulfate and concentrated. The crude material (353 mg, 0.62 mmol, 1 eq) was taken up in acetone (6.2 mL) and to this solution was added K₂CO₃ (428 mg, 5 eq) and MeI (386 μL, 10 eq). The mixture was allowed to stir overnight at reflux in a sealed vial then the solvent was evaporated, water was added and the aqueous phase was extracted 3× with EtOAc. The combined organic layers were washed with brine, dried over sodium sulfate and concentrated. The crude material was purified via column chromatography (0.75% MeOH in DCM) to give the product (189 mg, 44% yield over 2 steps). ¹H NMR (CDCl₃, 600 MHz) δ (ppm) 7.80 (s, 1H), 7.37-7.29 (m, 6H), 6.74 (d, J=8.4 Hz, 1H), 6.30 (d, J=7.2 Hz, 1H), 5.74 (br s, 1H), 5.22-5.15 (m, 2H), 4.70-4.66 (m, 1H) 3.87 (s, 3H) 2.70 (s, 3H) 1.48 (s, 9H), 1.44 (d, J=7.2 Hz, 3H). ¹³C NMR (CDCl₃, 600 MHz) δ (ppm) 172.6, 169.3, 158.2, 140.4, 135.4, 130.6, 129.3, 128.8, 128.6, 128.4, 114.2, 110.7, 86.2, 80.9, 67.4, 56.6, 55.4, 53.6, 48.5, 31.7, 28.5, 18.3. MS (ESI) m/z 605.1 (M+Na⁺).

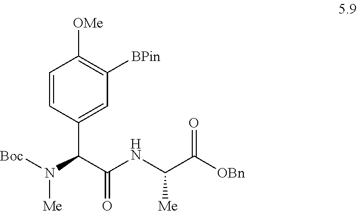

To a solution of compound 5.8 (185 mg, 0.36 mmol, 1 eq) in DMSO (7 mL) under Ar was added sequentially bispinacolatodiboron (95 mg, 1.05 eq), potassium acetate (353 mg, 10 eq) and PdCl₂(dppf) (15 mg, 0.05 eq). The mixture was allowed to stir for 2.5 hrs at 80° C. then cooled to room temperature diluted with water and extracted 3× with EtOAc. The combined organic layers were washed with brine dried over sodium sulfate and concentrated. The crude material was purified by abbreviated (to minimize the time of the compounds exposure to silica) column chromatography (35% EtOAc in Hex) giving compound 5.9 as a mixture of boronic acid and ester (118 mg, 64% yield). NMR spectra showed two sets of overlapping signals in a 3:1 ratio. ¹H NMR (CDCl₃, 600 MHz) δ (ppm) 7.61-7.59 (m, 1H), 7.37-7.31 (m, 5H), 6.81-6.76 (m, 1H), 6.31-6.18 (m, 1H), 5.74 (br, s), 5.20-5.12 (m, 2H), 4.73-4.66 (m, 1H), 3.83-3.80 (m, 3H), 2.68-2.67 (m, 3H) 1.47-1.40 (m, 12H), 1.34-1.33 (m, 9H). MS (ESI) m/z 605.3 (M+Na⁺).

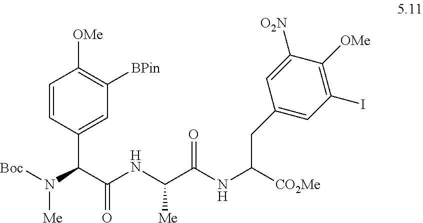

Compound 5.9 (118 mg, 0.19 mmol, 1 eq) was taken up in 95% EtOH (2 mL), 10% Pd/C (38 mg, ⅓ by weight) was added and the mixture was placed under an atmosphere of H₂. The reaction was allowed to proceed until TLC analysis indicated the complete consumption of starting material. The mixture was then filtered through celite and concentrated to yield compound 5.11. To a solution of this crude material (94 mg, 0.19 mmol, 1 eq) and compound 5.6 (101 mg, 0.27 mmol, 1.4 eq) in AcCN:DMF (2.2:1, 2 mL) was added sequentially HOBt (64 mg, 2.5 eq) and EDC (80 mg, 2.2 eq) and the reaction was allowed to stir overnight. Dilute NaHCO_{3(aq)} was then added to the reaction and the aqueous phase was extracted 3× with EtOAc. The combined organic layers were washed with 5% citric acid, water and brine then dried over sodium sulfate and concentrated. The crude material was purified via abbreviated column chromatography (3% MeOH in DCM) to give a semi-pure product (130 mg, 80%). MS (ESI) m/z 877.2 (M+Na⁺).

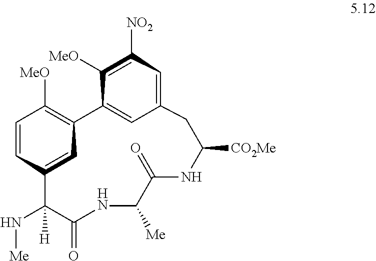

A solution of compound 5.11 (118 mg, 0.14 mol, 1 eq) and NaHCO₃ (118 mg, 10 eq) in DMF (4.2 mL) was purged several times via cycling with vacuum and Ar and sealed with a crimped septa. To this solution was added, via syringe, a solution of PdCl₂(dppf) (23.0 mg, 0.2 eq) in DMF (2.8 mL) that had been sparged with Ar for ˜15 minutes. The resulting mixture was submitted to several more cycles of vacuum and Ar then heated to 80° C. The mixture was cooled to room temperature and water was added. The aqueous phase was extracted with EtOAc 3× then washed with water and brine, dried over sodium sulfate and concentrated. The crude material was subjected to abbreviated column chromatography (4% MeOH in DCM) to remove most of the Pd species then used without further purification. The resulting semi-pure material (83 mg) was taken up in DCM (4.0 mL) and treated with TFA (0.8 mL). The reaction was monitored via TLC and when starting material was no longer present the volatiles were blown off under a stream of nitrogen. DCM was added and blown off under nitrogen twice more and the crude residue was dissolved EtOAc. The organic layer was washed with saturated NaHCO₃, dried over sodium sulfate and concentrated. The crude material was purified via pipette column chromatography (9% MeOH in DCM) to give the product (29.7 mg, 42% yield). MS (ESI) m/z 501.1 (M+H⁺).

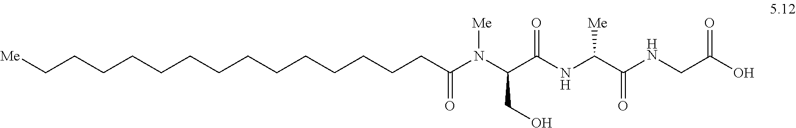 [SMILES: CCCCCCCCCCCCCCCC(=O)N(C)[C@H](CO)C(=O)N[C@H](C)C(=O)NCC(=O)O]

Compound 5.12 was synthesized via standard Fmoc/piperidine solid phase peptide synthesis. Fmoc-Gly-OH was loaded onto chlorotrityl chloride resin with DIEA, then the constituent amino acids, Fmoc-d-Ala-OH and Fmoc-N-Me-d-Ser-OH were coupled to the resin using HCTU/HOBT/DIEA in DMF followed by palmitic acid coupling with HCTU/HOBT/DIEA in DMF and enough DCM to completely dissolve the acid. Cleavage from the resin was achieved using 1% TFA in DCM using protocols detailed in the Novabiochem catalogue. The product was purified via HPLC (linear gradient, 0.66% B per minute, product eluted at 97% B).

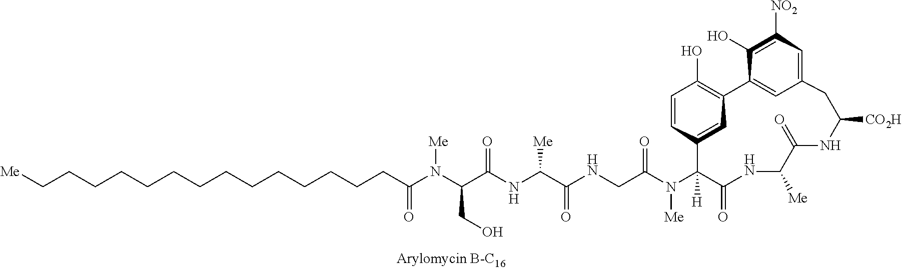

To a solution of Compound 5.12 (29.2 mg, 58.4 μmol) and compound 5.12 (50 mg, 1.5 eq) in THF (0.5 mL) at 0° C. was added DEPBT (28.0 mg, 1.6 eq) and NaHCO₃ (5.0 mg, 1 eq). The reaction was then allowed to warm to room temperature and stirred overnight. The THF was then blown off under a stream of nitrogen and the reaction was dried under vacuum. The crude reaction mixture was taken up in EtOAc, washed 2× with saturated NaHCO₃, then brine, dried over sodium sulfate and concentrated. The crude was purified via column chromatography (3% MeOH in DCM then 4.5% MeOH in DCM) to give the protected arylomycin. The protected arylomycin (10.0 mg, 9.4 μmol, 1 eq) was dissolved in CHCl₃ (2 mL) treated with ethanethiol (180 μL, 250 eq) and 1.0 M AlBr₃ in CH₂Br₂ (189 μL, 20 eq) and stirred in a vial open to air for 6 hrs. The reaction was quenched by the addition of MeOH and the volatiles were blown off under a stream of nitrogen. The crude was taken up in MeOH and dried twice more to remove lingering ethanethiol then it was dissolved in MeOH centrifuged and purified via HPLC (linear gradient, 1.0% B per minute, product eluted at 82% B) to give the product (5.8 mg, 67% yield). ESI HRMS calcd for C₄₇H₇₀N₆O₁₁ [(M+H)⁺] 926.4869. found 926.4873.

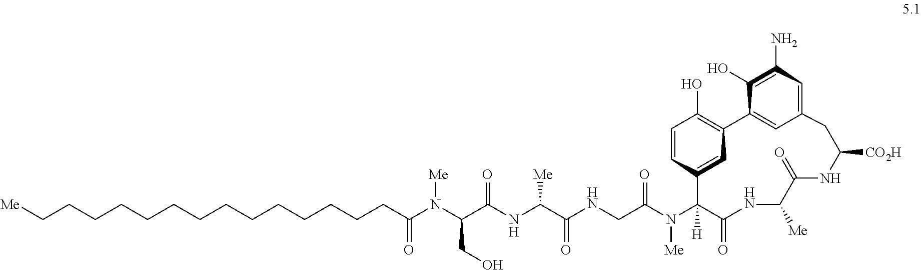

The protected arylomycin (6.3 mg, 6.0 μmol, 1 eq) was dissolved in ethanethiol (300 μL) and 1.0 M AlBr₃ in CH₂Br₂ (120 μL, 20 eq) and stirred in a vial for 5 hrs under Ar. The reaction was quenched by the addition of MeOH and the volatiles were blown off under a stream of nitrogen. The crude was taken up in MeOH and dried twice more to remove lingering ethanethiol then it was dissolved in MeOH centrifuged and purified via HPLC (linear gradient, 1.0% B per minute, product eluted at 75% B) to give the product (1.0 mg, 19% yield). ESI HRMS calcd for C₄₇H₇₀N₆O₁₁ [(M+H)⁺]896.5128. found 896.5123.

### General Procedure D: Macrocycle and Tail Coupling, Example—Arylomycin C16

Compound 52 (80 mg, 0.16 mmol) was taken up in AcCN (7.2 mL) and DMF (3.2 mL) and treated sequentially with HOBT (64 mg, 3 eq), compound 51 (81.3 mg, 1 eq) and EDC (90.3 mg, 3 eq). The reaction was allowed to stir overnight, after which water, saturated NaHCO₃, and EtOAc were added, the aqueous phase was extracted 3× with EtOAc and the combined organic layers were washed with 5% citric acid (pH—3) and brine. The organics were dried over sodium sulfate and concentrated. The crude was purified by column chromatography (5.5% MeOH in DCM) to give the product 53 (72.4 mg, 45% yield).

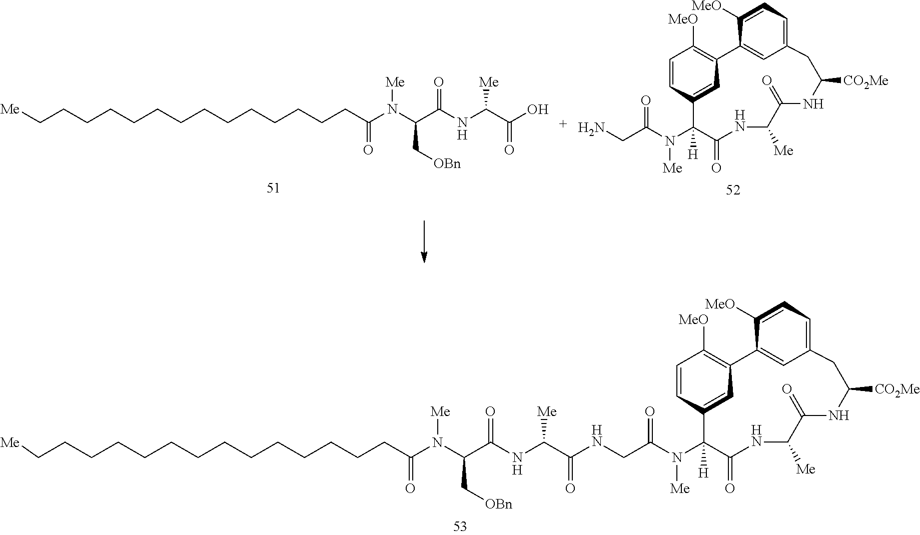

### General Procedure E: Global Deprotection, Example—Arylomycin C16

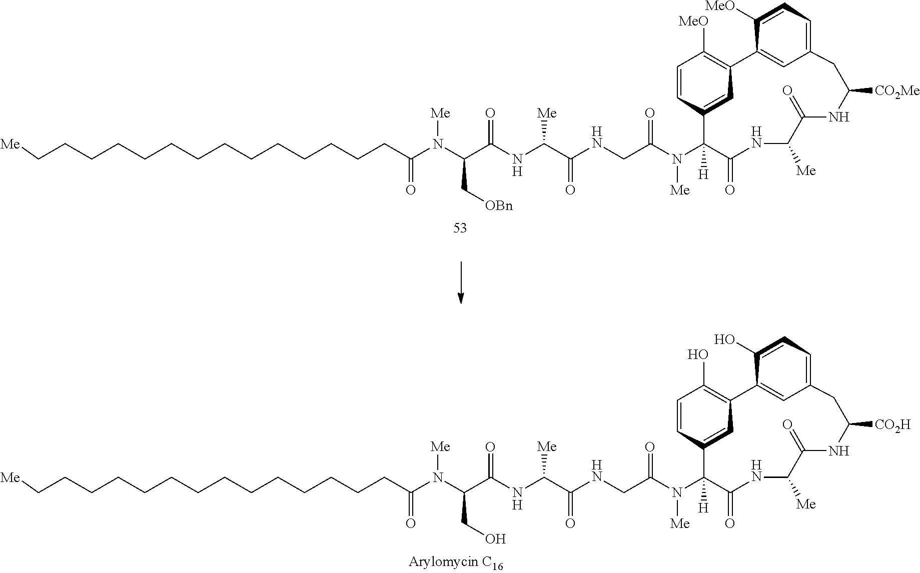

Compound 53 (72.4 mg, 72 μmol, 1 eq) was dissolved in ethanethiol (2 mL) under Ar and treated with 1.0 M AlBr₃ in CH₂Br₂ (1.79 mL, 25 eq). The reaction vial was sealed and heated to 50° C. and stirred for 4 h. The reaction was cooled to room temperature, MeOH was added (0.5 mL) and the volatiles were blown off under a stream of nitrogen. MeOH was added again and was blown off under a stream of nitrogen and the crude product was dried under vacuum. The crude product was then dissolved in MeOH and purified by HPLC (linear gradient, 0.67% B/min, product eluted at 80% B) to give Arylomycin C16. (32.6 mg, 51% yield). ESI HRMS calcd for C46H69N6O11 [(M+H)+]: 881.5019. found: 881.5021.

### General Procedure F: Macrocycle and Tail Coupling Example—Compound 56:

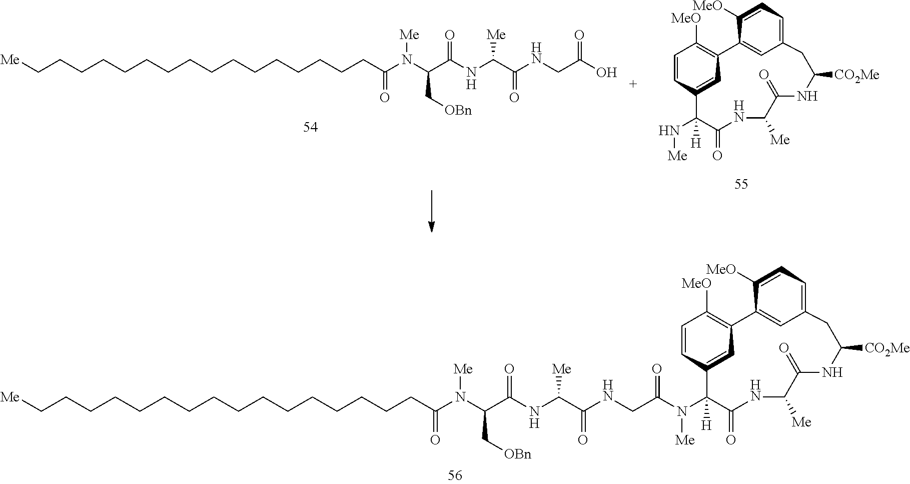

This procedure is based on the conditions previously reported.9 Compound 53 (23.5 mg, 52 μmol, 1 eq) and compound 54 (70 mg, 2.2 eq) were dissolved in THF (2 mL) under Ar and treated with TEA (7 μL, 1 eq) and DEPBT (39 mg, 2.5 eq). The reaction was allowed to stir overnight then the volatiles were blown off under a stream of nitrogen, the residue was dried under vacuum, and EtOAc and saturated NaHCO₃ were added. The aqueous layer was extracted, then the organic layer was washed with 0.1N HCl, dried over sodium sulfate and concentrated.

Compound 1 was synthesized using general procedures D and E.

ESI HRMS calcd for C46H70N6O10 [(M+H)+]: 867.5226. found: 867.5207.

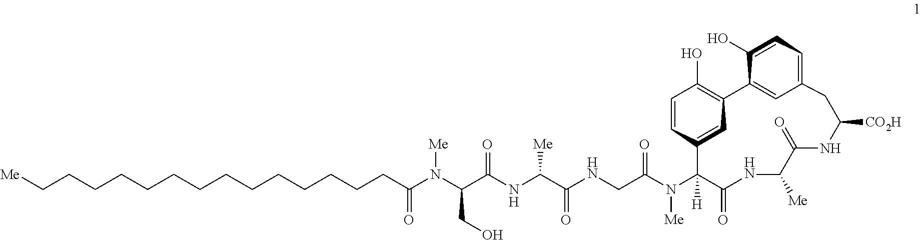

Compound 2 was synthesized using general procedures D and E.

ESI HRMS calcd for C38H53N6O11 [(M+H)+]: 769.3767. found: 769.3770.

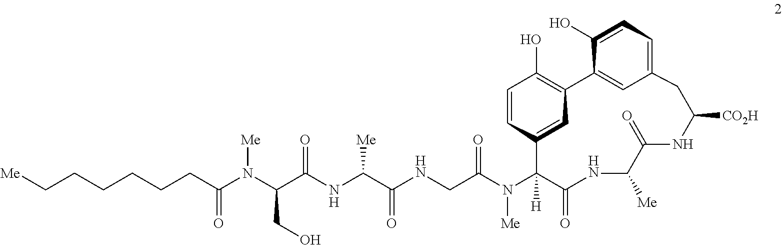

Compound 3 was synthesized using general procedures D and E.

ESI HRMS calcd for C40H57N6O11 [(M+H)+]: 797.408. found: 797.4070.

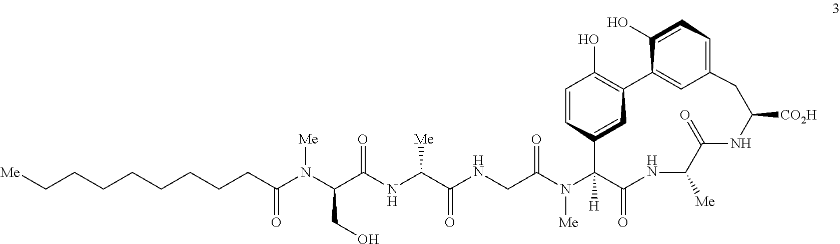

Compound 4 was synthesized using general procedures D and E.

ESI HRMS calcd for C42H61N6O11 [(M+H)+]: 825.4393. found: 825.4386.

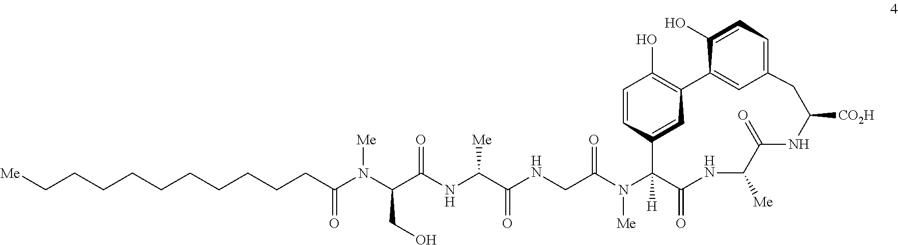

Compound 5 was synthesized as shown above then subjected to general procedure F to give the product (20.6 mg, 58% yield).

ESI HRMS calcd for C48H72N6O11 [(M+H)+]: 909.5332. found: 909.5328.

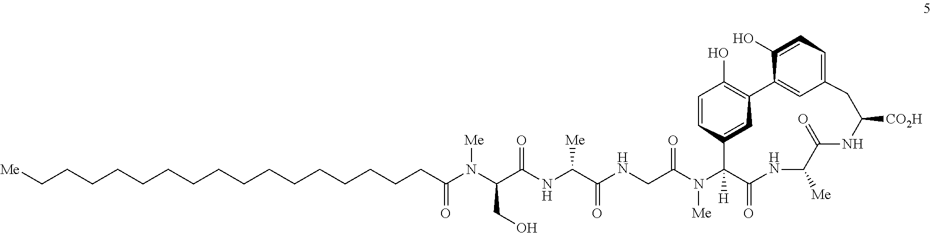

Compound 6 was synthesized using general procedures D and E. ESI HRMS calcd for C43H54N6O11 [(M+H)+]: 811.3297. found: 811.3300.

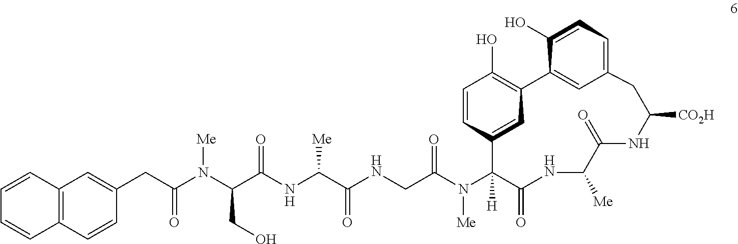

Compound 7 was synthesized using general procedures D and E. ESI HRMS calcd for C44H48N6O11 [(M+H)+]: 837.3454. found: 837.3443.

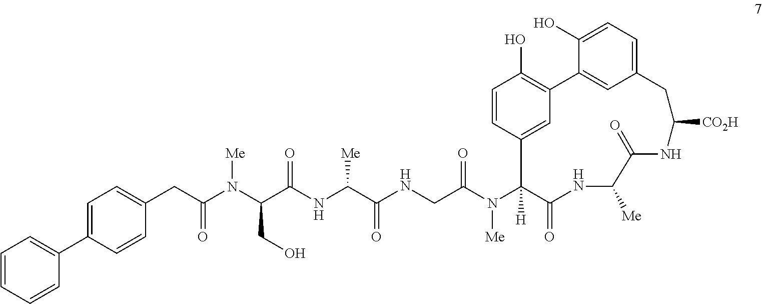 [SMILES: C[C@@H]1NC(=O)[C@@H](N(C)C(=O)CNC(=O)[C@@H](C)NC(=O)[C@@H](CO)N(C)C(=O)Cc2ccc(-c3ccccc3)cc2)c2ccc(O)c(c2)-c2cc(ccc2O)C[C@@H](C(=O)O)NC1=O]

Compound 8 was synthesized using general procedures D and E.

ESI HRMS calcd for C43H46N6O11 [(M+H)+]: 823.3297. found: 823.3296.

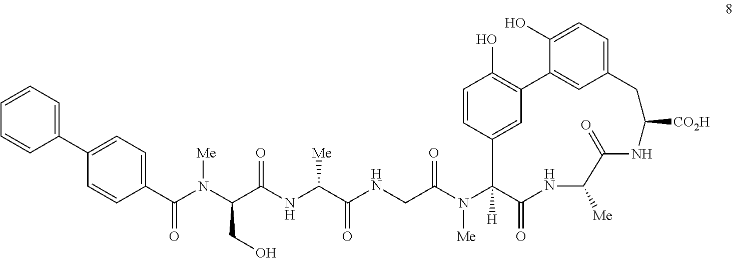 [SMILES: C[C@@H]1NC(=O)[C@@H](N(C)C(=O)CNC(=O)[C@@H](C)NC(=O)[C@@H](CO)N(C)C(=O)c2ccc(-c3ccccc3)cc2)c2ccc(O)c(c2)-c2cc(ccc2O)C[C@@H](C(=O)O)NC1=O]

Compound 9 was synthesized using general procedures D and E.

ESI HRMS calcd for C45H50N6O11 [(M+H)+]: 851.361. found: 851.359.

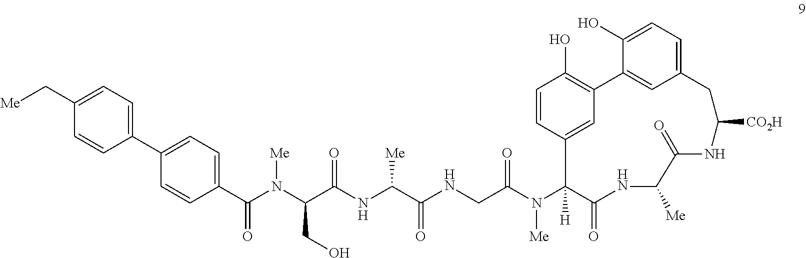 [SMILES: CCc1ccc(-c2ccc(C(=O)N(C)[C@H](CO)C(=O)N[C@H](C)C(=O)NCC(=O)N(C)[C@@H]3C(=O)N[C@@H](C)C(=O)N[C@H](C(=O)O)Cc4ccc(O)c(c4)-c4cc3ccc4O)cc2)cc1]

Compound 10 was synthesized using general procedures D and E.

ESI HRMS calcd for C47H54N6O11 [(M+H)+]: 879.3923. found: 879.3924.

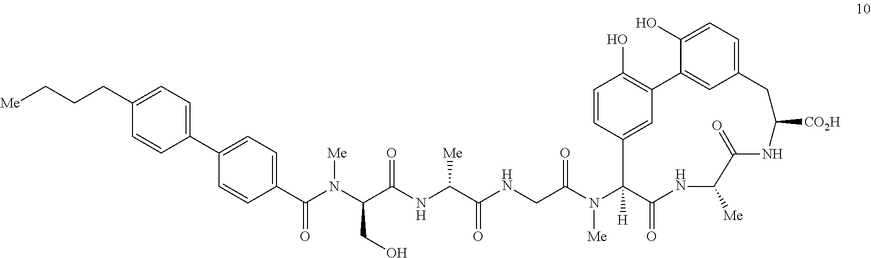 [SMILES: CCCCc1ccc(-c2ccc(C(=O)N(C)[C@H](CO)C(=O)N[C@H](C)C(=O)NCC(=O)N(C)[C@@H]3C(=O)N[C@@H](C)C(=O)N[C@H](C(=O)O)Cc4ccc(O)c(c4)-c4cc3ccc4O)cc2)cc1]

Compound 11 was synthesized using general procedures D and E.

ESI HRMS calcd for C49H58N6O11 [(M+H)+]: 907.4236. found: 907.4246.

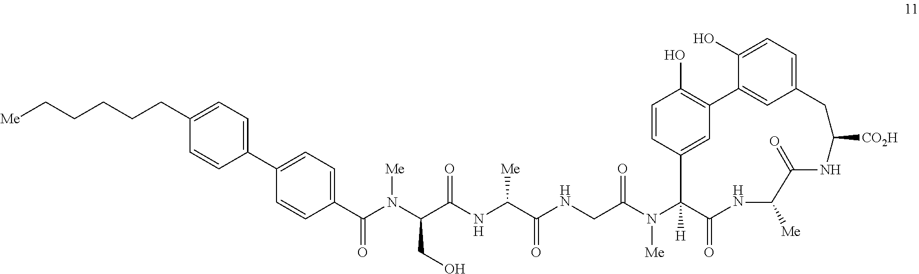 [SMILES: CCCCCCc1ccc(-c2ccc(C(=O)N(C)[C@H](CO)C(=O)N[C@H](C)C(=O)NCC(=O)N(C)[C@@H]3C(=O)N[C@@H](C)C(=O)N[C@H](C(=O)O)Cc4ccc(O)c(c4)-c4cc3ccc4O)cc2)cc1]

Compound 12 was synthesized using general procedures D and E.

ESI HRMS calcd for C51H62N6O11 [(M+H)+]: 935.4549. found: 935.4548.

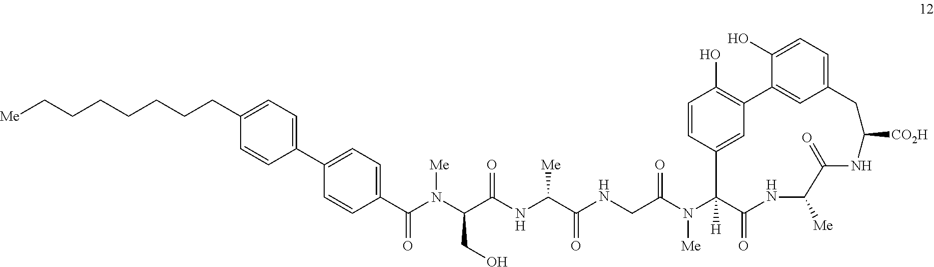 [SMILES: CCCCCCCCc1ccc(-c2ccc(C(=O)N(C)[C@H](CO)C(=O)N[C@H](C)C(=O)NCC(=O)N(C)[C@@H]3C(=O)N[C@@H](C)C(=O)N[C@H](C(=O)O)Cc4ccc(O)c(c4)-c4cc3ccc4O)cc2)cc1]

Compound 13 was synthesized using general procedures D and E. ESI HRMS calcd for C43H54N6O11 [(M+H)+]: 831.3923. found: 831.3917.

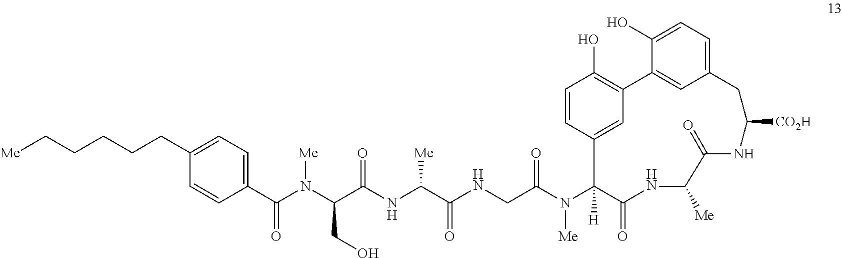 [SMILES: CCCCCCc1ccc(C(=O)N(C)[C@H](CO)C(=O)N[C@H](C)C(=O)NCC(=O)N(C)[C@@H]2C(=O)N[C@@H](C)C(=O)N[C@H](C(=O)O)Cc3ccc(O)c(c3)-c3cc2ccc3O)cc1]

Compound 14 was synthesized using general procedures D and E.

EST HRMS calcd for C45H58N6O11 [(M+H)+]: 859.4236. found: 859.4231.

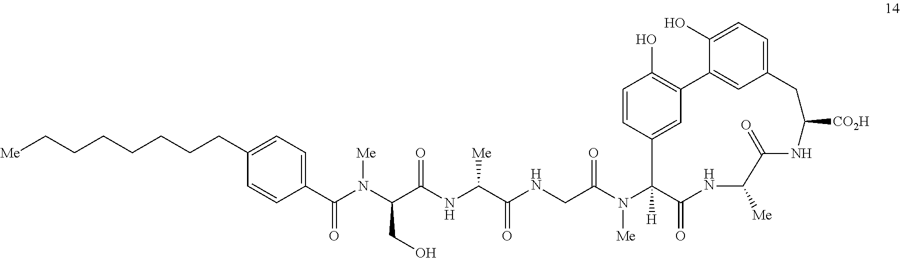 [SMILES: CCCCCCCCc1ccc(C(=O)N(C)[C@H](CO)C(=O)N[C@H](C)C(=O)NCC(=O)N(C)[C@@H]2C(=O)N[C@@H](C)C(=O)N[C@H](C(=O)O)Cc3ccc(O)c(c3)-c3cc2ccc3O)cc1]

Compound 15 was synthesized using general procedures D and E.

ESI HRMS calcd for C47H62N6O11 [(M+H)+]: 887.4549. found: 887.4539.

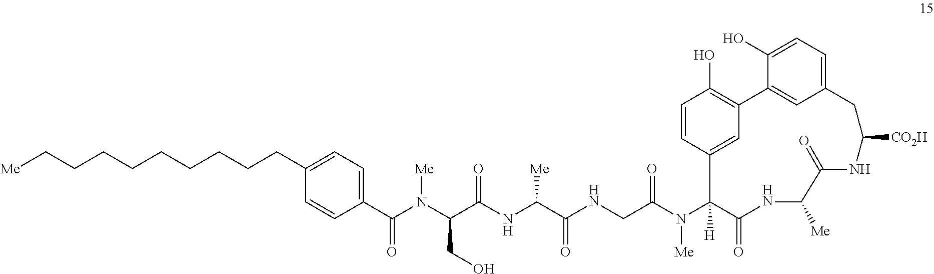 [SMILES: CCCCCCCCCCc1ccc(C(=O)N(C)[C@H](CO)C(=O)N[C@H](C)C(=O)NCC(=O)N(C)[C@@H]2C(=O)N[C@@H](C)C(=O)N[C@H](C(=O)O)Cc3ccc(O)c(c3)-c3cc2ccc3O)cc1]

Compound 16 was synthesized using general procedures D and E.

ESI HRMS calcd for C45H66N6O11 [(M+H)+]: 867.4862. found: 867.4873.

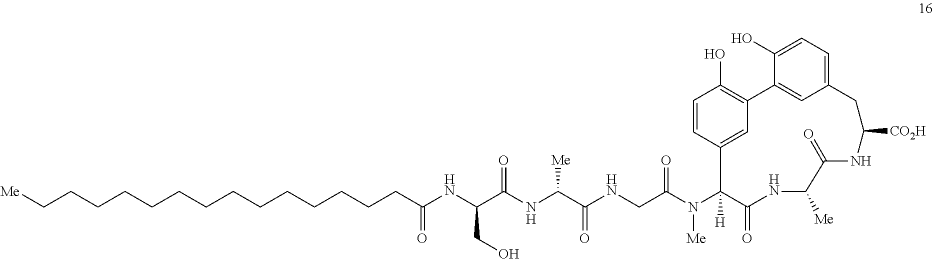 [SMILES: CCCCCCCCCCCCCCCC(=O)N[C@H](CO)C(=O)N[C@H](C)C(=O)NCC(=O)N(C)[C@@H]1C(=O)N[C@@H](C)C(=O)N[C@H](C(=O)O)Cc2ccc(O)c(c2)-c2cc1ccc2O]

Compound 17 was synthesized using general procedures D and E.

ESI HRMS calcd for C47H70N6O10 [(M+H)+]: 895.5175. found: 895.5190.

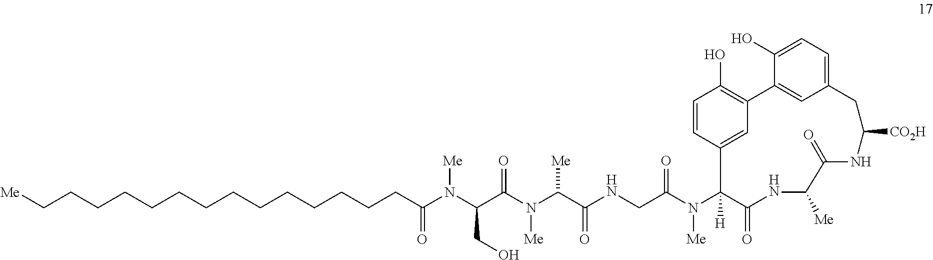 [SMILES: CCCCCCCCCCCCCCCC(=O)N(C)[C@H](CO)C(=O)N(C)[C@H](C)C(=O)NCC(=O)N(C)[C@@H]1C(=O)N[C@@H](C)C(=O)N[C@H](C(=O)O)Cc2ccc(O)c(c2)-c2cc1ccc2O]

Compound 18 was synthesized using general procedures D and E.

ESI HRMS calcd for C47H68N6O11 [(M+H)+]: 893.5019. found: 893.5014.

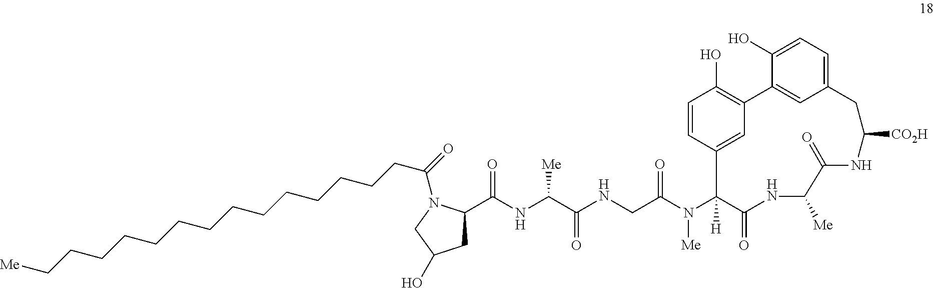

Diastereomer A of compound 19 was synthesized using general procedures D and E.

ESI HRMS calcd for C43H63N5O9 [(M+H)+]: 794.4698. found: 794.4705.

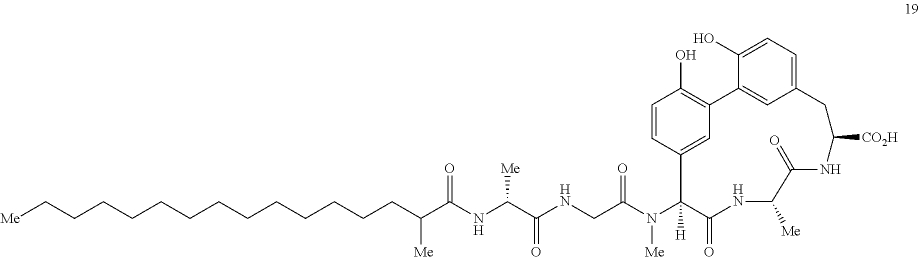 [SMILES: CCCCCCCCCCCCCCC(C)C(=O)N[C@H](C)C(=O)NCC(=O)N(C)[C@@H]1C(=O)N[C@@H](C)C(=O)N[C@H](C(=O)O)Cc2ccc(O)c(c2)-c2cc1ccc2O]

Diastereomer B of compound 19 was synthesized using general procedures D and E.

ESI HRMS calcd for C43H63N5O9 [(M+H)+]: 794.4698. found: 794.4689.

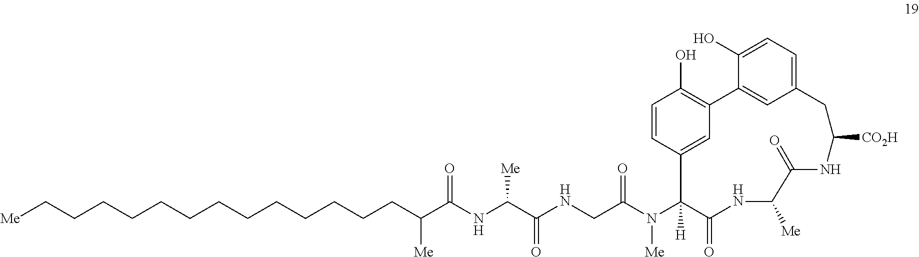 [SMILES: CCCCCCCCCCCCCCC(C)C(=O)N[C@H](C)C(=O)NCC(=O)N(C)[C@@H]1C(=O)N[C@@H](C)C(=O)N[C@H](C(=O)O)Cc2ccc(O)c(c2)-c2cc1ccc2O]

Compound 20 was synthesized racemically using general procedures E and F.

ESI FIRMS calcd for C47H70N6O11 [(M+H)+]: 895.5175. found: 895.5180.

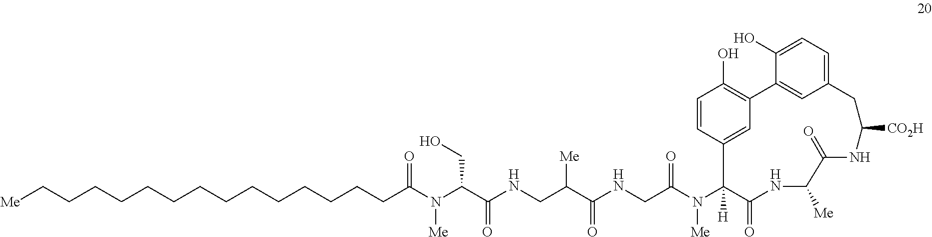 [SMILES: CCCCCCCCCCCCCCCC(=O)N(C)[C@H](CO)C(=O)NCC(C)C(=O)NCC(=O)N(C)[C@@H]1C(=O)N[C@@H](C)C(=O)N[C@H](C(=O)O)Cc2ccc(O)c(c2)-c2cc1ccc2O]

Compound 21 was synthesized racemically using general procedures E and F. ESI HRMS calcd for C48H72N6O11 [(M+H)+]: 909.5332. found: 909.5334.

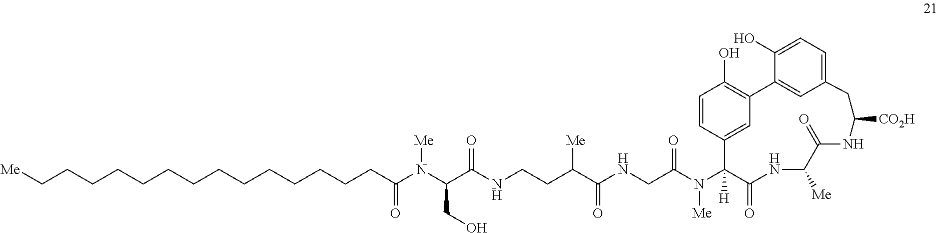 [SMILES: CCCCCCCCCCCCCCCC(=O)N(C)[C@H](CO)C(=O)NCCC(C)C(=O)NCC(=O)N(C)[C@@H]1C(=O)N[C@@H](C)C(=O)N[C@H](C(=O)O)Cc2ccc(O)c(c2)-c2cc1ccc2O]

Compound 22 was synthesized using general procedures D and E.

ESI HRMS calcd for C47H70N6O11 [(M+H)+]: 895.5175. found: 895.5178.

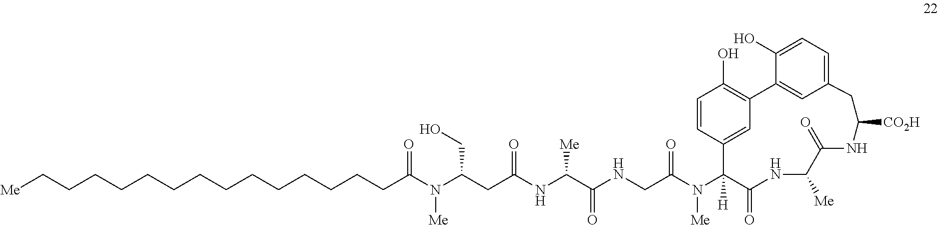 [SMILES: CCCCCCCCCCCCCCCC(=O)N(C)[C@H](CO)CC(=O)N[C@H](C)C(=O)NCC(=O)N(C)[C@@H]1C(=O)N[C@@H](C)C(=O)N[C@H](C(=O)O)Cc2ccc(O)c(c2)-c2cc1ccc2O]

Compound 23 was synthesized using general procedures D and E.

ESI HRMS calcd for C48H72N6O11 [(M+H)+]: 909.5332. found: 909.5305.

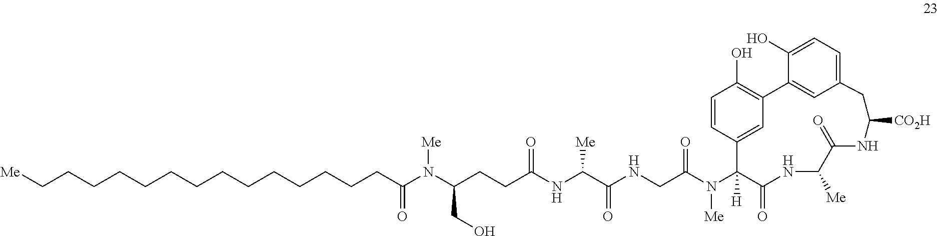 [SMILES: CCCCCCCCCCCCCCCC(=O)N(C)[C@H](CO)CCC(=O)N[C@H](C)C(=O)NCC(=O)N(C)[C@@H]1C(=O)N[C@@H](C)C(=O)N[C@H](C(=O)O)Cc2ccc(O)c(c2)-c2cc1ccc2O]

### General Procedure G: Compound 61

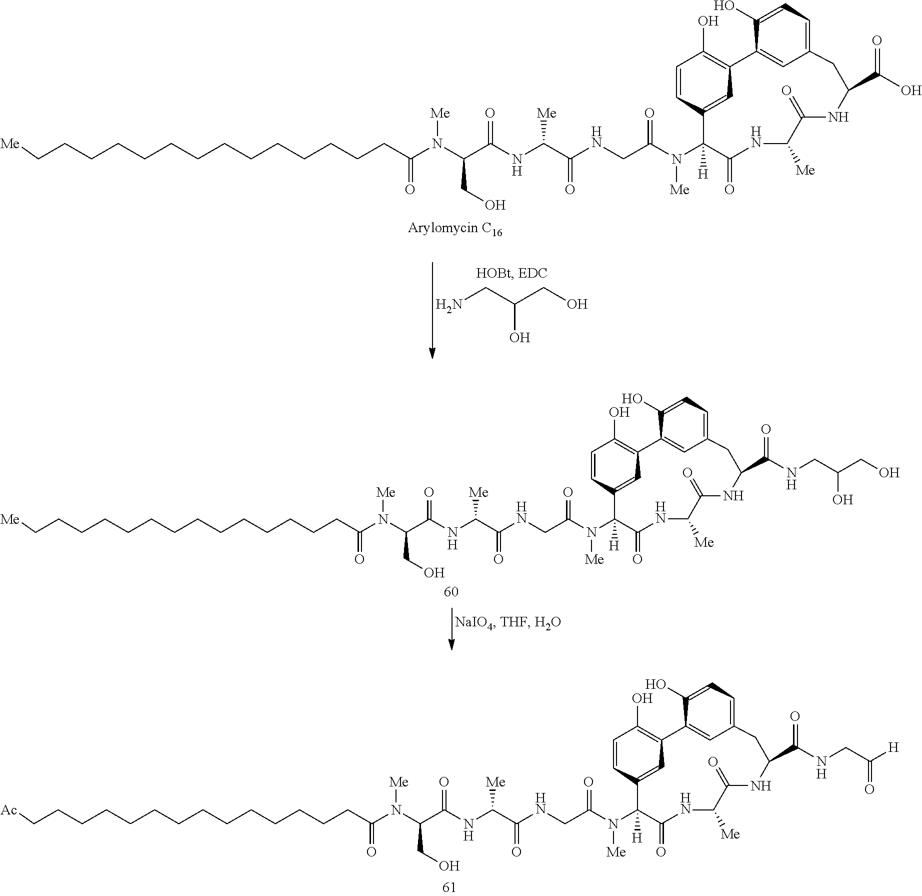

Arylomycin C₁₆ (4 mg, 4.55 μmol) was dissolved in 0.4 mL of anhydrous DMF and treated sequentially with HOBT (3.1 mg, 23 μmol, 5 eq) and 3-aminopropane-1,2-diol (4.1 mg, 45 μmol, 10 eq). The solution was then cooled to 0° C. and EDC (5.3 mg, 28 μmol, 6 eq) was added in one portion. The reaction was allowed to warm to room temperature and stirred overnight. Purification of the reaction mixture directly by prep-HPLC gave 60 (3 mg, 70% yield) as a white solid.

To a stirred solution of compound 60 (3 mg, 3.15 μmol) in 0.4 mL of THF/H₂O (10/1) was added NaIO₄ (2 mg, 9.45 μmol, 3 eq) at 0° C. and the mixture was stirred for an hour at the same temperature. The reaction mixture was then purified by prep-HPLC to give compound 61 (1.7 mg, 60% yield) as a white solid. MS (ESI) for (C₄₈H₇₂N₇₀O₁₁): m/z 922.5 (M+H).

### General Procedure H. Compound 62

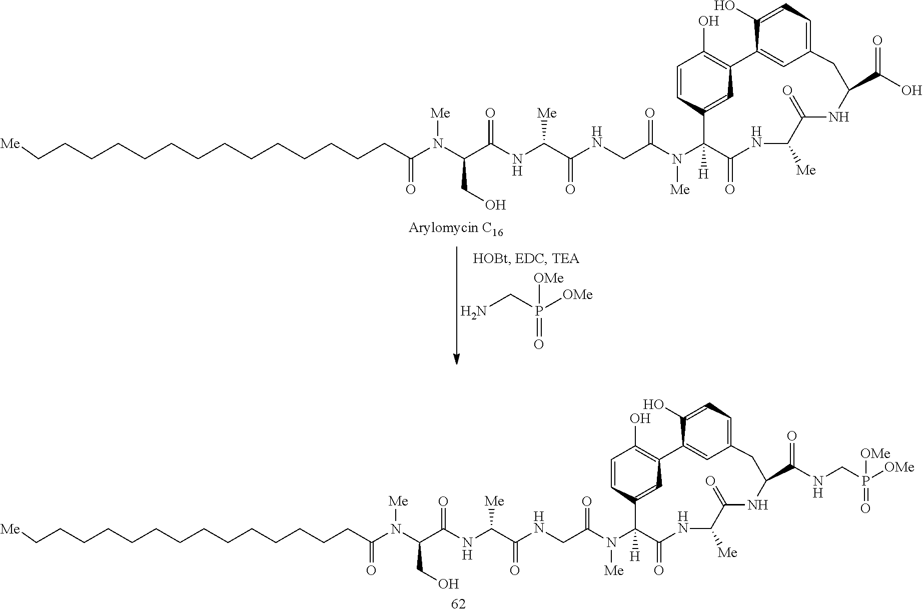

Arylomycin C₁₆ (3 mg, 3.4 μmol) was dissolved in 0.3 mL of anhydrous DMF and treated sequentially with HOBT (2 mg, 15 μmol, 5 eq) TEA (3.4 mg, 34 μmol, 10 eq) and dimethyl aminomethylphosphonate (9.4 mg, 68 μmol, 20 eq). The solution was then cooled to 0° C. and EDC (3.9 mg, 20.4 μmol, 6 eq) was added in one portion. The reaction was allowed to warm to room temperature and stirred overnight. Purification of the reaction mixture directly by prep-HPLC gave 62 (2.2 mg, 64% yield) as a white solid. MS (ESI) for (C₄₉H₇₇N₇O₁₃P): m/z 1002.5 (M+H).

### General Procedure I: Compound 63

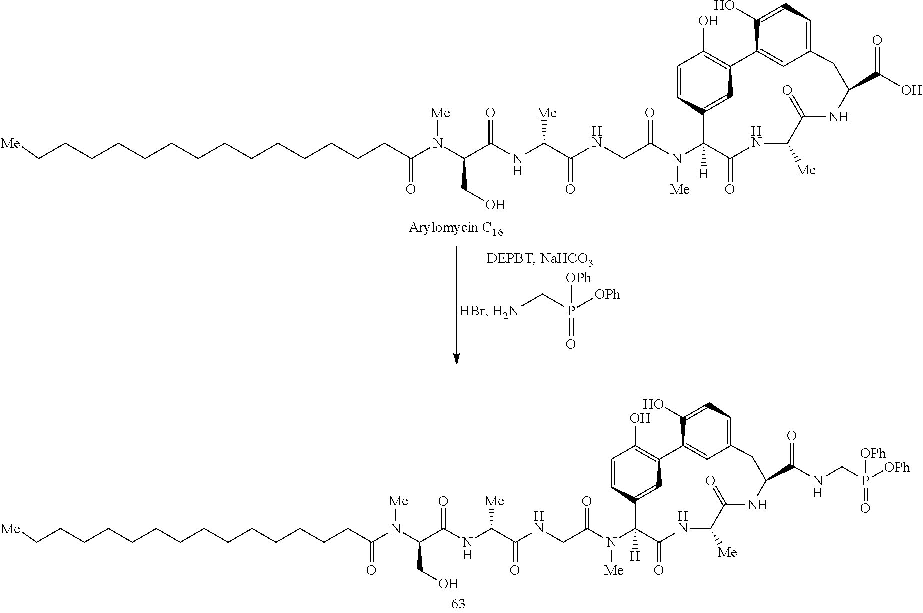

To a solution of Arylomycin C₁₆ (3 mg, 3.4 μmol) in anhydrous THF (0.5 mL) was added diphenyl aminomethylphosphonate (12 mg, 34 μmol, 10 eq), 3-(diethyloxyphosphoryloxy)-1,2,3-benzotriazin-4(3H)-one (DEPBT) (21 mg, 68 μmol, 20 eq), and NaHCO₃ (6 mg, 68 μmol, 20 eq) at 0° C. The resulting mixture was allowed to warm to room temperature and stirred for 24 hrs. The reaction was diluted with 30 mL of EtOAc. The organic layer was washed with a saturated aqueous NaHCO₃ solution and brine, then dried over Na₂SO₄ and concentrated under reduced pressure. Purification by prep-HPLC gave 63 (2 mg, 52% yield) as a white solid. MS (ESI) for (C₅₉H₈₁N₇O₁₃P): m/z 1126.6 (M+H).

### Example 2

### Bacteriological Materials and Methods

This Example illustrates procedures for testing and manipulating the response of bacteria to arylomycins.

### Strains and Culture Conditions

Standard methods were used to culture bacteria for all experiments and to construct mutant strains. Escherichia coli MG1655, Pseudomonas aeruginosa PAO1, and Klebsiella pneumoniae ATCC 43816 were grown in Luria-Bertani (LB) broth at 37° C. Yersinia pestis Kim+6 was grown in LB broth at 28° C. Staphylococcus epidermidis RP62A, Staphylococcus haemolyticus, a clinical isolate obtained from the London Health Services Centre, London, Ontario, Canada, Staphylococcus aureus NCTC 8325, and Corynebacterium efficiens DSM 44549 were grown in trypicase soy broth (TSB) at 37° C. Streptococcus pneumoniae R800, was grown in Todd Hewitt broth without shaking at 37° C. Streptococcus agalactiae COH-1 and Streptococcus pyogenes M1-5448 were grown in Brain-Heart Infusion (BHI) broth at 37° C. Lactobacillus gasseri ATCC 19992, Lactobacillus acidophilus ATCC 4356, and Lactobacillus plantarum ATCC 8014 were grown at 37° C. on Man-Rogosa-Sharp agar in a sealed candle jar to provide a CO2 enriched atmosphere. Lactococcus Lactis subsp. lactis ATCC 11454 and Lactococcus Lactis subsp. cremoris ATCC 19257 were grown in Trypticase Soy Yeast broth at 37° C. and 28° C. respectively. Rhodococcus equi ATCC 6939 was grown in cation-adjusted Mueller Hinton Broth II (MHBII) at 37° C. Rhodococcus opacus DSM 1069, Corynebacterium glutamicum DSM 44475, Brevibacillus brevis ATCC 8246, and Bacillus subtilis 168 were grown in Mueller Hinton Broth II at 28° C. Francisella tularensis (19 strains including Types A and B) were grown on chocolatized 9% sheep blood (CHAB) at 37° C. in a BSL level 3 facility. Clostridia difficile WAL14572, Clostridia bolteae WAL16351, Clostridia perfringens WAL 14572, Bacteroides fragilis ATCC 25285 and Prevotella copris WAL16310 were grown on reinforced clostridial medium (Oxoid CM149) under anaerobic conditions at 37° C. Helicobacter pylori SS1 was grown in BHI broth supplemented with 5% fetal bovine serum in 10% CO2 at 37° C. Chlamydia trachomatis was propagated and titered in HeLa229 cells grown in Dulbecco's modified Eagle's medium (high glucose; Invitrogen, Carlsbad, Calif.) as described previously (Lad et al., 2007). All liquid cultures were grown with 275 rpm shaking, and all media was obtained from Difco™.

Selection of Arylomycin Resistant S. epidermidis and Sequencing of Signal Peptidase Genes

S. epidermidis (˜1×109 cfu) were plated on tryptic soy agar (TSA) containing 2 μg/ml arylomycin C16. Resistant colonies visible at 24 hours were re-streaked onto TSA containing 2 μg/ml arylomycin C16 to confirm the resistant phenotype.

Isolation of genomic DNA and sequencing of SPases genes was performed using available procedures. In particular, genomic DNA was obtained by lysing S. epidermidis with lysostaphin (Sigma), followed by Proteinase K (Roche) treatment, and heat lysis at 95° C. Signal peptidase genes were amplified using the primer pairs Se_SpsIB_F/Se_SpsIB_R and Se_SpsI_F/Se_SpsI_R (see Table 10 of Primers, below). Sequencing was performed using primers Se_SpsIB_R and Se_SpsI_F.

TABLE 10
Primers for Detecting and Synthesizing Bacterial Spase
Nucleic Acids

| Primer Name | Primer Sequence 5′ --> 3′ | SEQ ID NO: |
|---|---|---|
| Ec_lepB_usNF | TCCCGTTCGCTGGCTGCCTGTG | |
| Ec_lepB_CR_Kan | CGGCGGCTTTGTTGAATAAATCGTTAAT | |
| | GGATGCCGCCAATGCG | |
| Ec_lepB_CF_Kan | GAGACACAACGTGGCTTTCCCATTAAT | |
| | AGCCATCTTCGTTCACG | |
| Ec_lepB_dsCR | TTGGTTTCTAGACCAGCGTATTGCCACG | |
| | GACC | |
| Ec_lepB_Nconf | TTGGTTTCTAGACTTTATCGACACCCCGG | |
| Kan_ICF2 | GGTTGTAACACTGGCAGAGC | |
| Ec_lepB_QC_P84A_F | CGTTCGTTTATTTATGAAGCGTTCCAGA | |
| | TCCCGTCAGGT | |
| Ec_lepB_QC_P84A_R | ACCTGACGGGATCTGGAACGCTTCATA | |
| | AATAAACGAACG | |
| Ec_lepB_QC_P84C_F | CGTTCGTTTATTTATGAATGCTTCCAGA | |
| | TCCCGTCAGGT | |
| Ec_lepB_QC_P84C_R | ACCTGACGGGATCTGGAAGCATTCATA | |
| | AATAAACGAACG | |
| Ec_lepB_QC_P84D_F | CGTTCGTTTATTTATGAAGATTTCCAGA | |
| | TCCCGTCAGGT | |
| Ec_lepB_QC_P84D_R | ACCTGACGGGATCTGGAAATCTTCATA | |
| | AATAAACGAACG | |
| Ec_lepB_QC_P84E_F | CGTTCGTTTATTTATGAAGAGTTCCAGA | |
| | TCCCGTCAGGT | |
| Ec_lepB_QC_P84E_R | ACCTGACGGGATCTGGAACTCTTCATA | |
| | AATAAACGAACG | |
| Ec_lepB_QC_P84F_F | CGTTCGTTTATTTATGAATTCTTCCAGA | |
| | TCCCGTCAGGT | |
| Ec_lepB_QC_P84F_R | ACCTGACGGGATCTGGAAGAATTCATA | |
| | AATAAACGAACG | |
| Ec_lepB_QC_P84G_F | CGTTCGTTTATTTATGAAGGCTTCCAGA | |
| | TCCCGTCAGGT | |
| Ec_lepB_QC_P84G_R | ACCTGACGGGATCTGGAAGCCTTCATA | |
| | AATAAACGAACG | |
| Ec_lepB_QC_P84H_F | CGTTCGTTTATTTATGAACATTTCCAGA | |
| | TCCCGTCAGGT | |
| Ec_lepB_QC_P84H_R | ACCTGACGGGATCTGGAAATGTTCATA | |
| | AATAAACGAACG | |
| Ec_lepB_QC_P84I_F | CGTTCGTTTATTTATGAAATCTTCCAGA | |
| | TCCCGTCAGGT | |
| Ec_lepB_QC_P84I_R | ACCTGACGGGATCTGGAAGATTTCATA | |
| | AATAAACGAACG | |
| Ec_lepB_QC_P84K_F | CGTTCGTTTATTTATGAAAAATTCCAGA | |
| | TCCCGTCAGGT | |
| Ec_lepB_QC_P84K_R | ACCTGACGGGATCTGGAATTTTTCATAA | |
| | ATAAACGAACG | |
| Ec_lepB_QC_P84L_F | CGTTCGTTTATTTATGAACTGTTCCAGA | |
| | TCCCGTCAGGT | |
| Ec_lepB_QC_P84L_R | ACCTGACGGGATCTGGAACAGTTCATA | |
| | AATAAACGAACG | |
| Ec_lepB_QC_P84M_F | CGTTCGTTTATTTATGAAATGTTCCAGA | |
| | TCCCGTCAGGT | |
| Ec_lepB_QC_P84M_R | ACCTGACGGGATCTGGAACATTTCATA | |
| | AATAAACGAACG | |
| Ec_lepB_QC_P84N_F | CGTTCGTTTATTTATGAAAACTTCCAGA | |
| | TCCCGTCAGGT | |
| Ec_lepB_QC_P84N_R | ACCTGACGGGATCTGGAAGTTTTCATA | |
| | AATAAACGAACG | |
| Ec_lepB_QC_P84Q_F | CGTTCGTTTATTTATGAACAGTTCCAGA | |
| | TCCCGTCAGGT | |
| Ec_lepB_QC_P84Q_R | ACCTGACGGGATCTGGAACTGTTCATA | |
| | AATAAACGAACG | |
| Ec_lepB_QC_P84R_F | CGTTCGTTTATTTATGAACGGTTCCAGA | |
| | TCCCGTCAGGT | |
| Ec_lepB_QC_P84R_R | ACCTGACGGGATCTGGAACCGTTCATA | |
| | AATAAACGAACG | |
| Ec_lepB_QC_P84S_F | GTGCGTTCGTTTATTTATGAATCGTTCC | |
| | AGATCCCGTCAGGTTCG | |
| Ec_lepB_QC_P84S_R | CGAACCTGACGGGATCTGGAACGATTC | |
| | ATAAATAAACGAACGCAC | |
| Ec_lepB_QC_P84T_F | CGTTCGTTTATTTATGAAACCTTCCAGA | |
| | TCCCGTCAGGT | |
| Ec_lepB_QC_P84T_R | ACCTGACGGGATCTGGAAGGTTTCATA | |
| | AATAAACGAACG | |
| Ec_lepB_QC_P84V_F | CGTTCGTTTATTTATGAAGTGTTCCAGA | |
| | TCCCGTCAGGT | |
| Ec_lepB_QC_P84V_R | ACCTGACGGGATCTGGAACACTTCATA | |
| | AATAAACGAACG | |
| Ec_lepB_QC_P84W_F | CGTTCGTTTATTTATGAATGGTTCCAGA | |
| | TCCCGTCAGGT | |
| Ec_lepB_QC_P84W_R | ACCTGACGGGATCTGGAACCATTCATA | |
| | AATAAACGAACG | |
| Ec_lepB_QC_P84Y_F | CGTTCGTTTATTTATGAATATTTCCAGA | |
| | TCCCGTCAGGT | |
| Ec_lepB_QC_P84Y_R | ACCTGACGGGATCTGGAAATATTCATA | |
| | AATAAACGAACG | |
| Pa_lepB_usNF3_BamHI | TTGGTTGGATCCTGGTGCTCGACTTCTT | |
| | CGATCG | |
| Pa_lepB_dsCR_SpeI | TTGGTTACTAGTGTCGGACCTCATGTCA | |
| | GTGTAG | |
| Pa_lepB_QC_P84S_F | CGTTCCTTCCTGGTCGAGAGCTTCCAGA | |
| | TTCCCTCGGGG | |
| Pa_lepB_QC_P84S_R | CCCCGAGGGAATCTGGAAGCTCTCGAC | |
| | CAGGAAGGAACG | |
| Pa_lepB_seqF | GTGGCGATCCAGGCAGCCATC | |
| Sa_spsB_usNF_EcoRI | TTGGTTGAATTCGATCTGTAAACGATTG | |
| | GTGAACAC | |
| Sa_spsB_dsCR_EcoRI | TTGGTTGAATTCGTTCGCTATAACTACC | |
| | AACTTCTTGG | |
| Sa_spsB_QC_P29S_F | GTAGGTAAATTTATTGTTACGTCATATA | |
| | CAATTAAAGGTGAATC | |
| Sa_spsB_QC_P29S_R | GATTCACCTTTAATTGTATATGACGTAA | |
| | CAATAAATTTACCTAC | |
| Se_spsI_F | CAAGGAAAGCGTGTCGTTGTTGTACC | |
| Se_spsI_R | CCAATCATTCTTGCTGCAGTAGGTCTAA | |
| | CG | |
| Se_spsIB_F | TGATGGTGATACGATTCCACCGGGAGC | |
| Se_spsIB_R | GCATGGCTGTTGACTTTCCTGTACCTGC | |
| Ec_lepB_Δ2_75_NF_NcoI | TTGGTTCCATGGTGCGTTCGTTTATTTA | |
| | TGAAC | |
| Ec_lepB_CR_BamHI | TTGGTTGGATCCTGGCATTTAATGGATG | |
| | CCGCCAATGC | |
| Sa_spsIB_NF_KpnI | TTGGTTGGTACCTTGAAAAAAGAAATA | |
| | TTGGAATGG | |
| Sa_spsIB_CR_XhoI | TTGGTTCTCGAGTTAATTTTTAGTATTTT | |
| | CAGGATTGAAAT | |

### Construction of Mutant Strains.

E. coli with a kanamycin marked lepB gene was constructed using allelic exchange methods described by Cirz et al. (PLoS Biol. 3, e176 (2005)) and the following primers: Ec_lepB_usNF, Ec_lepB_CRKan, Ec_lepB_CF-Kan, Ec_lepB_dsCR, Ec_lepB_Nconf, and Kan_ICF2. The kanamycin marked SPase gene was moved into wild type MG1655 by P1 phage transduction. Point mutations at codon 84 of SPase were introduced by amplifying a pair of overlapping DNA fragments from the cassette used for construction of the wild type cassette using primer pairs Ec_lepB_usNF/Ec_lepB_QC_P83x_R and Ec_lepB_QC_P83x_F/Ec_lepB_dsCR. Overlapping PCR of the two fragments yielded the complete SPase/kanamycin resistance cassette containing the desired mutation. P. aeruginosa mutants were constructed using the allelic exchange plasmid pKNG101 using methods described by Kaniga et al. (Gene 109, 137-141 (1991)) and the primers Pa_lepB_usNF3-BamHI and Pa_lepB_dsCR-SpeI. Point mutations were introduced using overlap PCR as described for E. coli with primer pairs Pa_lepB_usNF3-BamHI/Pa_lepB_QC_P84S_R and Pa_lepB_dsCRSpeI/Pa_lepB_QC_P84S_F. S. aureus mutants were constructed using the allelic exchange vector pMAD as described by Arnaud et al. (Appl. Environ. Microbiol. 70, 6887-6891 (2004)) and the primers Sa_spsB_usNF_EcoRI and Sa_spsB_dsCR_EcoRI. Point mutations were introduced using overlap PCR as described for E. coli with primer pairs Sa_spsB_usNF_EcoRI/Sa_spsB_QC_P29S_R and a_spsB_dsCR_EcoRI/Sa_spsB_QC_P29S_F.

### Growth Curves.

For each of the twenty strains of E. coli, each harboring one of the twenty amino acids at SPase residue 84, saturated overnight cultures were diluted 100-fold into fresh LB broth and grown to an OD600 nm of 0.4-0.6. These cultures were diluted into pre-warmed LB to a final density of OD600 nm 0.001 (106 cfu/mL). Growth was measured by plating serial dilutions of the cultures at 30 min. intervals for three hours and counting the resulting colonies. Doubling times were determined from the exponential curve of viable cells versus time. Averages and standard deviations were determined for three independent growth curves.

S. aureus and S. epidermidis Competitive Growth Experiments.

Competitive growth experiments were run in triplicate by diluting equal volumes of saturated S. epidermidis SpsIB(WT) and S. epidermidis SpsIB(S29P) cultures 2000-fold into TSB and growing the resulting culture overnight to saturation (˜10 doublings). This saturated culture was diluted 1000-fold and grown overnight to saturation an additional three to four times for a total of 40 to 50 doublings. Serial dilutions of each saturated culture were plated onto TSA and onto TSA containing 2 μg/mL arylomycin C16 to quantify the number of total cells and arylomycin C16 resistant cells respectively. Similar experiments were performed between wild type S. epidermidis and the S. epidermidis SpsIB(S31P) mutant and between wild type S. aureus and the S. aureus SpsB(P29S) mutant.

### Minimum Inhibitory Concentration (MIC) Experiments

With the exception of C. trachomatis and H. pylori, minimum inhibitory concentrations (MICs) of arylomycin C16 were determined by a modified Clinical and Laboratory Standards Institute (CLSI) micro-broth dilution method in 100 μL of media containing 2-fold dilutions of arylomycin C16. Inocula were formed by resuspending bacteria growing on solid media into the same broth used in the MIC experiment and diluted a final concentration of 1×10⁷ colony forming units/ml. 5 ul of this suspension was added wells containing 100 ul of media and arylomycin C16. MICs of E. coli, P. aeruginosa, K. pneumoniae and Y. pestis were determined in LB. MICs of S. aureus, S. epidermidis, S. haemolyticus, R. equi, R. opacus, C. glutamicum, C. efficiens, B. brevis, B. subtilis, F. tularensis, were determined in Cation-adjusted Mueller Hinton broth. MICs of S. pyogenes, S. agalactiae, and S. pneumoniae were determined in Todd Hewitt broth. MICs of both L. lactis strains were determined in TSYE broth. MICs of arylomycin C16 for L. gasseri, L. acidophilus, and L. plantarum were determined in cation-adjusted Mueller Hinton broth supplemented with 10% Man-Rogosa-Sharpe broth adjusted to pH 6.7 (Klare et al., 2005), and the 96-well plates were incubated a sealed in a jar containing a lit candle to provide a CO₂ enriched atmosphere. MICs of arylomycin for strains of Clostridia and Bacteroidetes were determined using the CLSI approved Wadsworth agar dilution technique. Unless noted otherwise MIC experiments were performed at the optimal growth temperatures of each strain and the MIC determined after 24 hours of growth.

MICs were defined as the lowest arylomycin C16 concentrations yielding no increase in OD_{590 nm} above background after 24 hours of incubation. If the MIC breakpoint was uncertain by OD measurement, viable cells were determined by plating serial dilutions, and MIC was defined as the lowest concentration at which less than 5-fold outgrowth occurred by 24 hours. The MIC of arylomycins C16 for H. pylori were determined by diluting a culture at ˜108 cfu 200-fold into fresh media containing 2-fold dilutions of arylomycin C16. After 24 hours, serial dilutions were plated onto Columbia agar with 5% lysed horse blood and viable cells determined after 5 days of growth. The MIC was defined as the amount of arylomycin C16 required to achieve a 1000-fold decrease in viable cells. To determine MIC of arylomycin C16 for C. trachomatis, HeLa 229 cells were grown to 30% confluence, transferred to 12-well plates treated with 2.0 μg/ml fibronectin, and allowed to adhere overnight. Adherent cells were treated with one infectivity unit of C. trachomatis L2 cells (see, Lad et al., J. Bacteriol. 189: 6619-25 (2007)) and with various concentrations of arylomycin C16. After 24 hours cells were fixed with 3% paraformaldehyde and visualized by fluorescence microscopy using monoclonal antibodies to the chlamydial major outer membrane protein (MOMP) provided by L. M. de la Maza. The MIC was defined as the lowest concentration of arylomycin C16 that resulted in no increase in fluorescence relative to background at 24 hours.

The minimal inhibitory concentration (MIC) of each derivative was determined using a standard broth dilution method. Test strains included wild type S. epidermidis (strain RP62A), as well as mutant strains of S. aureus (strain 8325), E. coli (strain MGI655), and P. aeruginosa (strain PAGI) that were rendered sensitive to the arylomycins by mutation of the resistance-conferring Pro to a residue that does not confer resistance (P29S in the S. aureus protein, and P84L in the E. coli, and P. aeruginosa proteins). MICs were also determined with the isogenic wild type strains of S. aureus, E. coli, and P. aeruginosa, as well as an isogenic mutant strain of S. epidermidis where the resistance-conferring Pro has been introduced (S29P).

### Cloning for Protein Expression.

Plasmids for expression of the Δ2-75 variant of E. coli SPase were constructed by amplifying genomic DNA encoding the Δ2-75 variant of WT and (P83S) E. coli SPase from the corresponding E. coli strains with primers Ec_lepB_Δ2_75 NF_NcoI and Ec_lepB_CR_BamHI and cloning the resulting ORFs into pET15b (Novagen) at the NcoI and BamHI restriction sites, yielding plasmids pET15blepBTrunc and pET15b-lepBTrunc(P83S). The plasmid pET23-lepB for the expression of full length His6×tagged E. coli SPase protein was kindly provided by Dr. Mark Paetzel (Simon Fraser University). To express the P83S variant of E. coli SPase, the LepB(P83S) mutation was introduced into pET23-lepB using QuikChange site directed mutagenesis (Stratagene) with the primers described above yielding plasmid pET23-lepB(P83S). Plasmids for expression of the His6×-tagged S. aureus SPase were constructed by amplifying the genomic DNA encoding SpsB from S. aureus NCTC 8325 and the corresponding SpsB(P29S) chromosomal mutant using primers Sa-SPase-KpnI-F and Sa-SPase-XhoI-R and cloning the resulting DNA was into the plasmid pCDF1 (Novagen) at the KpnI and XhoI restriction sites resulting in the addition of an N-terminal His6×-Tag to the spsB open reading frame.

### Protein Expression.

The E. coli Δ 2-75 SPase proteins were expressed in and purified from BL21(DE3) harboring pET15b-lepBTrunc or pET15b-lepBTrunc(P83S) and stored in a manner similar to that described by Paetzel et al. (Proteins 23, 122-125 (1995)). Full length His-tagged E. coli SPase proteins were expressed in BL21(DE3) containing the plasmid pET23-lepB or pET23-lepB(P83S) and purified as described by Klenotic et al. (J. Biol. Chem. 275, 6490-6498 (2000)), with the exception the Q-column step was omitted and that 1% Elugent (Calbiochem) was used in place of Triton X-100 as the detergent while washing and eluting from the Ni-NTA Superflow resin (Qiagen). Full length His-tagged S. aureus SPase proteins were expressed in and purified from BL21(DE3) containing the plasmid pCDF1-SaSpsB or pCDF1-SaSpsB(P29S) in a manner analogous to the purification of Streptococcus pneumoniae SPase described by Peng et al. (J. Bacteriol. 183, 621-627 (2001)), with the following exceptions. SPase protein was solubilized using 300 mM NaCl, 20 mM Tris pH 8.06, 5 mM imidazole, 10% glycerol, 1% Triton X-100, prior to purification in Ni-NTA Superflow resin and resin bound protein was washed in a similar buffer containing 1% Elugent in place of Triton X-100 prior to protein eluted in wash buffer supplemented with 300 mM imidazole. SDS-PAGE followed by Coomassie staining revealed a single band of approximately 21 kD. All protein concentrations were determined by BCA assay.

### In Vitro KD Measurements

Construction of expression vectors and the subsequent production of the various SPase variants used in this study are described above. Steady state binding of arylomycin C16 was determined by measuring the previously described increase in arylomycin fluorescence (λex=320 nm, λem=410 nm) upon binding E. coli Δ2-75 SPase (see, Paetzel et al., J. Biol. Chem. 279, 30781-30790 (2004)). The binding buffer for full length and truncated E. coli proteins was as follows: 100 mM NaCl, 20 mM Tris-HCl pH 7.4, 1 mM EDTA, 1% n-octyl-β-glucopyranoside (Anatrace). This buffer was supplemented with 10% glycerol for experiments with S. aureus SPase protein.

### SPase Sequence Analysis

The amino acid sequences of the SPases from E. coli, S. aureus, B. fragilis and C. efficiens were concatenated and used as the query sequence in a BLAST against all of the fully sequenced genomes of Bacteriodetes, Actinobacteria, Firmicutes, Proteobacteria, and Chlamydiae/Verrucomicrobia available in the NCBI Microbial Genome Database. The amino acid sequence of BLAST hits with an E-value less than 0.1 were aligned using MUSCLE (Edgar, Nucleic Acids Res. 32, 1792-1797 (2004)), and all sequences lacking the catalytic Ser or Lys residues were removed. Poorly aligned regions were removed using the “Block Mapping and Gathering using Entropy” program found at http://mobyle.pasteur.fr/cgi-bin/portal.py, with Gap Rate Cutoff 0.3 and the Entropy Cutoff of 0.7. Phylogenetic analysis was conducted using PhyML with SPR branch improvement (Guindon and Gascuel, Syst. Biol. 52, 696-704 (2003)). SPases from Gram-positive and Gram-negative organisms were kept separate during alignment and phylogenetic analysis to improve the quality of these analyses.

Also several SPases from the Gram-negative Proteobacteria were removed prior to analysis of SPase phylogeny, since they did not show an obvious relation to any of the other Gram-negative or Grampositive SPases examined. Phylogenetic trees were displayed using the Interactive Tree of Life (Letunic and Bork, Bioinformatics 23, 127-128 (2007)).

### Conservation of Residue 29

A single alignment of SPases from all of the Gram-negative and Gram-Positive genomes examined was made using MUSCLE, and poorly aligned regions were removed using “Block Mapping and Gathering using Entropy” with a gap cutoff of 0.3 and an entropy cutoff of 0.7. The resulting alignment was primarily within the previously described Boxes A-E (Dalbey et al., Protein Sci. 6: 1129-38 (1997)), which are highly conserved across all bacterial SPases. Aligned and trimmed sequences were then grouped by the Phylum of the organisms in which they are found. Each alignment (one for each of the five Phyla) was submitted to the “Score Sequence Conservation” program (Capra and Singh, Bioinformatics 23, 1875-1882 (2007); http://compbio.cs.princeton.edu/conservation/score.html), and the sequence conservation was scored using the Jensen-Shannon divergence method with weighted sequences and a window size of 1. The per residue conservation scores were averaged across the entire alignment, across each set of residues comprising Boxes B-E, and across the regions five amino acids centered on residue 29.

16sRNA Sequence Analysis

Aligned 16sRNA sequences were analyzed were obtained from the Ribosomal Database Project (Cole et al., Nucleic Acids Res. 37, D141-145 (2009)). The “Block Mapping and Gathering using Entropy” program was used with a Gap Rate Cutoff of 0.7 and an Entropy Cutoff of 0.7 to remove poorly aligned regions. Phylogenetic analysis was performed using PhyML 3.0, with the HKY85 substitution model and SPR tree improvement, and the resulting tree was displayed using the Interactive Tree of Life.

### Example 3

### The Antibiotic Activity of Arylomycins is Masked by SPase Mutations

The Example describes experimental results demonstrating that many strains of bacteria have naturally developed SPase mutations that confer resistance to arylomycins.

### Point Mutations in SPase Confer Arylomycin Resistance

S. epidermidis is atypical in its sensitivity to the arylomycins (Roberts et al., J. Am. Chem. Soc. 129: 15830-15838 (2007)). To investigate whether S. epidermidis lacks specific resistance mechanisms inherent to other bacteria, selection experiments were performed to isolate mutants that were able to grow in the presence of 2 μg/ml arylomycin C16 (8×MIC). Mutants were obtained at a frequency of 4 per 109 viable cells and fell into two phenotypic classes: the majority (˜75%) had a 32-fold elevated MIC compared to the wild type strain, and the remainder had a greater than 256-fold elevated MIC. Consistent with this low frequency of resistance, arylomycin resistance was correlated with either of two mutations in SpsIB, one of the two SPases found in S. epidermidis. The 32-fold increase in resistance was associated with a Ser to Pro mutation at position 29 (10/11 clones sequenced); while the >256-fold increase in resistance was associated with a Ser to Pro mutation at position 31 (9/11 clones sequenced). None of the resistant mutants in either class exhibited growth defects under the standard laboratory conditions employed (data not shown).

These data indicate that the whole cell antibiotic activity of the arylomycins results from their inhibition of SPase and also that mutations in SPase are the dominant mechanism whereby S. epidermis evolves resistance.

To investigate whether naturally resistant bacteria harbor the same mutations that confer resistance in S. epidermidis, the amino acid sequences of SPases were examined in the closely related organism S. aureus, as well as in the more distantly related Gram-negative organisms E. coli and P. aeruginosa (Table 11).

TABLE 11
Resistance to Arylomycin A2 C16
Correlates with Proline at Position -5 and/or -7 in Bacterial SPase*

| Bacterial Strain | SPase Type | SPase Alignment | MIC (μg/mL) |
|---|---|---|---|
| S. epidermidis RP62A | WT | VGKSYSIKGDS | 0.25 |
| S. epidermidis PAS9001 | S29P | 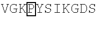 | 8 |
| S. epidermidis PAS9002 | S31P | 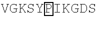 | >128 |
| S. aureus NTCT 8325 | WT | VAKPYTVKGDS | >128 |
| S. aureus PAS8001 | P29S | 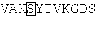 | 2 |
| E. coli MG1655 | WT | IYEPFQIPSGS | >128 |
| E. coli PAS0232 | P84S | 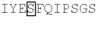 | 2 |
| P. aeruginosa PAO1 | WT | LFEPFQIPSGS | >128 |
| P. aeruginosa PAS2006 | P84S | 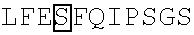 | 8 |

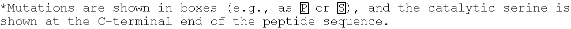

As shown in Table 11, at the position corresponding to the S. epidermidis SPase residue 29, Pro is found in the single SPase of S. aureus, in the single SPase of E. coli, and in one of the two SPases of P. aeruginosa (Pro29 in S. aureus, Pro84 in E. coli and P. aeruginosa). However, at the position corresponding to SPase residue 31 in S. epidermidis, Pro is not found in any of these S. aureus, E. coli, and P. aeruginosa SPase sequences. Moreover, an E. coli strain with Pro at Spase position 31 could not be constructed, suggesting that SPase position 31 mutations are not tolerated in some organisms.

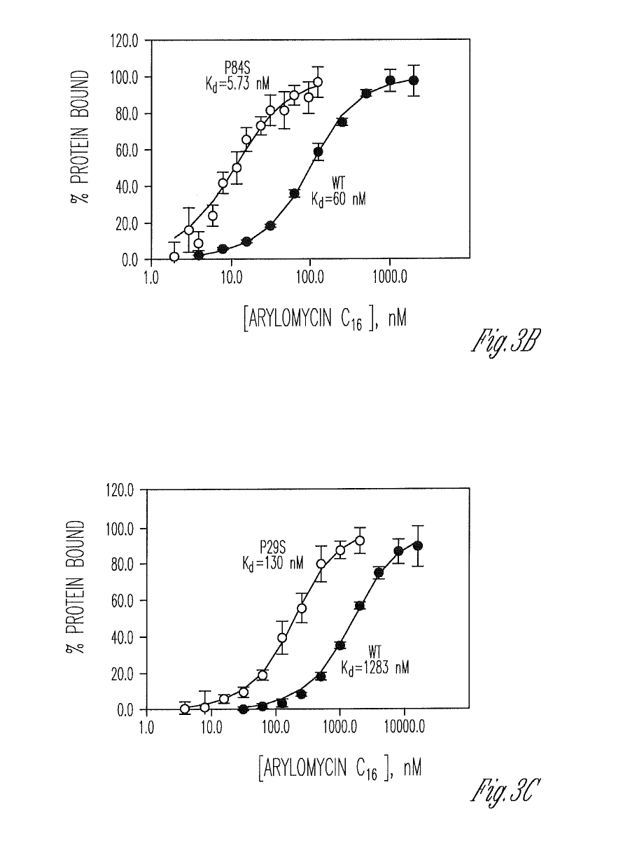

To determine whether the innate arylomycin resistance observed in E. coli, P. aeruginosa, and S. aureus results from the identified Pro residues, mutant strains of these bacteria were constructed in which the Pro was replaced by Ser (the corresponding residue in wild type S. epidermidis SpsIB). In each organism, mutation of Pro to Ser conferred a high degree of sensitivity to arylomycin C16 (Table 11). No growth defects were apparent in the mutant strains (FIG. 2), suggesting that the increased sensitivity does not result from decreased fitness or compromised SPase activity.

The sensitivity of the E. coli and P. aeruginosa serine mutants to arylomycin C16 indicates that the presence of the Pro residue correlates with arylomycin resistance and that the arylomycins penetrate the formidable outer-membrane of Gram-negative bacteria. Consistent with efficient outer-membrane penetration, experiments demonstrated that permeabilizing these bacteria with polymyxin B nonapeptide had only a negligible effect on the MIC values (≦4-fold decrease).

To determine whether the identified Pro is unique in its ability to confer arylomycin resistance, mutant strains of E. coli were constructed in which each of the other 19 amino acids was introduced into the E. coli SPase at the same position (residue 84). Based on the growth rates observed in arylomycin-free media, most amino acids at this position were well tolerated (FIG. 2), indicating that the nature of the amino acid at residue 84 does not generally affect fitness under the conditions employed, although a minor growth defect was observed when Arg, Lys, Glu, and Cys was present at position 84 and a temperature sensitive phenotype was observed when His and Phe were present at position 84. In contrast, when arylomycin C16 was added to the media, the MIC values observed are highly dependent on the identity of the amino acid at residue 84, but Pro was the only amino acid that imparted high-level arylomycin resistance (MIC>256 μg/ml) (FIG. 2). All of the other amino acids lead to arylomycin sensitivity (MIC values of ≦16 μg/ml), with the hydrophobic amino acids conferring somewhat greater sensitivities.

### Resistance-Conferring Mutations Reduce the Affinity of Arylomycin for SPase

Based on the previously reported crystal structure of the E. coli SPase complex (Paetzel et al., J. Biol. Chem. 279, 30781-30790 (2004))arylomycin A2 binds in a manner that mimics that proposed for natural peptide substrates, and the resistance-conferring Pro residue (Pro84), is positioned within the E. coli SPase substrate binding pocket, but distal to the catalytic residues (FIG. 3A). To test whether the resistance-conferring mutations directly interfere with arylomycin C16 binding in vitro, equilibrium binding constants were determined using recombinant SPase enzymes reconstituted in micelles that mimic a lipid bilayer environment. The affinities of arylomycin C16 were measured for a truncated wild type E. coli SPase that lacks the N-terminal membrane helices but that still associates with micelles, and for a P84S variant of this truncated E. coli SPase enzyme, (Kuo et al., Arch. Biochem. Biophys. 303, 274-280 (1993)). FIG. 3D shows the binding affinity of arylomycin C16 for the wild type and P84S variants of the soluble N-terminally truncated E. coli SPase. Arylomycin C16 bound the truncated wild type protein with a KD of 979±69 nM, which is similar to the value reported for arylomycin A2 (Paetzel et al., Biol. Chem. 279, 30781-30790 (2004)). In contrast, a significantly lower KD of 39±15 nM was observed for the serine-containing P84S variant of this E. coli SPase.

To control for artifacts associated with deletion of the N-terminal helices, which might interact with the lipid tail of the inhibitor or help to co-localize the protein and the inhibitor within the membrane-like micellular environment, the affinities of arylomycin C16 for the wild type and P84S variants of a detergent-solubilized full-length E. coli SPase were determined (FIG. 3B). While arylomycin C16 bound the full-length proteins with higher affinities than the corresponding soluble fragments, the affinity of arylomycin for the full-length Ser-variant (KD=5.7±1.0 nM) was again an order-of-magnitude higher than that for the corresponding Pro-variant (KD=60±16 nM).

Lastly, to characterize a representative Gram-positive SPase, the affinity of arylomycin C16 for the full-length wild type and P29S mutant of S. aureus SPase was measured (FIG. 3C). As with the E. coli SPase, arylomycin C16 bound the Ser-variant of S. aureus SPase an order-of-magnitude more tightly than the Pro-variant, with KD values of 130±53 and 1283±278 nM, respectively. Thus, the Pro residues responsible for resistance in E. coli and S. aureus appear to act by interfering with arylomycin binding.

### Distribution of Resistance-Conferring Residues in Nature

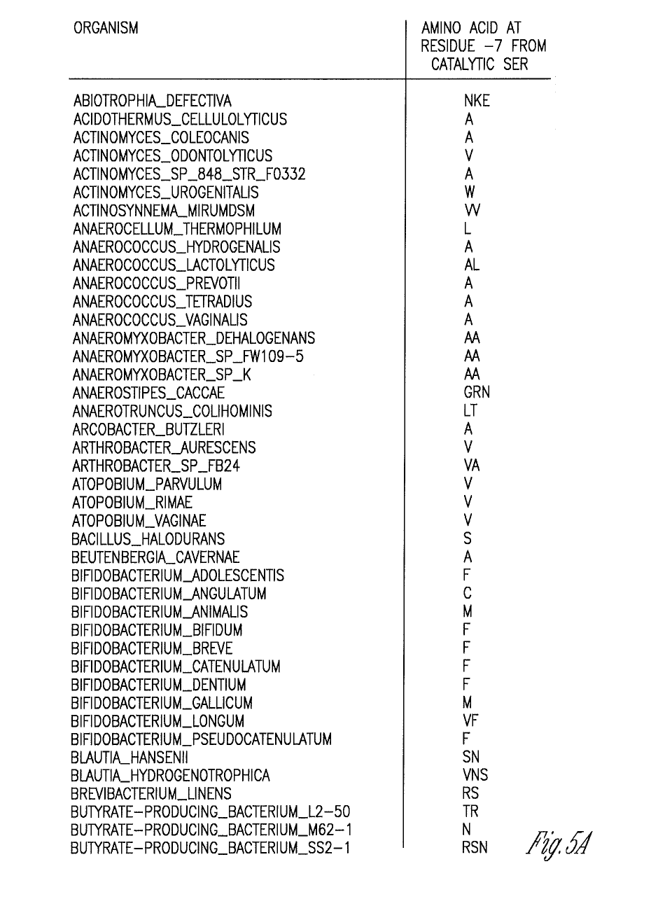

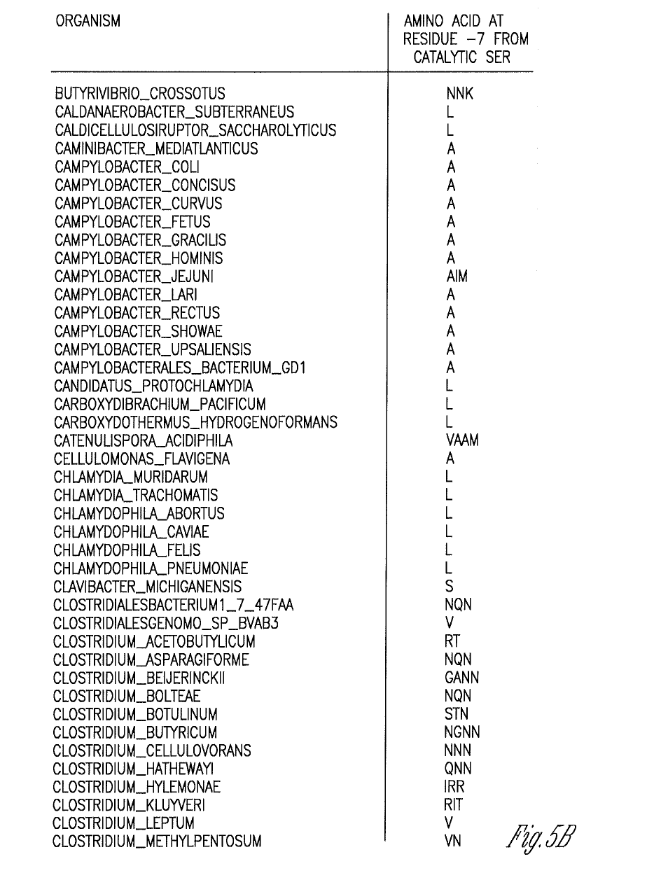

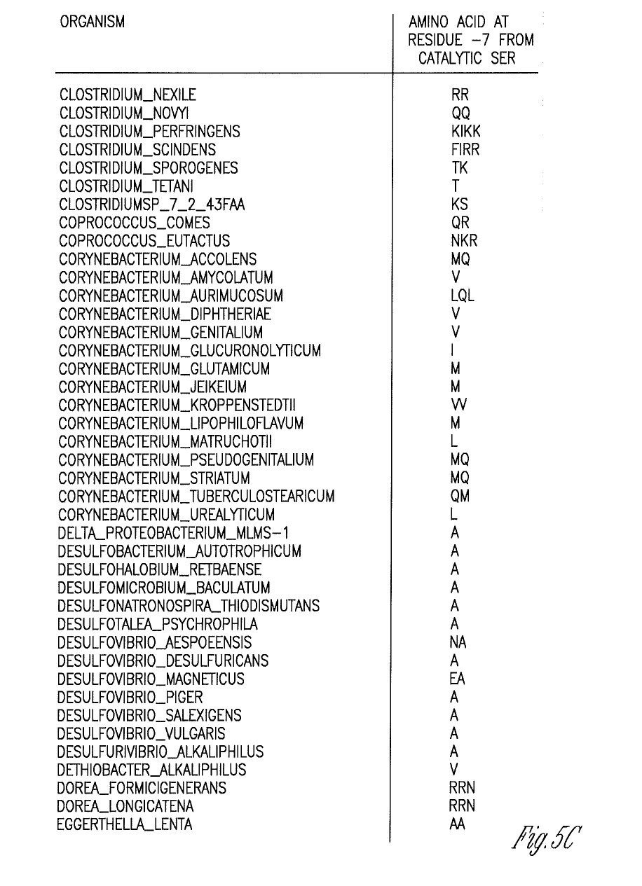

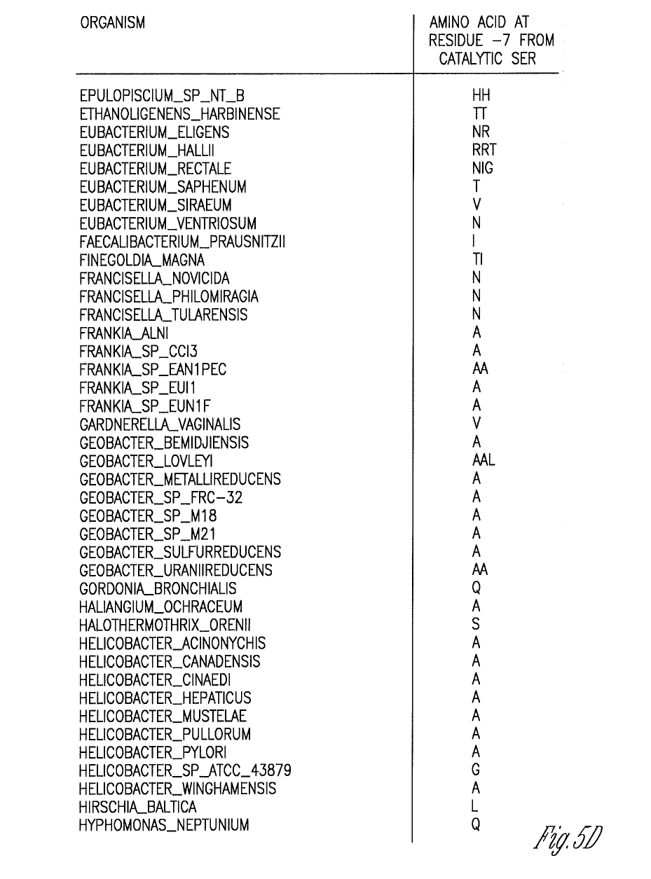

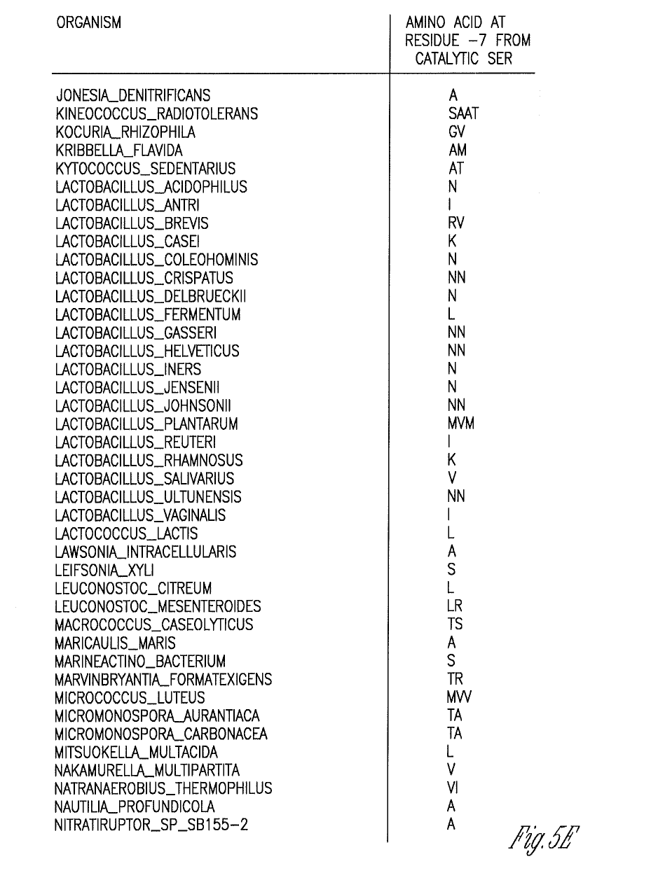

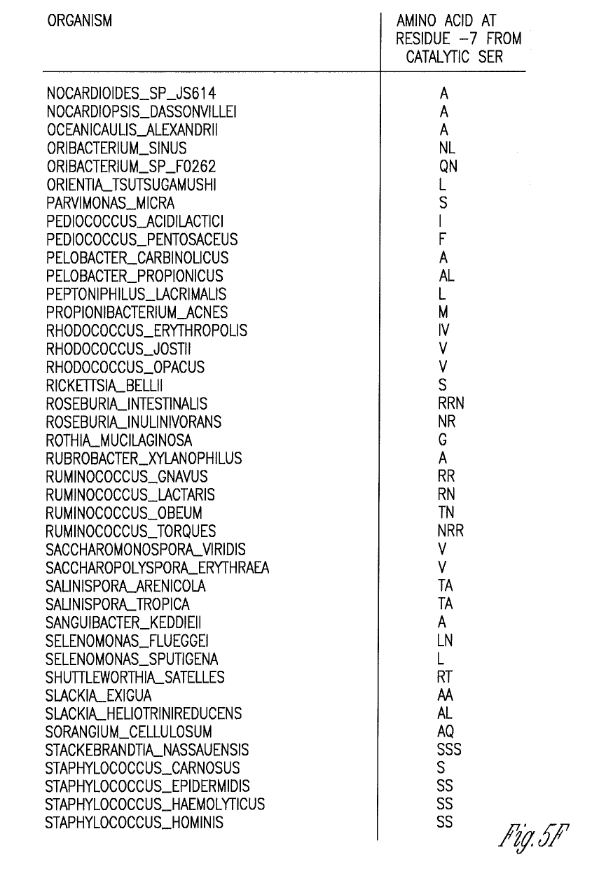

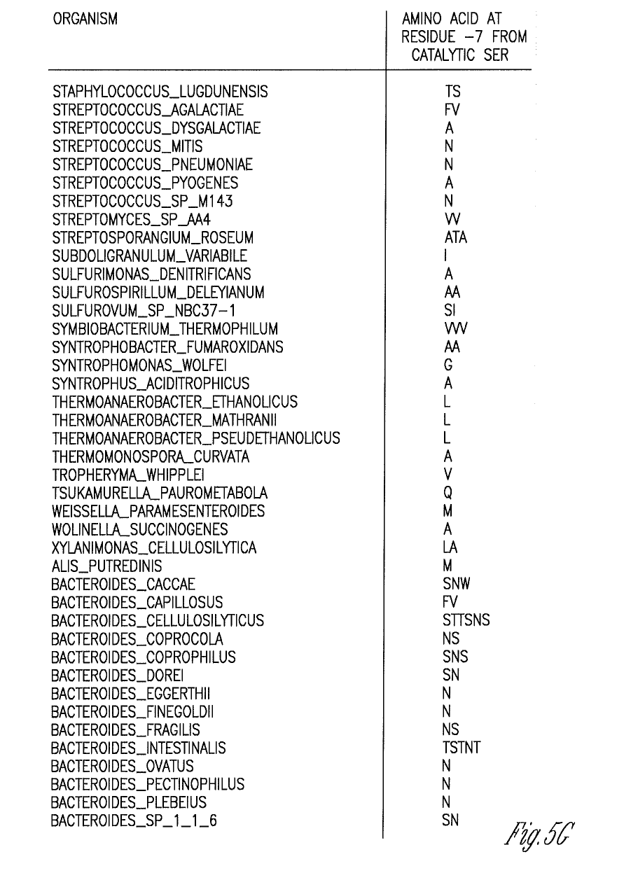

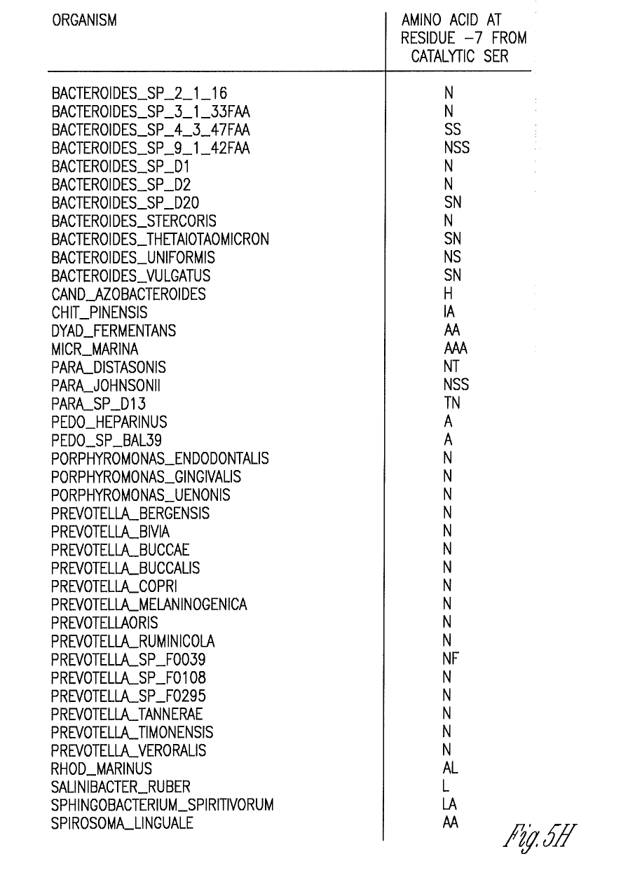

To better understand the distribution of this resistance determinant in nature, the phylogenetic relationship of the fully sequenced bacteria from five phyla was determined, as reflected by their 16S rRNA sequences. This phylogeny was then compared to the number of SPases in each organism and to the presence or absence of Pro at the position corresponding to residue 29 in S. epidermidis (unless otherwise specified, S. epidermidis numbering is used hereafter). In general, Gram-negative bacteria from the Chlamydiae/Verrucomicrobia, Proteobacteria, and Bacteroidetes phyla have a single SPase, and in each phylum, Pro29 is present in the SPases of a subset of organisms (FIG. 5).

Accordingly, almost all of the sequenced α-, β-, γ-Proteobacteria have SPases with Pro29 (115/123, 64/65, and 178/183 of the sequenced organisms, respectively), whereas most of the sequenced δ- and ε-Proteobacteria have SPases with Ala29 (32/35 and 27/29, respectively). Similarly, within the Bacteroidetes phylum, each of the sequenced Flavobacteria has one SPase where Pro is always present at position 29, whereas each of the Bacteroidia typically has an SPase with Asn29 and sometimes a second SPase with Ser29. Finally, among the few Chlamydiae/Verrucomicrobia that have been sequenced, each of the Chlamydia has one SPase with Leu29 (7/7), while each of the Verrucomicrobia has at least one SPase with Pro29 (8/8). The phylogeny of the SPase genes themselves largely mirrors that of the 16S rRNA sequences, indicating that relatively little horizontal transfer of SPase genes has occurred and confirming that Pro29 was installed independently into the SPases of these lineages. Interestingly, horizontal gene transfer is responsible for the scattered instances of Proteobacteria that do encode multiple SPases, including the second SPase of P. aeruginosa that has at Leu29. These additional SPases are not closely related to any of the sequenced SPases examined in this analysis, and they may not be functionally equivalent to the other Proteobacterial SPases.

In contrast to the Gram-negative bacteria, the Gram-positive Firmicutes and Actinobacteria commonly encode multiple SPases, and comparison of the 16S rRNA and SPase phylogenies indicates that duplication of SPase genes has occurred multiple times in these lineages (FIG. 5). The distribution of Pro29 is also more irregular, which appears to have resulted from reduced conservation leading to the frequent introduction and removal of Pro at this position.

Moreover, the region defined by residues 27-31 appears to be poorly conserved within the Gram-positive SPases relative to the same region in the Gram-negative proteins or to the regions that comprise the core and active site of the protein (Table 12). Table 13 shows the relative conservation of regions of SPase genes from five bacterial phyla as reflected by the average Jensen-Shannon divergence score of the comprising residues (Capra and Singh, Bioinformatics 23, 1875-1882 (2007)). Boxes B-E were defined previously as the conserved regions that form the substrate binding site and active site (Dalbey et al., Protein Sci. 6: 1129-38 (1997)). The rank of residue 29 indicates the degree of conservation at this position relative each the other 137 residues used in the SPase sequence alignment, where 1 indicates the most conserved residue.

TABLE 12
Conservation of Regions of SPase genes from Five Bacterial Phyla

| | Entire | | | | | | Rank |
|---|---|---|---|---|---|---|---|
| Bacterial | Aligned | | | | | Residues | Residue |
| Phyla | Sequence | Box B | Box C | Box D | Box E | 27-31 | 29 |
| Actinobacteria | 0.473 | 0.572 | 0.464 | 0.632 | 0.602 | 0.416 | 76/137 |
| Firmicutes | 0.431 | 0.491 | 0.370 | 0.626 | 0.587 | 0.248 | 134/137 |
| Bacteroidetes | 0.516 | 0.615 | 0.529 | 0.645 | 0.594 | 0.431 | 98/137 |
| Chlamydia/ | 0.553 | 0.681 | 0.599 | 0.636 | 0.616 | 0.567 | 47/137 |
| Verrucomicrobia | | | | | | | |
| Proteobacteria | 0.534 | 0.629 | 0.585 | 0.654 | 0.664 | 0.568 | 30/137 |

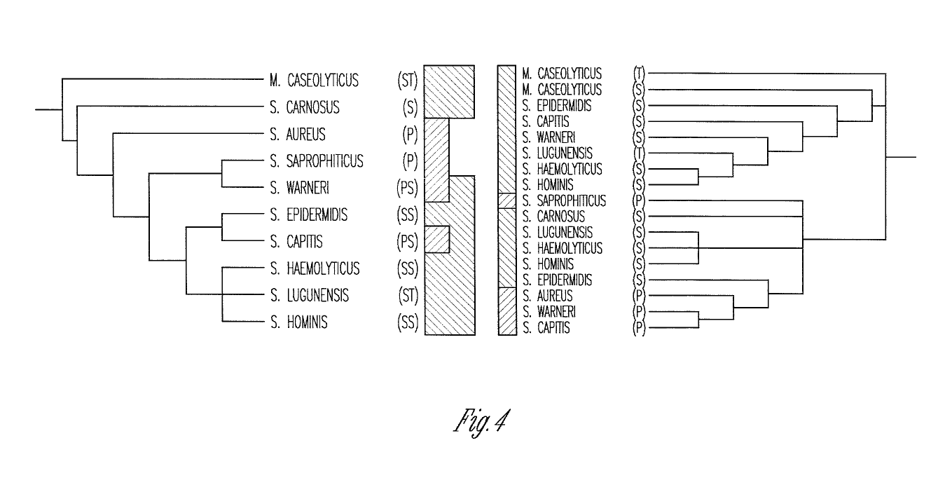

As shown in Table 12, the region defined by residues 27-31 appears to be poorly conserved within the Gram-positive SPases relative to the same region in the Gram-negative proteins. However, Pro29 is particularly common among the SPases of a subset of the Gram-positive bacteria Bacilli, including the genres Bacillus, Listeria, and Staphylococcus. Interestingly, although SPases with Pro29 appear to have been present and maintained during speciation of the Bacillus and Listeria, the common Staphylococci ancestor appears to have had two SPases each with Ser29, as is still the case with S. epidermidis. S. aureus appears to have deleted one SPase and introduced Pro29 into the other (FIG. 4).
The Arylomycins have a Broad Spectrum of Antibiotic Activity

To further explore the spectrum of arylomycins and to test the contribution of the Pro29 to arylomycin resistance in a wider range of bacteria, the arylomycin susceptibilities were determined of representative organisms from the above phylogenetic analysis (Table 13). Bacteria from all five phyla were sampled, and when possible important human pathogens were included.

TABLE 13
Associations between arylomycin C16 sensitivity and
SPase genotype(s) among different wild type bacteria

| Species | Reside 29 | MIC (μg/ml) |
|---|---|---|
| Staphylococcus epidermidis | S,S | 0.25 |
| Staphylococcus haemolyticus | S,S | 2 |
| Rhodococcus opacus | V | 2 |
| Corynebacterium glutamicum | M | 2 |
| Helicobacter pylori | A | 4 |
| Yersinia pestis | P | 4 |
| Chlamydia trachomatis | L | 6 |
| Francisella tularensis | N | 4-16, >64* |
| Streptococcus pneumoniae | N | 16 |
| Streptococcus pyogenes | A | 16 |
| Lactococcus lactis | L | 16, >128* |
| Rhodococcus erythropolis | V,I | 16 |
| Corynebacterium efficiens | P | >64 |
| Staphylococcus aureus | P | 16-32, >128* |
| Brevibacillus brevis | P,P,P,P,V | >64 |
| Enterococcus faecalis | P,P,P,S | >64 |
| Bacillus subtilis | P,P,P,D | >128 |
| Streptococcus agalactiae | F,V | >128 |
| Escherichia coli | P | >128 |
| Pseudomonas aeruginosa | P,L | >128 |
| Klebsiella pneumoniae | P | >128 |
| Lactobacillus gasseri | N,N | >128 |
| Lactobacillus acidophilus | N | >128 |
| Lactobacillus plantarum | M,M,V | >128 |
| Clostridium difficile | P,P,P | >16 |
| Clostridium bolteae | N,N,Q | >16 |
| Clostridium perfringens | K,K,K,I | >16 |
| Bacteroides fragilis | S,N | >16 |
| Prevotella copris | N | >16 |

*Multiple values indicate heterogeneity within different strains of a species, as discussed in the text.

As shown in Table 13, arylomycin C16 is active against the ε-Proteobacteria H. pylori (whose SPase has Ala29) with an MIC of 4 μg/ml. Similarly, the intracellular Gram-negative pathogen C. trachomatis (Leu29) is eradicated from human HeLa 229 cells with an MIC of 6 μg/ml. Notably, no adverse effects on the human cells were observed up to 20 μg/ml of arylomycin, the highest concentrations examined. Francisella tularensis (Asn29) is a potential biological warfare agent and a member of the only genera of γ-Proteobacteria that does not have Pro29. Nineteen clinical isolates of Francisella tularensis (Asn29) were examined and it was determined that 8 were inhibited with MICs of 4 to 16 μg/ml, one with an MIC of 32 μg/ml, and the remainder with MICs in excess of 64 μg/ml. Klebsiella pneumoniae encodes a single SPase that has Pro29 and is resistant to the arylomycins. Interestingly, although it has a single SPase with a Pro29, Yersinia pestis, the causative agent of plague, is sensitive to arylomycin C16.

The Gram-positive Firmicutes Streptococcus pneumoniae, Streptococcus pyogenes, and Staphylococcus haemolyticus are all human pathogens that lack SPases with Pro29. Each of these species is sensitive to arylomycin C16. However, B. subtilis and E. faecalis, Firmicutes with multiple SPases having Pro29, are resistant. In contrast, the Gram-positive Actinobacteria Rhodococcus equi and Rhodococcus opacus (Schimana et al., J. Antibiot. (Tokyo) 55, 565-570 (2002)) lack Pro29 and are each sensitive to arylomycin C16. While the actinobacteria Corynebacterium glutamicum has a single SPase with Met29 and an arylomycin C16 MIC of 2 μg/ml, the related actinobacteria Corynebacterium efficiens has a single SPase with Pro29 and an MIC of 16 μg/ml.

However, while Lactococcus lactic spp. cremonis, which has one SPase with Leu29, is sensitive to arylomycin C16, the highly related Lactococcus lactic spp. lactii also has a single SPase with Leu29 and is resistant. Additionally, a variety of other Lactobacillales, and all investigated Clostridia and Bacteriodetes, are resistant to the arylomycins despite the fact that many lack SPases with Pro29 (MIC>64 μg/ml for the Lactobacillales and >16 μg/ml for the Clostridia and Bacteriodetes). Finally, a broader survey of S. aureus strains revealed that two strains, despite a prediction of arylomycin-resistance because they have SPases with Pro29, are actually arylomycin-sensitive: COL a tetracycline and penicillin resistant strain (MIC=12 μg/ml), and Rosenbach 328, an MRSA strain (Pantosti and Venditti, Eur. Respir. J. 34, 1190-1196 (2009)) (MIC=25 μg/ml).

Table 14 shows the activity of arylomycin A-C₁₆ and arylomycin B—C₁₆ against Streptococcus pnemoniae, Streptococcus pyogenes, and Streptococcus agalactiae.

TABLE 14

| | MIC | MIC |
|---|---|---|
| | (μg/ml) arylomycin | (μg/ml) arylomycin |
| Strain | A-C16 | B-C16 |
| S. pneumoniae | 8 | 16 |
| S. pyogenes | 8 | 4 |
| S. agalactiae | >128 | 8 |

Representative SPase Sequences of Arylomycin-Susceptible Bacteria
1_Rhodococcus equi ATCC 33707_GI# 296036237_Catalytic
Ser Residue 69
VADAPQDPDVTPDETEQEQTGGRSRRRRGKDKKPRSFWREIPILIVVALLLSFLLQTFIA
RVYLIPSESMEPTLHGCPGCTGDRIVVEKISYRFGDPKPGDVVVFRGPESWSEGYSSTRS
DNVVVRGLQEVGSLVGVVPPDENDLVKRVIATGGQTVECCDDQGRVLVDGKPLDEPYITM
DFPFIPGVQTCDTAVKSGRCFGPVTVPDGHLWVMGDNRSNSADSRYHVSDEMQGTIPVDN
VIGKATFIVLPPGRWGSISSPDIRQQ*
1_Rhodococcus equi ATCC 33707_GI# 296036237_Catalytic
Ser nucleotides 205-207
GTGGCAGATGCACCGCAGGACCCGGACGTGACGCCGGACGAGACCGAGCAGGAGCAGACC
GGGGGACGGTCCCGCAGGCGCAGGGGGAAGGACAAGAAACCTCGATCGTTCTGGCGTGAG
ATCCCGATCCTCATCGTCGTCGCCTTGCTGTTGAGTTTCCTGCTGCAGACGTTCATCGCC
CGGGTCTACCTCATTCCGTCCGAGTCGATGGAGCCGACGCTGCACGGTTGCCCCGGGTGC
ACCGGTGACCGCATCGTCGTCGAGAAGATCAGTTACCGATTCGGCGACCCGAAGCCCGGC
GACGTCGTGGTCTTCCGCGGCCCGGAGTCGTGGTCCGAGGGATATTCGTCGACACGCTCG
GACAACGTGGTGGTCCGCGGTCTGCAGGAGGTCGGCTCGCTCGTCGGTGTCGTGCCGCCG
GACGAGAACGATCTCGTCAAACGCGTCATCGCGACGGGTGGGCAGACCGTCGAGTGCTGC
GACGACCAGGGCCGTGTCCTGGTCGACGGAAAGCCGCTCGACGAGCCGTACATCACGATG
GACTTCCCTTTCATCCCCGGCGTGCAGACGTGTGACACCGCCGTGAAGTCCGGACGCTGC
TTCGGACCCGTCACGGTTCCCGATGGGCACCTGTGGGTGATGGGCGACAACCGCAGCAAT
TCCGCGGATTCGCGGTACCACGTCTCCGACGAGATGCAGGGCACGATTCCGGTGGACAAT
GTGATCGGTAAGGCGACCTTCATCGTCCTGCCCCCGGGCCGGTGGGGATCGATCTCGTCT
CCCGACATCCGGCAGCAGTGA
2_Rhodococcus opacus B4_GI# 226366004_Catalytic Ser
Residue 72
VTDSSKERALSSESETTGDSAATSAVNGGAAETEKKPRSFLRELPILILVALVLSFLLQT
FVARVYLIPSESMEPTLHGCAGCTGDRIVVEKIGYRFGDPQPGDVIVFRGPDSWSQDFVS
TRSSNVVIRGAQEVGSLVGLVPPDENDLVKRVIATGGQTVECCDDQGRILVDGQPIDEPY
VVMDFPFVPGSQACDTALKSARCFGPVTVPEGHLWVMGDNRSNSADSRYHVGDDMQGTIP
LDNVIGKAVFIALPPSRMGTISSPDIQGK*
2_Rhodococcus opacus B4_GI# 226366004_Catalytic Ser
nucleotides 214-216
GTGACAGATTCTTCGAAGGAGCGGGCATTGTCGTCGGAATCCGAGACCACCGGCGATTCG
GCCGCCACCTCCGCAGTGAACGGCGGTGCGGCGGAGACCGAGAAGAAACCCCGCTCCTTC
CTCCGCGAGTTGCCGATCCTGATCCTGGTCGCGCTCGTCCTGAGTTTCCTGCTGCAGACG
TTCGTCGCCCGCGTGTATCTCATTCCGTCGGAGTCGATGGAACCGACGCTGCACGGGTGC
GCGGGCTGCACCGGCGACCGCATCGTGGTCGAGAAGATCGGCTACCGTTTCGGGGACCCG
CAACCCGGTGACGTCATCGTGTTCCGCGGGCCCGACTCGTGGTCACAGGATTTCGTCTCC
ACCCGTTCCTCCAACGTGGTGATCCGCGGTGCGCAGGAAGTCGGTTCCCTCGTCGGACTC
GTCCCGCCGGACGAGAACGACCTCGTCAAGCGTGTGATCGCCACCGGCGGTCAGACCGTC
GAATGCTGCGACGACCAGGGCCGCATCCTGGTGGACGGACAACCGATCGACGAGCCCTAC
GTCGTCATGGACTTCCCCTTCGTCCCCGGCTCCCAGGCCTGCGACACGGCGCTGAAGTCG
GCGCGCTGCTTCGGTCCCGTCACCGTCCCCGAGGGGCACCTGTGGGTGATGGGCGACAAC
CGCAGCAACTCCGCGGACTCCCGCTACCACGTCGGCGACGACATGCAAGGCACCATCCCG
CTCGACAACGTGATCGGCAAGGCGGTCTTCATCGCGTTGCCGCCGTCGCGAATGGGCACG
ATCAGTTCACCCGATATCCAGGGCAAGTGA
3_Corynebacterium diphtheriae NCTC 13129_GI#
38234095_Catalytic Ser Residue 90
MKRSVFSFCMMQQASLGVFHSMAETAARVLKVSSANNETVSPTEGVETHDKEKKQLPWFV
EIPVVVVVTLLVITLLQTFVGRVYMIPSQSMEPTLHGCAGCTGDRIYVDKLAYRFGEPEA
GDVVVFAGTESWNTGFTTSRSENPLVRGIQNAGAFVGLVAPDENDLVKRIVATGGQTVQC
LEGDEGVKVDGKVIDSSYTLMPPAYPVDQTTGSEACGGFYFGPIKVPEGNYFMMGDNRTN
SADSRYHIGDQYQGTIPKENLKGKVQFKIFPFNRIGAVEDYDIQQ*
3_Corynebacterium diphtheriae NCTC 13129_GI#
38234095_Catalytic Ser nucleotides 268-270
ATGAAGCGCTCAGTTTTCTCTTTTTGTATGATGCAGCAAGCGTCGCTGGGCGTTTTTCAT
TCGATGGCTGAAACAGCTGCTAGAGTTCTCAAAGTGAGTTCAGCTAATAACGAGACTGTG
TCCCCCACGGAAGGCGTCGAAACGCACGACAAGGAAAAGAAGCAACTGCCATGGTTTGTG
GAAATCCCTGTCGTCGTAGTGGTGACCCTTCTTGTGATCACCTTGCTTCAAACGTTCGTT
GGACGGGTCTATATGATCCCAAGTCAGTCAATGGAGCCGACACTTCATGGATGTGCAGGG
TGTACCGGAGACCGAATTTATGTAGATAAGCTGGCTTATCGTTTTGGTGAACCAGAAGCC
GGCGACGTTGTAGTTTTTGCAGGTACAGAATCATGGAACACCGGATTTACCACTTCACGG
TCAGAAAATCCTCTGGTTCGTGGAATACAAAATGCGGGTGCTTTCGTCGGATTAGTAGCA
CCAGACGAAAACGACCTTGTAAAACGCATCGTAGCAACAGGGGGTCAAACGGTGCAGTGC
CTTGAAGGCGATGAAGGTGTCAAAGTAGACGGTAAAGTCATCGACTCGTCATATACTCTG
ATGCCACCAGCGTATCCGGTCGACCAGACCACAGGATCAGAGGCGTGCGGCGGCTTTTAC
TTCGGACCTATCAAGGTACCTGAAGGAAATTACTTCATGATGGGCGATAACCGGACAAAC
TCCGCGGATTCTCGTTACCACATTGGTGATCAGTATCAAGGCACCATCCCTAAAGAAAAC
CTCAAGGGGAAAGTTCAGTTCAAGATTTTCCCATTTAACCGTATTGGTGCAGTCGAGGAT
TACGATATCCAACAGTGA
4_Lactococcus lactis subsp. cremoris MG1363_GI#
125625303_Catalytic Ser Residue 35
MMKFLKEWGLFIFIIAAVLLSRVFIWSLVVVDGHSMDPTLADKERLVIVRTTKINRFDIV
VAKENAADGSTKDIVKRVVGMPGDTIKFDHDQLTINNKVYPENYLKDYQKQLADGQLEKT
YGNYPLTKALTDQNRSLFVSLAQSTKAFTTDSTGNPTFTVKVPDGQYFLMGDNRVVSQDS
RAVGSFKRSAIIGEAKLRVWPLNKISFF*
4_Lactococcus lactis subsp. cremoris MG1363_GI#
125625303_Catalytic Ser nucleotides 103-105
ATGATGAAATTTTTAAAAGAATGGGGATTATTTATCTTTATAATTGCCGCTGTCCTTCTC
TCGCGCGTCTTTATTTGGTCACTAGTTGTCGTTGATGGCCATTCAATGGACCCTACTTTA
GCCGATAAAGAAAGACTTGTAATTGTTAGAACGACAAAAATTAATCGTTTTGATATTGTA
GTTGCTAAAGAAAACGCGGCTGATGGTTCAACCAAAGATATTGTCAAACGTGTCGTTGGG
ATGCCTGGGGACACTATAAAATTCGACCATGACCAACTTACTATCAATAATAAGGTTTAT
CCAGAAAACTATCTCAAAGACTATCAAAAACAATTGGCTGATGGTCAATTGGAAAAAACT
TACGGGAACTATCCTTTGACAAAAGCATTAACTGATCAAAATCGTAGTTTATTTGTAAGC
TTAGCTCAGAGCACCAAAGCTTTTACAACGGATAGTACTGGTAATCCAACCTTTACAGTC
AAAGTCCCTGACGGACAATACTTCTTGATGGGAGATAATCGTGTTGTGTCTCAAGATAGC
CGAGCAGTTGGAAGTTTCAAACGTTCAGCGATTATTGGTGAAGCCAAATTACGAGTTTGG
CCACTCAATAAAATTTCTTTCTTTTAA
5_Corynebacterium glutamicum ATCC 13032_GI#
19553237_Catalytic Ser Residue 67
VTDFSSASNADDSTQDGRPGRRAGKSKKESKPTPWYIEIPVVVVLTLALIFVLQTFVGRM
YMIPSGSMEPTLHGCEGCTGDRILVEKVSYYFTDPEPGDVVVFKGTDSWNVGFTTQRSDN
SVIRGLQNLGSYVGLVAPDENDLVKRIIATGGQTVSCQAGDPGIMVDGKEVDDSYTLQPA
QFPIDETSGSTECGGNYFGPITVPGGNYFMMGDNRTNSMDSRYHLGDQYQGTIPEENIKG
KVQAIILPFSRIGGVDDPAIKG*
5_Corynebacterium glutamicum ATCC 13032_GI#
19553237_Catalytic Ser nucleotides 199-201
GTGACTGATTTTTCTAGTGCTTCAAATGCTGACGATTCCACGCAGGACGGTCGTCCTGGT
CGACGTGCTGGAAAGTCTAAGAAGGAATCGAAGCCAACTCCGTGGTACATCGAAATTCCA
GTGGTTGTGGTTTTGACCCTCGCGCTGATTTTCGTGCTCCAGACGTTTGTCGGACGCATG
TACATGATTCCGAGTGGTTCGATGGAACCTACTTTGCACGGATGTGAGGGCTGCACGGGT
GACCGCATCCTGGTGGAGAAGGTTTCTTACTACTTCACGGATCCAGAGCCGGGCGATGTT
GTGGTGTTCAAGGGTACTGATTCCTGGAACGTTGGATTCACTACGCAGCGTTCCGATAAT
TCGGTGATCCGCGGCCTGCAGAACCTGGGTTCTTACGTGGGTCTTGTCGCACCTGATGAA
AATGACCTGGTCAAGCGCATTATCGCCACCGGCGGTCAGACTGTTTCGTGCCAAGCCGGT
GATCCTGGAATCATGGTTGACGGCAAGGAAGTCGATGACAGCTACACGCTGCAACCTGCG
CAATTCCCCATCGATGAGACCTCCGGTTCCACCGAATGCGGCGGCAACTATTTCGGCCCC
ATCACCGTGCCTGGCGGCAACTACTTCATGATGGGTGACAACCGCACCAACTCCATGGAT
TCCCGCTACCACCTGGGCGATCAGTACCAAGGAACCATCCCTGAGGAAAACATCAAGGGC
AAAGTTCAAGCAATTATCCTGCCATTTAGCCGAATCGGTGGCGTCGACGACCCTGCCATC
AAAGGCTAG
6_Francisella tularensis subsp. holarctica_GI#
89255957_Catalytic Ser Residue 99
MEILNYILNLSFTFWLLFLTIASGLIYIIDFVFFQKSRLAAYTDELKGLSKKQKRQFYKD
RGLKAPFIADQARSLFSVFFVVFLLRTFLIGNFLIPTASMTPTLPVGDFIFVNKTAYGIR
APFTNETLIKVGEPKRGDIVVFHFPVNPNVDFVKRVIGLPGDVISYKDKMLTINGKKLEY
TNCNRDAMNYYNQSLAAGSGDTVCTENLDGVKHEVDWIESIKGTDFENLKVPAGQYFVMG
DNRDNSEDSRYWGFVPDKDLVGKAKVVWMSWDKIDKKVRWDEIGKVF*
6_Francisella tularensis subsp. holarctica_GI#
89255957_Catalytic Ser nucleotides 295-297
ATGGAAATCTTAAACTATATTTTAAACTTGAGCTTTACTTTTTGGCTTTTATTCTTAACC
ATTGCCAGTGGTTTAATTTATATTATTGATTTTGTGTTCTTCCAAAAATCAAGATTAGCA
GCATATACAGATGAATTAAAAGGTCTTTCTAAGAAGCAAAAACGTCAGTTCTATAAAGAT
AGAGGATTAAAAGCACCTTTTATTGCTGATCAGGCGAGATCTTTATTTAGTGTATTTTTT
GTAGTTTTTCTACTTAGAACCTTCTTGATTGGTAATTTTTTAATTCCAACTGCATCAATG
ACACCAACACTTCCAGTTGGTGATTTTATTTTTGTCAATAAAACTGCTTATGGTATCAGA
GCACCATTTACCAATGAGACTTTAATAAAAGTTGGTGAACCCAAAAGAGGTGATATTGTA
GTATTTCATTTTCCAGTTAATCCTAATGTTGATTTTGTAAAACGAGTGATCGGTTTGCCT
GGCGATGTAATTTCGTATAAAGACAAAATGTTGACAATAAATGGTAAAAAACTTGAATAT
ACTAATTGTAATCGTGATGCAATGAACTATTATAATCAGTCTTTAGCTGCTGGTAGTGGC
GATACAGTATGTACGGAAAACCTTGATGGAGTTAAACATGAGGTTGATTGGATAGAGTCT
ATAAAGGGAACTGATTTTGAAAACCTTAAAGTCCCAGCAGGTCAATACTTTGTCATGGGA
GATAATCGTGATAATAGTGAAGATAGTCGTTATTGGGGTTTTGTACCTGACAAAGATCTA
GTTGGTAAAGCAAAAGTTGTTTGGATGAGCTGGGATAAGATAGATAAAAAGGTTCGCTGG
GATGAAATTGGTAAGGTCTTTTAA
7_Campylobacter jejuni RM1221_GI# 57237697_Catalytic
Ser Residue 38
MEILKKLYKFSQSWTGTVVIVLLVIFFFIQAFVIPSGSMKNTLLVGDFLFVKKFSYGIPT
PHIPWLEIPVLPDFNKDGHLIKAQGSQRGDIVVFRNPRNEKEHFVKRCVGTGGDRIVYAN
KTLYVRMHEGDEFMKEHYPNDLVTLGGQIYVKEPYKQKGIHYDPKKDIESDILRFLSIGD
FAMSPTYIKELGNHIGFSGGNAYVFDVPENEYFMMGDNRDYSYDSRFWGSVPYRLIVGKP
WFVYFSWDKDKNVRWERIGRFVDTLENDEQYIHDHDDEDKLS*
7_Campylobacter jejuni RM1221_GI# 57237697_Catalytic
Ser nucleotides 112-114
ATGGAAATTTTAAAGAAATTATATAAATTTTCACAGTCTTGGACTGGAACTGTAGTTATT
GTTCTTTTGGTGATTTTTTTCTTTATACAAGCTTTTGTTATTCCTTCTGGTTCTATGAAA
AACACCTTATTGGTAGGGGATTTTTTATTTGTTAAAAAATTTAGCTATGGTATCCCAACT
CCTCATATTCCTTGGTTGGAAATTCCTGTTTTGCCAGATTTCAATAAAGATGGGCATTTG
ATAAAAGCACAAGGGTCACAAAGAGGAGATATAGTTGTTTTTAGAAATCCTAGAAATGAA
AAAGAACACTTTGTAAAGCGTTGTGTAGGCACAGGAGGAGATAGGATAGTTTATGCAAAT
AAAACACTTTATGTAAGAATGCATGAGGGTGATGAATTTATGAAAGAACATTATCCGAAT
GATCTTGTTACTCTTGGAGGGCAAATTTATGTAAAAGAACCTTATAAACAAAAAGGTATT
CATTATGATCCAAAAAAAGATATAGAAAGCGATATTTTACGCTTTCTTAGCATAGGTGAT
TTTGCTATGTCTCCAACTTATATTAAAGAACTTGGAAATCATATAGGTTTTAGCGGCGGA
AATGCTTATGTTTTTGATGTGCCTGAAAATGAGTATTTCATGATGGGTGATAATCGCGAT
TATTCTTATGATAGTCGTTTTTGGGGTTCTGTTCCTTATAGGTTGATAGTAGGTAAACCT
TGGTTTGTATATTTCTCTTGGGATAAAGATAAAAATGTTCGCTGGGAAAGGATAGGGCGT
TTTGTTGATACCTTGGAAAATGATGAACAATATATCCATGATCATGATGATGAGGATAAA
TTAAGCTAA
8_Helicobacter pylori HPAG1_GI# 108562981_Catalytic
Ser Residue 38
MKFLRSVYAFCSSWVGTIVIVLLVIFFVAQAFIIPSRSMVGTLYEGDMLFVKKFSYGIPI
PKIPWIELPIMPDFKNNGHLIEGDRPKRGEVVVFIPPHEKKSYYVKRNFAIGGDEVLFTN
EGFYLHPFESGNDKDYIAKHYPNAMTKEFMGKIFVLNPYKSKHPGIHYQKDNETFHLMEQ
LATQGAEANISMQLIQMEGEKVFYKKINSDEFFMIGDNRDNSSDSRFWGSVAYKNIVGSP
WFVYFSLSLKNSLEMDAENNPKKRYLVRWERMFKSVEGLEKIIKKEKATH*
8_Helicobacter pylori HPAG1_GI# 108562981_Catalytic
Ser nucleotides 112-114
ATGAAATTTTTACGCTCTGTTTATGCATTTTGCTCCAGTTGGGTAGGGACGATTGTTATT
GTGCTGTTGGTTATCTTTTTTGTTGCGCAAGCTTTCATCATTCCCTCTCGCTCTATGGTA
GGCACGCTCTATGAGGGCGACATGCTCTTTGTCAAAAAATTTTCTTACGGCATACCCATT
CCTAAAATCCCATGGATTGAGCTTCCTATTATGCCTGATTTTAAAAATAACGGGCATTTG
ATAGAGGGGGATCGCCCTAAGCGCGGCGAAGTGGTCGTATTTATCCCCCCCCATGAAAAA
AAATCTTACTATGTCAAAAGGAATTTTGCCATTGGGGGCGATGAGGTGCTATTCACTAAT
GAGGGGTTTTATTTGCACCCTTTTGAGAGCGGCAACGATAAAGATTATATTGCTAAACAT
TACCCTAACGCCATGACTAAAGAATTTATGGGTAAAATTTTTGTTTTAAACCCTTATAAA
AGTAAGCATCCGGGTATCCATTACCAAAAAGACAATGAAACCTTCCACTTAATGGAGCAG
TTAGCCACTCAAGGTGCGGAAGCTAATATCAGCATGCAACTCATTCAAATGGAGGGCGAA
AAGGTGTTTTACAAGAAAATCAATAGCGATGAATTTTTCATGATCGGCGATAACAGAGAC
AATTCTAGCGACTCGCGCTTTTGGGGGAGTGTGGCTTATAAAAACATCGTGGGTTCGCCA
TGGTTTGTTTATTTCAGTTTGAGTTTAAAAAATAGCCTGGAAATGGATGCAGAAAACAAC
CCCAAAAAACGCTATTTGGTGCGTTGGGAACGCATGTTTAAAAGCGTTGAAGGCTTAGAA
AAAATCATTAAAAAAGAAAAAGCAACGCATTAA
9_Propionibacterium acnes J139_GI#
282854577_Catalytic Ser Residue 69
VADDYRARRAANGDTRDSDDATARGEQASGWQRFRSGAIEVVLIVVGALIISAVLRGFVA
QMFVIPSKSMQNTLQVGDRVIAVKAADFHRGDVVVFKDTEHWLPAVQDRRSVPGQILEFV
GLLPNKSSNYLIKRVIGMPGDTVACCNVNGQVTVNGKALDERSYLYSENGEMVKPSAMEF
RVTVPRGRMFVLGDHRNASGDSRYHLQDLDPGEYTGAPAFVPLDDVVGPAKAILMPLNRI
EGLGTPNTFRGIPDRSSSAPAKARICVGNTCCPK*
9_Propionibacterium acnes J139_GI#
282854577_Catalytic Ser nucleotides 205-207
GTGGCGGATGACTACCGGGCGAGGCGGGCTGCAAACGGCGACACCAGGGACTCTGACGAT
GCAACAGCACGTGGGGAACAGGCGTCTGGGTGGCAGCGCTTTCGGTCGGGGGCCATCGAA
GTTGTTCTCATCGTCGTTGGTGCCCTCATCATCTCAGCTGTGCTGCGTGGTTTCGTCGCT
CAGATGTTTGTCATCCCGTCGAAGTCCATGCAAAACACCTTGCAGGTGGGTGACCGCGTG
ATCGCGGTGAAAGCCGCCGATTTTCATCGGGGCGACGTCGTCGTGTTCAAAGACACCGAA
CATTGGTTACCTGCTGTTCAGGATCGCCGCTCTGTTCCAGGACAGATCCTCGAATTCGTC
GGGTTGTTGCCTAACAAGAGCTCGAACTACCTCATTAAGCGAGTGATCGGCATGCCTGGG
GACACCGTTGCCTGCTGCAACGTCAACGGCCAGGTGACCGTCAACGGTAAGGCGCTTGAC
GAGCGGTCATACCTGTACTCCGAAAATGGTGAAATGGTTAAACCCTCGGCGATGGAATTC
CGGGTCACTGTTCCTCGGGGGCGGATGTTCGTCTTGGGGGACCATCGCAATGCCTCGGGT
GACTCGCGCTATCACCTCCAAGACCTTGATCCGGGTGAGTATACGGGCGCTCCTGCGTTT
GTGCCGCTCGATGACGTCGTTGGGCCGGCAAAGGCCATTCTTATGCCTCTCAATCGCATT
GAGGGACTGGGGACTCCTAACACTTTCCGGGGAATCCCGGATAGGTCGTCGTCAGCTCCA
GCCAAGGCGCGCATCTGCGTCGGTAACACGTGCTGCCCTAAGTGA
10_Chlamydia trachomatis 434/Bu_GI#
166154241_Catalytic Ser Residue 113
MTSSYMSRLYSLNKSRRILHSSFRLLKSTKMLSHPETQKELQEVLKQLEEAILDQNREDA
SLFAKQAQAIQKRFPKSKLRATFDLIYALTFAAILAFLIRQFWFELYEVPTGSMRPTILE
QDRILVSKTTFGLRLPFSNRSIGYTPEAITRGELVVFTVGDLPIPNADTKYFGIIPGKKR
YIKRCMGKPGDTVYFYGGKIYGIDCDGEPIFPQNTENLYHVPYISFDGTPEILTHSEEQT
DVIFNQFHTPCGKISLPQQASYGQFFYKNAWHNDTPYALKDPHNEPVSYADLFGIKNFAM
VRILTKKQAALTHVLPSPLSDTYLEIAHTPNVSYPHPHLRPFETQLIPTIEPMKTLLPLR
KEHIHLIRNNLTTSRFTVVDGYAYKYQPAPMNTSGMVRMFALPMPNIPDGCYEFSKGDVF
KINMGGFRTKLKQPHPLTQLSNSQVIDLFNCGISFHTIYIPKNPQYAPFPNRYAFFNQGN
LFVMDSPVFIDSDPALQKFIVSEEEKELQSSEDKPYIAFIDRGPPPESTEEFVSFITNFG
LKIPEGHVLVLGDNCPMSADSRDFGFVPVENLLGSPVGIFWPINRLGLLSSNITPLSLPG
YLVNGLALGAFLYCIGLWYYRKNHRLFP*
10_Chlamydia trachomatis 434/Bu_GI#
166154241_Catalytic Ser nucleotides 337-339
ATGACGAGCAGTTACATGAGTCGCTTATATTCCCTGAATAAGAGTCGTCGCATTCTTCAT
TCTTCCTTTAGATTGCTGAAAAGCACAAAAATGCTCTCTCATCCGGAAACTCAAAAAGAA
CTACAAGAAGTCTTGAAACAGCTTGAAGAGGCTATTTTGGATCAGAATAGGGAAGATGCT
TCCCTTTTTGCTAAGCAAGCTCAAGCCATACAAAAAAGATTCCCTAAATCCAAACTCCGA
GCTACTTTTGATCTTATCTATGCTTTGACGTTTGCTGCCATTCTTGCTTTTTTAATCCGC
CAGTTCTGGTTTGAGCTATATGAAGTTCCTACAGGATCTATGCGGCCTACTATTCTTGAA
CAAGATCGTATTCTTGTTTCCAAAACAACATTTGGACTCCGGCTACCTTTTAGTAACAGA
AGTATTGGCTATACACCTGAGGCTATCACTCGAGGAGAACTGGTAGTCTTCACTGTTGGA
GATCTTCCTATCCCTAATGCCGACACTAAGTATTTTGGAATCATCCCTGGGAAAAAACGC
TATATAAAACGGTGCATGGGTAAACCTGGAGATACCGTATATTTTTATGGAGGGAAAATT
TATGGGATCGATTGCGACGGAGAGCCCATCTTCCCCCAAAATACAGAGAATCTCTACCAC
GTCCCCTATATTTCTTTTGACGGAACTCCAGAAATTCTTACCCATTCAGAAGAGCAAACA
GATGTGATCTTTAACCAATTTCACACACCTTGTGGAAAGATTTCTCTCCCTCAACAGGCT
TCTTATGGACAATTTTTCTATAAGAATGCTTGGCATAATGATACTCCCTATGCTTTAAAA
GATCCTCATAATGAGCCTGTTAGCTATGCCGATCTATTCGGAATAAAAAATTTTGCAATG
GTTCGCATCCTTACCAAAAAACAAGCTGCTCTTACTCATGTCCTTCCCTCTCCTCTTTCG
GACACCTACCTAGAAATTGCCCACACTCCTAATGTTTCCTATCCTCACCCTCACTTACGT
CCATTTGAAACACAGCTTATTCCTACTATCGAACCTATGAAAACCTTGCTTCCTTTAAGG
AAGGAACATATTCATTTGATTCGTAATAACCTCACAACATCCCGTTTTACAGTTGTAGAT
GGATATGCTTACAAGTACCAACCTGCTCCCATGAATACCTCAGGCATGGTCAGGATGTTT
GCCCTACCTATGCCAAATATTCCTGACGGATGTTATGAATTTTCTAAAGGAGACGTGTTT
AAAATCAATATGGGTGGCTTTCGAACAAAACTCAAACAGCCGCATCCTTTAACGCAATTA
AGCAATTCTCAGGTCATTGACTTATTTAATTGCGGCATTAGTTTCCACACGATCTATATT
CCTAAAAACCCTCAATATGCTCCGTTCCCTAATCGCTATGCATTTTTCAATCAAGGGAAC
CTGTTCGTTATGGATTCTCCAGTTTTTATTGATAGCGATCCTGCCTTACAGAAATTCATT
GTGTCTGAAGAGGAAAAAGAACTTCAATCATCTGAAGACAAACCTTACATCGCATTTATT
GACAGAGGTCCTCCTCCAGAATCTACAGAGGAATTTGTTTCCTTTATTACTAATTTCGGT
CTTAAAATTCCGGAAGGCCACGTGCTTGTCTTAGGAGATAATTGTCCTATGAGCGCTGAT
AGCCGTGATTTTGGTTTTGTTCCCGTTGAAAATCTTTTGGGATCTCCTGTTGGGATCTTC
TGGCCTATTAATCGTCTAGGATTGTTATCTTCCAATATAACGCCCTTGAGTTTACCTGGC
TACCTCGTAAATGGATTGGCTCTAGGAGCTTTTCTTTACTGCATAGGATTATGGTACTAT
CGAAAAAACCATAGGCTATTCCCTTAA
11_Chlamydophila pneumoniae CWL029_GI#
15618034_Catalytic Ser Residue 108
MKQHYSLNKSRHILRSTYKLLKSKKLAHSPADKKQLQELLEQLEEAIFEHDQETASDLAQ
QALAFSNRYPNSFGRKTYELIKALLFAGVVAFLVRQFWFELYEVPTGSMRPTILEQDRIL
VSKTTFGLHCPFAKKPLAFNPESVTRGGLVVFTVGDLPIPDADTKYFGLIPGKKRYIKRC
MGRPGDFLYFYGGKIYGLDDAGKRIEFPSVHGLENLYHVPYISFDGTTSSHTEGQKTIID
FKQFNQSYGRLIFPQTSMYGQFFDHKEWHQDEPNKLKDPHLSPVSYADLFGMGNYAMVRI
LTEHQARTSHLLPNPGSPTKVYLEICHTANLSYPKPLLRHYEHQLSPAIQPMKTLLPLRK
EHLHLIRNNLTTSRFIVAQGCAYKYHQFKINTSGIAKAYAILLPKVPDGCYEYSKGEAYQ
IGFGEIRYKLKSSHPLTQLNDKQVIELFNCGINFSSIYNPVNPLQAPLPNRYAFFNQGNL
YIMDSPVFIKNDPTLQKFVTSETEKQEGSSETQPYIAFVDKGLPPEDFKEFVEFIHNFGI
QVPKGHVLVLGDNYPMSADSREFGFVPMENLLGSPLCTFWPIGRMGRLTGVSAPTTLSGY
LVSGIALATGLSLIGYVYYQKRRRLFPKKEEKNHKK*
11_Chlamydophila pneumoniae CWL029_GI#
15618034_Catalytic Ser nucleotides 322-324
ATGAAACAACACTATTCTCTAAATAAAAGTCGTCATATCCTCCGCAGTACTTATAAGCTT
TTAAAAAGTAAAAAACTCGCCCATTCCCCTGCAGATAAAAAGCAACTGCAAGAACTACTA
GAACAACTAGAAGAGGCTATCTTTGAACATGATCAAGAAACTGCAAGCGACTTAGCTCAG
CAAGCATTAGCATTTTCCAACCGTTATCCTAATTCCTTCGGACGCAAAACCTATGAGCTT
ATCAAGGCCCTTCTTTTTGCTGGTGTTGTAGCCTTCTTAGTTCGGCAATTTTGGTTTGAA
CTTTATGAAGTGCCTACAGGATCCATGAGGCCTACAATTTTAGAACAGGATCGGATTCTT
GTATCCAAAACAACATTTGGTCTCCATTGCCCTTTTGCTAAGAAACCACTTGCCTTCAAT
CCTGAATCCGTAACTCGCGGGGGTCTTGTTGTTTTCACTGTAGGCGACCTCCCTATCCCA
GATGCTGATACAAAGTACTTCGGATTGATTCCAGGAAAAAAGCGTTACATTAAACGTTGC
ATGGGAAGACCTGGGGACTTCTTATATTTCTATGGAGGAAAAATTTATGGTCTTGATGAT
GCAGGTAAACGCATAGAGTTTCCTTCTGTCCATGGTTTAGAAAACTTATATCACGTCCCC
TATATATCCTTTGATGGCACTACCAGCAGCCATACAGAAGGGCAGAAAACAATTATAGAT
TTTAAGCAGTTCAATCAAAGTTATGGTCGGCTGATTTTCCCTCAAACCTCCATGTATGGA
CAATTCTTTGACCATAAAGAATGGCATCAAGACGAGCCTAATAAATTAAAAGATCCTCAT
CTTTCGCCAGTCAGCTATGCCGATCTTTTTGGTATGGGTAACTATGCTATGGTGCGCATC
TTAACAGAACATCAGGCACGAACATCCCATCTACTTCCGAATCCAGGAAGTCCAACTAAA
GTCTACTTAGAAATTTGCCATACAGCGAACCTTTCCTACCCAAAGCCTCTGTTGCGTCAC
TATGAGCATCAGCTCTCGCCTGCGATTCAACCTATGAAGACTTTACTTCCTTTGCGTAAG
GAACATTTGCACTTAATTCGGAACAATCTTACTACCTCTCGTTTTATTGTTGCTCAAGGA
TGTGCGTATAAATACCATCAATTCAAGATTAACACTTCAGGAATTGCCAAAGCCTATGCA
ATTCTCCTGCCCAAGGTCCCTGATGGTTGTTATGAATATTCTAAAGGCGAAGCGTATCAA
ATTGGCTTTGGAGAGATTCGTTATAAGCTAAAATCTTCTCACCCCCTTACTCAGCTCAAT
GATAAGCAAGTGATTGAACTTTTTAACTGCGGGATCAACTTTAGTTCTATTTATAATCCT
GTGAATCCGCTGCAAGCACCTTTACCTAACCGTTATGCATTCTTTAACCAAGGGAATCTT
TATATCATGGATTCTCCTGTATTTATAAAGAATGATCCAACTCTGCAAAAATTTGTGACT
TCTGAAACGGAAAAGCAAGAGGGGTCTTCAGAGACACAACCCTATATAGCTTTTGTTGAC
AAGGGACTCCCTCCAGAAGATTTTAAAGAATTCGTGGAGTTTATACATAATTTTGGTATT
CAAGTTCCTAAAGGTCATGTTCTCGTCTTGGGAGATAACTACCCTATGAGTGCGGATAGT
CGAGAATTTGGCTTTGTTCCTATGGAAAATCTCTTAGGATCTCCTCTATGTACATTCTGG
CCTATTGGACGCATGGGACGGTTAACTGGAGTTTCTGCTCCAACAACACTCTCAGGTTAT
CTTGTTAGTGGGATAGCATTAGCGACGGGTCTCTCTCTCATTGGATATGTCTACTATCAA
AAACGACGCAGACTCTTTCCTAAGAAAGAGGAGAAAAACCACAAGAAATAA
12_Staphylococcus carnosus subsp. carnosus TM300_GI#
224476066_Catalytic Ser Residue 36
VKKEIKEWIIAIAIALVLVLVITNFIAKSYTVRGDSMYPTLKDGEKVIVNMIGFKTGGLE
KGNVIVFHATKNSDYVKRVIGMPGDSIEYKHDQLYVNGKKVKEPYLDYNEKHKSYDEITG
SFKVKNLPNANGSNTIPKNKLLVLGDNREVSKDSRSFGLIDEDQVVGKVSLRYWPFTSFK
VNFNPDTKY*
12_Staphylococcus carnosus subsp. carnosus TM300_GI#
224476066_Catalytic Ser nucleotides 106-108
GTGAAGAAAGAAATTAAAGAGTGGATAATAGCCATAGCAATAGCTTTGGTATTAGTTCTA
GTCATAACAAATTTCATTGCGAAATCATATACGGTTCGTGGTGATTCAATGTATCCAACG
CTAAAAGACGGAGAAAAAGTTATCGTTAATATGATTGGATTTAAAACTGGCGGTTTAGAA
AAAGGTAATGTGATTGTATTCCACGCTACTAAAAACAGCGACTACGTTAAACGTGTTATC
GGTATGCCTGGTGACAGTATTGAATATAAACATGATCAATTGTATGTTAATGGTAAAAAA
GTGAAAGAACCTTATTTAGATTATAATGAAAAACATAAAAGCTATGATGAAATTACAGGT
AGCTTTAAAGTGAAAAATTTACCTAATGCAAATGGTTCAAACACAATTCCTAAAAACAAA
CTTCTTGTATTAGGAGATAACCGTGAAGTCAGTAAAGACAGCCGTTCATTCGGTTTAATT
GATGAAGATCAAGTTGTTGGTAAAGTAAGCTTGCGTTATTGGCCGTTTACATCTTTCAAA
GTAAACTTTAATCCGGATACAAAATATTAA
13_Staphylococcus haemolyticus JCSC1435_GI#
70726986_Catalytic Ser Residue 36
LKKEIVEWIVAIAVGLLLVWVMVNFVAKSYTIKGDSMDPTLKDGEHVMVNILGYKVGDIK
KGNVIVFHANQQDDYVKRVIGVPGDNVIYKNDKLYVNGKKINEPYLDYNEKRKQGEYITG
SFETKDLLNANPKSNIIPKGKYLVLGDNREVSKDSRAFGLIDRDQIVGKVSFRFWPFSEF
KFNFNPDNEK*
13_Staphylococcus haemolyticus JCSC1435_GI#
70726986_Catalytic Ser nucleotides 106-108
TTGAAGAAAGAAATAGTTGAATGGATTGTTGCCATAGCGGTAGGTTTATTACTTGTATGG
GTAATGGTTAACTTCGTAGCTAAATCATATACTATAAAAGGCGATTCAATGGATCCAACA
CTAAAAGATGGCGAACACGTCATGGTTAACATTCTAGGATATAAAGTTGGAGACATAAAA
AAAGGTAATGTAATCGTATTTCATGCGAATCAACAAGACGATTATGTTAAACGTGTCATT
GGTGTACCTGGCGATAACGTTATTTATAAAAATGATAAACTATATGTTAATGGTAAAAAG
ATAAATGAACCTTATCTTGATTACAATGAAAAACGTAAACAAGGTGAATATATTACGGGT
TCATTTGAAACTAAAGATTTACTAAATGCAAATCCTAAATCAAATATCATACCAAAAGGT
AAATACTTAGTTTTAGGTGATAACAGAGAAGTCAGTAAGGATAGTAGGGCGTTTGGTTTA
ATTGATAGAGATCAAATTGTTGGTAAAGTATCATTTAGATTTTGGCCATTCAGTGAATTT
AAGTTTAATTTTAATCCAGATAATGAAAAATAA
14_Staphylococcus haemolyticus JCSC1435_GI#
70727661_Catalytic Ser Residue 36
LKKEIIEWIVAIGGALLIVGIVLKFIGTSYTVSGSSMYPTFQDRNKVIVSKISKTLNHID
NGDVVVFHEDAQRDFIKRVIGTPGDKVEYEGDQLYVNDKKVSEPYLDYNKKHKQGKYLTG
TFKTSQVNGANGKNKIPKDKYLVLGDNRQNSVDSRLAEVGLVDKDQLVGKVVLRYWPFNK
WEAGFNPGTF*
14_Staphylococcus haemolyticus JCSC1435_GI#
70727661_Catalytic Ser nucleotides 106-108
TTGAAAAAAGAGATAATTGAATGGATTGTAGCCATTGGTGGCGCACTCTTAATTGTAGGT
ATTGTATTAAAGTTTATTGGAACATCATACACAGTATCAGGTTCATCGATGTATCCAACT
TTCCAAGATAGAAATAAAGTGATAGTTAGTAAGATTTCGAAAACATTGAACCACATTGAT
AATGGTGATGTCGTTGTCTTCCATGAAGATGCACAACGTGATTTTATTAAGCGTGTGATT
GGTACGCCAGGTGATAAAGTTGAGTATGAAGGTGATCAATTATATGTTAATGACAAAAAG
GTATCAGAGCCTTATTTAGATTATAATAAGAAGCATAAACAAGGTAAGTATTTAACAGGT
ACATTTAAAACAAGCCAAGTGAACGGAGCAAATGGTAAAAATAAAATTCCTAAAGATAAG
TATTTAGTTTTAGGTGATAACAGACAAAATAGTGTAGATAGCCGTTTGGCTGAAGTTGGT
TTAGTAGATAAAGACCAACTTGTAGGTAAAGTTGTTTTAAGATATTGGCCATTTAATAAA
TGGGAAGCAGGTTTTAACCCAGGCACATTTTAG
15_Staphylococcus epidermidis ATCC 12228_GI#
27467580_Catalytic Ser Residue 36
LKKEILEWIVAIAVAIALIAIITKFVGKSYSIKGDSMDPTLKDGERVVVNIIGYKLGGVE
KGNVIVFHANKKDDYVKRVIGTPGDSVEYKNDTLYVNGKKQSEPYLNYNEKRKQTEYITG
SFKTKNLPNANPQSNVIPKGKYLVLGDNREVSKDSRSFGLIDKDQIVGKVSLRYWPFSEF
KSNFNPNNTKN*
15_Staphylococcus epidermidis ATCC 12228_GI#
27467580_Catalytic Ser nucleotides 106-108
TTGAAAAAAGAAATTTTAGAGTGGATTGTTGCCATAGCCGTTGCCATTGCACTTATTGCC
ATAATCACTAAATTTGTCGGAAAATCATATTCTATTAAAGGTGATTCAATGGATCCTACA
TTAAAAGATGGGGAGCGTGTAGTGGTAAATATTATTGGCTATAAATTAGGTGGCGTTGAA
AAAGGAAATGTCATTGTATTTCATGCTAATAAAAAAGATGATTATGTTAAAAGAGTTATT
GGAACTCCAGGAGATAGTGTTGAATATAAAAATGATACACTCTATGTTAATGGTAAAAAG
CAATCAGAACCATACTTGAACTATAATGAAAAACGTAAGCAAACTGAGTATATCACAGGT
AGTTTCAAAACAAAAAATTTACCAAATGCTAATCCTCAATCTAATGTTATTCCTAAAGGT
AAATATTTAGTTTTGGGGGATAACCGTGAGGTAAGTAAAGATAGTCGTTCATTCGGTTTA
ATTGACAAAGACCAAATTGTTGGAAAGGTATCGCTCAGATATTGGCCTTTCAGTGAATTT
AAATCTAACTTTAATCCAAATAACACTAAAAATTAA
16_Staphylococcus epidermidis ATCC 12228_GI#
27469315_Catalytic Ser Residue 36
MKKEIIEWIVAIIVAIVIVTLVQKFLFASYTVKGASMHPTFENREKVIVSRIAKTLDHID
TGDVVIFHANAKQDYIKRLIGKPGDSVEYKKDQLYLNGKKVDEPYLSENKKHKVGEYLTE
NFKSKDLKGTNGNMKIPSGKYLVLGDNRQNSIDSRMDEVGLLDKNQVVGKVVLRYWPFNR
WGGSFNPGTFPN*
16_Staphylococcus epidermidis ATCC 12228_GI#
27469315_Catalytic Ser nucleotides 106-108
ATGAAGAAAGAAATAATAGAATGGATTGTAGCCATAATCGTTGCAATTGTTATCGTCACA
CTTGTGCAAAAGTTTTTATTTGCTTCTTATACAGTCAAAGGAGCGTCTATGCATCCAACA
TTTGAAAATAGAGAAAAAGTGATAGTAAGTCGTATAGCAAAAACACTTGATCATATTGAT
ACAGGAGATGTAGTGATTTTTCATGCTAACGCGAAGCAAGATTATATTAAGCGACTTATT
GGTAAACCAGGTGATTCAGTAGAATATAAAAAAGATCAACTATATTTAAACGGTAAAAAA
GTAGATGAGCCTTATTTAAGTGAAAATAAAAAACATAAAGTTGGAGAATATCTAACGGAA
AACTTTAAGTCTAAAGATCTTAAGGGTACGAATGGCAATATGAAAATTCCTAGTGGTAAA
TACTTGGTTTTAGGTGATAATCGTCAAAACAGTATTGACAGTCGCATGGATGAAGTAGGT
CTTTTAGATAAAAATCAAGTTGTTGGAAAAGTAGTTTTGAGATACTGGCCATTTAATCGG
TGGGGCGGTAGTTTTAATCCTGGAACATTTCCTAACTAA
17_Staphylococcus hominis SK119_GI#
228474322_Catalytic Ser Residue 36
LKKEITEWIVAIAVGLLLVWLVVTFVAKSYTIKGDSMDPTLKDGQHVMVNILGYKVGNIK
KGNVIVFHANQSDDYVKRVIGVPGDSVTYKKDQLYINGKKVNEPYLDYNEKHKQGEYITG
SFETKDLLNAHPNSNVIPKNKYLVLGDNREVSKDSRAFGLIDKQQIVGKVSFRFWPLNNF
KFNFNPDK*
17_Staphylococcus hominis SK119_GI#
228474322_Catalytic Ser nucleotides 106-108
TTGAAAAAAGAAATAACAGAATGGATTGTTGCGATAGCTGTAGGTTTATTGCTCGTATGG
CTTGTAGTCACTTTTGTTGCCAAATCCTATACAATAAAAGGTGACTCAATGGATCCAACA
TTAAAAGATGGGCAACATGTGATGGTTAACATTTTAGGTTATAAGGTAGGAAACATAAAA
AAAGGAAATGTTATTGTCTTCCATGCTAATCAATCTGATGACTATGTTAAAAGAGTAATA
GGCGTACCAGGAGATAGTGTGACATATAAAAAAGATCAGCTATATATTAATGGGAAAAAG
GTAAATGAGCCTTACTTAGACTATAATGAAAAACATAAACAAGGAGAGTACATTACTGGA
TCTTTTGAAACTAAGGATCTTCTTAATGCTCATCCTAACTCTAACGTTATTCCTAAAAAT
AAATACTTAGTATTAGGAGATAACCGTGAAGTTAGTAAAGATAGTAGAGCGTTTGGATTA
ATAGATAAACAACAAATCGTCGGTAAAGTATCATTTAGATTTTGGCCATTAAATAATTTT
AAATTTAATTTTAATCCAGATAAGTAG
18_Staphylococcus lugdunensis HKU09-01_GI#
289551204_Catalytic Ser Residue 36
VKKELTEWLIAIAVGIILVILIINFVAKSYTIKGDSMNPTLKDGDHVLVNIIGYKVGTVK
KGNVIVFHANQKDDYVKRVIGTPGDKVYYRDDQLIINGKKVKEPYLEYNMKRKQGEYITG
SLDIKDLAGAKHNSNVIPQHKYLVLGDNREVSKDSRAFGLIDEKQIVGKVSLRFWPLTDF
KFNFNPDMS*
18_Staphylococcus lugdunensis HKU09-01_GI#
289551204_Catalytic Ser nucleotides 106-108
GTGAAAAAGGAATTGACAGAATGGTTAATAGCTATAGCGGTAGGTATTATTTTAGTCATA
CTAATCATTAATTTTGTAGCGAAATCATATACCATTAAAGGAGACTCAATGAATCCAACA
TTAAAAGATGGCGATCATGTTCTGGTCAATATTATCGGCTATAAAGTAGGCACTGTGAAA
AAGGGGAATGTCATTGTCTTCCATGCTAACCAAAAGGATGATTATGTTAAACGCGTTATA
GGCACACCAGGTGACAAAGTATACTATCGAGATGATCAACTTATTATAAACGGAAAAAAA
GTAAAAGAACCTTATCTCGAATACAATATGAAACGTAAGCAAGGAGAGTATATTACTGGA
TCTTTAGATATAAAAGATTTGGCCGGTGCAAAACATAATTCTAATGTCATACCTCAACAT
AAATACCTCGTGTTAGGAGACAATCGTGAGGTAAGTAAAGATAGCCGTGCTTTTGGCCTT
ATCGATGAAAAGCAAATTGTCGGTAAAGTGTCTTTAAGATTTTGGCCATTAACTGATTTT
AAATTTAATTTTAACCCTGATATGAGCTAA
19_Staphylococcus lugdunensis HKU09-01_GI#
289551814_Catalytic Ser Residue 36
VKKEILEWIVSIAVALIIVGIVVKFIGVTYSVSGDSMYPTFKDREKVVVSKISKTLDHID
NGDIVVFKEDKDRDFIKRLIGKPGDKVEYKGDQLYVNNKKIDEPYLKYNKEHKNGKYLTG
SFKSSDLQNANGETKIPKDKYLVLGDNRQNSLDSRFPQVGLIDKEQIVGKVVLRFWPFGE
WTTKFNPGTFDK*
19_Staphylococcus lugdunensis HKU09-01_GI#
289551814_Catalytic Ser nucleotides 106-108
GTGAAAAAAGAGATCTTAGAGTGGATTGTGTCTATAGCAGTTGCACTTATCATTGTAGGT
ATAGTTGTTAAATTTATTGGAGTTACATATTCAGTTTCGGGAGATTCAATGTATCCAACA
TTTAAAGATAGAGAAAAAGTAGTAGTGAGTAAAATTTCCAAAACGTTAGACCATATTGAT
AATGGTGATATCGTTGTCTTTAAAGAAGATAAAGATAGAGACTTTATTAAACGTTTAATT
GGTAAACCTGGAGACAAAGTTGAGTATAAAGGTGACCAACTATATGTTAATAATAAAAAA
ATTGATGAGCCTTATTTAAAATATAACAAAGAGCATAAAAATGGTAAGTATCTGACAGGT
TCTTTCAAATCGAGTGATTTGCAAAATGCTAATGGTGAGACGAAGATTCCTAAAGACAAA
TATTTAGTGTTAGGTGATAATCGTCAAAACAGTTTAGATAGTCGTTTTCCACAGGTAGGG
CTTATTGATAAAGAACAAATTGTAGGTAAAGTTGTGTTACGTTTCTGGCCATTTGGTGAG
TGGACAACAAAATTTAATCCTGGAACATTTGATAAGTAA
20_Streptococcus agalactiae COH1_GI#
77408620_Catalytic Ser Residue 58
MKRQISSDKLSQELDRVTYQKRFWSVIKNTIYILMAVASIAILIAVLWLPVLRIYGHSMN
KTLSAGDVVFTVKGSNFKTGDVVAFYYNNKVLVKRVIAESGDWVNIDSQGDVYVNQHKLK
EPYVIHKALGNSNIKYPYQVPDKKIFVLGDNRKTSIDSRSTSVGDVSEEQIVGKISFRIW
PLGKISSIN*
20_Streptococcus agalactiae COH1_GI#
77408620_Catalytic Ser nucleotides 172-174
ATGAAAAGACAGATTAGTTCAGATAAATTATCTCAAGAACTGGATCGCGTAACTTATCAG
AAACGCTTTTGGAGTGTCATTAAAAATACCATATACATCTTGATGGCGGTTGCCTCAATA
GCCATTTTAATTGCGGTTTTATGGTTGCCTGTATTAAGAATCTACGGACATTCAATGAAT
AAGACTTTAAGTGCAGGTGATGTAGTCTTTACAGTAAAAGGTTCAAATTTTAAAACTGGA
GACGTTGTCGCGTTTTACTACAATAATAAGGTCCTAGTCAAGCGGGTTATTGCAGAGTCA
GGAGACTGGGTTAATATTGATTCTCAAGGGGATGTTTACGTGAATCAACATAAGTTGAAA
GAACCATATGTTATTCATAAAGCACTCGGTAATAGTAATATAAAATACCCATATCAAGTA
CCTGATAAAAAAATTTTTGTATTAGGAGACAACCGAAAAACTTCAATTGATTCTCGAAGT
ACTTCTGTAGGAGATGTTTCAGAAGAACAAATTGTAGGTAAAATTTCTTTCAGAATATGG
CCTCTAGGTAAGATTAGTAGTATCAATTAA
21_Streptococcus agalactiae COH1_GI#
77409453_Catalytic Ser Residue 35
MKEFIKEWGVFILILSLFLLSRIFLWQFVKVDGHSMDPTLADKEQLVVLKQTKINRFDIV
VANEEEGGQKKKIVKRVIGMPGDVIKYKNDTLTINNKKTEEPYLKEYTKLFKKDKLQEKY
SYNPLFQDLAQSSTAFTTDSNGSSEFTTVVPKGHYYLVGDDRIVSKDSRAVGSFKKSTIV
GEVKFRFWPIRRFGTIN*
21_Streptococcus agalactiae COH1_GI#
77409453_Catalytic Ser nucleotides 103-105
ATGAAAGAATTTATTAAAGAATGGGGTGTCTTTATCCTCATCCTCTCACTTTTTTTACTA
TCGCGTATCTTTTTATGGCAATTCGTTAAAGTTGACGGACACTCCATGGATCCAACTTTA
GCTGACAAGGAACAGCTAGTAGTTCTCAAACAAACAAAAATCAATCGATTCGATATTGTA
GTGGCTAACGAAGAAGAAGGCGGCCAAAAGAAAAAAATTGTTAAACGTGTCATTGGTATG
CCAGGTGATGTCATCAAATATAAAAATGACACCTTAACTATTAACAATAAAAAAACAGAA
GAACCTTACCTCAAGGAATATACTAAATTATTTAAAAAGGATAAATTACAGGAAAAATAT
TCGTATAACCCACTTTTCCAAGACCTAGCACAAAGCTCTACCGCTTTCACCACTGACAGC
AATGGCAGCAGCGAATTTACTACTGTCGTGCCTAAAGGCCACTACTATCTTGTTGGTGAT
GACCGAATTGTCTCTAAAGATAGTCGTGCCGTCGGTTCCTTCAAAAAATCAACGATTGTG
GGAGAGGTTAAATTCCGCTTCTGGCCAATTCGTCGTTTTGGAACTATCAACTAA
22_Streptococcus dysgalactiae subsp. equisimilis
GGS_124_GI# 251783284_Catalytic Ser Residue 35
MKHFIKEWGPFTLFLILFGLSRLFLWQAVKVDGHSMDPTLAHGERLIVLNQARIDRFDIV
VAREEENGQKKEIVKRVVGMPGDTIAYNDDTLYINGKKTDEPYLVNYLKEFKKDKLQKTY
AYNSLFQQLAETSDAFTTNAEGQTRFEISVPEGEYLLLGDDRIVSRDSREVGSFKKEKLI
GEVKARFWPLNKMTLFK*
22_Streptococcus dysgalactiae subsp. equisimilis
GGS_124_GI# 251783284_Catalytic Ser nucleotides
103-105
ATGAAACATTTTATTAAAGAATGGGGCCCATTTACCCTCTTTCTCATCCTCTTCGGTTTA
TCTCGTCTTTTCTTGTGGCAAGCTGTTAAAGTTGATGGCCACTCCATGGACCCTACGTTA
GCCCATGGGGAACGTCTCATTGTTTTAAACCAAGCTAGAATTGACCGTTTCGATATTGTC
GTTGCCCGTGAGGAAGAAAATGGGCAGAAAAAAGAAATTGTCAAACGAGTTGTCGGCATG
CCAGGTGATACCATTGCCTACAACGATGATACGCTTTACATTAATGGTAAAAAAACAGAT
GAGCCTTACCTAGTTAACTACCTTAAAGAGTTCAAAAAGGACAAGCTTCAAAAGACTTAC
GCTTACAATAGTCTATTTCAGCAATTAGCTGAAACATCGGATGCCTTCACCACTAATGCT
GAAGGTCAAACACGTTTTGAAATCAGTGTACCAGAAGGTGAATACCTCCTTCTTGGAGAT
GACCGAATTGTCTCACGCGACAGCCGTGAAGTTGGTAGTTTTAAAAAAGAAAAACTTATC
GGTGAAGTCAAGGCTCGCTTCTGGCCACTCAATAAAATGACTCTTTTTAAGTAA
23_Streptococcus mitis B6_GI# 289168569_Catalytic Ser
Residue 38
MNSFKNFLKEWGLFLLILSLLALSRIFFWSNVRVEGHSMDPTLADGEILFVVKHLPIDRF
DIVVAHEEDGNKDIVKRVIGMPGDTIRYENDKLYINDKETDEPYLADYIKRFKDDKLQST
YSGKGFEGNKGTFFRSIAEKAQAFTVDVNYNTNFSFTVPEGEYLLLGDDRLVSSDSRHVG
TFKAKDITGEAKFRFWPITRIGTF*
23_Streptococcus mitis B6_GI# 289168569_Catalytic Ser
nucleotides 112-114
ATGAATTCATTTAAAAATTTCCTAAAAGAGTGGGGATTGTTCCTCCTGATTCTGTCATTA
CTAGCTTTGAGCCGTATCTTTTTTTGGAGTAATGTCCGCGTAGAAGGGCATTCCATGGAT
CCGACCCTAGCGGATGGCGAAATTCTCTTCGTTGTCAAACACCTTCCTATTGACCGTTTT
GATATCGTGGTGGCCCATGAGGAAGATGGCAATAAGGACATCGTCAAGCGCGTGATTGGA
ATGCCTGGCGATACTATCCGTTACGAAAACGATAAACTTTACATCAATGATAAAGAGACG
GACGAACCTTACCTAGCTGACTATATCAAACGTTTCAAGGATGACAAACTCCAAAGCACC
TACTCAGGCAAGGGCTTTGAAGGAAATAAAGGAACCTTCTTTAGAAGTATTGCGGAAAAA
GCTCAAGCCTTCACAGTTGATGTCAACTATAACACCAACTTTAGCTTTACTGTTCCAGAA
GGAGAATACCTTCTCCTCGGAGACGACCGCTTGGTTTCTAGCGACAGCCGTCACGTAGGT
ACCTTCAAAGCAAAAGATATCACAGGGGAAGCTAAATTCCGCTTCTGGCCAATCACCCGT
ATCGGAACATTTTAA
24_Streptococcus oralis ATCC 35037_GI#
293364826_Catalytic Ser Residue 38
MNSFKTFLKEWGVFFLIIALVGLSRIFLWSNVRVEGHSMDPTLADGEVLFVVKHLPIDRF
DIVVAHEEDGNKDIVKRVIGMPGDTIRYENDKLFINGEETNEPYLAEYLNLFKTEKLQNT
YTGKGFEGNKGVYFRELAQKAQAFTVDVNSNTRFSFTVPQGEYLLLGDDRLVSSDSRHVG
TFKASDIKGEAKFRFWPLNRIGTF*
24_Streptococcus oralis ATCC 35037_GI#
293364826_Catalytic Ser nucleotides 112-114
ATGAATTCGTTTAAAACATTTCTAAAAGAATGGGGAGTTTTCTTCCTGATTATCGCACTG
GTCGGTCTTAGCCGCATCTTTCTTTGGAGCAATGTCCGTGTGGAAGGACACTCTATGGAC
CCTACCCTAGCTGACGGAGAAGTTCTCTTCGTTGTTAAACACCTCCCAATTGACCGCTTC
GACATCGTGGTTGCGCATGAGGAAGACGGAAATAAAGACATTGTCAAAAGGGTTATCGGT
ATGCCTGGTGATACCATCCGCTACGAAAATGACAAACTCTTTATCAACGGTGAAGAAACG
AATGAACCCTACCTAGCTGAGTACCTCAACTTGTTCAAAACAGAAAAGTTGCAAAACACC
TATACTGGAAAAGGATTTGAAGGCAATAAGGGAGTTTACTTTAGAGAACTTGCTCAAAAA
GCACAAGCCTTTACGGTCGATGTCAATTCCAACACCAGATTCAGCTTTACTGTCCCTCAA
GGCGAATACCTTCTCCTTGGTGACGATCGTCTAGTCTCTAGCGACAGCCGCCATGTCGGT
ACCTTCAAGGCCAGCGATATCAAAGGCGAAGCAAAATTCCGTTTCTGGCCACTTAACCGT
ATCGGAACTTTTTAA
25_Streptococcus pneumoniae R6_GI# 15902408_Catalytic
Ser Residue 38
MNLFKNFLKEWGLFLLILSLLALSRIFFWSNVRVEGHSMDPTLADGEILFVVKHLPIDRF
DIVVAHEEDGNKDIVKRVIGMPGDTIRYENDKLYINDKETDEPYLADYIKRFKDDKLQST
YSGKGFEGNKGTFFRSIAQKAQAFTVDVNYNTNFSFTVPEGEYLLLGDDRLVSSDSRHVG
TFKAKDITGEAKFRFWPITRIGTF*
25_Streptococcus pneumoniae R6_GI# 15902408_Catalytic
Ser nucleotides 112-114
ATGAATTTATTTAAAAATTTCTTAAAAGAGTGGGGATTATTCCTCCTGATTCTGTCATTA
CTAGCTTTGAGCCGTATCTTTTTTTGGAGCAATGTTCGCGTAGAAGGACATTCCATGGAT
CCGACCCTAGCGGATGGTGAAATCCTCTTTGTTGTTAAGCACCTCCCTATTGACCGTTTT
GATATCGTGGTGGCCCATGAGGAAGATGGCAATAAGGACATCGTCAAGCGCGTGATTGGA
ATGCCTGGCGACACCATTCGTTACGAAAATGATAAACTCTACATCAATGACAAAGAAACG
GACGAGCCTTATCTAGCAGACTATATCAAACGCTTCAAGGATGACAAACTCCAAAGCACT
TACTCAGGCAAGGGCTTTGAAGGAAATAAAGGAACTTTCTTTAGAAGTATCGCTCAAAAA
GCCCAAGCCTTCACAGTTGATGTCAACTACAACACCAACTTTAGCTTTACTGTTCCAGAA
GGAGAATACCTTCTCCTCGGAGATGACCGCTTGGTTTCGAGCGACAGCCGCCACGTAGGT
ACCTTCAAAGCAAAAGATATCACAGGGGAAGCTAAATTCCGCTTCTGGCCAATCACCCGT
ATCGGAACATTTTAA
26_Streptococcus pyogenes M1 GAS_GI#
15675668_Catalytic Ser Residue 35
MKQFIKEWGPFTLFLILFGLSRLFLWQAVKVDGHSMDPTLAHGERLIVFNQARIDRFDIV
VAQEEENGQKKEIVKRVIGLPGDTISYNDDTLYINGKKTVEPYLAEYLKQFKNDKLQKTY
AYNTLFQQLAETSDAFTTNSEGQTRFEMSVPKGEYLLLGDDRIVSRDSREVGSFKKENLI
GEVKARFWPLNKMTVFN*
26_Streptococcus pyogenes M1 GAS_GI#
15675668_Catalytic Ser nucleotides 103-105
ATGAAACAGTTTATTAAAGAATGGGGCCCATTCACTCTCTTTTTAATTCTCTTTGGTCTA
TCTCGTCTTTTTTTGTGGCAGGCTGTTAAAGTAGACGGCCATTCTATGGACCCAACTCTA
GCTCATGGCGAACGCCTTATCGTTTTTAATCAAGCTAGAATTGATCGCTTTGATATTGTA
GTTGCTCAGGAAGAAGAAAACGGACAAAAGAAAGAAATCGTAAAAAGAGTTATTGGATTG
CCAGGCGATACCATTTCTTATAATGATGACACACTTTATATTAATGGTAAAAAAACAGTT
GAGCCGTATTTGGCTGAGTATCTAAAACAATTTAAAAACGATAAACTCCAAAAAACTTAC
GCCTATAATACCCTATTCCAACAGTTAGCAGAAACATCTGATGCTTTTACAACTAATTCT
GAGGGACAAACACGCTTTGAGATGAGTGTTCCAAAAGGAGAATACCTTCTTCTTGGTGAT
GATCGTATTGTTTCCAGGGATAGTCGCGAAGTTGGTAGTTTCAAAAAAGAAAACCTTATC
GGTGAAGTGAAAGCTCGTTTTTGGCCACTCAATAAAATGACCGTTTTTAATTAG
SPase Sequences of Arylomycin-Resistant Bacteria
1_Escherichia coli str. K-12 substr. MG1655_GI#
16130493_Catalytic Ser Residues 91 (SEQ ID NO:)
MANMFALILVIATLVTGILWCVDKFFFAPKRRERQAAAQAAAGDSLDKATLKKVAPKPGW
LETGASVFPVLAIVLIVRSFIYEPFQIPSGSMMPTLLIGDFILVEKFAYGIKDPIYQKTL
IETGHPKRGDIVVFKYPEDPKLDYIKRAVGLPGDKVTYDPVSKELTIQPGCSSGQACENA
LPVTYSNVEPSDFVQTFSRRNGGEATSGFFEVPKNETKENGIRLSERKETLGDVTHRILT
VPIAQDQVGMYYQQPGQQLATWIVPPGQYFMMGDNRDNSADSRYWGFVPEANLVGRATAI
WMSFDKQEGEWPTGLRLSRIGGIH*
1_Escherichia coli str. K-12 substr. MG1655_GI#
16130493_Catalytic Ser nucleotides 271-273 (SEQ ID NO:)
ATGGCGAATATGTTTGCCCTGATTCTGGTGATTGCCACACTGGTGACGGGCATTTTATGG
TGCGTGGATAAATTCTTTTTCGCACCTAAACGGCGGGAACGTCAGGCAGCGGCGCAGGCG
GCTGCCGGGGACTCACTGGATAAAGCAACGTTGAAAAAGGTTGCGCCGAAGCCTGGCTGG
CTGGAAACCGGTGCTTCTGTTTTTCCGGTACTGGCTATCGTATTGATTGTGCGTTCGTTT
ATTTATGAACCGTTCCAGATCCCGTCAGGTTCGATGATGCCGACTCTGTTAATTGGTGAT
TTTATTCTGGTAGAGAAGTTTGCTTATGGCATTAAAGATCCTATCTACCAGAAAACGCTG
ATCGAAACCGGTCATCCGAAACGCGGCGATATCGTGGTCTTTAAATATCCGGAAGATCCA
AAGCTTGATTACATCAAGCGCGCGGTGGGTTTACCGGGCGATAAAGTCACTTACGATCCG
GTCTCAAAAGAGCTGACGATTCAACCGGGATGCAGTTCCGGCCAGGCGTGTGAAAACGCG
CTGCCGGTCACCTACTCAAACGTGGAACCGAGCGATTTCGTTCAGACCTTCTCACGCCGT
AATGGTGGGGAAGCGACCAGCGGATTCTTTGAAGTGCCGAAAAACGAAACCAAAGAAAAT
GGAATTCGTCTTTCCGAGCGTAAAGAGACACTGGGTGATGTGACGCACCGCATTCTGACA
GTGCCGATTGCGCAGGATCAGGTGGGGATGTATTACCAGCAGCCAGGGCAACAACTGGCA
ACCTGGATTGTTCCTCCGGGACAATACTTCATGATGGGCGACAACCGCGACAACAGCGCG
GACAGCCGTTACTGGGGCTTTGTGCCGGAAGCGAATCTGGTCGGTCGGGCAACGGCTATC
TGGATGAGCTTCGATAAGCAAGAAGGCGAATGGCCGACTGGTCTGCGCTTAAGTCGCATT
GGCGGCATCCATTAA
2_Salmonella enterica subsp. enterica serovar Typhi str.
CT18_GI# 16761494_Catalytic Ser Residues 91
(SEQ ID NO:)
MANMFALILVIATLVTGILWCVDKFVFAPKRRARQAAAQTASGDALDNATLNKVAPKPGW
LETGASVFPVLAIVLIVRSFLYEPFQIPSGSMMPTLLIGDFILVEKFAYGIKDPIYQKTL
IETGHPKRGDIVVFKYPEDPKLDYIKRAVGLPGDKITYDPVAKEVTIQPGCSSGQACENA
LPVTYSNVEPSDFVQTFARRNGGEATSGFFEVPLNETKENGIRLTERKETLGDVTHRILM
VPIAQDQLGMYYQQPGQPLATWVVPPGQYFMMGDNRDNSADSRYWGFVPEANLVGKAVAI
WMSFDKQEGEWPTGVRLSRIGGIH*
2_Salmonella enterica subsp. enterica serovar Typhi str.
CT18_GI# 16761494_Catalytic Ser nucleotides 271-273
(SEQ ID NO:)
ATGGCGAACATGTTTGCCCTGATTCTGGTGATAGCCACACTGGTGACGGGCATTTTATGG
TGCGTTGATAAGTTTGTTTTCGCGCCAAAACGTCGGGCGCGCCAGGCTGCCGCGCAAACG
GCGTCGGGAGATGCGCTGGATAACGCTACGCTCAATAAAGTGGCGCCTAAGCCGGGCTGG
CTGGAGACCGGGGCGTCGGTTTTCCCGGTTCTGGCGATCGTTCTGATCGTTCGTTCATTT
CTTTATGAACCCTTTCAGATCCCGTCAGGCTCAATGATGCCGACACTGCTTATCGGCGAT
TTTATTCTGGTGGAAAAATTTGCCTACGGCATTAAAGATCCGATCTACCAGAAAACCCTG
ATTGAAACCGGTCATCCAAAGCGCGGGGATATTGTGGTATTTAAATATCCGGAAGATCCT
AAGTTAGATTACATCAAACGCGCCGTCGGTTTGCCGGGCGATAAAATCACTTATGATCCG
GTTGCGAAAGAGGTGACGATTCAGCCTGGCTGTAGCTCCGGTCAGGCGTGCGAAAATGCG
CTGCCGGTTACCTACTCTAACGTTGAGCCGAGCGATTTTGTACAGACCTTTGCCCGCCGT
AACGGCGGAGAAGCGACCAGCGGTTTCTTTGAGGTTCCGCTAAACGAGACAAAAGAAAAC
GGCATTCGCCTGACCGAACGTAAAGAGACGTTAGGCGATGTGACCCACCGCATCCTGATG
GTGCCGATAGCCCAGGATCAGTTGGGCATGTATTACCAACAGCCAGGACAACCGCTGGCG
ACCTGGGTTGTACCGCCGGGGCAATATTTCATGATGGGCGACAACCGCGATAACAGCGCG
GATAGTCGTTACTGGGGATTTGTTCCGGAAGCGAATCTGGTCGGTAAAGCGGTCGCTATC
TGGATGAGCTTTGACAAGCAGGAAGGGGAGTGGCCGACAGGCGTACGCCTGAGTCGTATC
GGCGGTATTCACTAA
3_Klebsiella pneumoniae subsp. pneumoniae MGH 78578_GI#
152971424_Catalytic Ser Residues 91 (SEQ ID NO:)
MANMFALILVIATLVTGVLWCLDKFIFAPKRRERQAAAQAATGEQLDKKTLKKVGPKPGW
LETGASVFPVLAIVLVVRSFIYEPFQIPSGSMMPTLLIGDFILVEKFAYGIKDPIYQKTL
IETGHPKRGDIVVFKYPEDPRLDYIKRAVGLPGDKVTYDPVAKQVTIQPGCSSGQACGNA
LPVTYSNVEPSDFVQTFSRSNGGEASSGFWQLPKGETKADGIRLTERQETLGDVTHRILM
VPIAQDQVGMYYHQSGLPLATWIVPPGQYFMMGDNRDNSADSRYWGFVPEANLVGKATAI
WMSFEKQEGEWPTGVRLSRIGGIH*
3_Klebsiella pneumoniae subsp. pneumoniae MGH 78578_GI#
152971424_Catalytic Ser nucleotides 271-273 (SEQ ID NO:)
ATGGCGAACATGTTTGCCCTGATCCTGGTGATTGCAACCCTGGTGACGGGCGTTTTATGG
TGCCTGGACAAGTTCATTTTTGCACCGAAACGTCGTGAACGTCAGGCCGCTGCTCAGGCA
GCGACCGGCGAGCAACTGGACAAGAAGACGCTGAAGAAAGTCGGCCCGAAACCGGGCTGG
CTGGAAACCGGCGCATCGGTTTTCCCGGTGCTGGCGATCGTTCTGGTGGTACGTTCATTT
ATTTATGAGCCTTTCCAGATCCCTTCAGGTTCGATGATGCCAACGCTGCTCATCGGCGAT
TTTATTCTGGTGGAGAAATTTGCCTACGGCATTAAAGATCCTATCTACCAGAAAACGCTG
ATCGAGACCGGCCATCCGAAGCGCGGCGACATCGTGGTATTTAAATATCCGGAAGACCCG
CGTCTGGACTACATTAAGCGCGCGGTGGGGTTACCGGGTGATAAGGTCACCTACGATCCG
GTTGCCAAACAGGTCACTATTCAGCCGGGCTGCAGTTCCGGACAGGCCTGCGGCAACGCG
CTGCCGGTGACCTATTCCAACGTGGAGCCGAGCGATTTTGTTCAGACCTTCTCCCGCAGC
AACGGCGGCGAAGCGAGCAGCGGTTTCTGGCAGTTGCCGAAGGGCGAAACCAAAGCCGAC
GGCATTCGTCTTACCGAGCGTCAGGAGACATTGGGCGACGTGACGCACCGAATTCTGATG
GTGCCGATTGCCCAGGATCAGGTTGGGATGTACTACCATCAGTCCGGTCTGCCGCTGGCC
ACCTGGATTGTGCCGCCCGGTCAGTACTTCATGATGGGCGACAACCGGGATAACAGCGCC
GACAGCCGGTACTGGGGCTTTGTGCCGGAAGCCAACCTGGTCGGAAAAGCAACCGCTATC
TGGATGAGTTTTGAAAAGCAGGAAGGTGAATGGCCGACCGGCGTGCGGTTATCGCGCATT
GGTGGAATTCATTAA
4_Mycobacterium tuberculosis H37Rv_GI#
15610040_Catalytic Ser Residues 96 (SEQ ID NO:)
VTETTDSPSERQPGPAEPELSSRDPDIAGQVFDAAPFDAAPDADSEGDSKAAKTDEPRPA
KRSTLREFAVLAVIAVVLYYVMLTFVARPYLIPSESMEPTLHGCSTCVGDRIMVDKLSYR
FGSPQPGDVIVFRGPPSWNVGYKSIRSHNVAVRWVQNALSFIGFVPPDENDLVKRVIAVG
GQTVQCRSDTGLTVNGRPLKEPYLDPATMMADPSIYPCLGSEFGPVTVPPGRVWVMGDNR
THSADSRAHCPLLCTDDPLPGTVPVANVIGKARLIVWPPSRWGVVRSVNPQQGR*
4_Mycobacterium tuberculosis H37Rv_GI#
15610040_Catalytic Ser nucleotides 286-288 (SEQ ID NO:)
GTGACCGAAACCACGGACTCCCCATCGGAGCGCCAGCCGGGTCCGGCAGAGCCGGAGCTC
TCCTCCCGGGACCCGGACATTGCCGGCCAGGTCTTCGACGCAGCCCCGTTCGACGCAGCC
CCGGATGCGGACTCCGAAGGCGACTCCAAGGCGGCCAAAACGGACGAGCCGCGGCCCGCG
AAGCGATCAACGCTGCGGGAGTTCGCGGTGCTGGCGGTGATTGCTGTGGTGCTCTACTAC
GTCATGCTGACGTTTGTCGCGCGCCCTTATCTGATTCCGTCGGAATCGATGGAACCCACG
TTGCACGGGTGTTCGACGTGCGTCGGCGACCGCATCATGGTGGACAAACTCAGCTACCGC
TTCGGCTCACCGCAACCTGGCGACGTCATCGTCTTCAGGGGACCGCCGTCGTGGAACGTT
GGTTACAAGTCGATCCGTTCGCACAACGTCGCCGTGCGCTGGGTGCAGAACGCGTTGTCG
TTCATCGGTTTCGTGCCTCCCGACGAGAACGACCTGGTCAAGCGTGTCATCGCGGTCGGC
GGACAGACGGTTCAATGCCGGTCCGACACCGGCCTGACGGTCAACGGCAGGCCACTGAAG
GAGCCATACCTGGATCCGGCCACCATGATGGCCGACCCGTCGATATACCCGTGCCTGGGC
AGCGAGTTCGGGCCGGTCACCGTCCCGCCCGGGCGTGTCTGGGTGATGGGCGACAACCGC
ACCCATTCGGCGGATTCCCGCGCTCACTGCCCGTTGCTATGTACTGACGATCCGCTACCG
GGGACCGTGCCGGTGGCCAACGTCATCGGTAAGGCCAGGTTGATCGTGTGGCCGCCGTCG
CGTTGGGGTGTTGTGCGTTCGGTGAATCCCCAGCAAGGTCGGTAG
5_Yersinia pestis KIM 10_GI# 22123922_Catalytic Ser
Residues 98 (SEQ ID NO:)
MANMFALILAIATLLTGIIWCFERFKWGPARQAKIAAVNAQTAEIKAQTGCAVDNKTLAQ
AAKQPGWIETCASIFPVLALVFIVRSFIYEPFQIPSGSMMPTLLIGDFILVEKFAYGIKD
PITQTTLIPTGKPNRGDIAVFKYPLDPRLDYIKRVVGLPGDRVIYNPISKEVTVQPSCNT
GTSCDSALAITYSTSEPSEFVQTFRYSGNGESSAGFFPIPLNQAVPDGGVRLRERTESLG
PVAHHILTVPGRQDPLGSYYQQPDQPLGVWVVPEGHYFMMGDNRDNSADSRFWGFVPERN
LVGKATAIWMSFEKQEGEWPTGVRLSRIGGIH*
5_Yersinia pestis KIM 10_GI# 22123922_Catalytic Ser
nucleotides 292-294 (SEQ ID NO:)
ATGGCTAACATGTTTGCTTTGATTCTGGCAATAGCAACGCTGTTGACGGGGATTATCTGG
TGCTTCGAGCGGTTTAAATGGGGGCCAGCCCGTCAGGCAAAAATTGCGGCAGTTAATGCA
CAAACTGCGGAAATCAAGGCCCAAACCGGGTGTGCCGTAGATAATAAAACCTTAGCCCAA
GCTGCAAAGCAACCGGGTTGGATCGAGACATGTGCCTCTATCTTCCCGGTGCTGGCCTTG
GTCTTTATCGTGCGTTCGTTTATTTACGAGCCTTTCCAGATCCCTTCTGGTTCGATGATG
CCAACGCTGCTTATCGGTGATTTTATTTTGGTTGAGAAATTTGCTTATGGGATTAAAGAT
CCCATTACTCAGACCACATTAATTCCAACAGGTAAGCCAAACCGCGGTGACATTGCGGTG
TTTAAATATCCGTTGGATCCACGTTTGGATTATATCAAGCGTGTGGTGGGGCTGCCGGGG
GATCGGGTAATTTATAACCCGATAAGTAAAGAAGTCACGGTACAACCGTCATGTAATACC
GGTACTTCTTGTGATAGTGCGTTGGCCATCACTTACAGCACGTCTGAGCCAAGTGAGTTT
GTGCAGACATTCCGTTATAGCGGTAATGGCGAAAGCTCCGCAGGGTTCTTCCCAATCCCG
CTAAATCAGGCAGTACCTGATGGCGGTGTCCGGTTACGTGAGCGTACTGAAAGCCTCGGC
CCGGTAGCGCATCACATTCTGACCGTCCCAGGGCGGCAGGATCCGTTAGGCTCTTATTAT
CAGCAACCCGATCAACCGTTAGGGGTTTGGGTGGTACCGGAAGGCCATTACTTTATGATG
GGTGATAACCGGGATAACAGTGCAGATAGCCGCTTCTGGGGTTTTGTACCAGAACGTAAT
CTGGTAGGTAAGGCTACGGCTATTTGGATGAGTTTTGAAAAGCAAGAAGGTGAATGGCCA
ACGGGTGTGCGTTTAAGCCGAATTGGTGGAATTCACTAA
6_Staphylococcus aureus subsp. aureus COL_GI#
57651657_Catalytic Ser Residues 39 (SEQ ID NO:)
VSKLKKEILEWIISIAVAFVILFIVGKFIVTPYTIKGESMDPTLKDGERVAVNIVGYKTG
GLEKGNVVVFHANKNDDYVKRVIGVPGDKVEYKNDTLYVNGKKQDEPYLNYNLKHKQGDY
ITGTFQVKDLPNANPKSNVIPKGKYLVLGDNREVSKDSRAFGLIDEDQIVGKVSFRFWPF
SEFKHNFNPENTKN*
6_Staphylococcus aureus subsp. aureus COL_GI#
57651657_Catalytic Ser nucleotides 115-117 (SEQ ID NO:)
GTGTCAAAATTGAAAAAAGAAATATTGGAATGGATTATTTCAATTGCAGTCGCTTTTGTC
ATTTTATTTATAGTAGGTAAATTTATTGTTACGCCATATACAATTAAAGGTGAATCAATG
GATCCAACTTTGAAAGATGGCGAGCGAGTAGCTGTAAACATTGTTGGATATAAAACAGGT
GGTTTGGAAAAAGGTAATGTAGTTGTCTTCCATGCAAACAAAAATGATGACTATGTTAAA
CGTGTCATCGGTGTTCCTGGTGATAAAGTAGAATACAAAAATGATACATTATATGTCAAT
GGTAAAAAACAAGATGAACCATATTTAAACTACAATTTAAAACATAAACAAGGTGATTAC
ATTACTGGGACTTTCCAAGTTAAAGATTTACCGAATGCGAATCCTAAATCAAATGTCATT
CCAAAAGGTAAATATTTAGTGCTTGGAGATAATCGTGAAGTAAGTAAAGATAGCCGTGCG
TTTGGCCTCATTGATGAAGACCAAATTGTTGGTAAAGTTTCATTTAGGTTCTGGCCATTT
AGTGAATTTAAACATAATTTCAATCCTGAAAATACTAAAAATTAA
7_Vibrio cholerae O1 biovar El Tor str. N16961_GI#
15642458_Catalytic Ser Residues 90 (SEQ ID NO:)
MANTFSLILVIVTLVTGIVWTLEKLVWAKKRQQKQAHLQAQTPDMPASALDKVVAQPWWI
ENSVSIFPVIAFVLVLRSFIYEPFQIPSGSMMPTLLVGDFILVEKYAYGLKDPVWRTQLV
ETGKPERGDIVVFKYPVNPEIDYIKRVVGMPGDTVRYSAGKELCIQHQGESECQAVKLSN
VQESEFYQNEIPLIQLNEQLGKVEHNILVNPLSIDNVANYRPRSGVNEWVVPQGHYFVMG
DNRDNSADSRFWGFVPEQNLVGKAVAIWISFEFERAEDSVLPRWIPTGVRFNRVGGIH*
7_Vibrio cholerae O1 biovar El Tor str. N16961_GI#
15642458_Catalytic Ser nucleotides 268-270 (SEQ ID NO:)
ATGGCGAACACATTCTCACTGATTTTGGTGATCGTAACTCTGGTCACCGGTATCGTCTGG
ACACTGGAAAAGCTGGTGTGGGCGAAAAAACGCCAACAGAAACAAGCTCATTTACAGGCG
CAAACGCCCGATATGCCAGCCTCAGCGCTGGATAAAGTCGTGGCTCAGCCGTGGTGGATT
GAAAACAGTGTCTCGATTTTCCCTGTTATTGCTTTTGTGCTGGTACTGCGCTCGTTCATT
TATGAACCGTTCCAAATTCCATCCGGTTCGATGATGCCGACTCTGCTGGTCGGGGATTTT
ATTCTGGTTGAGAAATACGCTTACGGCTTGAAAGATCCTGTATGGCGCACTCAGTTAGTG
GAAACGGGTAAACCTGAGCGTGGTGATATTGTGGTGTTCAAATACCCAGTGAACCCTGAG
ATCGACTACATCAAACGTGTGGTGGGGATGCCCGGAGATACCGTACGTTACAGCGCAGGT
AAAGAGCTGTGTATTCAGCACCAAGGCGAGAGCGAATGCCAAGCAGTTAAACTCTCTAAC
GTGCAAGAGAGCGAGTTTTACCAAAATGAGATCCCCCTGATCCAGCTGAACGAACAGCTA
GGTAAGGTTGAGCACAATATTTTGGTTAACCCATTGAGCATTGATAACGTGGCGAATTAT
CGCCCACGCAGTGGCGTGAATGAATGGGTTGTACCACAAGGGCACTATTTTGTGATGGGT
GATAACCGTGACAACAGTGCTGACAGCCGTTTCTGGGGCTTTGTGCCAGAGCAGAATCTG
GTCGGAAAAGCTGTGGCTATCTGGATCAGTTTCGAGTTTGAACGCGCTGAAGACAGCGTA
CTTCCACGCTGGATTCCTACCGGAGTACGATTCAATCGTGTTGGTGGGATCCACTAA
8_Haemophilus influenzae 86-028NP_GI#
68248566_Catalytic Ser Residues 115 (SEQ ID NO:)
MSNLFFVILLAVGFGVWKVLDYFQLPNTFSILLLILTALSGVLWCYHRFVVLPKRHRQVA
RAEQRSGKTLSEEEKAKIEPISEASEFLSSLFPVLAVVFLVRSFLFEPFQIPSGSMESTL
RVGDFLVVNKYAYGVKDPIFQNTIIEGEKPQRGDVIVFKAPQQALIRTGLGATRAAFAEN
LALSSKDNMSGVDYIKRIVGKGGDRIIFDVEQKTLKIVYGKDGKPCEVDCETKAFEYTQN
PTNPAFPNELELTEKGDVTHNVLIGEYRRYSDLEFFPQEGMQTAEWLVPEGQYFVMGDHR
DHSDDSRFWGFVPEKNIVGKATYIWMSLEKEANEWPTGFRFDRFFTAIK*
8_Haemophilus influenzae 86-028NP_GI#
68248566_Catalytic Ser nucleotides 343-345 (SEQ ID NO:)
ATGTCAAATTTATTTTTTGTGATTTTATTGGCTGTCGGCTTTGGTGTGTGGAAAGTTTTA
GATTATTTTCAGTTGCCAAATACTTTTAGTATTTTGTTACTAATTTTGACCGCACTTTCT
GGCGTATTATGGTGTTATCATCGTTTTGTGGTGCTGCCAAAACGTCATCGTCAAGTGGCA
CGTGCAGAACAACGTTCTGGTAAAACCTTAAGTGAGGAAGAAAAAGCCAAAATTGAACCG
ATTTCTGAGGCTTCAGAATTTTTGTCTTCACTTTTTCCTGTGCTTGCAGTGGTATTTTTG
GTTCGTTCTTTTTTGTTTGAACCGTTTCAAATTCCCTCTGGCTCAATGGAGTCCACTTTA
CGCGTTGGCGATTTTTTAGTTGTGAATAAATATGCTTATGGTGTGAAAGATCCGATTTTC
CAAAACACCATTATTGAGGGCGAAAAACCACAACGTGGCGATGTGATTGTGTTTAAAGCA
CCACAACAAGCGTTAATTCGTACTGGTCTTGGGGCTACTCGAGCGGCTTTTGCAGAAAAT
TTAGCGTTAAGTTCAAAAGATAATATGTCTGGTGTGGATTATATTAAGCGTATTGTTGGA
AAGGGCGGAGATCGCATCATTTTTGATGTGGAACAAAAAACATTAAAAATTGTATATGGC
AAAGATGGTAAACCTTGTGAAGTTGATTGCGAAACCAAGGCGTTTGAATATACACAAAAT
CCAACAAATCCTGCTTTTCCGAATGAATTAGAATTGACTGAAAAAGGCGATGTAACACAT
AACGTGTTAATTGGTGAGTATCGTCGTTATTCAGACCTTGAATTTTTCCCACAAGAGGGA
ATGCAAACTGCAGAATGGCTTGTGCCAGAGGGGCAGTATTTTGTGATGGGGGATCATCGC
GATCACAGCGATGACAGTCGTTTTTGGGGCTTTGTGCCTGAAAAAAATATTGTGGGGAAA
GCCACTTATATTTGGATGAGCTTAGAAAAAGAAGCGAATGAATGGCCAACAGGTTTCCGT
TTTGATCGCTTCTTTACAGCAATAAAATAA
9_Pseudomonas aeruginosa PAO1_GI# 15595965_Catalytic
Ser Residues 90 (SEQ ID NO:)
MTLNFPLLLVIAVAVCGALALVDLVLFAPRRRAAISSYEGQVNEPDPAVLEKLNKEPLLV
EYGKSFFPVLFIVLVLRSFLVEPFQIPSGSMKPTLEVGDFILVNKFAYGIRLPVLDTKVI
PIGDPQRGDVMVFRYPSEPNINYIKRVVGLPGDTVRYTKEKRLYVNGELVAEKLVGEEPG
TLGSVTLYQEKLGQAEHLIRKEMSRYRIEPDRQWTIPAGHYFMMGDNRDNSNDSRYWNDP
KIPKDLLGMVPDRNIVGKAFAVWMSWPDPKMSNLPNFSRVGVIH*
9_Pseudomonas aeruginosa PAO1_GI# 15595965_Catalytic
Ser nucleotides 268-270 (SEQ ID NO:)
ATGACACTCAATTTCCCGTTGTTGCTGGTCATCGCCGTGGCTGTATGCGGCGCCCTGGCC
CTGGTCGACCTGGTGCTGTTCGCGCCGCGTCGGCGGGCCGCGATCTCTTCCTACGAAGGG
CAGGTGAACGAGCCCGATCCGGCAGTGCTGGAGAAGCTCAACAAGGAACCGCTGCTGGTG
GAGTACGGCAAGTCGTTCTTCCCGGTGCTGTTCATCGTGCTGGTGCTGCGTTCCTTCCTG
GTCGAGCCGTTCCAGATTCCCTCGGGGTCGATGAAACCTACCCTCGAGGTCGGCGATTTC
ATCCTGGTCAACAAGTTCGCCTACGGTATCCGCCTGCCGGTGCTGGACACCAAGGTGATC
CCGATCGGTGATCCGCAGCGCGGCGATGTCATGGTGTTCCGCTATCCCAGCGAACCGAAC
ATCAACTACATCAAGCGCGTGGTCGGCCTGCCCGGCGACACCGTGCGCTACACCAAGGAA
AAGCGCCTGTACGTCAACGGCGAGCTGGTGGCGGAGAAACTGGTCGGCGAGGAACCGGGC
ACCCTGGGCAGCGTGACCCTGTACCAGGAGAAGCTGGGCCAGGCCGAGCACCTGATCCGC
AAGGAAATGAGCCGCTATCGCATCGAGCCCGACCGCCAGTGGACCATTCCCGCCGGCCAC
TACTTCATGATGGGCGACAACCGCGACAACTCCAACGACAGCCGCTACTGGAACGATCCG
AAGATCCCCAAGGATCTGCTGGGCATGGTTCCGGACCGCAATATCGTCGGCAAGGCCTTC
GCCGTGTGGATGAGCTGGCCCGATCCGAAGATGAGCAACCTGCCGAACTTCTCCCGGGTC
GGCGTGATTCACTGA
10_Acinetobacter baumannii ATCC 19606_GI#
260556580_Catalytic Ser Residues 72 (SEQ ID NO:)
VDFDFNLILVPVTLILFAVWLLDKLVFKQRANKGRENENFVITWAYDFWPVLAVVLVLRS
FLYEPFNIPSDSMVPTLETGDFILVNKFDYGVRLPIVNKKVIDVGEPKRGDVIVFRYPPQ
PTISYIKRVIGLPGDHIVYDHGQLIINGQKIPKVPTQFSREKDALDTPTSIYHKETIGDH
TFTMRELEGVNVARQAPFINYVDNGKYANQDGLYWEVTVPKGHYFAMGDNRDQSADSRFW
GFVPEENLTGRAFYVWMHKEPGFHLPSFNRNGKID*
10_Acinetobacter baumannii ATCC 19606_GI#
260556580_Catalytic Ser nucleotides 214-216 (SEQ ID NO:)
GTGGATTTTGATTTTAATTTAATTCTTGTTCCTGTTACGCTGATTTTATTTGCAGTGTGG
TTGCTAGATAAGCTTGTTTTTAAACAGCGTGCAAATAAAGGGCGAGAGAACGAAAATTTT
GTTATTACATGGGCCTATGACTTTTGGCCGGTTTTAGCTGTTGTGCTTGTACTTCGCTCA
TTTCTTTATGAACCATTTAATATTCCATCAGACTCTATGGTTCCGACCTTAGAGACTGGC
GATTTTATTTTAGTTAATAAATTTGACTATGGTGTCCGTTTACCTATCGTCAATAAAAAA
GTGATTGATGTCGGTGAACCGAAACGTGGTGATGTCATTGTATTCCGTTATCCACCACAA
CCTACTATTAGTTATATTAAACGTGTAATTGGCTTACCTGGTGACCATATTGTTTATGAT
CATGGACAATTGATTATTAATGGTCAAAAAATTCCTAAAGTACCAACACAGTTTAGTCGC
GAAAAAGATGCTTTAGATACACCAACTTCTATTTATCATAAAGAAACAATTGGTGATCAT
ACTTTTACGATGCGTGAGCTTGAAGGCGTAAATGTTGCGCGTCAGGCGCCATTTATCAAC
TATGTTGATAATGGTAAATATGCAAACCAAGACGGTTTATATTGGGAAGTAACAGTTCCG
AAAGGACATTACTTTGCAATGGGGGATAACCGTGATCAAAGTGCTGACAGTCGTTTCTGG
GGCTTCGTACCTGAAGAAAATTTAACAGGCCGAGCTTTCTATGTCTGGATGCATAAAGAA
CCTGGTTTCCACCTGCCAAGCTTTAACCGAAATGGGAAAATAGATTAA
11_Bacillus anthracis str. Ames_GI#
30263049_Catalytic Ser Residues 40 (SEQ ID NO:)
MKENTKKELFSWAKTIGFTLVLIAIIRGVLFTPSLVQGESMMPTLENNERVLVNKIGYSI
SGLERFDIIVFHGKEGYDLVKRVIGLPGDTVEYKNDVLYVNGKAMEEPYLKEFKEKAAGR
VLTPDFTLEQITGKTKVPEGQVFVLGDNREVSKDGRMFGFISEDEIVGKGQAVFWPLKQV
RAL*
11_Bacillus anthracis str. Ames_GI#
30263049_Catalytic Ser nucleotides 118-120 (SEQ ID NO:)
ATGAAGGAAAATACGAAGAAAGAATTATTCTCATGGGCGAAAACGATAGGATTTACCCTT
GTATTAATCGCAATTATTCGCGGTGTTTTATTTACACCGTCATTAGTACAAGGCGAATCG
ATGATGCCGACTTTAGAAAATAACGAACGAGTTCTCGTCAATAAGATTGGTTATAGTATA
AGTGGATTAGAACGCTTTGATATTATCGTTTTCCATGGAAAAGAAGGATATGATTTAGTA
AAACGAGTAATTGGTTTACCAGGCGATACAGTTGAGTATAAAAATGATGTTTTATATGTA
AACGGCAAAGCGATGGAAGAACCATATTTAAAAGAGTTTAAAGAAAAAGCAGCAGGTCGT
GTATTAACTCCAGACTTTACGTTAGAACAAATTACAGGAAAAACGAAAGTGCCAGAAGGC
CAAGTGTTTGTATTAGGTGATAATCGTGAAGTTTCTAAAGACGGTCGTATGTTTGGATTT
ATTTCAGAAGATGAAATTGTCGGAAAAGGACAAGCTGTTTTCTGGCCGTTGAAACAAGTA
AGAGCGCTATAA
12_Neisseria meningitidis MC58_GI# 15676663_Catalytic
Ser Residues 123 (SEQ ID NO:)
MNTMLMSGAAAALLAGIILYFKSDKKRQENGEWSSGLEYAYILTAVGVFAALSLFMSFTA
VFLIFVVLCGTAWGVYKYRLKTHPEISESSHFGDYFGSFFPTVLVLFLIRSFIAEPFQIP
SSSMRPGLIKGDFILVGKFSYGLRVPVLNNIFIPTGKIERGDVVVFNYPLQPEMTYIKRI
VGIPGDVVEYRDKILTVNGKPTSDIPDGTYRYPDDTDPSEIHNTDMFRSGLDGKSFNILK
KEGQPAVSLPVLGKYTSDIMSENGYSIEQSGLEHCQYADDGSGFVCKVPEGRYFAMGDNR
DNSADSRYWGFVDDKLVVGKAMFILMNFGDFGRSGTAIR*
12_Neisseria meningitidis MC58_GI# 15676663_Catalytic
Ser nucleotides 367-369 (SEQ ID NO:)
ATGAACACAATGCTAATGTCGGGCGCGGCTGCCGCGCTGCTTGCCGGCATCATCCTTTAT
TTCAAAAGCGACAAGAAGCGGCAGGAAAACGGGGAATGGAGTTCCGGCCTTGAATACGCC
TATATCCTGACAGCGGTCGGCGTGTTTGCCGCTTTGTCCCTGTTTATGAGCTTTACCGCC
GTTTTCCTGATTTTCGTTGTATTGTGCGGTACGGCTTGGGGGGTATATAAATACCGCCTG
AAGACTCATCCCGAAATCTCGGAAAGCAGCCACTTCGGCGATTATTTCGGCAGTTTCTTC
CCTACCGTTTTGGTATTGTTCCTCATCCGGTCGTTTATCGCCGAACCGTTCCAAATCCCG
TCCAGCTCGATGCGCCCGGGCCTGATCAAGGGCGATTTCATTTTGGTCGGCAAATTTTCC
TACGGCCTGCGCGTACCCGTTTTAAACAATATATTTATTCCTACAGGCAAAATCGAACGG
GGCGATGTCGTTGTTTTTAATTATCCTCTGCAGCCGGAGATGACCTACATCAAGCGTATT
GTCGGCATTCCGGGCGATGTGGTCGAATATCGGGATAAGATTTTGACGGTAAATGGCAAA
CCCACTTCCGACATTCCTGACGGCACATACCGTTATCCCGACGACACCGACCCTTCCGAA
ATCCACAACACGGATATGTTCCGCAGCGGTCTAGACGGCAAATCCTTCAATATTCTGAAA
AAAGAAGGACAGCCTGCCGTTTCCCTGCCCGTATTGGGCAAATATACCTCCGATATTATG
TCTGAAAACGGATATTCCATAGAGCAAAGCGGTTTGGAACACTGCCAATATGCCGACGAC
GGCAGCGGTTTCGTGTGCAAAGTTCCCGAAGGACGCTATTTCGCTATGGGCGACAACCGC
GACAACAGTGCCGATTCGCGCTACTGGGGATTTGTGGATGACAAGCTGGTTGTCGGCAAG
GCAATGTTCATTTTGATGAACTTCGGCGATTTCGGCAGGTCCGGTACGGCAATCCGTTAG
13_Bacillus anthracis str. Ames_GI# 30263037_Catalytic
Ser Residues 35 (SEQ ID NO:)
MKQEIKRGWGKYILFVFVLVVAYHSFTLCKVEGKSMQPTLYEEDYVFVNKAAVHFSDLEH
GEIVIIKEEDESKYYVKRVIGLPGDVINITNGSVYVNDKKQEEPYTNKDLFNNTQVFYNF
QKTKIPPNKLFVMGDNRELSRDSRNGLGYIEEDNIIGKVEFVYYPFSKMKIIE*
13_Bacillus anthracis str. Ames_GI# 30263037_Catalytic
Ser nucleotides 103-105 (SEQ ID NO:)
ATGAAACAGGAGATTAAAAGAGGTTGGGGGAAATATATACTCTTCGTGTTTGTTTTGGTA
GTAGCTTATCATTCTTTTACTTTATGTAAAGTGGAAGGGAAATCAATGCAACCGACTTTA
TATGAAGAAGACTACGTATTTGTAAATAAAGCAGCAGTACATTTTTCCGATTTAGAGCAT
GGAGAAATTGTCATTATAAAGGAAGAGGATGAATCGAAATATTATGTAAAACGAGTAATA
GGACTTCCTGGTGACGTAATTAACATAACGAATGGATCTGTATATGTAAATGATAAAAAA
CAAGAAGAACCGTATACAAATAAAGATTTATTCAATAATACGCAAGTGTTTTATAACTTT
CAAAAGACAAAAATCCCACCAAATAAATTATTTGTAATGGGAGATAATCGTGAACTTAGT
AGAGATAGTCGAAACGGTTTAGGATATATTGAAGAAGATAATATAATAGGCAAAGTGGAA
TTTGTATATTATCCTTTTTCAAAAATGAAGATCATAGAATAA
14_Streptococcus mutans UA159_GI# 24380230_Catalytic
Ser Residues 35 (SEQ ID NO:)
MKRFLKEWGLFLVIIFALLLPRLFIWFPVQVDGHSMDPTLANGEHLIVVRTTSIKHFDIV
VAAEGNKNIVKRVIGMPGDTITYENDMLSINGKKVNETYLKQYKDKFAKDKLQKTYAYNQ
YFQELASQSTAFTTDEQGNASFTIKVPKGRYLLLGDDRIVSKDSRHVGTFAKNKIVGEVK
FRFWPLNAIRFISNK*
14_Streptococcus mutans UA159_GI# 24380230_Catalytic
Ser nucleotides 103-105 (SEQ ID NO:)
ATGAAAAGATTTTTAAAAGAATGGGGCCTTTTCTTGGTCATCATTTTCGCATTGCTACTC
CCGCGTCTCTTTATCTGGTTTCCTGTCCAAGTAGATGGACATTCAATGGATCCTACCTTA
GCCAATGGGGAGCATCTCATTGTCGTCAGGACAACTTCTATCAAACATTTTGACATTGTT
GTTGCTGCTGAAGGCAATAAAAATATTGTCAAACGTGTGATTGGCATGCCCGGTGATACC
ATTACCTATGAAAATGATATGCTTTCTATTAATGGGAAAAAAGTCAATGAAACTTATCTC
AAGCAATACAAGGATAAATTTGCCAAGGACAAACTCCAAAAGACTTATGCCTACAATCAG
TATTTCCAAGAATTAGCCTCACAATCAACAGCTTTCACAACAGACGAACAAGGAAACGCC
AGCTTTACGATTAAAGTACCAAAAGGACGTTACCTGCTTTTAGGTGATGATCGCATTGTC
TCTAAAGACAGCCGCCATGTTGGAACTTTTGCTAAGAATAAAATTGTTGGTGAAGTTAAA
TTCCGCTTTTGGCCTTTAAACGCTATTCGTTTCATTTCAAATAAATAA
15_Shigella flexneri 2a str. 301_GI#
24113910_Catalytic Ser Residues 91 (SEQ ID NO:)
MANMFALILVIATLVTGILWCVDKFFFAPKRRERQAAAQAAAGDSLDKATLKKVAPKPGW
LETGASVFPVLAIVLIVRSFIYEPFQIPSGSMMPTLLIGDFILVEKFAYGIKDPIYQKTL
IETGHPKRGDIVVFKYPEDPKLDYIKRAVGLPGDKVTYDPVSKELTIQPGCSSGQACENA
LPVTYSNVEPSDFVQTFSRRNGGEATSGFFEVPKNETKENGIRLSERKETLGDVTHRILT
VPIAQDQVGMYYQQPGQQLATWIVPPGQYFMMGDNRDNSADSRYWGFVPEANLVGRATAI
WMSFDKQEGEWPTGVRLSRIGGIH*
15_Shigella flexneri 2a str. 301_GI#
24113910_Catalytic Ser nucleotides 271-273 (SEQ ID NO:)
ATGGCGAATATGTTTGCCCTGATTCTGGTGATTGCCACACTGGTGACGGGCATTTTATGG
TGCGTGGATAAATTCTTTTTCGCACCTAAACGGCGGGAACGTCAGGCAGCGGCGCAGGCG
GCTGCCGGTGACTCACTGGATAAAGCAACGTTGAAAAAGGTTGCACCGAAGCCTGGCTGG
CTGGAAACCGGAGCTTCTGTTTTTCCGGTGCTGGCTATCGTATTGATTGTACGTTCGTTT
ATTTATGAACCGTTCCAGATCCCGTCAGGTTCGATGATGCCGACTCTGTTAATCGGTGAT
TTTATTCTGGTAGAGAAGTTTGCTTATGGCATTAAAGATCCTATCTACCAGAAAACGCTG
ATCGAAACCGGTCATCCGAAACGCGGCGATATCGTGGTCTTTAAATATCCGGAAGATCCA
AAGCTTGATTACATCAAGCGCGCGGTGGGTTTACCGGGCGATAAAGTCACTTACGATCCG
GTCTCAAAAGAGCTGACGATTCAACCGGGATGCAGTTCCGGCCAGGCGTGTGAAAACGCG
CTGCCGGTCACCTACTCAAACGTGGAACCGAGCGATTTCGTTCAGACCTTCTCACGCCGT
AATGGTGGGGAAGCGACCAGCGGATTCTTTGAAGTGCCGAAAAACGAAACCAAAGAAAAT
GGAATTCGTCTTTCCGAGCGTAAAGAGACACTGGGTGATGTGACGCACCGAATTCTGACA
GTGCCGATTGCGCAGGACCAGGTGGGGATGTATTACCAGCAGCCAGGGCAACAACTGGCA
ACCTGGATTGTTCCGCCGGGACAATACTTCATGATGGGCGACAACCGCGACAACAGCGCG
GACAGCCGTTACTGGGGCTTTGTGCCTGAAGCGAATCTGGTCGGTCGGGCCACGGCTATC
TGGATGAGCTTCGATAAGCAAGAAGGCGAATGGCCGACTGGTGTGCGCTTAAGTCGCATT
GGCGGCATCCATTAA
16_Citrobacter koseri ATCC BAA-895_GI#
157144497_Catalytic Ser Residues 91 (SEQ ID NO:)
MANMFALILVIATLVTGILWCVDKFIFAPKRRERQAAAQAAAGDSLDKATLKKVAPKPGW
LETGASVFPVLAIVLVVRSFIYEPFQIPSGSMMPTLLIGDFILVEKFAYGIKDPIYQKTL
IETGHPKRGDIVVFKYPEDPRLDYIKRAVGLPGDKVTYDPVAKEVTVQPGCRSGQACENA
LPVTYSDVQPSDFVQTFARRNGGEASSGFFEVPLNETKDNGIRLAERKETLGDVTHRILT
VPIAQDQAGMYYRQPGQQLATWIVPPGQYFMMGDNRDNSADSRYWGFVPEANLVGKATAI
WMSFDKQEGEWPTGVRLSRIGGIH*
16_Citrobacter koseri ATCC BAA-895_GI#
157144497_Catalytic Ser nucleotides 271-273 (SEQ ID NO:)
ATGGCGAATATGTTTGCCCTGATTCTGGTGATTGCCACACTGGTGACGGGCATTTTATGG
TGCGTTGATAAATTTATCTTCGCGCCAAAACGTCGGGAACGTCAGGCAGCGGCACAGGCC
GCTGCGGGTGATTCACTGGATAAAGCCACGTTGAAAAAAGTGGCGCCTAAGCCGGGCTGG
CTGGAAACAGGGGCTTCGGTTTTTCCGGTACTGGCGATTGTGCTGGTGGTGCGCTCATTT
ATCTATGAACCTTTCCAGATCCCGTCGGGTTCGATGATGCCGACGCTGTTAATCGGTGAC
TTTATTCTGGTGGAGAAATTCGCCTATGGAATTAAAGATCCGATTTACCAGAAAACGTTG
ATTGAAACGGGTCATCCGAAACGCGGTGATATCGTGGTCTTTAAATACCCGGAAGATCCG
CGCCTGGACTACATTAAACGCGCTGTCGGCCTGCCGGGTGACAAAGTGACGTACGATCCG
GTAGCCAAAGAGGTTACTGTACAGCCAGGATGCCGTTCCGGTCAGGCGTGTGAAAACGCG
CTGCCGGTGACTTACTCTGACGTTCAGCCCAGCGATTTCGTGCAGACCTTTGCCCGCCGT
AATGGGGGAGAAGCCAGCAGTGGGTTCTTCGAAGTGCCGTTAAACGAAACGAAAGATAAC
GGCATTCGTCTGGCGGAGCGTAAAGAGACGCTGGGAGACGTAACCCACCGTATTCTGACC
GTACCGATCGCGCAGGATCAGGCGGGGATGTATTACCGTCAGCCGGGGCAGCAACTGGCG
ACCTGGATCGTACCGCCAGGACAATACTTCATGATGGGTGATAACCGCGATAACAGCGCG
GACAGCCGTTACTGGGGATTTGTACCGGAAGCGAATCTGGTTGGTAAAGCGACCGCGATC
TGGATGAGTTTCGACAAACAGGAAGGTGAATGGCCGACCGGCGTACGCTTAAGCCGTATT
GGTGGGATCCATTAA
17_Bordetella pertussis Tohama I_GI#
33593416_Catalytic Ser Residues 101 (SEQ ID NO:)
MSWNFALILFVLLVITGVIWGLDLALFRKRRERRAQAAAAQVDAAGITDAEQAGRERREA
IDAARRAPWWIEYAVSFFPVILFVFVLRSFVVEPFHIPSGSMLPTLQSGDLILVNKFSYG
IRLPIIDRKIIETGSLERGDVVVFRYPVDTDVDYIKRIVGLPGDQVAYLDKKLYINGKLV
PHERDGDYFEPDRVSYIAQYKEKLGEVEHKILLDEQKIQDFGPIWKFPSIQNCQYARNGV
RCTVPPGHYFAMGDNRDNSADSRYWGFVPDGNIVGKAFFVWMNFSDLSRIGRFH*
17_Bordetella pertussis Tohama I_GI#
33593416_Catalytic Ser nucleotides 301-303 (SEQ ID NO:)
ATGAGTTGGAACTTTGCCCTGATACTTTTTGTACTGCTGGTGATTACCGGCGTTATCTGG
GGATTGGATCTGGCGCTGTTTCGCAAGCGACGCGAACGGCGGGCCCAGGCGGCGGCCGCG
CAAGTGGACGCCGCCGGCATCACGGATGCCGAGCAGGCCGGCCGCGAGCGGCGCGAGGCC
ATCGACGCGGCGCGCCGCGCGCCCTGGTGGATCGAGTATGCGGTCAGCTTCTTCCCGGTG
ATCCTGTTCGTGTTCGTGCTGCGCTCGTTCGTGGTCGAGCCGTTTCACATTCCGTCGGGG
TCCATGCTGCCCACGCTGCAATCGGGCGACCTGATCCTGGTGAACAAGTTCAGCTACGGC
ATCCGCCTGCCCATCATCGATCGCAAGATCATCGAGACGGGCTCGCTGGAGCGTGGCGAC
GTGGTGGTGTTCCGCTACCCGGTCGATACGGATGTCGACTACATCAAGCGCATCGTGGGT
CTGCCGGGCGACCAGGTGGCCTACCTGGACAAGAAGCTGTACATCAACGGAAAATTGGTG
CCGCATGAACGCGACGGGGATTATTTCGAGCCCGATCGCGTGTCCTATATTGCGCAATAC
AAGGAAAAACTGGGCGAAGTGGAGCATAAGATCCTGCTTGATGAGCAGAAAATACAGGAT
TTCGGCCCCATCTGGAAATTTCCCAGTATCCAGAACTGCCAGTACGCCCGCAACGGCGTG
CGCTGTACCGTCCCCCCCGGCCATTATTTCGCCATGGGAGACAACCGTGACAATAGTGCG
GACAGCCGCTACTGGGGATTCGTGCCAGACGGTAATATCGTGGGGAAGGCATTTTTTGTC
TGGATGAACTTCAGCGATTTGAGCCGCATTGGCCGCTTCCATTGA
18_Clostridium difficile 630_GI# 126698930_Catalytic
Ser Residues 37 (SEQ ID NO:)
MSVKKEIFDWIKSIAMAIVLAFVILQFIIPSIVSGESMYPTLDDKDYLILNRISYKVGKP
EKGDIVVFKTNLVDGETGKKKDLIKRVIATEGDRIKISNSKVYVNGKLLNEPYIHNNYTS
GDIDTVVPKGKLFAMGDNRENSNDSRFPDVGMVDEDEVLGKVMVRLLPLDNIGKVD*
18_Clostridium difficile 630_GI# 126698930_Catalytic
Ser nucleotides 109-111 (SEQ ID NO:)
ATGAGTGTTAAAAAAGAAATATTTGATTGGATTAAGTCAATAGCTATGGCTATTGTACTT
GCATTTGTAATTCTACAATTTATAATACCTTCTATTGTAAGTGGAGAATCAATGTATCCT
ACTTTAGATGATAAAGATTATCTGATTTTAAATAGGATATCATACAAGGTTGGTAAACCT
GAAAAAGGCGATATTGTAGTTTTTAAAACCAATTTAGTTGATGGAGAAACAGGAAAGAAA
AAAGACTTAATAAAAAGAGTTATAGCTACTGAAGGTGACAGAATAAAAATATCAAATTCT
AAAGTGTATGTAAATGGAAAATTATTAAATGAACCATATATACACAATAACTATACTTCT
GGAGATATAGATACTGTTGTTCCAAAAGGTAAACTATTTGCAATGGGAGATAATAGAGAA
AATAGTAATGATAGTAGATTCCCTGATGTAGGTATGGTTGATGAAGATGAAGTTCTTGGT
AAGGTTATGGTGAGACTATTACCTCTTGATAATATTGGGAAAGTAGACTAA
19_Clostridium difficile 630_GI# 126698133_Catalytic
Ser Residues 39 (SEQ ID NO:)
VGEAVKKEVVEWIKVIVIALVLAFAITRFIVPTIVKGESMYPTLVERDYLIVNRIAYKVG
EPKYKDIIVFKTDLTEENGKKKDLVKRVIGVPGDHVKIQDSKVYVNDKLLDETSYIHNNR
TDGDIDIVVPEGKLFAMGDNREKSLDSRYDEVGLVDEHTILGKVLVRLYPFSKIGTID*
19_Clostridium difficile 630_GI# 126698133_Catalytic
Ser nucleotides 115-117 (SEQ ID NO:)
GTGGGTGAAGCAGTTAAAAAAGAAGTTGTAGAATGGATAAAAGTGATTGTCATAGCTCTT
GTTTTGGCATTTGCAATAACTCGTTTTATAGTGCCAACAATAGTCAAAGGAGAATCAATG
TATCCTACATTAGTTGAACGTGATTATTTGATAGTTAACAGAATTGCGTACAAGGTAGGA
GAGCCAAAATACAAAGATATAATAGTATTCAAAACCGACTTAACAGAGGAAAATGGAAAG
AAAAAAGATTTAGTAAAAAGAGTTATCGGGGTTCCTGGTGACCATGTAAAAATACAAGAC
TCCAAGGTATATGTAAATGATAAGTTGTTAGATGAGACTTCCTATATACATAATAATCGT
ACTGATGGAGATATTGATATCGTAGTTCCAGAAGGAAAATTATTTGCAATGGGAGATAAT
AGAGAAAAAAGTTTAGATAGTAGATACGATGAGGTTGGATTGGTCGACGAGCATACCATT
TTAGGAAAGGTTCTAGTCAGATTGTATCCATTTTCTAAGATAGGAACTATTGACTAA
20_Clostridium difficile 630_GI# 126698134_Catalytic
Ser Residues 39 (SEQ ID NO:)
MNETIKEEIVEWIKIIITALFFAFIITRFIKPTLVNGESMYPTLKSHDYLVANRMTYKLS
EPKCGDIMIFKTDLLQENGRKKELVKRVIGVPGDHLKIKDSKVYINGKLLNEVSYIHDNY
TEGDIDMVIPKGKVFAMGDNREVSLDSRYKEVGLVDEENIKGKVILRVFPFTDIGIFE*
20_Clostridium difficile 630_GI# 126698134_Catalytic
Ser nucleotides 115-117 (SEQ ID NO:)
ATGAATGAAACTATTAAAGAAGAGATTGTAGAGTGGATAAAAATAATTATTACTGCACTT
TTTTTTGCATTTATTATAACTCGTTTTATAAAACCAACATTAGTAAATGGAGAATCAATG
TACCCAACACTTAAATCACATGATTATTTGGTAGCAAACAGGATGACATATAAGTTATCA
GAACCAAAATGTGGAGATATAATGATATTTAAGACTGATTTATTACAAGAGAATGGAAGG
AAAAAAGAGCTTGTAAAAAGGGTTATAGGTGTTCCTGGTGACCATCTAAAAATTAAGGAT
TCTAAGGTTTATATAAATGGTAAGTTATTAAATGAAGTTTCATATATACATGATAATTAT
ACTGAAGGCGATATTGATATGGTGATTCCTAAGGGAAAAGTATTTGCGATGGGAGACAAT
AGAGAAGTTAGTTTAGACAGTAGATATAAAGAAGTGGGATTAGTAGATGAAGAAAATATT
AAAGGAAAAGTTATTTTAAGAGTATTTCCTTTTACAGATATAGGTATTTTTGAGTAG
21_Enterococcus faecalis V583_GI# 29377531_Catalytic
Ser Residues 35 (SEQ ID NO:)
MSSLLKRLVQLVLLVVAVLLIRHYVFSPAAVNGSSMEPTLHNNDRLWVTSIKKPQRFDII
AFPSPRNGQRVAKRLIGLPGETVEYRDDTLYINGVSLSEDYLASAKRNVSKNENYTQDFT
LETLEATQSLTVPEGMYFVLGDNRPRSDDSRYFGFVKQASVEGVLTFRYYPLDKIGFP*
21_Enterococcus faecalis V583_GI# 29377531_Catalytic
Ser nucleotides 103-105 (SEQ ID NO:)
ATGTCCTCATTATTAAAACGATTGGTTCAGTTGGTTTTGTTAGTCGTCGCTGTCTTGCTG
ATTCGACACTATGTTTTCTCCCCTGCTGCGGTGAACGGCTCTTCAATGGAACCAACACTT
CATAACAACGACCGTTTATGGGTGACCTCGATTAAAAAACCACAGCGCTTTGATATTATC
GCTTTCCCTAGTCCTCGCAACGGCCAACGAGTAGCCAAACGTTTAATTGGTTTACCTGGC
GAAACAGTCGAGTATCGCGATGATACCCTTTATATTAATGGTGTATCACTCAGTGAAGAT
TACTTAGCAAGTGCTAAACGAAATGTCTCTAAAAATGAAAATTATACCCAAGATTTTACG
CTAGAGACCTTAGAAGCCACCCAATCCCTGACCGTTCCAGAAGGCATGTATTTTGTCTTG
GGGGATAATCGCCCGCGCTCAGACGACAGTCGTTATTTTGGCTTTGTTAAACAAGCGAGT
GTGGAAGGTGTTTTGACTTTTCGTTATTATCCATTAGATAAAATTGGCTTTCCATAA
22_Enterococcus faecalis V583_GI# 29375442_Catalytic
Ser Residues 101 (SEQ ID NO:)
MRTIRHIKRAFLKQKLPATYQLKKQKANTAMEYLLEQTDNHQSIRGPKRKMTAEEIKKKR
QAYQKKQRVQVVKFFMPAILFAIFVFFFVLKTSSYPIAGQSMKPTLNAGERVLVQRTKQV
ARYDVIAFKAPLASKGTYVKRIIGVPGDRIWVNEGKLYLSEEPIASDNEALPENASRFDL
SEEAAAQLRLFQKIPAGHYFVLGDNRTHSSDSRTFGFVEIQAIEGIVVFKMAPFKEIGKV
K*
22_Enterococcus faecalis V583_GI# 29375442_Catalytic
Ser nucleotides 301-303 (SEQ ID NO:)
ATGCGAACAATTCGCCACATTAAGCGCGCCTTCTTGAAGCAAAAGTTGCCTGCGACATAT
CAGCTAAAAAAGCAAAAGGCTAACACAGCAATGGAATATTTGCTTGAGCAAACAGATAAC
CATCAATCAATAAGAGGACCGAAAAGAAAAATGACCGCTGAAGAGATTAAAAAAAAGCGG
CAAGCCTACCAAAAGAAACAACGCGTCCAAGTCGTTAAATTTTTTATGCCAGCTATTCTT
TTCGCCATTTTTGTGTTCTTTTTTGTGTTAAAGACATCTAGCTACCCAATTGCTGGGCAA
TCCATGAAGCCGACACTTAACGCAGGGGAACGAGTCTTAGTACAACGGACGAAGCAAGTA
GCAAGGTACGATGTGATTGCATTTAAAGCACCGCTAGCTAGCAAAGGTACGTACGTCAAG
CGAATCATCGGGGTTCCTGGTGATCGAATTTGGGTAAACGAGGGAAAACTTTATCTTTCA
GAAGAACCTATAGCAAGCGATAATGAGGCACTGCCTGAGAATGCCAGTCGTTTTGACTTA
TCAGAAGAAGCGGCAGCCCAACTTCGCCTGTTTCAGAAGATTCCAGCTGGTCATTACTTT
GTCTTAGGGGACAATCGTACGCATTCAAGTGATAGTCGTACGTTCGGCTTTGTCGAGATA
CAAGCGATTGAAGGAATCGTGGTATTTAAAATGGCGCCGTTTAAGGAAATAGGGAAAGTA
AAATAA
23_Enterococcus faecalis V583_GI# 29375687_Catalytic
Ser Residues 39 (SEQ ID NO:)
MSLKSKELIKTVVFFACLALGLFLLRQFVFTPVVVRGHSMDPTLADGERVITLKNTEINR
FDIITFPAPDEPDKNYIKRVIGLPGDTIAYKDDTLYINGKEVDEPYLDEFKKALTDGQPL
TGDFSLKEKVPADSYFVLGDNRRNSKDGRVIGFIHKKDILGEVKFVMWPFSRFGPIPEVS
KQ*
23_Enterococcus faecalis V583_GI# 29375687_Catalytic
Ser nucleotides 115-117 (SEQ ID NO:)
ATGAGTTTGAAATCAAAAGAATTAATTAAAACAGTCGTCTTTTTTGCCTGTTTAGCTTTG
GGTCTGTTTTTACTGAGACAATTTGTATTTACGCCTGTCGTAGTGAGAGGTCATTCAATG
GATCCAACGTTAGCAGATGGTGAACGGGTAATTACGTTAAAAAACACAGAAATTAATCGT
TTCGATATTATTACTTTCCCAGCGCCAGATGAACCAGATAAAAATTATATTAAACGTGTG
ATTGGTTTACCTGGAGATACAATTGCGTACAAGGATGATACGTTGTACATCAATGGAAAA
GAAGTTGACGAACCCTATTTAGATGAATTTAAAAAAGCCTTAACAGATGGTCAACCTTTG
ACAGGCGATTTTTCATTAAAAGAAAAAGTACCAGCAGATAGCTACTTTGTTTTAGGTGAT
AATCGACGGAATTCAAAAGACGGTCGTGTCATTGGTTTTATTCATAAAAAAGATATTTTG
GGTGAAGTGAAATTTGTGATGTGGCCATTCTCACGGTTTGGTCCAATACCAGAAGTGTCA
AAACAATAA
24_Enterococcus faecalis V583_GI# 29376232_Catalytic
Ser Residues 42 (SEQ ID NO:)
LKKKRDYVGYLMYFLKILVPAIVAVFILRGFFLIPVRVDGHSMQKTLNQGDMIVMEKFSA
IKRFDVVVFKTDTGSILIKRVIGLPGEAVRYENDQLYVNNQPIAEPYLTKNRKKDHETMP
YTTNFDSKELLMQEKLPKDSYFVLGDNRRMSKDSRSFGAIHADQILGKAQFVYYPLTHMK
IIPK*
24_Enterococcus faecalis V583_GI# 29376232_Catalytic
Ser nucleotides 124-126 (SEQ ID NO:)
TTGAAGAAGAAACGTGATTATGTTGGGTATTTAATGTACTTTCTGAAAATTTTAGTACCA
GCAATCGTAGCCGTTTTTATTTTAAGAGGATTTTTCCTGATTCCTGTTCGGGTGGATGGC
CATTCTATGCAAAAAACCTTGAATCAAGGAGATATGATTGTGATGGAAAAATTCTCCGCC
ATTAAACGGTTTGATGTGGTGGTCTTTAAAACAGATACAGGATCGATTCTGATTAAACGT
GTGATTGGTTTACCAGGAGAAGCTGTGCGTTACGAAAACGATCAATTATATGICAATAAT
CAGCCAATCGCTGAACCGTATTTAACTAAAAACAGAAAAAAAGATCATGAAACGATGCCT
TACACTACGAATTTTGATTCAAAAGAATTGTTAATGCAAGAAAAATTACCTAAAGATAGC
TATTTTGTGCTTGGTGATAATCGCCGTATGTCCAAAGACAGCCGTTCTTTTGGTGCAATA
CATGCAGATCAAATCTTAGGGAAAGCACAATTTGTTTATTACCCACTCACTCATATGAAG
ATCATTCCTAAATAA
25_Listeria monocytogenes str. 4b F2365_GI#
46907497_Catalytic Ser Residues 49 (SEQ ID NO:)
MTDQYDKKPKKKSGAHQLLSWVLVIVAALAIALVIRNFVVAPVKVEGTSMVPTYQDGDRI
FIEKISKPDRFDIIVFDEPPMIGSGEHFIKRVIGLPGDKIAFKNGELYLNGKRKVENYLP
EGTLTLWNPDPTQKPYIADYTLEDMTGESTVPKGKLFVLGDNRGGSSDSRVFGFIDDSMV
NGTVIQFGK*
25_Listeria monocytogenes str. 4b F2365_GI#
46907497_Catalytic Ser nucleotides 145-147 (SEQ ID NO:)
ATGACAGATCAATATGACAAAAAGCCCAAGAAAAAAAGCGGGGCGCACCAATTATTAAGC
TGGGTGCTAGTTATCGTTGCAGCGCTTGCAATTGCACTTGTGATTCGTAACTTTGTAGTT
GCACCAGTAAAAGTAGAAGGAACATCTATGGTTCCAACATATCAAGATGGCGATAGAATT
TTCATTGAAAAAATTTCCAAGCCTGATCGTTTCGACATTATCGTGTTTGATGAACCTCCA
ATGATTGGTTCAGGAGAGCATTTCATCAAGCGAGTGATTGGTTTGCCGGGAGATAAAATA
GCATTTAAAAACGGTGAATTATATTTAAATGGAAAACGAAAAGTAGAAAATTACTTGCCA
GAAGGAACATTAACCCTTTGGAATCCAGATCCAACGCAAAAACCATACATAGCGGATTAT
ACGCTGGAGGATATGACAGGCGAAAGTACTGTTCCGAAAGGGAAACTATTTGTACTTGGA
GATAATCGCGGCGGGAGTTCAGATAGTCGCGTTTTCGGATTTATTGATGATTCCATGGTA
AACGGTACAGTGATACAATTTGGAAAATAA
26_Listeria monocytogenes str. 4b F2365_GI#
46907496_Catalytic Ser Residues 42 (SEQ ID NO:)
MKSENKFFSGAFGWIKIILIALILAFGIRYFLISPVTVNGKSMDPTLHDGEHLFINKVSD
PKRFDIIVFPAPDEENAEYIKRVIGLPGDKVEYKEDQLYINGKKYDEPYLDSEKEALKNG
YLTTDAEGDPNFTMADIPNSDGSLTVPKGELFVLGDNRQVSKDSRYIGFISQDTVLGKVI
SFGKSLER*
26_Listeria monocytogenes str. 4b F2365_GI#
46907496_Catalytic Ser nucleotides 124-126 (SEQ ID NO:)
ATGAAAAGTGAAAACAAATTTTTTTCTGGGGCATTTGGATGGATAAAAATAATTCTCATC
GCGCTTATACTTGCTTTTGGTATTCGCTATTTTTTAATTTCTCCAGTTACTGTTAATGGG
AAATCAATGGACCCAACACTTCATGATGGGGAACATTTATTTATTAACAAGGTATCAGAT
CCGAAGCGTTTTGACATTATTGTATTTCCTGCGCCTGATGAGGAAAATGCAGAGTACATT
AAACGCGTCATTGGCCTTCCAGGAGATAAAGTGGAGTACAAAGAAGATCAACTTTATATT
AATGGAAAAAAATATGATGAACCTTATTTAGATTCAGAAAAAGAAGCTCTAAAAAACGGT
TATTTAACCACTGATGCAGAAGGCGATCCTAATTTTACGATGGCAGACATTCCAAACTCT
GACGGCTCTCTCACTGTCCCTAAAGGAGAACTTTTTGTTTTAGGAGATAATCGTCAAGTA
AGTAAAGATAGTCGCTACATTGGCTTTATATCACAGGATACCGTGCTTGGAAAAGTAATT
TCATTTGGAAAATCCTTAGAACGTTAA
27_Listeria monocytogenes str. 4b F2365_GI#
46907498_Catalytic Ser Residues 40 (SEQ ID NO:)
LKEKNLKRLWSWIWAAVLAVLIAVIIRFYLFVPILVDGISMMPTLHSDDRVIINRFGNVD
RFDVIVFRESDGKEYIKRVIGLPGDTVEYKEDQLYINGKKYNEPYLDTYKEKLKDGYLTD
DYSSKDQLDGGKIPKDTYFVLGDNRRASKDSRIIGPIPFSKVLGTTPICYWPIEDAK
LID*
27_Listeria monocytogenes str. 4b F2365_GI#
46907498_Catalytic Ser nucleotides 118-120 (SEQ ID NO:)
TTGAAGGAGAAGAATTTAAAACGGTTATGGTCATGGATTTGGGCGGCTGTTCTAGCAGTG
TTAATAGCTGTTATAATCCGTTTTTATTTATTTGTCCCTATTCTCGTCGATGGGATATCA
ATGATGCCTACACTTCATAGCGATGACCGTGTAATTATAAATCGCTTCGGAAATGTAGAT
CGTTTCGATGTGATTGTTTTCCGAGAATCAGATGGAAAAGAATACATCAAGCGAGTGATC
GGTTTGCCGGGTGATACAGTAGAATACAAAGAAGACCAACTTTACATCAATGGTAAAAAG
TATAATGAACCATATTTGGATACTTACAAAGAAAAGTTAAAAGATGGCTATTTAACAGAT
GATTACAGTTCGAAAGATCAACTAGATGGTGGCAAAATTCCAAAAGATACTTATTTTGTT
TTAGGTGACAATCGAAGAGCAAGCAAAGACAGTCGGATAATTGGGCCAATTCCATTTAGC
AAGGTGTTAGGAACAACACCGATTTGTTACTGGCCGATTGAAGATGCCAAACTTATAGAT
TAG

All patents and publications referred to herein are incorporated by reference herein to the same extent as if each individual publication was specifically and individually indicated to be incorporated by reference in its entirety.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A compound of formula (I) 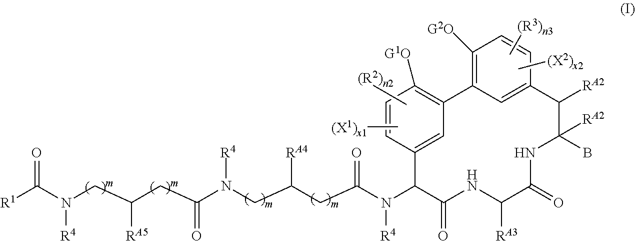
wherein
B is CO₂H, CH₂CO₂H, C(═O)NHCH₂C(═O)H, CH₂C(═O)H, C(═O)NHCH₂B(OR^{B})₂ or C(═O)NHCH₂P(═O)(OR^{B})₂ wherein R^{B} is H, (C₁-C₆)alkyl, or (C₆-C₁₀)aryl; or B is a group of formula 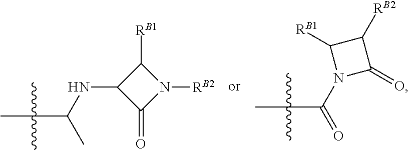
wherein R^{B1} and R^{B2} are each independently H, (C₁-C₆)alkyl, (C₃-C₆)cycloalkyl, OR^{C}, C(═O)NR^{C}₂, OC(═O)NR^{C}₂, C(═O)OR^{C}, OC(═O)OR^{C}, nitro, trifluoromethyl, trifluoromethoxy, (C₁-C₆)alkoxy, (C₁-C₆)thioalkoxy, NR^{C}₂, 5-7 membered heterocyclyl or 5-7 membered heteroaryl, or (C₆-C₁₀)aryl; le is independently at each occurrence H or (C₁-C₆)alkyl, and a wavy line indicates a point of attachment of B to a carbon of formula (I) hearing B;
R¹ comprises a group of formula (IIA), (IIB), or (IIC): 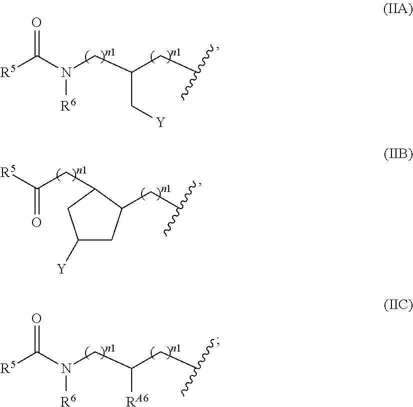
wherein each m is independently 0, 1, or 2, n1 is independently at each occurrence 0, 1, or 2; Y is (CH₂)₀₋₂H, (CH₂)₀₋₂OH, or (CH₂)₀₋₂OC(═O)(C₁-C₆)alkyl; R^{A6} is hydrogen, (C₁-C₆)alkyl, (C₃-C₇)cycloalkyl, 5- to 7-membered heteroaryl, 5- to 7-membered heterocyclyl, or (C₆-C₁₀)aryl, wherein any alkyl, cycloalkyl, heterocyclyl, aryl or heteroaryl may be substituted with 1 to 3 substituents, wherein each substituent is independently selected from the group consisting of halogen, amino, hydroxyl, aminocarbonyl, hydroxycarbonyl, nitro, cyano, trifluoromethyl, trifluoromethoxy, 5- to 7-membered heterocyclyl, (C₁-C₆)alkoxy, (C₁-C₆)alkyl, (C₃-C₇)cycloalkyl, (C₁-C₆)-mono- or di-alkylamino, (C₁-C₆)alkoxycarbonyl, (C₁-C₆)alkylhydroxycarbonyl, (C₁-C₆)alkylaminocarbonyl, (C₁-C₆)alkylsulfonylamino, and (C₆-C₁₀)-arylsulfonylamino; and a wavy line indicates a point of attachment of R¹ to an atom of formula (I) bearing R¹; and
R⁵ is a linear or branched alkyl chain of about 1-22 carbon atoms, bonded to the carbonyl carbon to which it is attached directly or by an O or NH, to provide an amide, carbamate, or urea linkage respectively; optionally comprising within the chain or at a chain terminus, any of the following groups:
(A) 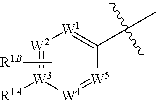
wherein W¹, W², W³, W⁴ and W⁵ are each independently C or N, provided that no more than two of W¹, W², W³, W⁴ and W⁵ are N; provided that when R^{1A} or R^{1B} is non-hydrogen, any W atom to which the R^{1A} or R^{1B} is respectively bonded is C, wherein there can be one or more R^{1B} bonded to the ring bearing the W atoms; R^{1A} is hydrogen, halogen, amino, hydroxyl, aminocarbonyl, hydroxycarbonyl, nitro, fluoroalkyl, cyano, (C₁-C₆)-thioether, fluoroalkoxy, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; R^{1B} is hydrogen, alkyl, halogen, amino, hydroxyl, aminocarbonyl, hydroxycarbonyl, nitro, fluoro alkyl, (C₁-C₆)-thioalkyl, fluoroalkoxy, cyano, (C₁-C₆)-alkyl, (C₁-C₆)-alkoxy, (C₁-C₆)-alkylamino, (C₁-C₆)-alkyl, 5- to 7-membered heteroaryl, 5- to 7-membered heterocyclyl, or (C₆-C₁₀)aryl; wherein any R^{1A} or R^{1B} can be further substituted with one to three (C₁-C₁₂)-alkyl or -alkoxy groups, which can further hear halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-alkylamino, 5- to 7-membered heteroaryl, 5- to 7-membered heterocyclyl, or (C₆-C₁₀)aryl groups; wherein a wavy line indicates a point of attachment;
(B) 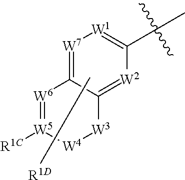
wherein W¹, W², W³, W⁴, W⁵, W⁶, and W⁷ are each independently C or N, provided that no more than three of W¹, W², W³, W⁴, W⁵, W⁶, and W⁷ are N; provided that when R^{1C} or R^{1D} is non-hydrogen, any W atom to which the R^{1C} or R^{1D} is respectively bonded is C, wherein either ring can bear one or more R^{1D}; R^{1C} is hydrogen, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; R^{1D} is hydrogen, alkyl, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein any R^{1C} or R^{1D} can be further substituted with one to three (C₁-C₁₂)-alkyl or -alkoxy groups, which can further bear halogen, amino, hydroxyl, aminocarbonyl, hydroxycarbonyl, nitro, fluoroalkyl, (C₁-C₆)thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein a wavy line indicates a point of attachment;
(C) 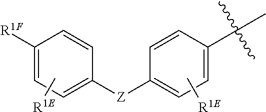
wherein Z is O, S, NH or CH₂; R^{1E} at each occurrence is independently hydrogen, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl)aryl; R^{1F} is hydrogen or alkyl, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl)aryl; wherein any R^{1E} or R^{1F} can be further substituted with one to three (C₁-C₁₂)-alkyl or -alkoxy groups, which can further bear halogen, amino, hydroxyl, aminocarbonyl, hydroxycarbonyl, nitro, fluoroalkyl, (C₁-C₆)thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein a wavy line indicates a point of attachment; or
(D) 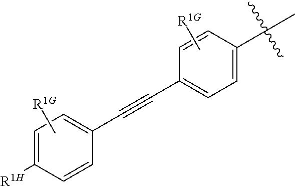
wherein R^{1G} at each occurrence is independently hydrogen, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; R^{1H} is hydrogen or alkyl, halogen, amino, hydroxyl, aminocarbonyl, hydroxycarbonyl, nitro, fluoroalkyl, (C₁-C₆)-thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein any R^{1G} or R^{1H} can be further substituted with one to three (C₁-C₁₂)-alkyl or -alkoxy groups, which can further bear halogen, amino, hydroxyl, aminocarbonyl, hydroxycarbonyl, nitro, fluoroalkyl, (C₁-C₆)thioalkyl, fluoroalkoxy, cyano, (C₁-C₆)-alkyl, (C₁-C₆)-alkoxy, (C₁-C₆)-mono- or di-alkylamino, (C₁-C₆)-alkyl, 5- to 7-membered heteroaryl, 5- to 7-membered heterocyclyl, or (C₆-C₁₀)aryl; wherein a wavy line indicates a point of attachment;
R² and R³ are each independently nitro, halo, cyano, hydroxy, glycosyloxy, amino, (C₁-C₄)alkoxy, (C₁-C₄)acyloxy, or (C₁-C₄)alkyl, wherein any carbon atom can be unsubstituted or substituted with J, wherein n² and n³ are independently 0, 1, 2, or 3; or wherein two R² groups taken together, and/or two R³ groups taken together, can comprise fused cycloalkyl, aryl, heterocyclyl, or heteroaryl ring or rings, any of which is substituted with 0-3 J;
R⁴ and R⁶ are each independently at every occurrence hydrogen, (C₁-C₆)alkyl, (C₃-C₇)cycloalkyl, 5- to 7-membered heteroaryl, 5- to 7-membered heterocyclyl, or (C₆-C₁₀)aryl, wherein any alkyl, cycloalkyl, heterocyclyl, aryl or heteroaryl can be substituted with 1 to 3 J;
R^{A1}, R^{A2}, R^{A3}, R^{A4}, R^{A5} are independently at each occurrence hydrogen, (C₁-C₆)alkyl, (C₃-C₇)cycloalkyl, 5- to 7-membered heteroaryl, 5- to 7-membered heterocyclyl, or (C₆-C₁₀)aryl, wherein any alkyl, cycloalkyl, heterocyclyl, aryl or heteroaryl can be substituted with 1 to 3 J;
J is halogen, R′, OR′, CN, CF₃, OCF₃, O, S, C(O), S(O), methylenedioxy, ethylenedioxy, (CH₂)₀₋ₚN(R′)₂, (CH₂)₀₋ₚSR′, (CH₂)₀₋ₚS(O)R′, (CH₂)₀₋ₚS(O)₂R′, (CH₂)₀₋ₚS(O)₂N(R′)₂, (CH₂)₀₋ₚSO₃R′, (CH₂)₀₋ₚC(O)R′, (CH₂)₀₋ₚC(O)CH₂C(O)R′, (CH₂)₀₋ₚC(S)R′, (CH₂)₀₋ₚC(O)OR′, (CH₂)₀₋ₚOC(O)R′, (CH₂)₀₋ₚC(O)N(R′)₂, (CH₂)₀₋ₚOC(O)N(R′)₂, (CH₂)₀₋ₚC(S)N(R′)₂, (CH₂)₀₋ₚNH—C(O)R′, (CH₂)₀₋ₚN(R′)N(R′)C(O)R′, (CH₂)₀₋ₚN(R′)N(R′)C(O)OR′, (CH₂)₀₋ₚN(R′)N(R′)CON(R′)₂, (CH₂)₀₋ₚN(R′)SO₂R′, (CH₂)₀₋ₚN(R′)SO₂N(R′)₂, (CH₂)₀₋ₚN(R′)C(O)OR′, (CH₂)₀₋ₚN(R′)C(O)R′, (CH₂)₀₋ₚN(R′)C(S)R′, (CH₂)₀₋ₚN(R′)C(O)N(R′)₂, (CH₂)₀₋ₚN(R′)C(S)N(R′)₂, (CH₂)₀₋ₚN(COR′)COR′, (CH₂)₀₋ₚN(OR′)₀₋ₚR′, (CH₂)₀₋ₚC(═NH)N(R′)₂, (CH₂)₀₋ₚC(O)N(OR′)R′, or (CH₂)₀₋ₚC(═NOR′)R′; wherein p is about 4,
each R′ is independently at each occurrence hydrogen, (C₁-C₁₂)-alkyl, (C₂-C₁₂)-alkenyl, (C₂-C₁₂)-alkynyl, (C₃-C₁₀)-cycloalkyl, (C₃-C₁₀)-cycloalkenyl, [(C₃-C₁₀)cycloalkyl or (C₃-C₁₀)-cycloalkenyl]-[(C₁-C₁₂)-alkyl or (C₂-C₁₂)-alkenyl or (C₂-C₁₂)-alkynyl], (C₆-C₁₀)-aryl, (C₆-C₁₀)aryl-[(C₁-C₁₂)-alkyl or (C₂-C₁₂)-alkenyl or (C₂-C₁₂)-alkynyl], mono- or bicyclic 3-10 membered heterocyclyl, mono- or bicyclic 3-10 membered heterocyclyl-[(C₁-C₁₂)-alkyl or (C₂-C₁₂)-alkenyl or (C₂-C₁₂)-alkynyl], mono- or bicyclic 5-10 membered heteroaryl, or mono- or bicyclic 5-10 membered heteroaryl-[(C₁-C₁₂)-alkyl or (C₂-C₁₂)-alkenyl or (C₂-C₁₂)-alkynyl];
or, when two R′ are hound to a nitrogen atom or to two adjacent nitrogen atoms, the two R′ groups together with the nitrogen atom or atoms to which they are bound can form a 3- to 8-membered monocyclic heterocyclic ring, or an 8- to 20-membered, bicyclic or tricyclic, heterocyclic ring system, wherein any ring or ring system can further contain 1-3 additional heteroatoms selected from the group consisting of N, NR′, 0, S, S(O) and S(O)₂;
wherein, in any bicyclic or tricyclic ring system, each ring is linearly fused, bridged, or spirocyclic, wherein each ring is either aromatic or nonaromatic, wherein each ring can be fused to a (C₆-C₁₀)aryl, mono- or bicyclic 5-10 membered heteroaryl, (C₃-C₁₀)cycloalkyl or mono- or bicyclic 3-10 membered heterocyclyl;
G¹ and G² are each independently a hydrogen or a glycosyl residue, or a group cleavable under physiological conditions to provide a compound of formula (I) wherein G¹ or G² respectively is hydrogen;
(X¹)_{X1} and (X²)_{X2} each signify that 0, 1, or 2 ring atoms of each respective ring can be nitrogen, provided that where a non-hydrogen substituent is bonded, X¹ or X², respectively, is C;
provided that when G¹ is a 6-deoxyhexopyranosyl residue, G² is H, R¹ is of formula (IIA), R² is hydrogen or hydroxy, R³ is hydrogen, R^{A1} and R^{A2} and R^{A4} are H, R^{A3} and R^{A5} are methyl, and B is CO₂H, or when G¹ and G² are H, R¹ is of formula (HA), R² is hydrogen, R³ is hydrogen or nitro, R^{A1} and R^{A2} and R^{A4} are H, R^{A3} and R^{A5} are methyl, and B is CO₂H, then R⁵ is not unsubstituted (C₁₀-C₁₆)-alkyl;
or a salt thereof.

2. The compound of claim 1 wherein when G¹ is a H or a 6-deoxyhexopyranosyl residue, G² is H, R¹ is of formula (IIA), R² is hydrogen or hydroxy, R³ is hydrogen or nitro, R^{A1} and R^{A2} and R^{A4} are H, R^{A3} and R^{A5} are methyl, and B is CO₂H, then R⁵ is not unsubstituted (C₁-C₂₂)alkyl.

3. The compound of claim 1 wherein the compound is of formula (IA) 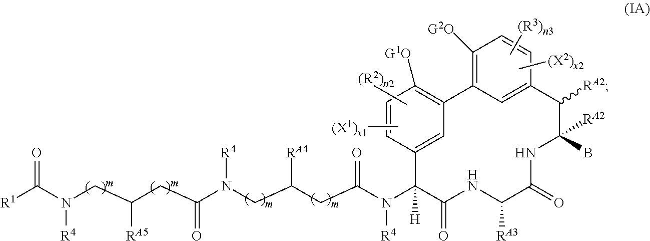
or a salt thereof.

4. The compound of claim 1 wherein R′ is a group of formula (IIAS), or (IIBS): 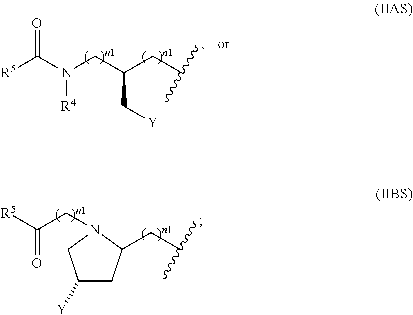
wherein a wavy line indicates a point of attachment of R¹ to an atom bonded to R¹ in formula (I);
or a salt thereof.

5. The compound of claim 1 wherein R⁵ is a (C₁-C₂₂) linear or branched alkyl.

6. The compound of claim 1 wherein R⁵ is a (C₁-C₂₂) linear or branched alkyl comprising one or more of groups (A), (B), (C), or (D), of claim 1.

7. The compound of claim 3 wherein R⁵ is a (C₁-C₂₂) linear or branched alkyl.

8. The compound of claim 3 wherein R⁵ is a (C₁-C₂₂) linear or branched alkyl, comprising one or more of groups (A), (B), (C), or (D), of claim 1.

9. The compound of claim 1 wherein R⁵ is any of the following groups 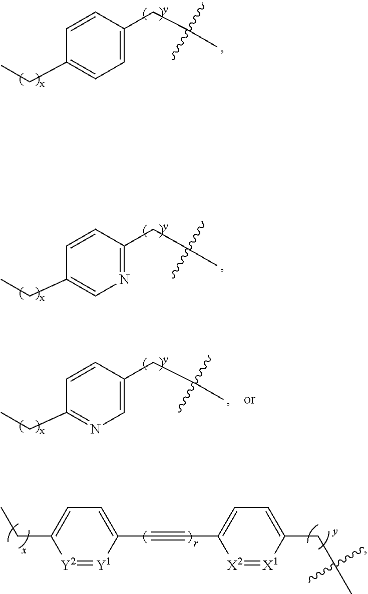
wherein x is 0-14, y is 0-14, provided that x+y<22, r is 0 or 1, and X¹, X², Y¹ and Y² are each independently C or N, provided that no more than one of X¹ and X², and no more than one of Y¹ and Y², is N, wherein a wavy line indicates a point of attachment of R⁵ to an atom bonded to R⁵ in formula (IIA), (IIB), or (IIC).

10. The compound of claim 1 wherein R⁵ is any of the following: methyl, ethyl, (C₃-C₂₂)-n-alkyl, (C₃-C₂₂)-isoalkyl, (C₄-C₂₂)-anteisoalkyl, naphthyl, (C₂-C₁₀) naphthyl, naphthylmethyl, (C₂-C₁₀) naphthylmethyl, biphenyl, (C₂-C₁₀)alkylbiphenyl, biphenylmethyl, (C₂-C₁₀)alkylbiphenylmethyl, (C₄-C₁₂)phenyl, (C₄-C₁₂)benzyl, (C₂-C₁₀)-1,2-diphenylethynyl, or (Z)— or (E)-(C₂-C₁₀)-1,2-diphenylethenyl, wherein a wavy line indicates a point of attachment of R⁵ to an atom bonded to R⁵ in formula (IIA), (IIB), or (IIC).

11. The compound of claim 1 wherein ring bearing one or more X¹ or X², respectively, is a phenyl, pyridyl, pyrazinyl, pyrimidyl, or pyridazinyl, optionally wherein R² and R³ are both hydrogen.

12. The compound of claim 1 wherein at least one of R² and R³ is hydrogen.

13. The compound of claim 1 wherein at least one of R² and R³ is nitro, halo, cyano, hydroxy, glycosyloxy, amino, (C₁-C₄)alkoxy, or (C₁-C₄)alkyl, and n² or n³ respectively, or both, is 1.

14. The compound of claim 1 wherein both G are hydrogen.

15. The compound of claim 1 wherein any of R^{A1}, R^{A2} and R^{A4} are hydrogen, any of R^{A3} and R^{A5} are methyl, or any combination thereof.

16. The compound of claim 1 wherein R^{A3} is hydrogen, methyl, ethyl, n-propyl, isopropyl, n-butyl, isobutyl, 3-hydroxypropyl, 4-hydroxybutyl, or 2,2,2-trifluoroethyl.

17. The compound of claim 1 wherein all of R⁴ and R⁶ are independently selected hydrogen or methyl.

18. The compound of claim 1 wherein the compound is any of the following compounds of formula (III) 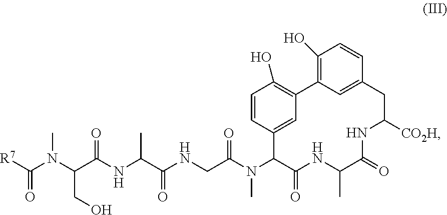
wherein R⁷ is (C₈-C₁₈)-n-alkyl, (C₈-C₁₈)-isoalkyl, (C₈-C₁₈)-anteisoalkyl, any of which includes a group (A), (B), (C), (D), or (E) of claim 1; or is 2-naphthyl, 6-(C₂-C₁₀)-2-naphthyl, 2-naphthylmethyl, 6-(C₂-C₁₀)-2-naphthylmethyl, 4-biphenyl, 4-biphenylmethyl, 4′-(C₂-C₁₀)alkyl-4-biphenyl, 4′-(C₂-C₁₀)alkyl-4-biphenylmethyl, p-(C₄-C₁₂)phenyl, p-(C₄-C₁₂)benzyl, or 4′-(C₂-C₁₀)-1,2-diphenylethynyl;
or a salt thereof.

19. The compound of claim 3 wherein the compound is any of the following compounds of formula (IV) 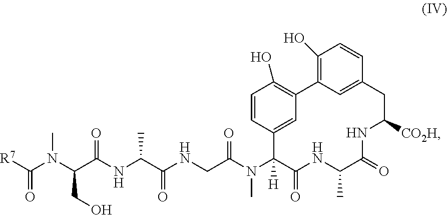
herein R⁷ is (C₈-C₁₈)-n-alkyl, (C₈-C₁₈)-isoalkyl, (C₈-C₁₈)-anteisoalkyl, any of which includes a group (A), (B), (C), (D), or (E) of claim 1; or is 2-naphthyl, 6-(C₂-C₁₀)-2-naphthyl, 2-naphthylmethyl, 6-(C₂-C₁₀)-2-naphthyl methyl, 4-biphenyl, 4-biphenylmethyl, 4′-(C₂-C₁₀)alkyl-4-biphenyl, 4′-(C₂-C₁₀)alkyl-4-biphenylmethyl, p-(C₄-C₁₂)phenyl, p-(C₄-C₁₂)benzyl, or 4′-(C₂-C₁₀)-1,2-diphenylethynyl;
or a salt thereof.

20. A compound comprising a hydrate, solvate, prodrug, or metabolite of a compound of claim 1.

21. A pharmaceutical composition comprising the compound of claim 1 and a pharmaceutically acceptable excipient.

22. Use of a compound of claim 1 for preparation of a medicament for treatment of a bacterial infection in a patient.

23. A method of treatment of a bacterial infection in an animal, comprising administering an effective amount of a compound of claim 1 to the animal at a frequency and for a duration sufficient to provide a beneficial effect to the animal.

24. The method of claim 23 wherein a causative bacterial species of the bacterial infection is of a genotype resistant to treatment with arylomycin A2.

25. The method of claim 23, wherein the bacterial infection is an infection involving Corynebacterium diphtheriae, Corynebacterium glutamicum, Campylobacter jejuni, Chlamydia trachomatis, Chlamydophila pneumoniae, Francisella tularensis, Helicobacter pylori, Lactococcus lactis subsp. cremoris, Lacto coccus lactis subsp. lactis, Propionibacterium acnes, Rhodococcus equi, Rhodococcus opacus, Staphylococcus capitis, Staphylococcus caprae, Staphylococcus carnosus, Staphylococcus cohnii, Staphylococcus epidermidis, Staphylococcus haemolyticus, Staphylococcus hominis, Staphylococcus hominis subsp. hominis, Staphylococcus hominis subsp. novobiosepticus, Staphylococcus lugdunensis, Streptococcus agalactiae, Streptococcus dysgalactiae, Streptococcus mitis, Streptococcus ovalis, Streptococcus pyogenes, Streptococcus pnemoniae, and/or Yersinia pestis.

26. The method of claim 23, wherein the bacterial infection is an infection involving a gram negative bacteria.

27. A method of treating a bacterial infection in an animal that includes administering arylomycin A and/or arylomycin B and/or a compound of claim 1 to the animal, wherein the infection involves a microbial species that expresses a signal peptidase without a proline residue within 10 amino acids N-terminal to the signal peptidase catalytic serine.

28. The method of claim 27, wherein the bacterial species encodes or expresses an SPase enzyme without a proline residue 5 to 7 amino acids N-terminal to the SPase catalytic serine.

29. The method of claim 27, wherein the bacterial infection is an infection involving Corynebacterium diphtheriae, Corynebacterium glutamicum, Campylobacter jejuni, Chlamydia trachomatis, Chlamydophila pneumoniae, Francisella tularensis, Helicobacter pylori, Lactococcus lactis subsp. cremoris, Lactococcus lactis subsp. lactis, Propionibacterium acnes, Rhodococcus equi, Staphylococcus carnosus, Staphylococcus cohnii, Staphylococcus haemolyticus, Staphylococcus hominis, Staphylococcus hominis subsp. hominis, Staphylococcus hominis subsp. novobiosepticus, Staphylococcus lugdunensis, Streptococcus agalactiae, Streptococcus dysgalactiae, Streptococcus mitis, Streptococcus oralis, Streptococcus pyogenes, and/or Streptococcus pnemoniae.

30. The method of claim 27, wherein the bacterial infection is an infection involving a gram negative bacteria.

31. The method of claim 27, wherein administering comprises topical administration.

32. A method of treating a bacterial infection in an animal comprising administering to the animal any one or any combination of the compounds of claim 1, wherein the bacterial infection comprises an infection by a bacteria that encodes or expresses an SPase enzyme that has a proline within about 10 amino acids N-terminal to the SPase catalytic serine.

33. The method of claim 32, wherein the bacteria encodes or expresses an SPase enzyme that does not have a proline 5 to 7 amino acids N-terminal to the SPase catalytic serine.

34. The method of claim 32, wherein the bacterial infection involves Staphylococcus capitis, Staphylococcus caprae and/or Yersinia pestis.

35. A method of treating a bacterial infection in an animal comprising administering to the animal arylomycin A or arylomycin B, wherein the microbial infection is an infection involving Corynebacterium diphtheriae, Corynebacterium glutamicum, Campylobacter jejuni, Chlamydia trachomatis, Chlamydophila pneumoniae, Francisella tularensis, Helicobacter pylori, Lactococcus lactis subsp. cremoris, Lactococcus lactis subsp. lactis, Propionibacterium acnes, Rhodococcus equi, Staphylococcus capitis, Staphylococcus caprae, Staphylococcus carnosus, Staphylococcus cohnii, Staphylococcus haemolyticus, Staphylococcus hominis, Staphylococcus hominis subsp. hominis, Staphylococcus hominis subsp. novobiosepticus, Staphylococcus lugdunensis, Streptococcus agalactiae, Streptococcus dysgalactiae, Streptococcus mitis, Streptococcus oralis, Streptococcus pyogenes, Streptococcus pnemoniae, and/or Yersinia pestis.

36. The method of claim 35, wherein administering comprises topical administration.

37. The method of claim 23, further comprising administering a second therapeutic agent.

38. The method of claim 37, wherein the second therapeutic agent is a non-arylomycin antibiotic.

39. The method of claim 38, wherein the non-arylomycin antibiotic is an aminoglycoside antibiotic, fluoroquinolone antibiotic, penicillin antibiotic, cephalosporin antibiotic, macrolide antibiotic, glycopeptide antibiotic, rifampicin, chloramphenicol, fluoramphenicol, colistin, mupirocin, bacitracin, daptomycin, or linezolid.

40. A method of detecting whether a test sample contains a bacterium sensitive to an arylomycin antibiotic comprising detecting whether a bacterial signal peptidase is present in the test sample, wherein the bacterial signal peptidase does not have a proline within about 10 amino acids N-terminal to the bacterial signal peptidase's catalytic serine.

41. The method of claim 40, furthering comprising detecting whether a test sample contains Yersinia pestis.

42. The method of claim 40, wherein the bacterial signal peptidase nucleic acid or the bacterial signal peptidase polypeptide is detected.

43. The method of claim 40, further comprising contacting the test sample with an anti-signal peptidase antibody and detecting whether the antibody forms a complex with a bacterial signal peptidase in the test sample.

44. The method of claim 43, wherein the antibody selectively binds to a signal peptidase that does not contain a proline with about 10 amino acids N-terminal to the bacterial signal peptidase's catalytic serine.

45. The method of claim 40, further comprising contacting the test sample with an anti-signal peptidase antibody that selectively binds to a signal peptidase that does contain a proline with about 10 amino acids N-terminal to the bacterial signal peptidase catalytic serine and detecting whether the antibody forms a complex with a bacterial signal peptidase in the test sample.

46. The method of claim 40, further comprising contacting a nucleic acid isolated from the test sample with a probe or primer that selectively hybridizes to a DNA encoding a bacterial signal peptidase that does not contain a proline with about 10 amino acids N-terminal to the bacterial signal peptidase's catalytic serine.

47. The method of claim 46, wherein the probe or the primer hybridizes to the DNA under stringent hybridization conditions.

48. The method of claim 46, wherein the probe or primer hybridizes to the signal peptidase DNA encoding a region comprising about 4 to about 15 amino acids of the bacterial signal peptidase that includes the catalytic serine.

49. The method of claim 40, wherein detecting comprises nucleic acid amplication, nucleic acid sequencing, or single nucleotide polymorphism detection.

50. The method of claim 40, wherein the signal peptidase does not have a proline seven amino acids N-terminal to the bacterial signal peptidase' catalytic serine.

51. The method of claim 40, wherein the signal peptidase does not have a proline five amino acids N-terminal to the bacterial signal peptidase' catalytic serine.

52. The method of claim 40, wherein the bacterium is a Eubacteria, and/or the antibody selectively binds to the signal peptidase from a selected species of Eubacteria and/or the probe or primer selectively hybridizes to a DNA encoding a signal peptidase from a selected species of Eubacteria.

53. A bacterium genetically engineered to encode and/or express a signal peptidase with a proline at 5 to 7 amino acids N-terminal to the signal peptidase's catalytic serine, wherein the bacterium is selected from the group consisting of Corynebacterium diphtheriae, Corynebacterium glutamicum, Campylobacter jejuni, Chlamydia trachomatis, Chlamydophila pneumoniae, Francisella tularensis, Helicobacter pylori, Lactococcus lactis subsp. cremoris, Lactococcus lactis subsp. lactis, Propionibacterium acnes, Rhodococcus equi, Rhodococcus opacus, Staphylococcus camosus, Staphylococcus cohnii, Staphylococcus epidermidis, Staphylococcus haemolyticus, Staphylococcus hominis, Staphylococcus hominis subsp. hominis, Staphylococcus hominis subsp. novobiosepticus, Staphylococcus lugdunensis, Streptococcus agalactiae, Streptococcus dysgalactiae, Streptococcus mitis, Streptococcus oralis, Streptococcus pyogenes, and Streptococcus pnemoniae.

54. A bacterium genetically engineered to encode and/or express a signal peptidase without a proline at 5 to 7 amino acids N-terminal to the signal peptidase's catalytic serine, wherein the bacterium is Escherichia coli, Klebsiella pneumonia, Salmonella entericia, Vibrio cholera, Pseudomonas aeruginosa, Acinetobacter baumanii, Neiserria meningitides, Haemophilia influenza, Citrobacter koseri, Shigella flexneri, Bordetella pertussis, Mycobacterium tuberculosis, Staphylococcus aurues, Bacillus anthracis, Streptococcus tuberculosis, Clostridium difficile, Enterococcus faecalis and/or Listeria monocytogenes.

55. A mutant signal peptidase with a Corynebacterium diphtheriae, Corynebacterium glutamicum, Campylobacter jejuni, Chlamydia trachomatis, Chlamydophila pneumoniae, Francisella tularensis, Helicobacter pylori, Lactococcus lactis subsp. cremoris, Lactococcus lactis subsp. lactis, Propionibacterium acnes, Rhodococcus equi, Rhodococcus opacus, Staphylococcus carnosus, Staphylococcus cohnii, Staphylococcus epidermidis, Staphylococcus haemolyticus, Staphylococcus hominis, Staphylococcus hominis subsp. hominis, Staphylococcus hominis subsp. novobiosepticus, Staphylococcus lugdunensis, Streptococcus agalactiae, Streptococcus dysgalactiae, Streptococcus mitis, Streptococcus oralis, Streptococcus pyogenes, or Streptococcus pnemoniae signal peptidase amino acid sequence that is modified to have a proline substitution at a position 5 or at a position 7 amino acids N-terminal to the signal peptidase's catalytic serine.

56. A mutant signal peptidase with a Escherichia coli, Klebsiella pneumonia, Salmonella entericia, Vibrio cholera, Pseudomonas aeruginosa, Acinetobacter baumanii, Neiserria meningitides, Haemophilus influenza, Citrobacter koseri, Shigella flexneri, Bordetella pertussis, Mycobacterium tuberculosis, Staphylococcus aurues, Bacillus anthracis, Streptococcus mutans, Clostridium difficile, Enterococcus faecalis or Listeria monocytogenes signal peptidase amino acid sequence that is modified by replacement of a proline with a selected amino acid, wherein the proline was at 5 to 7 amino acids N-terminal to the signal peptidase's catalytic serine.

57. The mutant signal peptidase of claim 56, wherein the selected amino acid is a serine.

58. A method of identifying an antibiotic effective for treating a bacterial infection involving an arylomycin-resistant bacterial species, comprising contacting the arylomycin-resistant bacteria with a test agent and observing whether the test agent inhibits growth of the arylomycin-resistant bacteria, wherein the arylomycin-resistant bacteria encodes or expresses a signal peptidase enzyme that has a proline residue 5 to 7 amino acids N-terminal to the signal peptidase catalytic serine.

59. A method of identifying a compound that has antibiotic activity against bacteria comprising contacting a culture of bacteria with the test compound and identifying whether the test compound inhibits growth of the bacteria, wherein the bacteria in the culture expresses a modified SPase that has a natural bacterial SPase amino acid sequence modified at position −5 to −7 relative to the catalytic serine by substitution or replacement of an amino acid at that position.

60. The method of claim 59, wherein an amino acid at position −5 and/or at position −7 relative to the catalytic serine is replaced with a proline.

61. The method of claim 59, wherein the amino acid at position −5 and/or at position −7 relative to the catalytic serine is a proline replaced by a selected amino acid.

62. The method of claim 61, wherein the selected amino acid is a serine.

63. The method of claim 58, wherein the test compound that inhibits the growth of the bacteria has antibiotic activity.
